(19) United States
(12) Patent Application Publication
(10) Pub. No.: US 2022/0249652 A1
(43) Pub. Date: Aug. 11, 2022

(54) INFLUENZA VIRUS NEURAMINIDASE AND USES THEREOF

(71) Applicant: ICAHN SCHOOL OF MEDICINE AT MOUNT SINAI, New York, NY (US)

(72) Inventors: Peter PALESE, New York, NY (US); Adolfo GARCIA-SASTRE, New York, NY (US); Florian KRAMMER, New York, NY (US); Felix BRÖCKER, Berlin (DE); Allen ZHENG, New York, NY (US); Weina SUN, New York, NY (US)

(73) Assignee: ICAHN SCHOOL OF MEDICINE AT MOUNT SINAI, New York, NY (US)

(21) Appl. No.: 17/621,816

(22) PCT Filed: Jun. 25, 2020
(86) PCT No.: PCT/US2020/039588

§ 371 Date: Dec. 22, 2021

Related U.S. Application Data
(60) Provisional application No. 62/971,629, filed on Feb. 7, 2020.
(60) Provisional application No. 62/867,077, filed on Jun. 26, 2019.

(51) Int. Cl. A61K 39/145; C07K 14/005; C12N 7/00; C12N 9/24

(52) U.S. Cl. CPC A61K 39/145; C07K 14/005; A61K 2039/5252; C12N 9/2402; C12Y 302/01018; C12N 7/00

(57) ABSTRACT

In one aspect, provided herein are mutated influenza virus neuraminidase polypeptides, wherein the mutated influenza virus neuraminidase polypeptides comprise a first cytoplasmic domain, a first transmembrane domain, a first stalk domain, and a first globular head domain of a first neuraminidase of a first influenza virus with an insertion of 15 to 45 or 1 to 50 amino acid residues in the first stalk domain of the first neuraminidase. In another aspect, provided herein is an influenza virus comprising such a mutated influenza virus neuraminidase polypeptide, a genome comprising a nucleotide sequence encoding such a mutated influenza virus neuraminidase polypeptide or both. In another aspect, provided herein is an immunogenic composition comprising such an influenza virus, and optionally an adjuvant.

This application claims benefit of U.S. provisional application No. 62/867,077, filed on Jun. 26, 2019, and U.S. provisional application No. 62/971,629 filed on Feb. 7, 2020, each of which is incorporated herein by reference in their entirety.

This invention was made with government support under grant no. AI097092 awarded by the National Institutes of Health. The government has certain rights in the invention.

This application incorporates by reference a Sequence Listing submitted with this application as text file entitled “06923-304-228_SEQ_LISTING.txt” created on Jun. 22, 2020 and having a size of 66,854 bytes.

### 1. INTRODUCTION

In one aspect, provided herein are mutated influenza virus neuraminidase polypeptides, wherein the mutated influenza virus neuraminidase polypeptides comprise a first cytoplasmic domain, a first transmembrane domain, a first stalk domain, and a first globular head domain of a first neuraminidase of a first influenza virus with an insertion of 15 to 45 or 1 to 50 amino acid residues in the first stalk domain of the first neuraminidase. In another aspect, provided herein is an influenza virus comprising such a mutated influenza virus neuraminidase polypeptide, a genome comprising a nucleotide sequence encoding such a mutated influenza virus neuraminidase polypeptide or both. In another aspect, provided herein is an immunogenic composition comprising such an influenza virus, and optionally an adjuvant. In another aspect, provided herein is an influenza virus comprising a chimeric influenza virus HA gene segment and a chimeric influenza virus NA gene segment in which the packaging signals of the gene segments have been swapped, and an immunogenic composition comprising such an influenza virus. In another aspect, provided herein is a method for immunizing against influenza virus in a subject (e.g., a human subject) comprising administering the immunogenic composition to the subject.

### 2. BACKGROUND

Influenza viruses are enveloped RNA viruses that belong to the family of Orthomyxoviridae (Palese and Shaw (2007) Orthomyxoviridae: The Viruses and Their Replication, 5th ed. Fields' Virology, edited by B. N. Fields, D. M. Knipe and P. M. Howley. Wolters Kluwer Health/Lippincott Williams & Wilkins, Philadelphia, USA, p 1647-1689). The natural host of influenza A viruses are mainly avians, but influenza A viruses (including those of avian origin) also can infect and cause illness in humans and other animal hosts (bats, canines, pigs, horses, sea mammals, and mustelids). For example, the H5N1 avian influenza A virus circulating in Asia has been found in pigs in China and Indonesia and has also expanded its host range to include cats, leopards, and tigers, which generally have not been considered susceptible to influenza A (CIDRAP—Avian Influenza: Agricultural and Wildlife Considerations). The occurrence of influenza virus infections in animals could potentially give rise to human pandemic influenza strains.

Influenza A and B viruses are major human pathogens, causing a respiratory disease that ranges in severity from sub-clinical infection to primary viral pneumonia which can result in death. The clinical effects of infection vary with the virulence of the influenza strain and the exposure, history, age, and immune status of the host. The cumulative morbidity and mortality caused by seasonal influenza is substantial due to the relatively high attack rate. In a normal season, influenza can cause between 3-5 million cases of severe illness and up to 650,000 deaths worldwide (World Health Organization (2008) Influenza (Seasonal): Signs and Symptoms; November 2008). In the United States, influenza viruses infect an estimated 10-15% of the population (Glezen and Couch R B (1978) Interpandemic influenza in the Houston area, 1974-76. N Engl J Med 298: 587-592; Fox et al. (1982) Influenza virus infections in Seattle families, 1975-1979. II. Pattern of infection in invaded households and relation of age and prior antibody to occurrence of infection and related illness. Am J Epidemiol 116: 228-242) and are associated with approximately 30,000 deaths each year (Thompson W W et al. (2003) Mortality Associated with Influenza and Respiratory Syncytial Virus in the United States. JAMA 289: 179-186; Belshe (2007) Translational research on vaccines: influenza as an example. Clin Pharmacol Ther 82: 745-749).

In addition to annual epidemics, influenza viruses are the cause of infrequent pandemics. For example, influenza A viruses can cause pandemics such as those that occurred in 1918, 1957, 1968, and 2009. Due to the lack of pre-formed immunity against the major viral antigen, hemagglutinin (HA), pandemic influenza can affect greater than 50% of the population in a single year and often causes more severe disease than epidemic influenza. A stark example is the pandemic of 1918, in which an estimated 50-100 million people were killed (Johnson and Mueller (2002) Updating the Accounts: Global Mortality of the 1918-1920 “Spanish” Influenza Pandemic Bulletin of the History of Medicine 76: 105-115). Since the emergence of the highly pathogenic avian H5N1 influenza virus in the late 1990s (Claas et al. (1998) Human influenza A H5N1 virus related to a highly pathogenic avian influenza virus. Lancet 351: 472-7), there have been concerns that it may be the next pandemic virus. Further, H7, H9 and H10 strains are candidates for new pandemics since these strains infect humans on occasion.

Seasonal vaccination is currently the most effective intervention against influenza (Gross et al., Ann Intern Med, 1995, 123(7): p. 518-27; Ogburn et al., J Reprod Med, 2007, 52(9): p. 753-6; Jefferson et al., Lancet, 2005. 366(9492): p. 1165-74; Beyer et al., Vaccine, 2013, 31(50): p. 6030-3; Nichol et al., N Engl J Med, 1995. 333(14): p. 889-93; Jefferson et al., Lancet, 2005. 365(9461): p. 773-80), yet overall vaccine effectiveness was only 36% in the recent 2017-2018 season (Flannery et al., MMWR Morb Mortal Wkly Rep, 2018. 67(6): p. 180-185). However, current vaccination approaches rely on achieving a good match between circulating strains and the isolates included in the vaccine. Such a match is often difficult to attain due to a combination of factors. First, influenza viruses are constantly undergoing change: every 3-5 years the predominant strain of influenza A virus is replaced by a variant that has undergone sufficient antigenic drift to evade existing antibody responses. Isolates to be included in vaccine preparations must therefore be selected each year based on the intensive surveillance efforts of the World Health Organization (WHO) collaborating centers. Second, to allow sufficient time for vaccine manufacture and distribution, strains must be selected approximately six months prior to the initiation of the influenza season. Often, the predictions of the vaccine strain selection committee are inaccurate, resulting in a substantial drop in the efficacy of vaccination.

The possibility of a novel subtype of influenza virus entering the human population also presents a significant challenge to current vaccination strategies. Since it is impossible to predict what subtype and strain of influenza virus will cause the next pandemic, current, strain-specific approaches cannot be used to prepare a pandemic influenza vaccine in advance of a pandemic. Thus, there is a need for vaccines that cross-protect subjects against different strains and/or subtypes of influenza virus.

### 3. SUMMARY

In one aspect, provided herein is a mutated influenza virus neuraminidase polypeptide, and wherein the mutated influenza virus neuraminidase polypeptide comprises a first cytoplasmic domain, a first transmembrane domain, a first stalk domain, and a first globular head domain of a first neuraminidase of a first influenza virus with an insertion of 15 to 45 or 15 to 50 amino acid residues in the first stalk domain of the first neuraminidase. In one embodiment, the insertion is of 15 to 30 amino acid residues. In another embodiment, the insertion is of 15, 20, 25 or 30 amino acid residues. In some embodiments, the amino acid residues inserted correspond to amino acid residues found in a second stalk domain of a second neuraminidase of a second influenza virus, wherein the first influenza virus is from a different subtype than second influenza virus. In certain embodiments, the amino acid residues inserted correspond to amino acid residues found in a second stalk domain of a second neuraminidase of a second influenza virus, wherein the first influenza virus is from a different strain than second influenza virus. In some embodiments, the first influenza virus, the second influenza virus or both are influenza A viruses. In certain embodiments, the first influenza virus, the second influenza virus, or both are influenza B viruses. In a specific embodiment, the second influenza virus is influenza A virus H1N1pdm09 A/California/04/2009 (Cal09) virus. In some embodiments, the first influenza A virus neuraminidase is a neuraminidase of influenza A virus of subtype N1, N2, N3, N4, N5, N6, N7, N8, N9, N10, or N11. In a specific embodiment, the first influenza virus is influenza A virus H1N1 A/Puerto Rico/8/1934 (PR8) or influenza A virus H3N2 A/New York/61/2012 (NY12). In specific embodiments, the first influenza virus is a seasonal influenza virus strain. In a specific embodiment, the inserted amino acid sequence is encoded by a nucleotide sequence comprising the sequence set forth in SEQ ID NO: 29.

In a specific embodiment, provided herein is a mutated influenza virus neuraminidase polypeptide comprising the amino acid sequence of SEQ ID NO: 4. In another specific embodiment, provided herein is a mutated influenza virus neuraminidase polypeptide comprising the amino acid sequence of SEQ ID NO: 8 or 10.

In another aspect, provided herein is an influenza virus comprising a mutated influenza virus neuraminidase polypeptide described herein. In a specific embodiment, the virion of the influenza virus has incorporated in it a mutated influenza virus neuraminidase polypeptide. In another specific embodiment, the influenza virus comprises a genome comprising a nucleotide sequence encoding a mutated influenza virus neuraminidase polypeptide described herein. In another specific embodiment, the influenza virus comprises a genome comprising a nucleotide sequence encoding a mutated influenza virus neuraminidase polypeptide described herein and the virion of the influenza virus has incorporated in it the mutated influenza virus neuraminidase polypeptide. In certain embodiments, the influenza virus is an influenza B virus. In some embodiments, the influenza virus is an influenza A virus. In certain embodiments, the influenza virus is influenza A virus H3N2 A/New York/61/2012 (NY12). In some embodiments, the influenza virus is influenza A virus H1N1 A/Puerto Rico/8/1934 (PR8). In specific embodiments, the influenza virus is a seasonal influenza virus strain. In certain embodiments, the influenza virus is a ressortant virus. The influenza virus may be a live attenuated virus or an inactivated virus.

In another aspect, provided herein is a recombinant influenza virus comprising a mutated influenza virus neuraminidase polypeptide, and wherein the mutated influenza virus neuraminidase polypeptide comprises a first cytoplasmic domain, a first transmembrane domain, a first stalk domain, and a first globular head domain of a first neuraminidase of a first influenza B virus with an insertion of 15 to 50 amino acid residues in the first stalk domain of the first neuraminidase. In one embodiment, the insertion is of 15 to 46 amino acid residues. In another embodiment, the insertion is of 46 amino acid residues. In certain embodiments, the amino acid residues inserted correspond to amino acid residues found in a second stalk domain of a second neuraminidase of a second influenza A virus. In some embodiments, the amino acid residues inserted correspond to amino acid residues found in a second stalk domain of a second neuraminidase of a second influenza A virus and amino acid residues found in a third stalk domain of at third neuraminidase of a third influenza A virus. In a specific embodiment, the second influenza virus is influenza virus A/Hong Kong/4801/2014 and the third influenza virus is influenza virus A/California/04/2009. In another specific embodiment, the inserted amino acid residues comprise the amino acid sequence encoded by the nucleotide sequence set forth in SEQ ID NO: 34. In another specific embodiment, the mutated influenza virus neuraminidase segment is encoded by a nucleotide sequence comprising the sequence set forth in SEQ ID NO: 32. In a specific embodiment, the first influenza B virus neuraminidase is a neuraminidase of influenza virus B/Brisbane/60/2008. In certain embodiments, the other influenza B virus gene segments are from influenza virus B/Malaysia/2506/2004. In some embodiments, the recombinant influenza virus is an influenza virus B/Malaysia/2506/2004.

In another aspect, provided herein is a recombinant influenza virus comprising a genome that comprises an NA segment, wherein the NA segment comprises a nucleotide sequence encoding a mutated influenza virus neuraminidase polypeptide, and wherein the mutated influenza virus neuraminidase polypeptide comprises a first cytoplasmic domain, a first transmembrane domain, a first stalk domain, and a first globular head domain of a first neuraminidase of a first influenza B virus with an insertion of 15 to 50 amino acid residues in the first stalk domain of the first neuraminidase. In certain embodiments, the virion of the recombinant influenza virus comprises the mutated influenza virus neuraminidase polypeptide. In one embodiment, the insertion is of 15 to 46 amino acid residues. In another embodiment, the insertion is of 46 amino acid residues. In certain embodiments, the amino acid residues inserted correspond to amino acid residues found in a second stalk domain of a second neuraminidase of a second influenza A virus. In some embodiments, the amino acid residues inserted correspond to amino acid residues found in a second stalk domain of a second neuraminidase of a second influenza A virus and amino acid residues found in a third stalk domain of a third neuraminidase of a third influenza A virus. In a specific embodiment, the second influenza virus is influenza virus A/Hong Kong/4801/2014 and the third influenza virus is influenza virus A/California/04/2009. In another specific embodiment, the inserted amino acid residues comprise the amino acid sequence encoded by the nucleotide sequence set forth in SEQ ID NO: 34. In another specific embodiment, the mutated influenza virus neuraminidase segment is encoded by a nucleotide sequence comprising the sequence set forth in SEQ ID NO: 32. In a specific embodiment, the first influenza B virus neuraminidase is a neuraminidase of influenza virus B/Brisbane/60/2008. In certain embodiments, the other influenza B virus gene segments are from influenza virus B/Malaysia/2506/2004. In some embodiments, the recombinant influenza virus is an influenza virus B/Malaysia/2506/2004.

In another aspect, provided herein is an influenza virus comprising a first chimeric influenza virus gene segment, a second chimeric influenza virus gene segment, and influenza virus NS, PB1, PB2, PA, M, and NP gene segments, wherein: (a) the first chimeric influenza virus gene segment encodes a mutated influenza virus neuraminidase (NA) and the first chimeric influenza virus gene segment comprises: (i) a 3′ non-coding region of a hemagglutinin (HA) influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the mutated influenza virus NA polypeptide, wherein the mutated influenza virus neuraminidase polypeptide comprises a first cytoplasmic domain, a first transmembrane domain, a first stalk domain, and a first globular head domain of a first neuraminidase of a first influenza virus with an insertion of 15 to 50 amino acid residues in the first stalk domain of the first neuraminidase; (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, synomyous mutations are introduced into the 3′ proximal nucleotides, the 5′ proximal nucleotides, or both in the open reading frames of the mutated influenza virus neuraminidase and HA. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. A person skilled in the art would be able to determine the non-coding regions, proximal coding regions, open reading frames, the proximal nucleotides of the influenza virus NA and HA gene segments using techniques and information known to one of skill in the art, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, any start codon in the 3′ proximal coding region of the NA influenza virus gene segment or HA influenza virus gene segment is mutated from ATG to TTG. In a specific embodiment, the first influenza virus is an influenza A virus. In some embodiments, the amino acid residues inserted correspond to amino acid residues found in a second stalk domain of a second neuraminidase of a second influenza A virus, wherein the first influenza A virus is from a different subtype than second influenza A virus. In certain embodiments, the amino acid residues inserted correspond to amino acid residues found in a second stalk domain of a second neuraminidase of a second influenza A virus, wherein the first influenza A virus is from a different strain than second influenza A virus. In some embodiment, the first influenza A virus neuraminidase is a neuraminidase of influenza A virus of subtype N1, N2, N3, N4, N5, N6, N7, N8, N9, N10, or N11. In specific embodiments, the first influenza virus is influenza A virus H1N1 A/Puerto Rico/8/1934 (PR8) or influenza A virus A/Hong Kong/4801/2014. In a specific embodiment, the first chimeric influenza virus gene segment comprises the nucleotide sequence set forth in SEQ ID NO: 27 and the second chimeric influenza virus gene segment comprises the nucleotide sequence set forth in SEQ ID NO: 23. In another specific embodiment, the first chimeric influenza virus gene segment comprises the nucleotide sequence set forth in SEQ ID NO: 28 and the second chimeric influenza virus gene segment comprises the nucleotide sequence set forth in SEQ ID NO: 25.

In another aspect, provided herein is a recombinant influenza virus or a composition comprising the recombinant influenza virus, wherein the recombinant influenza virus comprises a first chimeric influenza virus gene segment, a second chimeric influenza virus gene segment, and influenza virus NS, PB1, PB2, PA, M, and NP gene segments, wherein (a) the first chimeric influenza virus gene segment encodes an influenza virus NA polypeptide and the first chimeric influenza virus gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the influenza virus NA polypeptide, (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, the 3′ proximal nucleotides, the 5′ proximal nucleotides, or both in the open reading frames are mutated (e.g., substituted) in regions implicated in genome packaging in order to abrogate their residual packaging function. In a specific embodiment, the mutations introduced into the 3′ and/or 5′ proximal nucleotides of the open reading frame of the influenza virus gene segment(s) are silent or synonymous mutations. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. A person skilled in the art would be able to determine the non-coding regions, proximal coding regions, open reading frames, the proximal nucleotides of the influenza virus NA and HA gene segments using techniques and information known to one of skill in the art, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, any start codon in the 3′ proximal coding region of the NA influenza virus gene segment is mutated from ATG to TTG. In another specific embodiment, the NA open reading frame and HA open reading frame are from one strain or subtype of influenza virus and the packaging signals of the chimeric gene segments comprising those open reading frames are from a different strain or subtype of influenza virus. For example, the NA and HA open reading frames may be from A/Hong Kong/4801/2014 (HK14) and the packaging signals may be from A/Puerto Rico/8/1934 (PR8), such as described in Section 6, infra. In a particular embodiment, the NA open reading frame and HA open reading frame are from one strain or subtype of influenza virus and the packaging signals of the chimeric gene segments comprising those open reading frames are from a different strain or subtype of influenza virus and those packaging signals from the same strain or subtype of influenza virus as influenza virus NS, PB1, PB2, PA, M, and NP gene segments. In one embodiment, the first chimeric influenza virus gene segment comprises the packaging signals described in FIGS. 4A-4B of International Patent Application Publication No. WO 2011/014645 and the open reading frame of an influenza virus NA, and the second chimeric influenza virus gene segment comprises the packaging signals described in FIGS. 32A-32C of International Patent Application Publication No. WO 2011/014645 and the open reading frame encoding for an influenza virus HA polypeptide. In a specific embodiment, the first chimeric influenza virus gene segment comprises the nucleotide sequence set forth in SEQ ID NO:24, and the second chimeric influenza virus gene segment comprises the nucleotide sequence set forth in SEQ ID NO:23. In another specific embodiment, the first chimeric influenza virus gene segment comprises the nucleotide sequence set forth in SEQ ID NO:26, and the second chimeric influenza virus gene segment comprises the nucleotide sequence set forth in SEQ ID NO:25.

In another aspect, provided herein is an immunogenic composition comprising an influenza virus described herein. In a specific embodiment, the immunogenic composition further comprises an adjuvant, such as, e.g., an aluminum salt (alum), 3 De-O-acylated monophosphoryl lipid A (MPL), MF59 AS01, AS03, or AS04. In a specific embodiment, the immunogenic composition is a seasonal vaccine. In certain embodiments, the immunogenic composition comprises a live attenuated influenza virus described herein. In some embodiments, the immunogenic composition comprises an inactivated influenza virus described herein. In certain embodiments, the immunogenic composition is a split virus vaccine.

In another aspect, provided herein are methods for immunizing against influenza virus in a subject (e.g., human subject), comprising administering to the subject an immunogenic composition described herein. In another aspect, provided herein are methods for preventing influenza virus disease in a subject, comprising administering to the subject an immunogenic composition described herein. In a specific embodiment, the subject is a human subject.

In another aspect, provided herein are methods for inducing an immune response against influenza virus NA, the methods comprising administering to a subject (e.g., human subject) a recombinant influenza virus described herein or an immunogenic composition described herein. In a specific embodiment, the subject is a human subject.

In another aspect, provided herein are methods for enhancing a humoral immune response against influenza virus NA (e.g., clinically relevant influenza virus NA), comprising administering to a subject (e.g., human subject) a recombinant influenza virus described herein or an immunogenic composition described herein. In a specific embodiment, the humoral immune response against influenza virus NA is enhanced relative to the humoral response against influenza virus NA elicited following administration of a recombinant influenza virus in which the packaging signals of the influenza virus NA gene segment have not been exchanged with the packaging signals of influenza virus HA gene segment. In another embodiment, the humoral immune response against influenza virus NA is enhanced relative to the humoral response against influenza virus NA elicited following administration of a recombinant influenza virus in which the NA has not been mutated as described herein. In a specific embodiment, the enhanced humoral response against influenza virus NA is a stronger inhibition of neuraminidase enzymatic activity as assessed by a technique known in the art or described herein (e.g., Section 6.4, infra), higher antibody-dependent cellular cytotoxicity activity as assessed by a technique known in the art or described herein (see, e.g., Section 6.4, infra), or both. In certain embodiments, a stronger inhibition of neuraminidase enzymatic activity is 1.2, 1.3, 1.5, 1.75, 2, 2.5, 3, 3.5, 4, 4.5 fold or higher inhibition of neuraminidase enzymatic activity. In certain embodiments, higher ADCC is 1.2, 1.3, 1.5, 1.75, 2, 2.5, 3, 3.5, 4, 4.5 fold or higher ADCC activity. In some embodiments, the enhanced humoral response against influenza virus NA is a stronger inhibition of neuraminidase enzymatic activity, higher antibody-dependent cellular cytotoxicity activity, or both as described herein (see, e.g., Section 6.4, infra). In certain embodiments, the enhanced humoral response against influenza virus NA is an overall stronger anti-NA humoral response as described in Section 6.4, infra. In a specific embodiment, the subject is a human subject.

In one embodiment, provided herein are methods for enhancing a humoral immune response against influenza virus NA (e.g., clinically relevant influenza virus NA), comprising administering to a subject (e.g., human subject) a recombinant influenza virus or a composition comprising the recombinant influenza virus, wherein the recombinant influenza virus comprises a first chimeric influenza virus gene segment and a second chimeric influenza virus gene segment, wherein (a) the first chimeric influenza virus gene segment encodes an influenza virus NA polypeptide and the first chimeric influenza virus gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the influenza virus NA polypeptide, (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, the 3′ proximal nucleotides, the 5′ proximal nucleotides, or both in the open reading frames are mutated (e.g., substituted) in regions implicated in genome packaging in order to abrogate their residual packaging function. In a specific embodiment, the mutations introduced into the 3′ and/or 5′ proximal nucleotides of the open reading frame of the influenza virus gene segment(s) are silent or synonymous mutations. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. A person skilled in the art would be able to determine the non-coding regions, proximal coding regions, open reading frames, the proximal nucleotides of the influenza virus NA and HA gene segments using techniques and information known to one of skill in the art, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, any start codon in the 3′ proximal coding region of the NA or HA influenza virus gene segment is mutated from ATG to TTG. In another specific embodiment, the NA open reading frame and HA open reading frame are from one strain or subtype of influenza virus and the packaging signals of the chimeric gene segments comprising those open reading frames are from a different strain or subtype of influenza virus. For example, the NA and HA open reading frames may be from A/Hong Kong/4801/2014 (HK14) and the packaging signals may be from A/Puerto Rico/8/1934 (PR8), such as described in Section 6, infra. In a particular embodiment, the NA open reading frame and HA open reading frame are from one strain or subtype of influenza virus and the packaging signals of the chimeric gene segments comprising those open reading frames are from a different strain or subtype of influenza virus and those packaging signals from the same strain or subtype of influenza virus as influenza virus NS, PB1, PB2, PA, M, and NP gene segments. In one embodiment, the first chimeric influenza virus gene segment comprises the packaging signals described in FIGS. 4A-4B of International Patent Application Publication No. WO 2011/014645 and the open reading frame of an influenza virus NA, and the second chimeric influenza virus gene segment comprises the packaging signals described in FIGS. 32A-32C of International Patent Application Publication No. WO 2011/014645 and the open reading frame encoding for an influenza virus HA polypeptide. In a specific embodiment, the first chimeric influenza virus gene segment comprises the nucleotide sequence set forth in SEQ ID NO:24, and the second chimeric influenza virus gene segment comprises the nucleotide sequence set forth in SEQ ID NO:23. In another specific embodiment, the first chimeric influenza virus gene segment comprises the nucleotide sequence set forth in SEQ ID NO:26, and the second chimeric influenza virus gene segment comprises the nucleotide sequence set forth in SEQ ID NO:25.

In another embodiment, provided herein are methods for enhancing a humoral immune response against influenza virus NA (e.g., clinically relevant influenza virus NA), comprising administering to a subject (e.g., human subject) a recombinant influenza virus or a composition comprising the recombinant influenza virus, wherein the recombinant influenza virus comprises a first chimeric influenza virus gene segment, a second chimeric influenza virus gene segment, and influenza virus NS, PB1, PB2, PA, M, and NP gene segments, wherein: (a) the first chimeric influenza virus gene segment encodes a mutated influenza virus neuraminidase (NA) and the first chimeric influenza virus gene segment comprises: (i) a 3′ non-coding region of a hemagglutinin (HA) influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the mutated influenza virus NA polypeptide, wherein the mutated influenza virus neuraminidase polypeptide comprises a first cytoplasmic domain, a first transmembrane domain, a first stalk domain, and a first globular head domain of a first neuraminidase of a first influenza virus with an insertion of 15 to 50 amino acid residues in the first stalk domain of the first neuraminidase; (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, synomyous mutations are introduced into the 3′ proximal nucleotides, the 5′ proximal nucleotides, or both in the open reading frames of the mutated influenza virus neuraminidase and HA. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. A person skilled in the art would be able to determine the non-coding regions, proximal coding regions, open reading frames, the proximal nucleotides of the influenza virus NA and HA gene segments using techniques and information known to one of skill in the art, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, any start codon in the 3′ proximal coding region of the NA or HA influenza virus gene segment is mutated from ATG to TTG. In a specific embodiment, the first influenza virus is an influenza A virus. In some embodiments, the amino acid residues inserted correspond to amino acid residues found in a second stalk domain of a second neuraminidase of a second influenza A virus, wherein the first influenza A virus is from a different subtype than second influenza A virus. In certain embodiments, the amino acid residues inserted correspond to amino acid residues found in a second stalk domain of a second neuraminidase of a second influenza A virus, wherein the first influenza A virus is from a different strain than second influenza A virus. In some embodiment, the first influenza A virus neuraminidase is a neuraminidase of influenza A virus of subtype N1, N2, N3, N4, N5, N6, N7, N8, N9, N10, or N11. In specific embodiments, the first influenza virus is influenza A virus H1N1 A/Puerto Rico/8/1934 (PR8) or influenza A virus A/Hong Kong/4801/2014. In a specific embodiment, the first chimeric influenza virus gene segment comprises the nucleotide sequence set forth in SEQ ID NO: 27 and the second chimeric influenza virus gene segment comprises the nucleotide sequence set forth in SEQ ID NO: 23. In another specific embodiment, the first chimeric influenza virus gene segment comprises the nucleotide sequence set forth in SEQ ID NO: 28 and the second chimeric influenza virus gene segment comprises the nucleotide sequence set forth in SEQ ID NO: 25.

In another aspect, provided herein are methods for increasing the concentration of antibody that binds to influenza virus NA, the methods comprising administering to a subject (e.g., human subject) a recombinant influenza virus described herein or an immunogenic composition described herein. In a specific embodiment, the concentration of antibody that binds to influenza virus NA is increased relative to the concentration of antibody that binds to influenza virus NA elicited following administration of a recombinant influenza virus in which the packaging signals of the influenza virus NA gene segment have not been exchanged with the packaging signals of influenza virus HA gene segment. In another embodiment, the concentration of antibody that binds to influenza virus NA is increased relative to the concentration of antibody that binds to influenza virus NA elicited following administration of a recombinant influenza virus in which the NA has not been mutated as described herein. In certain embodiments, the concentration of antibody that binds to influenza virus NA is 1.5, 1.75, 2, 2.5, 3. 3.5, 4, 4.5 fold or higher than the concentration of antibody that binds to NA elicited following administration of a recombinant influenza virus in which the packaging signals of the influenza virus NA gene segment have not been exchanged with the packaging signals of influenza virus HA gene segment. In specific embodiments, the concentration of antibody that binds to influenza virus HA is decreased relative to the concentration of antibody that binds to HA elicited following administration of a recombinant influenza virus in which the packaging signals of the influenza virus NA gene segment have not been exchanged with the packaging signals of influenza virus HA gene segment, such as described in Section 6.4, infra. In certain embodiments, the concentration of antibody that binds to influenza virus HA is 1.25, 1.5, 1.75, 2, 2.5, 3. 3.5, 4, 4.5 fold or lower than the concentration of antibody that binds to HA elicited following administration of a recombinant influenza virus in which the packaging signals of the influenza virus NA gene segment have not been exchanged with the packaging signals of influenza virus HA gene segment.

In one embodiment, provided herein are methods for increasing the concentration of antibody that binds to influenza virus NA, comprising administering to a subject (e.g., human subject) a recombinant influenza virus or a composition comprising the recombinant influenza virus, wherein the recombinant influenza virus comprises a first chimeric influenza virus gene segment and a second chimeric influenza virus gene segment, wherein (a) the first chimeric influenza virus gene segment encodes an influenza virus NA polypeptide and the first chimeric influenza virus gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the influenza virus NA polypeptide, (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, the 3′ proximal nucleotides, the 5′ proximal nucleotides, or both in the open reading frames are mutated (e.g., substituted) in regions implicated in genome packaging in order to abrogate their residual packaging function. In a specific embodiment, the mutations introduced into the 3′ and/or 5′ proximal nucleotides of the open reading frame of the influenza virus gene segment(s) are silent or synonymous mutations. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In another specific embodiment, any start codon in the 3′ proximal coding region of the NA or HA influenza virus gene segment is mutated from ATG to TTG. In another specific embodiment, the NA open reading frame and HA open reading frame are from one strain or subtype of influenza virus and the packaging signals of the chimeric gene segments comprising those open reading frames are from a different strain or subtype of influenza virus. For example, the NA and HA open reading frames may be from A/Hong Kong/4801/2014 (HK14) and the packaging signals may be from A/Puerto Rico/8/1934 (PR8), such as described in Section 6, infra. In a particular embodiment, the NA open reading frame and HA open reading frame are from one strain or subtype of influenza virus and the packaging signals of the chimeric gene segments comprising those open reading frames are from a different strain or subtype of influenza virus and those packaging signals from the same strain or subtype of influenza virus as influenza virus NS, PB1, PB2, PA, M, and NP gene segments. In one embodiment, the first chimeric influenza virus gene segment comprises the packaging signals described in FIGS. 4A-4B of International Patent Application Publication No. WO 2011/014645 and the open reading frame of an influenza virus NA, and the second chimeric influenza virus gene segment comprises the packaging signals described in FIGS. 32A-32C of International Patent Application Publication No. WO 2011/014645 and the open reading frame encoding for an influenza virus HA polypeptide. In a specific embodiment, the first chimeric influenza virus gene segment comprises the nucleotide sequence set forth in SEQ ID NO:24, and the second chimeric influenza virus gene segment comprises the nucleotide sequence set forth in SEQ ID NO:23. In another specific embodiment, the first chimeric influenza virus gene segment comprises the nucleotide sequence set forth in SEQ ID NO:26, and the second chimeric influenza virus gene segment comprises the nucleotide sequence set forth in SEQ ID NO:25. In specific embodiments, the concentration of antibody that binds to influenza virus HA is decreased relative to the concentration of antibody that binds to HA elicited following administration of a recombinant influenza virus in which the packaging signals of the influenza virus NA gene segment have not been exchanged with the packaging signals of influenza virus HA gene segment, such as described in Section 6.4, infra. In certain embodiments, the concentration of antibody that binds to influenza virus HA is 1.25, 1.5, 1.75, 2, 2.5, 3. 3.5, 4, 4.5 fold or lower than the concentration of antibody that binds to HA elicited following administration of a recombinant influenza virus in which the packaging signals of the influenza virus NA gene segment have not been exchanged with the packaging signals of influenza virus HA gene segment. In a specific embodiment, the subject is human.

In another embodiment, provided herein are methods for increasing the concentration of antibody that binds to influenza virus NA, comprising administering to a subject (e.g., human subject) a recombinant influenza virus or a composition comprising the recombinant influenza virus, wherein the recombinant influenza virus comprises a first chimeric influenza virus gene segment, a second chimeric influenza virus gene segment, and influenza virus NS, PB1, PB2, PA, M, and NP gene segments, wherein: (a) the first chimeric influenza virus gene segment encodes a mutated influenza virus neuraminidase (NA) and the first chimeric influenza virus gene segment comprises: (i) a 3′ non-coding region of a hemagglutinin (HA) influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the mutated influenza virus NA polypeptide, wherein the mutated influenza virus neuraminidase polypeptide comprises a first cytoplasmic domain, a first transmembrane domain, a first stalk domain, and a first globular head domain of a first neuraminidase of a first influenza virus with an insertion of 15 to 50 amino acid residues in the first stalk domain of the first neuraminidase; (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, synonymous mutations are introduced into the 3′ proximal nucleotides, the 5′ proximal nucleotides, or both in the open reading frames of the mutated influenza virus neuraminidase and HA. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. A person skilled in the art would be able to determine the non-coding regions, proximal coding regions, open reading frames, the proximal nucleotides of the influenza virus NA and HA gene segments using techniques and information known to one of skill in the art, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, any start codon in the 3′ proximal coding region of the NA or HA influenza virus gene segment is mutated from ATG to TTG. In a specific embodiment, the first influenza virus is an influenza A virus. In some embodiments, the amino acid residues inserted correspond to amino acid residues found in a second stalk domain of a second neuraminidase of a second influenza A virus, wherein the first influenza A virus is from a different subtype than second influenza A virus. In certain embodiments, the amino acid residues inserted correspond to amino acid residues found in a second stalk domain of a second neuraminidase of a second influenza A virus, wherein the first influenza A virus is from a different strain than second influenza A virus. In some embodiment, the first influenza A virus neuraminidase is a neuraminidase of influenza A virus of subtype N1, N2, N3, N4, N5, N6, N7, N8, N9, N10, or N11. In specific embodiments, the first influenza virus is influenza A virus H1N1 A/Puerto Rico/8/1934 (PR8) or influenza A virus A/Hong Kong/4801/2014. In a specific embodiment, the first chimeric influenza virus gene segment comprises the nucleotide sequence set forth in SEQ ID NO: 27 and the second chimeric influenza virus gene segment comprises the nucleotide sequence set forth in SEQ ID NO: 23. In another specific embodiment, the first chimeric influenza virus gene segment comprises the nucleotide sequence set forth in SEQ ID NO: 28 and the second chimeric influenza virus gene segment comprises the nucleotide sequence set forth in SEQ ID NO: 25. In specific embodiments, the concentration of antibody that binds to influenza virus HA is decreased relative to the concentration of antibody that binds to HA elicited following administration of a recombinant influenza virus in which the packaging signals of the influenza virus NA gene segment have not been exchanged with the packaging signals of influenza virus HA gene segment, such as described in Section 6.4, infra. In certain embodiments, the concentration of antibody that binds to influenza virus HA is 1.25, 1.5, 1.75, 2, 2.5, 3. 3.5, 4, 4.5 fold or lower than the concentration of antibody that binds to HA elicited following administration of a recombinant influenza virus in which the packaging signals of the influenza virus NA gene segment have not been exchanged with the packaging signals of influenza virus HA gene segment. In a specific embodiment, the subject is human.

### 3.1 Terminology

As used herein, the term “nucleic acid” is intended to include DNA molecules (e.g., cDNA or genomic DNA) and RNA molecules (e.g., mRNA) and analogs of the DNA or RNA generated using nucleotide analogs. The nucleic acid can be single-stranded or double-stranded.

As used herein, the terms “purified” and “isolated” when used in the context of a polypeptide (including an antibody) that is obtained from a natural source, e.g., cells, refers to a polypeptide which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a polypeptide (including an antibody) that is chemically synthesized refers to a polypeptide which is substantially free of chemical precursors or other chemicals which are involved in the syntheses of the polypeptide. In a specific embodiment, a mutated influenza virus NA polypeptide is chemically synthesized. In another specific embodiment, a mutated influenza virus NA polypeptide is recombinantly produced. In another specific embodiment, a mutated influenza virus NA polypeptide is isolated.

As used herein, terms “subject” or “patient” are used interchangeably to refer to an animal (e.g., birds, reptiles, and mammals). In a specific embodiment, a subject is a bird. In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human. In another embodiment, a subject is a human infant. In another embodiment, a subject is a human child. In another embodiment, a subject is a human adult. In another embodiment, a subject is an elderly human. In another embodiment, a subject is a premature human infant.

As used herein, the term “premature human infant” refers to a human infant born at less than 37 weeks of gestational age.

As used herein, the term “seasonal influenza virus strain” refers to a strain of influenza virus to which a subject population is exposed to on a seasonal basis. In specific embodiments, the term seasonal influenza virus strain refers to a strain of influenza A virus. In specific embodiments, the term seasonal influenza virus strain refers to a strain of influenza virus that belongs to the H1 or the H3 subtype, i.e., the two subtypes that presently persist in the human subject population. In other embodiments, the term seasonal influenza virus strain refers to a strain of influenza B virus.

The terms “tertiary structure” and “quaternary structure” have the meanings understood by those of skill in the art. Tertiary structure refers to the three-dimensional structure of a single polypeptide chain. Quaternary structure refers to the three dimensional structure of a polypeptide having multiple polypeptide chains.

As used herein, in some embodiments, the phrase “wild-type” in the context of a viral polypeptide refers to a viral polypeptide that is found in nature and is associated with a naturally occurring virus.

As used herein, in some embodiments, the phrase “wild-type” in the context of a virus refers to the types of a virus that are prevalent, circulating naturally and producing typical outbreaks of disease. In other embodiments, the term “wild-type” in the context of a virus refers to a parental virus.

### 4. BRIEF DESCRIPTION OF THE DRAWINGS

FIGS. 1A-1F. Design and rescue of influenza viruses with extended N1 neuraminidase stalk domains. (FIG. 1A) Estimated lengths of the ectodomains of N1 proteins with different stalk lengths compared with the ectodomain of H1 hemagglutinin. The structure of the NA stalk has not been determined and is indicated by four bars. The lengths of the ectodomains are estimates from molecular dynamics simulations, as reported before (45). The depiction of H1 is based on the crystal structure of the PR8 HA (PDB number 1RU7 (46)) and the depictions of N1 are based on the crystal structure of the NA of A/California/04/2009 (Cal09) virus (PDB number 3TI3 (47)). The structures are not to scale and were visualized with UCSF Chimera (48). (FIG. 1B; SEQ ID NOs.: 35 and 36) The four domains of the NA protein are indicated (CT, cytoplasmic tail; TM, transmembrane domain). The diagram is not to scale. The amino acid sequences comprising the stalk region are defined as previously described (42). Asterisks denote conserved amino acids. A 15-amino acid region of the Cal09 NA stalk that is not present in the PR8 NA is shown. (FIG. 1C; SEQ ID NOs.: 37-39) Alignment of the three NA proteins with different stalk lengths. The 15 amino acid N2 insert is derived from the NA stalk domain of the A/New York/61/2012 (H3N2) virus. (FIG. 1D) Hemagglutination (HA) titers of allantoic fluids from plaque-purified viruses. Data points represent individual plaques (n=5 per virus). Horizontal bars show the mean value, whiskers the standard deviation. Phosphate-buffered saline (PBS) control wells showed no hemagglutination (not shown). (FIG. 1E) Western blots of proteins from concentrated viruses (left: anti-NA, right: anti-HA). One or two micrograms total protein content of each virus preparation were analyzed, as indicated above the blots. Protein marker sizes in kilodaltons are indicated to the left of the blots. The bands corresponding to the HA0 (uncleaned HA) and HA2 (cleavage product of HA0) are indicated with arrows (the antibody is specific to the C-terminal portion of the HA protein and therefore does not react with the HA1 polypeptide). (FIG. 1F) Immunofluorescence microscopy of MDCK cells infected with the indicated viruses and stained with anti-N1 monoclonal antibody 4A5 (11).

FIGS. 2A-2D. The extended stalk domain enhances IgG responses to the N1 neuraminidase. (FIG. 2A) Immunization regime. Mice received three doses containing 10 of formalin-inactivated viruses. Serum obtained four weeks after the third immunization was analyzed for antibodies against N1 neuraminidase and H1 hemagglutinin proteins. (FIGS. 2B, 2C) Serum IgG levels to recombinant tetrameric N1 neuraminidase (FIG. 2B) and recombinant trimeric H1 hemagglutinin (FIG. 2C) from PR8 virus, as measured by ELISA. AUC, area under the curve. (FIG. 2D) Hemagglutination inhibition (HI) titers against wildtype PR8 virus. Statistical significance was inferred by one-way ANOVA with Bonferroni correction and P values are indicated in the graphs. Note that the Ins30 group only comprises 9 sera, as one animal in that group died unrelated to the experiment.

FIGS. 3A-3B. The extended stalk domain enhances ADCC active antibody responses to the N1 neuraminidase. (FIG. 3A) Results of the neuraminidase inhibition assay. Both subpanels show the same data. The left subpanel shows % inhibition over the serum dilution with data points representing mean values of 9 (Ins30 group) or 10 (all other groups) individual mice ±standard deviation. The right subpanel shows the 50% inhibitory concentrations (IC₅₀ values) calculated from the curves of the left subpanel. (FIG. 3B) Results from antibody-dependent cellular cytotoxicity (ADCC) reporter assays. From left to right, the subpanels show assays performed with HEK 293T cells transfected with a pCAGGS expression plasmid for the N1 protein of the PR8 virus and MDCK cells infected either with an H1N1 virus (PR8) or an H3N1 virus (H3 from A/Hong Kong/4801/2014 and all other proteins from PR8). Data points represent pooled sera measured in triplicates, horizontal bars show the mean values and the whiskers indicate the standard deviation.

FIGS. 4A-4G. Design, rescue and immunogenicity of influenza viruses with extended N2 neuraminidase stalk domains. (FIG. 4A; SEQ ID NOs.: 40-42) The four domains of the NA protein are indicated (CT, cytoplasmic tail; TM, transmembrane domain). The diagram is not to scale. The N2-Del25 protein has a deletion of 25 amino acids. The 15 amino acid insert of the N2-Ins15 protein is derived from the N1 protein of the Cal09 virus (see FIG. 1B). Asterisks denote conserved amino acids. (FIG. 4B) Hemagglutination (HA) titers of allantoic fluids from plaque-purified viruses measured in duplicates. Phosphate-buffered saline (PBS) control wells showed no hemagglutination (not shown). (FIG. 4C) Western blots of proteins from concentrated viruses. One microgram of total protein content (left blots) or amounts that were normalized to achieve equal intensities for the NP protein (right blots) of each virus were analyzed, as indicated above the blots. The normalization factors for Del25, wt and Ins15 viruses were 0.802, 1.0, and 0.765 respectively. Approximate protein sizes in kilodaltons are indicated to the right of the blots. (FIG. 4D) S Immunization regime. Mice received an amount of formalin-inactivated virus equivalent to 10 μg of N2-wt virus as determined by normalization to NP protein. Sera obtained four weeks after immunization were analyzed for IgGs against N2 neuraminidase and H3 hemagglutinin by ELISA and for HI-reactive antibodies. (FIGS. 4E, 4F) Serum IgG levels to recombinant tetrameric N2 neuraminidase (FIG. 4E) or recombinant trimeric H3 hemagglutinin (FIG. 4F) from HK14 virus, as measured by ELISA. AUC, area under the curve. Statistical significance was inferred by one-way ANOVA with Bonferroni correction, and P values are indicated in the graphs. n.s., not significant. (FIG. 4G) Hemagglutination inhibition (HI) titers against HK2014-wt virus. Pooled sera were analyzed in triplicates.

FIGS. 5A-5B. Chimeric Segment Design and Expression Levels of HA and NA. HK14 chimeric segment design (FIG. 5A). Swap viruses express more NA and less HA (FIG. 5B).

FIGS. 6A-6B. Immunization with swap viruses significantly improves NA-specific antibody response. FIG. 6A seroreactivity to recombinant HK NA and recombinant HK14 NA. FIG. 6B seroreactivity to recombinant PR8 NA and recombinant PR8 HA.

FIGS. 7A-7B. Protective Anti-NA Antibody Response. Anti-NA antibody response elicited from HK14 swap immunization is more protective against H1N2 challenge than that elicited from HK14 wt immunization. The weight loss and survival of mice following passive immunization with sera and virus challenge are provided in FIG. 7A and FIG. 7B, respectively.

FIGS. 8A-8B. Extending the stalk domain of influenza B NA enhances its immunogenicity. FIG. 8A seroreactivity to recombinant Brisbane NA and FIG. 8B seroreactivity to recombinant Brisbane HA.

FIGS. 9A-9D. Design and rescue of PR8 virus with swapped HA and NA packaging signals. (FIG. 9A) Design of influenza A virus genomic segments with rewired packaging signals that code for PR8 HA and NA. PR8 NA-HA-NA is comprised of the PR8 HA ORF flanked by the 3′ terminal 173 base-pairs and the 5′ terminal 209 base-pairs of PR8 NA. PR8 HA-NA-HA is comprised of the PR8 NA ORF flanked by the 3′ terminal 99 base-pairs and the 5′ terminal 150 base-pairs of PR8 HA. Serial synonymous mutations were made at the 3′ and 5′ ends of the ORFs in order to abrogate the residual packaging capabilities of these regions. The ATGs (in positive sense) upstream of the HA and NA translation start sites were mutated to TTGs to prevent premature translation. (FIG. 9B) Genomic composition of recombinant viruses containing either wild-type or rewired (swap) PR8 HA and NA segments. (FIG. 9C) Hemagglutination (HA) titers of allantoic fluid containing virus grown in eggs in triplicate. No hemagglutination was observed in PBS control wells. (FIG. 9D) Western blots of proteins from concentrated PR8 wt, PR8 swap, and NDV viruses for influenza virus HA, NA, and NP proteins. One microgram of total protein content from each viral preparation was loaded.

FIGS. 10A-10D. Rewiring HA and NA packaging signals enhances anti-PR8 N1 antibody response in whole virus vaccination. (FIG. 10A) Mice were vaccinated twice with 10 μg formalin-inactivated purified PR8-wt or PR8-swap virus. Mice were bled four weeks post-boost and sera were isolated for downstream analysis. IgG levels to recombinant tetrameric PR8 N1 protein (FIG. 10B) and trimeric PR8 H1 protein (FIG. 10C) were measured by ELISA. (FIG. 10D) Sera from wt and swap immunized mice were pooled and IgG1 and IgG2a-specific ELISAs were performed with recombinant PR8 N1 protein. Log₁₀-transformed area under the curve (AUC) values were compared for all ELISAs. p-values listed for each comparison were obtained by one-way ANOVA with Bonferroni correction.

FIGS. 11A-11F. Design and rescue of rewired PR8 virus expressing HK14 HA and NA. (FIG. 11A) Design of influenza A virus genomic segments with rewired packaging signals that code for HK14 HA and NA. HK14 NA-HA-NA is comprised of the HK14 HA ORF flanked by the 3′ terminal 173 base-pairs and the 5′ terminal 209 base-pairs of PR8 NA. HK14 HA-NA-HA is comprised of the HK14 NA ORF flanked by the 3′ terminal 99 base-pairs and the 5′ terminal 150 base-pairs of PR8 HA. The ATGs (in positive sense) upstream of the HA and NA translation start sites were mutated to TTGs to prevent premature translation. (FIG. 11B) Hemagglutination (HA) titers of allantoic fluid containing virus grown in eggs in triplicate. No hemagglutination was observed in PBS control wells. (FIG. 11C) Western blots of proteins from concentrated HK14 wt, HK14 swap, and NDV viruses for influenza virus HA, NA and NP proteins. One microgram of total protein content from each viral preparation was loaded. (FIGS. 11D, 11E) Computational sections through cryo-electron tomograms of purified viruses show that there are more NA molecules and fewer HA molecules on particles released after infection with HK14 swap virus than with HK14 wt virus. Regions predominantly containing HA glycoproteins are outlined. Regions predominantly containing NA glycoproteins are outlined in blue. Magnification shows that the HA has a classic bi-lobed peanut shape, while the NA has a globular head with a thin stalk. (FIG. 11F) Visual quantification of surface glycoproteins shows that most of the analyzed viral particles in the wild-type sample have >75% HA content, whereas most of the particles in the swap sample have >75% NA content.

FIGS. 12A-12E. Rewiring HA and NA packaging signals enhances anti-N2 antibody response. (FIG. 12A) Mice were vaccinated twice with 10 μg either formalin-inactivated purified HK14 wt or HK14 swap virus. Mice were bled four weeks post-boost and sera were isolated for downstream analysis. IgG levels to recombinant tetrameric HK14 N2 protein (FIG. 12B) and trimeric HK14 H3 protein (FIG. 12C) were measured by ELISA. Log₁₀-transformed area under the curve (AUC) values were compared. (FIG. 12D) Levels of neuraminidase inhibiting antibodies were measured by ELLA using a recombinant H1N2 virus expressing PR8 H1 and HK14 N2 (H1N2). Log₁₀-reciprocal 50% inhibitory concentration (IC50) values were compared. p-values listed for all comparisons were obtained by one-way ANOVA with Bonferroni correction or t test. (FIG. 12E) Levels of antibody-dependent cellular cytotoxicity (ADCC)-active antibodies were assessed by ADCC reporter assay performed on MDCK cells infected with H1N2 virus. Sera from each group were pooled and run in duplicate.

FIG. 13. Anti-NA antibody response elicited by swap virus vaccination protects from matched NA influenza virus challenge. Equal amounts of sera isolated from immunized mice were pooled within each group. Pooled sera were passively transferred intraperitoneally to 5 naïve mice per group. Two hours post-transfer, mice were infected with five times the median lethal dose (LD₅₀) of a recombinant PR8 virus expressing PR8 HA and HK14 NA. Weight loss and survival were measured following infection. Mice that lost >75% of their body weight were euthanized. See FIGS. 7A and 7B.

### 5. DETAILED DESCRIPTION

### 5.1 Mutated Influenza Virus Neuraminidase Polypeptides

Provided herein are mutated influenza virus neuraminidase (NA) polypeptides. A full-length influenza virus neuraminidase typically comprises a cytoplasmic domain, a transmembrane domain, a stalk domain, and a globular head domain. Techniques known to one of skill in the art may be used to delinate the different domains of an influenza virus neuraminidase. In certain embodiments, a mutated influenza virus neuraminidase polypeptide described herein maintains the structure of a full-length influenza virus neuraminidase. That is, in certain embodiments, the mutated influenza virus neuraminidase polypeptides described herein comprise a stable cytoplasmic domain, a transmembrane domain, a stalk domain, and a globular head domain. In certain embodiments, a mutated influenza virus neuraminidase polypeptide described herein comprises a full-length influenza virus neuraminidase, e.g., comprises a cytoplasmic domain, a transmembrane domain, a stalk domain, and a globular head domain, with amino acid residues (e.g., 15 to 45 amino acid residues or 15 to 50 amino acid residues) inserted in the stalk domain. In certain embodiments, a mutated influenza virus neuraminidase polypeptide described herein comprises a transmembrane domain, a stalk domain, and a globular head domain with amino acid residues (e.g., 15 to 45 amino acid residues or 15 to 50 amino acid residues) inserted in the stalk domain. The amino acid residues inserted in the stalk domain may be random amino acid residues or amino acid residues found in one, two or more influenza virus neuraminidases. In a specific embodiment, a mutated influenza virus neuraminidase polypeptide described herein has sialidase activity and supports viral replication.

In one aspect, provided herein is a mutated influenza virus neuraminidase polypeptide comprising a first cytoplasmic domain, a first transmembrane domain, a first stalk domain, and a first globular head domain of a first neuraminidase of a first influenza virus with an insertion of amino acid residues in the first stalk domain that results in the mutated influenza virus neuraminidase having an approximately 10 Å to 100 Å, approximately 20 Å to 100 Å, approximately 30 Å to 100 Å, approximately 40 Å to 100 Å, approximately 50 Å to 100 Å, approximately 60 Å to 100 Å, approximately 70 Å to 100 Å, or approximately 80 Å to 100 Å increase in height relative to the height of the first neuraminidase. In some embodiments, the insertion results in the mutated influenza virus neuraminidase polypeptide having an approximately 10 Å to 90 Å, approximately 20 Å to 90 Å, approximately 30 Å to 90 Å, approximately 40 Å to 90 Å, approximately 50 Å to 90 Å, approximately 60 Å to 90 Å, approximately 70 Å to 90 Å, or approximately 80 Å to 90 Å increase in height relative to the height of the first neuraminidase. In certain embodiments, the insertion results in the mutated influenza virus neuraminidase polypeptide having an approximately 10 Å to 80 Å, approximately 20 Å to 80 Å, approximately 30 Å to 80 Å, approximately 40 Å to 80 Å, approximately 50 Å to 80 Å, approximately 60 Å to 80 Å, or approximately 70 Å to 80 Å increase in height relative to the height of the first neuraminidase. In some embodiments, the insertion results in the mutated influenza virus neuraminidase polypeptide having an approximately 10 Å to 70 Å, approximately 20 Å to 70 Å, approximately 30 Å to 70 Å, approximately 40 Å to 70 Å, approximately 50 Å to 70 Å, or approximately 60 Å to 70 Å increase in height relative to the height of the first neuraminidase. In certain embodiments, the insertion results in the mutated influenza virus neuraminidase polypeptide having an approximately 10 Å to 50 Å, approximately 20 Å to 50 Å, approximately 30 Å to 50 Å, or approximately 40 Å to 50 Å increase in height relative to the height of the first neuraminidase. In some embodiments, the insertion results in the mutated influenza virus neuraminidase polypeptide having an approximately 10 Å to 40 Å, approximately 20 Å to 40 Å, or approximately 30 Å to 40 Å increase in height relative to the height of the first neuraminidase. In some embodiments, the insertion results in the mutated influenza virus neuraminidase polypeptide having an approximately 10 Å to 30 Å or approximately 20 Å to 30 Å increase in height relative to the height of the first neuraminidase. In certain embodiments, the insertion results in the mutated influenza virus neuraminidase polypeptide having an approximately 10 Å to 20 Å increase in height relative to the height of the first neuraminidase. In some embodiments, the insertion results in the mutated influenza virus neuraminidase polypeptide having an approximately 41 Å, 41 Å 42 Å, 43 Å, 44 Å, 45 Å, 46 Å, 47 Å, 48 Å, 49 Å, 50 Å, 51 Å, 52 Å, 52 Å, 54 Å, 55 Å, 56 Å, 57 Å, 58 Å, 59 Å, 60 Å, 61 Å, 62 Å, 63 Å, 64 Å, or 65 Å increase in height relative to the height of the first neuraminidase. In certain embodiments, the insertion results in the mutated influenza virus neuraminidase polypeptide having an approximately 30 Å, 31 Å 32 Å, 33 Å, 34 Å, 35 Å, 36 Å, 37 Å, 38 Å, 39 Å or 40 Å increase in height relative to the height of the first neuraminidase. In some embodiments, the insertion results in the mutated influenza virus neuraminidase polypeptide having an approximately 10 Å, 11 Å 12 Å, 13 Å, 14 Å, 15 Å, 16 Å, 17 Å, 18 Å, 19 Å or 20 Å increase in height relative to the height of the first neuraminidase. In specific embodiments, each amino acid in the first stalk domain is estimated to contribute approximately 1.2 Å to the total height of the neuraminidase. In specific embodiments, each amino acid inserted in the first stalk domain is estimated to contribute approximately 1.2 Å to the total height of the neuraminidase.

In another aspect, provided herein is a mutated influenza virus NA polypeptide comprising a first neuraminidase stalk domain with amino acid residues inserted such that the stalk domain of the mutated influenza virus NA polypeptide is extended from the surface of an influenza virus membrane such that it surpasses the height of the hemagglutinin of the influenza virus. In certain embodiments, 15 to 50, 15 to 45, 15 to 30, or 20 to 30 amino acid residues are inserted into the first neuraminidase stalk domain. In some embodiments, 15 to 50, 20 to 50, 25 to 50, 30 to 50 or 40 to 50 amino acid residues are inserted into the first neuraminidase stalk domain. In some embodiments, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues are inserted into the first neuraminidase stalk domain. In certain embodiments, 15, 16, 17, 18, 19, 20, 21, 22, 23, 24, 25, 26, 27, 28, 29, or 30 amino acid residues are inserted into the first neuraminidase stalk domain. In certain embodiments, the increase in height of the NA does not result in a statistically significant reduction in anti-HA antibody generated by an influenza virus comprising the mutated influenza virus NA polypeptide, such as described in Section 6.1, infra. In some embodiments, the increase in height of the NA does not result in a statistically significant reduction in anti-HA antibody generated by an influenza virus comprising the mutated influenza virus NA polypeptide, but increases (e.g., a statistically significant increase) the anti-NA antibody generated by such virus, such as described in Section 6.1, infra.

In another aspect, provided herein is a mutated influenza virus neuraminidase polypeptide comprising a first cytoplasmic domain, a first transmembrane domain, a first stalk domain, and a first globular head domain of a first neuraminidase of a first influenza virus with an insertion of amino acid residues in the first stalk domain that results in the mutated influenza virus neuraminidase having a height approximately 10 Å to 50 Å, approximately 20 Å to 50 Å, approximately 30 Å to 50 Å, or approximately 40 Å to 50 Å higher than the height of the hemagglutinin of the first influenza virus. In some embodiments, the insertion results in the mutated influenza virus neuraminidase polypeptide having a height approximately 41 Å, 41 Å 42 Å, 43 Å, 44 Å, 45 Å, 46 Å, 47 Å, 48 Å, 49 Å, or 50 Å higher than the height of the hemagglutinin of the first influenza virus. In certain embodiments, the insertion results in the mutated influenza virus neuraminidase polypeptide having a height approximately 30 Å, 31 Å 32 Å, 33 Å, 34 Å, 35 Å, 36 Å, 37 Å, 38 Å, 39 Å or 40 Å higher than the height of the hemagglutinin of the first influenza virus. In some embodiments, the insertion results in the mutated influenza virus neuraminidase polypeptide having a height approximately 10 Å, 11 Å 12 Å, 13 Å, 14 Å, 15 Å, 16 Å, 17 Å, 18 Å, 19 Å or 20 Å higher than the height of the hemagglutinin of the first influenza virus. In specific embodiments, each amino acid in the first stalk domain is estimated to contribute approximately 1.2 Å to the total height of the neuraminidase. In specific embodiments, each amino acid inserted in the first stalk domain is estimated to contribute approximately 1.2 Å to the total height of the neuraminidase.

In another aspect, provided herein is a mutated influenza virus neuraminidase polypeptide comprising a first cytoplasmic domain, a first transmembrane domain, a first stalk domain, and a first globular head domain of a first neuraminidase of a first influenza virus with an insertion of 15 to 45 or 15 to 50 amino acid residues in the first stalk domain of the first neuraminidase. In certain embodiments, a mutated influenza virus neuraminidase polypeptide described herein comprises a first cytoplasmic domain, a first transmembrane domain, a first stalk domain, and a first globular head domain of a first neuraminidase of a first influenza virus with an insertion of 15 to 30 amino acid residues in the first stalk domain of the first neuraminidase. In certain embodiments, a mutated influenza virus neuraminidase polypeptide described herein comprises a first cytoplasmic domain, a first transmembrane domain, a first stalk domain, and a first globular head domain of a first neuraminidase of a first influenza virus with an insertion of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues in the first stalk domain of the first neuraminidase. In a specific embodiment, the insertion is encoded by a nucleotide sequence comprising the sequence set forth in SEQ ID NO: 29.

In some embodiments, random amino acid residues that do not affect the conformation/structure of the first neuraminidase are inserted into the first stalk domain of the first neuraminidase. In certain embodiments, amino acid residues of a conserved T cell epitope are inserted into the first stalk domain of the first neuraminidase as long as the insertion does not affect the conformation/structure of the first neuraminidase. In a specific embodiment, amino acid residues of a conserved CD8 T cell epitope (e.g., an RSV CD8(+) T cell epitope F(85-93)) are inserted into the first stalk domain of the first neuraminidase as long as the insertion does not affect the conformation/structure of the first neuraminidase. In other embodiments, amino acid residues of a conserved T cell epitope, such as a CD8 T cell epitope (e.g., an RSV CD8(+) T cell epitope F(85-93)), are not inserted into the first stalk domain of the first neuraminidase. In certain embodiments, amino acid residues found in the stalk domain of a second neuraminidase of a second influenza virus are inserted into the first stalk domain of the first neuraminidase that do not affect the conformation/structure of the first neuraminidase. In some embodiments, amino acid residues found in the stalk domain of a two or more neuraminidases of a two or more influenza viruses are inserted into the first stalk domain of the first neuraminidase that do not affect the conformation/structure of the first neuraminidase. One might want to refrain from inserting amino acid residues, such as cysteine, proline or both, in the first stalk domain of the first neuraminidase that may impact the folding of the mutated influenza virus NA polypeptide. In addition, one might want to refrain from inserting amino acid residues in the first stalk domain of the first neuraminidase that impacts the coding for N-linked glycosylation sites (N-X-S/T). In selecting the amino acid residues to insert into the first stalk domain of the first neuraminidase, care should be taken to maintain the conformation/structure of the first neuraminidase. See, e.g., Section 6. In specific embodiments, the amino acid residues inserted into the first stalk domain of the first neuraminidase are not consecutive amino acid residues in the stalk domain of a second neuraminidase. In some embodiments, the amino acid residues inserted into the first stalk domain of the first neuraminidase are consecutive amino acid residues in the stalk domain of a second neuraminidase of a second influenza virus. In a specific embodiment, the selection of amino acid residues to insert into the first stalk domain of a first neuraminidase may be identified as described in Section 6, infra. The effect of the amino acid residue(s) inserted on the conformation/structure of the first neuraminidase may be determined by assays known to one of skill in the art, e.g., structure programs, crystallography, or functional assays. In a specific embodiment, the methods described in Section 6 are used to generate and evaluate a mutated influenza virus NA polypeptide.

In another aspect, provided herein is a mutated influenza virus neuraminidase polypeptide described herein comprises a first cytoplasmic domain, a first transmembrane domain, a first stalk domain, and a first globular head domain of a first neuraminidase of a first influenza virus with an insertion of 15 to 45 or 15 to 50 amino acid residues in the first stalk domain of the first neuraminidase, wherein the 15 to 45 or 15 to 50 amino acid residues inserted are from a second stalk domain of a second neuraminidase of a second influenza virus, and wherein the first influenza virus is from a different subtype than second influenza virus. In certain embodiments, a mutated influenza virus neuraminidase polypeptide described herein comprises a first cytoplasmic domain, a first transmembrane domain, a first stalk domain, and a first globular head domain of a first neuraminidase of a first influenza A virus with an insertion of 15 to 30 amino acid residues in the first stalk domain of the first neuraminidase, wherein the 15 to 30 amino acid residues inserted are from a second stalk domain of a second neuraminidase of a second influenza virus, and wherein the first influenza virus is from a different subtype than second influenza virus. In certain embodiments, a mutated influenza virus neuraminidase polypeptide described herein comprises a first cytoplasmic domain, a first transmembrane domain, a first stalk domain, and a first globular head domain of a first neuraminidase of a first influenza virus with an insertion of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues in the first stalk domain of the first neuraminidase, wherein the amino acid residues inserted are from a second stalk domain of a second neuraminidase of a second influenza virus, and wherein the first influenza virus is from a different subtype than second influenza virus.

In another aspect, provided herein is a mutated influenza virus neuraminidase polypeptide described herein comprises a first cytoplasmic domain, a first transmembrane domain, a first stalk domain, and a first globular head domain of a first neuraminidase of a first influenza virus with an insertion of 15 to 45 or 15 to 50 amino acid residues in the first stalk domain of the first neuraminidase, wherein the 15 to 45 or 15 to 50 amino acid residues inserted are from a second stalk domain of a second neuraminidase of a second influenza virus, and wherein the first influenza virus is from a different strain than second influenza virus. In certain embodiments, a mutated influenza virus neuraminidase polypeptide described herein comprises a first cytoplasmic domain, a first transmembrane domain, a first stalk domain, and a first globular head domain of a first neuraminidase of a first influenza A virus with an insertion of 15 to 30 amino acid residues in the first stalk domain of the first neuraminidase, wherein the 15 to 30 amino acid residues inserted are from a second stalk domain of a second neuraminidase of a second influenza virus, and wherein the first influenza virus is from a different strain than second influenza virus. In certain embodiments, a mutated influenza virus neuraminidase polypeptide described herein comprises a first cytoplasmic domain, a first transmembrane domain, a first stalk domain, and a first globular head domain of a first neuraminidase of a first influenza virus with an insertion of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues in the first stalk domain of the first neuraminidase, wherein the amino acid residues inserted are from a second stalk domain of a second neuraminidase of a second influenza virus, and wherein the first influenza virus is from a different strain than second influenza virus.

In certain embodiments, a mutated influenza virus NA polypeptide provided herein comprises a signal peptide. Typically, the signal peptide is cleaved during or after polypeptide expression and translation to yield a mature mutated influenza virus NA polypeptide. In certain embodiments, also provided herein are mature mutated influenza virus NA polypeptides that lack a signal peptide. In embodiments where a mutated influenza virus NA polypeptide provided herein comprises a signal peptide, the signal peptide might be based on any influenza virus signal peptide known to those of skill in the art. In certain embodiments, the signal peptides are based on influenza A signal peptides. In some embodiments, the signal peptides are based on influenza B signal peptides.

In some embodiments, the first influenza virus of a mutated influenza virus neuraminidase polypeptide described herein is an influenza A virus. In certain embodiments, the first influenza virus of a mutated influenza virus neuraminidase polypeptide described herein is an influenza B virus. In some embodiments, the first neuraminidase of a mutated influenza virus neuraminidase polypeptide described herein is an N1, N2, N3, N4, N5, N6, N7, N8, N9, N10, or N11 influenza virus neuraminidase. In certain embodiments, the first neuraminidase of a mutated influenza virus neuraminidase polypeptide described herein is an N1, N2, N3, N4, N5, N6, N7, N8, or N9 influenza virus neuraminidase. In some embodiments, the first neuraminidase of a mutated influenza virus neuraminidase polypeptide described herein is a Group 1 influenza virus neuraminidase, e.g., N1, N4, N5, and N8 influenza virus neuraminidase subtypes. In certain embodiments, the first neuraminidase of a mutated influenza virus neuraminidase polypeptide described herein is a Group 2 influenza virus neuraminidase, e.g., N2, N3, N6, N7, and N9 influenza virus neuraminidase subtypes. In certain embodiments, the first neuraminidase of a mutated influenza virus neuraminidase polypeptide described herein is an N2 subtype. In some embodiments, the first neuraminidase of a mutated influenza virus neuraminidase polypeptide described herein is an influenza B virus.

In certain embodiments, the first neuraminidase of a mutated influenza virus neuraminidase polypeptide described herein is a human influenza virus neuraminidase. Human influenza virus neuraminidase polypeptides are known in the art. In certain embodiments, the first neuraminidase of a mutated influenza virus neuraminidase polypeptide described herein is a swine influenza virus neuraminidase. Swine influenza virus neuraminidase polypeptides are known in the art. In certain embodiments, the first neuraminidase of a mutated influenza virus neuraminidase polypeptide described herein is an equine influenza virus neuraminidase. Equine influenza virus neuraminidase polypeptides are known in the art. In certain embodiments, the first neuraminidase of a mutated influenza virus neuraminidase polypeptide described herein is an avian influenza virus neuraminidase polypeptide. For example, the first neuraminidase of a mutated influenza virus polypeptide described herein may be from a neuraminidase of an H6N1, H7N1, H7N3 or H9N2 influenza virus.

In certain embodiments, the first neuraminidase of a mutated influenza virus neuraminidase polypeptide described herein is the neuraminidase of influenza virus H1N1 strain A/Puerto Rico/8/1934 (PR8). In other embodiments, the first neuraminidase of a mutated influenza virus neuraminidase polypeptide described herein is not the neuraminidase of influenza virus H1N1 strain A/Puerto Rico/8/1934 (PR8). In some embodiments, the first neuraminidase of a mutated influenza virus neuraminidase polypeptide described herein is the neuraminidase of influenza virus H3N2 A/New York/61/2012 (NY12). In some embodiments, the first neuraminidase of a mutated influenza virus neuraminidase polypeptide described herein is not the neuraminidase of influenza virus H3N2 A/New York/61/2012 (NY12). In certain embodiments, the first neuraminidase of a mutated influenza virus neuraminidase polypeptide described herein is the neuraminidase of influenza virus A/WSN/33. In other embodiments, the first neuraminidase of a mutated influenza virus neuraminidase polypeptide described herein is not the neuraminidase of influenza virus A/WSN/33. In certain embodiments, the first neuraminidase of a mutated influenza virus neuraminidase polypeptide described herein is the neuraminidase of influenza virus A/Hong Kong/4801/2014 (HK14). In other embodiments, the first neuraminidase of a mutated influenza virus neuraminidase polypeptide described herein is not the neuraminidase of influenza virus A/Hong Kong/4801/2014 (HK14). In certain embodiments, the first neuraminidase of a mutated influenza virus neuraminidase polypeptide described herein is the neuraminidase of influenza virus B/Phuket/3073/2013. In other embodiments, the first neuraminidase of a mutated influenza virus neuraminidase polypeptide described herein is not the neuraminidase of influenza virus B/Phuket/3073/2013. In certain embodiments, the first neuraminidase of a mutated influenza virus neuraminidase polypeptide described herein is the neuraminidase of influenza virus B/Brisbane/60/2008. In other embodiments, the first neuraminidase of a mutated influenza virus neuraminidase polypeptide described herein is not the neuraminidase of influenza virus B/Brisbane/60/2008.

In some embodiments, the second influenza virus of a mutated influenza virus neuraminidase polypeptide described herein is an influenza A virus. In certain embodiments, the second influenza virus of a mutated influenza virus neuraminidase polypeptide described herein is an influenza B virus. In some embodiments, the second neuraminidase of a mutated influenza virus neuraminidase polypeptide described herein is an N1, N2, N3, N4, N5, N6, N7, N8, N9, N10, or N11 influenza virus neuraminidase. In certain embodiments, the second neuraminidase of a mutated influenza virus neuraminidase polypeptide described herein is an N1, N2, N3, N4, N5, N6, N7, N8, or N9 influenza virus neuraminidase. In some embodiments, the second neuraminidase of a mutated influenza virus neuraminidase polypeptide described herein is a Group 1 influenza virus neuraminidase, e.g., N1, N4, N5, and N8 influenza virus neuraminidase subtypes. In certain embodiments, the second neuraminidase of a mutated influenza virus neuraminidase polypeptide described herein is a Group 2 influenza virus neuraminidase, e.g., N2, N3, N6, N7, and N9 influenza virus neuraminidase subtypes. In certain embodiments, the second neuraminidase of a mutated influenza virus neuraminidase polypeptide described herein is an N2 subtype.

In certain embodiments, the second neuraminidase of a mutated influenza virus neuraminidase polypeptide described herein is a human influenza virus neuraminidase. Human influenza virus neuraminidase polypeptides are known in the art. In certain embodiments, the second neuraminidase of a mutated influenza virus neuraminidase polypeptide described herein is a swine influenza virus neuraminidase. Swine influenza virus neuraminidase polypeptides are known in the art. In certain embodiments, the second neuraminidase of a mutated influenza virus neuraminidase polypeptide described herein is an equine influenza virus neuraminidase. Equine influenza virus neuraminidase polypeptides are known in the art. In certain embodiments, the second neuraminidase of a mutated influenza virus neuraminidase polypeptide described herein is an avian influenza virus neuraminidase polypeptide.

In certain embodiments, the second neuraminidase of a mutated influenza virus neuraminidase polypeptide described herein is the neuraminidase of influenza virus H1N1pdm09 A/California/04/2009 (Cal09). In some embodiments, the second neuraminidase of a mutated influenza virus neuraminidase polypeptide described herein is the neuraminidase of influenza virus A/Goose/Guang-dong/1/96 H5N1. In other embodiments, the second neuraminidase of a mutated influenza virus neuraminidase polypeptide described herein is not the neuraminidase of influenza virus A/Goose/Guang-dong/1/96 H5N1. In certain embodiments, the second neuraminidase of a mutated influenza virus neuraminidase polypeptide described herein is the neuraminidase of influenza virus A/WSN/33 H1N1. In other embodiments, the second neuraminidase of a mutated influenza virus neuraminidase polypeptide described herein is not the neuraminidase of influenza virus A/WSN/33 H1N1. In some embodiments, the second neuraminidase of a mutated influenza virus neuraminidase polypeptide described herein is the neuraminidase of influenza virus A/Tokyo/67 H2N2 or influenza virus A/Tern/Australia/G70C/75 H11N9. In other embodiments, the second neuraminidase of a mutated influenza virus neuraminidase polypeptide described herein is not the neuraminidase of influenza virus A/Tokyo/67 H2N2 or influenza virus A/Tern/Australia/G70C/75 H11N9.

In some embodiments, the amino acid residues of two or more neuraminidases from two or more influenza viruses are inserted into the first stalk domain. In certain embodiments, the amino acid residues inserted into the first stalk domain are from the neuraminidases of influenza virus A/Goose/Guangdong/1/96 H5N1 and influenza virus A/WSN/33 H1N1. In other embodiments, the amino acid residues inserted into the first stalk domain are not from the neuraminidases of influenza virus A/Goose/Guangdong/1/96 H5N1 and influenza virus A/WSN/33 H1N1. In certain embodiments, the amino acid residues inserted into the first stalk domain are from the neuraminidases of influenza virus A/Hong Kong/4801/2014 H3N2 and influenza virus A/California/04/2009 H1N1. In other embodiments, the amino acid residues inserted into the first stalk domain are not from the neuraminidases of influenza virus A/Hong Kong/4801/2014 H3N2 and influenza virus A/California/04/2009 H1N1.

In certain embodiments, the amino acid residues inserted into the first stalk domain are from the neuraminidases of influenza virus A/Tokyo/67 H2N2 and influenza virus A/Tern/Australia/G70C/75 H11N9. In other embodiments the amino acid residues inserted into the first stalk domain are not from the neuraminidases of influenza virus A/Tokyo/67 H2N2 and influenza virus A/Tern/Australia/G70C/75 H11N9.

In some embodiments, the first and second influenza viruses of a mutated influenza virus neuraminidase polypeptide described herein are influenza A viruses. In certain embodiments, the first and second influenza viruses of a mutated influenza virus neuraminidase polypeptide described herein are influenza B viruses. In some embodiments, the first and second neuraminidases of a mutated influenza virus neuraminidase polypeptide described herein are N1, N2, N3, N4, N5, N6, N7, N8, N9, N10, or N11 influenza virus neuraminidases. In certain embodiments, the first and second neuraminidases of a mutated influenza virus neuraminidase polypeptide described herein are N1, N2, N3, N4, N5, N6, N7, N8, or N9 influenza virus neuraminidases. In some embodiments, the first and second neuraminidases of a mutated influenza virus neuraminidase polypeptide described herein are Group 1 influenza virus neuraminidases, e.g., N1, N4, N5, and N8 influenza virus neuraminidase subtypes. In certain embodiments, the first and second neuraminidases of a mutated influenza virus neuraminidase polypeptide described herein are Group 2 influenza virus neuraminidases, e.g., N2, N3, N6, N7, and N9 influenza virus neuraminidase subtypes. In some embodiments, the first and second neuraminidases of a mutated influenza virus neuraminidase polypeptide described herein are influenza B virus neuraminidases.

In certain embodiments, the first and second neuraminidases of a mutated influenza virus neuraminidase polypeptide described herein are human influenza virus neuraminidases. Human influenza virus neuraminidase polypeptides are known in the art. In certain embodiments, the first and second neuraminidases of a mutated influenza virus neuraminidase polypeptide described herein are swine influenza virus neuraminidases. Swine influenza virus neuraminidase polypeptides are known in the art. In certain embodiments, the first and second neuraminidases of a mutated influenza virus neuraminidase polypeptide described herein are equine influenza virus neuraminidases. Equine influenza virus neuraminidase polypeptides are known in the art. In certain embodiments, the first and second neuraminidases of a mutated influenza virus neuraminidase polypeptide described herein are avian influenza virus neuraminidases.

In a specific embodiment, the first neuraminidase of a mutated influenza virus neuraminidase polypeptide described herein is the neuraminidase of influenza virus H1N1 strain A/Puerto Rico/8/1934 (PR8) and the second neuraminidase is the neuraminidase of influenza virus H1N1pdm09 A/California/04/2009 (Cal09). In another specific embodiment, the first neuraminidase of a mutated influenza virus neuraminidase polypeptide described herein is the neuraminidase of influenza virus H3N2 A/New York/61/2012 (NY12) and the second neuraminidase is the neuraminidase of influenza virus H1N1pdm09 A/California/04/2009 (Cal09).

In certain embodiments, the first neuraminidase of a mutated influenza virus neuraminidase polypeptide described herein is the neuraminidase of influenza virus H1N1 strain A/Puerto Rico/8/1934 (PR8) and the second neuraminidase is the neuraminidase of influenza virus A/Goose/Guangdong/1/96 H5N1 or influenza virus A/WSN/33 H1N1. In other embodiments, the first neuraminidase of a mutated influenza virus neuraminidase polypeptide described herein is the neuraminidase of influenza virus H1N1 strain A/Puerto Rico/8/1934 (PR8) and the second neuraminidase is not the neuraminidase of influenza virus A/Goose/Guangdong/1/96 H5N1 or influenza virus A/WSN/33 H1N1.

In certain embodiments, the first neuraminidase of a mutated influenza virus neuraminidase polypeptide described herein is the neuraminidase of influenza virus influenza virus A/WSN/33 H1N1 and the second neuraminidase is the neuraminidase of influenza virus A/Tokyo/67 H2N2 or influenza virus A/Tern/Australia/G70C/75 H11N9. In other embodiments, the first neuraminidase of a mutated influenza virus neuraminidase polypeptide described herein is the neuraminidase of influenza virus influenza virus A/WSN/33 H1N1 and the second neuraminidase is not the neuraminidase of influenza virus A/Tokyo/67 H2N2 or influenza virus A/Tern/Australia/G70C/75 H11N9.

In some embodiments, the amino acid residues of two or more neuraminidases from two or more influenza viruses are inserted into the first stalk domain. In certain embodiments, the first neuraminidase of a mutated influenza virus neuraminidase polypeptide described herein is the neuraminidase of influenza virus H1N1 strain A/Puerto Rico/8/1934 (PR8) and the amino acid residues inserted into the first stalk domain are from the neuraminidases of influenza virus A/Goose/Guangdong/1/96 H5N1 and influenza virus A/WSN/33 H1N1. In other embodiments, the first neuraminidase of a mutated influenza virus neuraminidase polypeptide described herein is the neuraminidase of influenza virus H1N1 strain A/Puerto Rico/8/1934 (PR8) and the amino acid residues inserted into the first stalk domain are not from the neuraminidases of influenza virus A/Goose/Guangdong/1/96 H5N1 and influenza virus A/WSN/33 H1N1.

In certain embodiments, the first neuraminidases of a mutated influenza virus neuraminidase polypeptide described herein is the neuraminidase of influenza virus influenza virus A/WSN/33 H1N1 and the amino acid residues inserted into the first stalk domain are from the neuraminidases of influenza virus A/Tokyo/67 H2N2 and influenza virus A/Tern/Australia/G70C/75 H11N9. In other embodiments, the first neuraminidases of a mutated influenza virus neuraminidase polypeptide described herein is the neuraminidase of influenza virus influenza virus A/WSN/33 H1N1 and the amino acid residues inserted into the first stalk domain are not from the neuraminidases of influenza virus A/Tokyo/67 H2N2 and influenza virus A/Tern/Australia/G70C/75 H11N9.

GenBank™ Accession No. AAA43397.1 provides an exemplary amino acid sequence for a human influenza virus neuraminidase. GenBank™ Accession No. ABG23658.1 (GI: 108946273), GenBank™ Accession No. NP 040981.1 (GI: 8486128), GenBank™ Accession No. AAA43412.1 (GI: 324508), GenBank™ Accession No. ABE97720.1 (GI: 93008579), GenBank™ Accession No. ABE97719.1 (GI: 93008577), and GenBank™ Accession No. ABE97718.1 (GI: 93008575) provide exemplary amino acid sequences for human influenza virus neuraminidases. GenBank™ Accession No. CRI06477.1 provides an exemplary amino acid sequence for a swine influenza virus neuraminidase. GenBank™ Accession No. AAQ90293.1 provides an exemplary amino acid sequence for an equine influenza virus neuraminidase. GenBank™ Accession No. AEX30531.1 (GI: 371449652), GenBank™ Accession No. AEX30532.1 (GI: 371449654), GenBank™ Accession No. AIA62041.1 (GI: 641454926), GenBank™ Accession No. AII30325.1 (GI: 670605039), GenBank™ Accession No. AG018161.1 (GI: 513130855), and GenBank™ Accession No. AAS89005.1 (GI: 46360357) provide exemplary amino acid sequences for avian influenza virus neuraminidases. Sequences of influenza virus genes may also be found in the Influenza Research Database. For example, influenza virus neuraminidase sequences may be found in the Influenza Research Database under Accession No. FJ66084 and Accession No. KF90392. In certain embodiments, an influenza virus neuraminidase comprises the amino acid sequence of an influenza virus A/Puerto Rico/8/1934 (PR8) or A/Hong Kong/4801/2014 (HK14) neuraminidase. An amino acid sequence for an influenza virus A/Hong Kong/4801/2014 (HK14) neuraminidase may be found under GISAID Accession No. EPI1026710. In specific embodiments, an influenza virus neuraminidase comprises the amino acid sequence set forth in SEQ ID NO: 6 or 12. In specific embodiments, an influenza virus neuraminidase is encoded by a nucleotide sequence comprising the nucleotide sequence set forth in SEQ ID NO: 5 or 11.

When designing a mutated influenza virus neuraminidase polypeptide, care should be taken to maintain the stability of the resulting protein. In this regard, it is recommended that cysteine residues capable of forming disulfide bonds be maintained since they contribute to the stability of the neuraminidase protein. See, e.g., Basler et al., 1999, Journal of Virology, 73(10):8095-8103 for non-limiting examples of influenza virus neuraminidase cysteine residues capable of forming disulfide bonds. The stability of influenza neuraminidase polypeptides can be assessed using techniques known in the art, such as sensitivity of the neuraminidase molecules to Ca²⁺, as described in, e.g., Baker and Gandhi, 1976, Archives of Virology, 52:7-18.

In certain embodiments, a mutated influenza virus NA polypeptide provided herein is monomeric. In certain embodiments, a mutated influenza virus NA polypeptide provided herein is multimeric. In certain embodiments, a mutated influenza virus NA polypeptide provided herein is tetrameric. In some embodiments, a mutated influenza virus NA polypeptide described herein is able to form a multimer (e.g., a tetramer).

In another specific embodiment, a mutated influenza virus NA polypeptide is a mutated influenza virus NA polypeptide described in Section 6, infra. In another specific embodiment, a mutated influenza virus NA polypeptide is a mutated influenza virus NA polypeptide comprising the amino acid sequence set forth in SEQ ID NO: 4, 8 or 10.

In specific embodiments, a mutated influenza virus NA polypeptide provided herein is capable of forming a three dimensional structure that is similar to the three dimensional structure of a wild-type influenza NA. Structural similarity might be evaluated based on any technique deemed suitable by those of skill in the art. For instance, reaction, e.g. under non-denaturing conditions, of a mutated influenza virus NA polypeptide with an antibody or antiserum that recognizes a native influenza NA might indicate structural similarity. In certain embodiments, the antibody or antiserum is an antibody or antiserum that reacts with a non-contiguous epitope (i.e., not contiguous in primary sequence) that is formed by the tertiary or quaternary structure of a NA.

In certain embodiments, a mutated influenza virus NA polypeptide described herein retains one, two, or more, or all of the functions of a wild-type influenza NA. In a specific embodiment, a mutated influenza virus NA polypeptide described herein cleaves sialic acid. Assays known to one skilled in the art can be utilized to assess the ability of a mutated influenza virus NA polypeptide to cleave sialic acid. In another specific embodiment, a mutated influenza virus NA polypeptide described herein cleaves sialic acide and supports viral replication.

It will be understood by those of skill in the art that a mutated influenza virus NA polypeptide provided herein can be prepared according to any technique known by and deemed suitable to those of skill in the art, including the techniques described herein. In certain embodiments, a mutated influenza virus NA polypeptide described herein is isolated.

### 5.2 Nucleic Acids Encoding Influenza Virus Neuraminidase Polypeptides

Provided herein are nucleic acid sequences that encode influenza virus neuraminidase polypeptides described herein. Due to the degeneracy of the genetic code, any nucleic acid sequence that encodes a mutated influenza virus neuraminidase (NA) polypeptide described herein is encompassed herein. In specific embodiments, provided herein is a nucleic acid sequence comprising a nucleotide sequence encoding a mutated influenza virus neuraminidase polypeptide (with or without the signal peptide). In a specific embodiment, a nucleic acid sequence comprises a nucleotide sequence described herein. In another specific embodiment, a nucleic acid sequence comprises a nucleotide sequence encoding the amino acid sequence set forth in SEQ ID NO: 4, 8, or 10. In another specific embodiment, a nucleic acid sequence comprises the nucleotide sequence set forth in SEQ ID NO: 3, 7 or 9. In certain embodiment, the nucleotide sequence encoding the mutated influenza virus NA polypeptide comprises a nucleotide sequence encoding a signal peptide (e.g., a signal peptide from the NA of the same influenza virus as the influenza virus engineered to express the mutated influenza virus NA polypeptide). In some embodiments, the nucleic acid sequence further comprises the 5′ non-coding region and 3′ non-coding region of an influenza virus NA (e.g., the 5′ non-coding region and 3′ non-coding region from the NA of the same influenza virus as the influenza virus engineered to express the mutated influenza virus NA polypeptide). In certain embodiments, the nucleic acid sequence comprising a nucleotide sequence encoding a mutated influenza virus neuraminidase polypeptide further comprises the 5′ non-coding region and 3′ non-coding region of an influenza virus NA (e.g., the 5′ non-coding region and 3′ non-coding region from the NA of the same influenza virus as the influenza virus engineered to express the mutated influenza virus NA polypeptide).

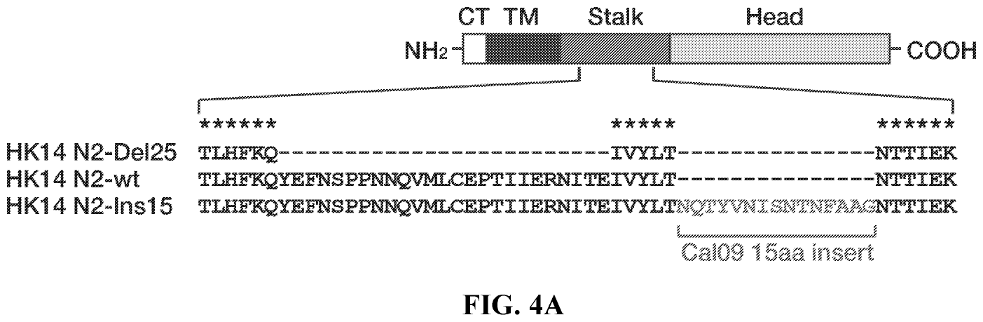

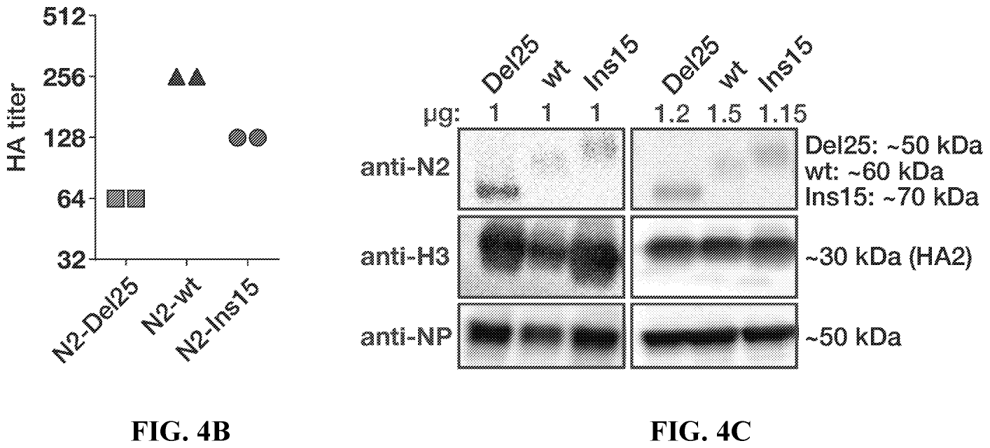

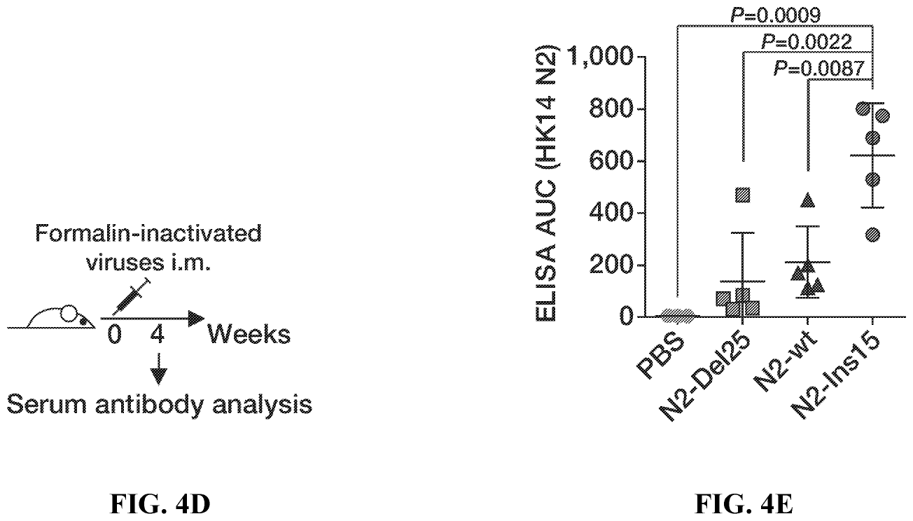

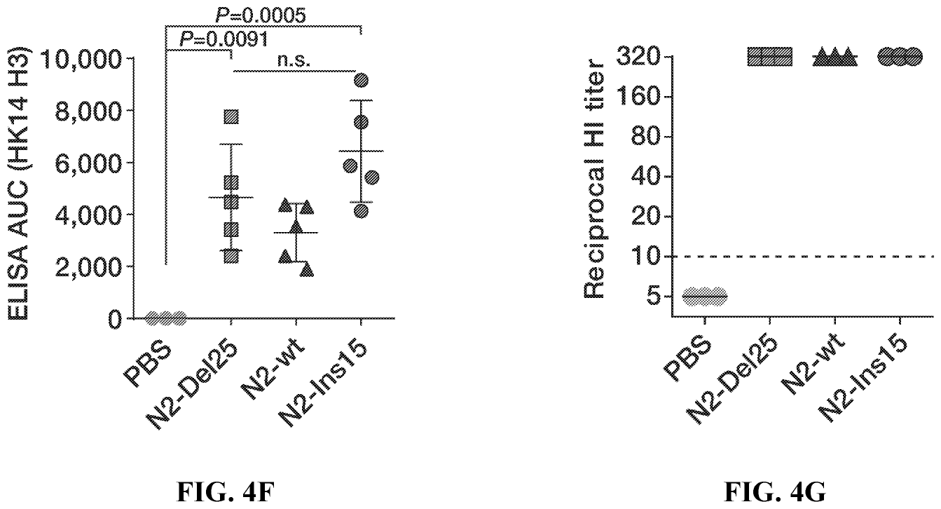

In a specific aspect, an NA segment provided herein that encodes a mutated influenza virus NA polypeptide described herein comprises the packaging signals of another influenza virus gene segment, such as described in, e.g., International Patent Application Publication No. WO 2011/014645; Gao & Palese 2009, PNAS 106:15891-15896; U.S. Pat. No. 8,828,406, each of which is incorporated herein in its entirety. In a specific embodiment, an NA segment provided herein that encodes a mutated influenza virus NA polypeptide described herein comprises the packaging signals of an influenza virus hemagglutinin (HA) gene segment. In another specific embodiment, a chimeric NA segment provided herein that encodes a mutated influenza NA polypeptide described herein comprises the packaging signals found in the 3′ non-coding region, 3′ proximal coding region sequence, the 5′ proximal coding region sequence and the 5′ non-coding region of an influenza virus HA gene segment, wherein any start codon in the 3′ proximal coding region of the first type of influenza virus gene segment is mutated, such as described in, e.g., International Patent Application Publication No. WO 2011/014645; Gao & Palese 2009, PNAS 106:15891-15896; U.S. Pat. No. 8,828,406, each of which is incorporated herein in its entirety. In certain embodiments, the 3′ proximal nucleotides, the 5′ proximal nucleotides, or both in the open reading frame encoding the mutated influenza virus neuraminidase polypeptide are mutated (e.g., substituted). In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the chimeric NA gene segment are silent or synonymous mutations. In another specific embodiment, an NA segment provided herein that encodes a mutated influenza virus NA polypeptide described herein comprises the packaging signals described in FIGS. 4A-4B of International Patent Application Publication No. WO 2011/014645 and U.S. Pat. No. 8,828,406, each of which is incorporated herein in its entirety. In another specific embodiment, an NA segment provided herein that encodes a mutated influenza virus NA comprises the sequence set forth in SEQ ID NO: 27. In another specific embodiment, an NA segment provided herein that encodes a mutated influenza virus NA comprises the sequence set forth in SEQ ID NO: 28.

In a specific aspect, provided herein is a chimeric influenza virus NA gene segment, wherein the chimeric influenza virus NA gene segment encodes a mutated influenza virus NA described herein and the chimeric influenza virus NA gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the mutated influenza virus NA, (iv) a 5′ proximal coding region of the HA influenza virus gene segment; and (v) the 5′ non-coding region of the HA influenza virus gene segment. In certain embodiments, the 3′ proximal nucleotides, the 5′ proximal nucleotides, or both in the open reading frame are mutated (e.g., substituted). In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the chimeric influenza virus NA gene segment are silent or synonymous mutations. In particular embodiments, the silent or synonymous mutations are in regions implicated in genome packaging in order to abrogate their residual packaging function. In another specific embodiment, any start codon in the 3′ proximal coding region of the NA influenza virus gene segment is mutated from ATG to TTG. In a specific embodiment, the NA open reading frame is from one strain or subtype of influenza virus and the packaging signals of the chimeric influenza virus NA gene segment comprising that open reading frame are from a different strain or subtype of influenza virus. For example, the NA open reading frame may be from A/Hong Kong/4801/2014 (HK14) and the packaging signals may be from A/Puerto Rico/8/1934 (PR8), such as described in Section 6, infra. In one embodiment, provided herein is a chimeric influenza virus NA gene segment comprising the packaging signals described in FIGS. 4A-4B of International Patent Application Publication No. WO 2011/014645 and U.S. Pat. No. 8,828,406 and the open reading frame encoding for a mutated influenza virus NA polypeptide described herein. In a specific embodiment, provided herein is a chimeric influenza virus NA gene segment comprising the nucleotide sequence set forth in SEQ ID NO:27. In another specific embodiment, provided herein is a chimeric influenza virus NA gene segment comprising the nucleotide sequence set forth in SEQ ID NO:28.

In another aspect, provided herein is a chimeric influenza virus NA gene segment, wherein the chimeric influenza virus NA gene segment encodes an influenza virus NA polypeptide and the chimeric influenza virus NA gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the influenza virus NA polypeptide, (iv) a 5′ proximal coding region of the HA influenza virus gene segment; and (v) the 5′ non-coding region of the HA influenza virus gene segment. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the 3′ proximal nucleotides, the 5′ proximal nucleotides, or both in the open reading frames are mutated (e.g., substituted). In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. In another specific embodiment, any start codon in the 3′ proximal coding region of the NA influenza virus gene segment is mutated from ATG to TTG. In another specific embodiment, the NA open reading frame is from one strain or subtype of influenza virus and the packaging signals of the chimeric influenza virus NA gene segment comprising the open reading frame is from a different strain or subtype of influenza virus. For example, the NA open reading frame may be from A/Hong Kong/4801/2014 (HK14) and the packaging signals may be from A/Puerto Rico/8/1934 (PR8), such as described in Section 6, infra. In one embodiment, the chimeric influenza virus NA gene segment comprises the packaging signals described in FIGS. 4A-4B of International Patent Application Publication No. WO 2011/014645. In a specific embodiment, provided herein is a chimeric influenza virus NA gene segment comprising the nucleotide sequence set forth in SEQ ID NO:24. In another specific embodiment, provided herein is a chimeric influenza virus NA gene segment comprising the nucleotide sequence set forth in SEQ ID NO:26.

In another aspect, provided herein is a chimeric influenza virus HA gene segment, wherein the chimeric influenza virus HA gene segment encodes an influenza virus HA and the chimeric influenza virus HA gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the 3′ proximal nucleotides, the 5′ proximal nucleotides, or both in the open reading frames are mutated (e.g., substituted). In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. In another specific embodiment, any start codon in the 3′ proximal coding region of the HA influenza virus gene segment is mutated from ATG to TTG. In another specific embodiment, the HA open reading frame is from one strain or subtype of influenza virus and the packaging signals of the chimeric influenza virus HA gene segment comprising the open reading frame is from a different strain or subtype of influenza virus. For example, the HA open reading frame may be from A/Hong Kong/4801/2014 (HK14) and the packaging signals may be from A/Puerto Rico/8/1934 (PR8), such as described in Section 6, infra. In another specific embodiment, the chimeric influenza virus HA gene segment comprises the packaging signals described in FIGS. 32A-32C of International Patent Application Publication No. WO 2011/014645. In another specific embodiment, provided herein is a chimeric influenza virus HA gene segment comprising the nucleotide sequence set forth in SEQ ID NO:23. In another specific embodiment, provided herein is a chimeric influenza virus HA gene segment comprising the nucleotide sequence set forth in SEQ ID NO:25.

Also provided herein are nucleic acid sequences capable of hybridizing to a nucleic acid encoding a mutated influenza virus neuraminidase (NA) polypeptide. In certain embodiments, provided herein are nucleic acid sequences capable of hybridizing to a fragment of a nucleic acid sequence encoding a mutated influenza virus neuraminidase (NA) polypeptide. In other embodiments, provided herein are nucleic acid sequences capable of hybridizing to the full length of a nucleic acid sequence encoding a mutated influenza virus neuraminidase (NA) polypeptide. General parameters for hybridization conditions for nucleic acids are described in Sambrook et al., Molecular Cloning—A Laboratory Manual (2nd Ed.), Vols. 1-3, Cold Spring Harbor Laboratory, Cold Spring Harbor, N.Y. (1989), and in Ausubel et al., Current Protocols in Molecular Biology, vol. 2, Current Protocols Publishing, New York (1994). Hybridization may be performed under high stringency conditions, medium stringency conditions, or low stringency conditions. Those of skill in the art will understand that low, medium and high stringency conditions are contingent upon multiple factors all of which interact and are also dependent upon the nucleic acids in question. For example, high stringency conditions may include temperatures within 5° C. melting temperature of the nucleic acid(s), a low salt concentration (e.g., less than 250 mM), and a high co-solvent concentration (e.g., 1-20% of co-solvent, e.g., DMSO). Low stringency conditions, on the other hand, may include temperatures greater than 10° C. below the melting temperature of the nucleic acid(s), a high salt concentration (e.g., greater than 1000 mM) and the absence of co-solvents.

In some embodiments, a nucleic acid sequence comprising a nucleotide sequence encoding a mutated influenza virus neuraminidase (NA) polypeptide is isolated. In certain embodiments, a chimeric gene segment described herein is isolated. In certain embodiments, an “isolated” nucleic acid sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences in which the nucleic acid sequence is separated from cellular components of the cells from which it is isolated or recombinantly produced. Thus, nucleic acid sequence that is substantially free of cellular material includes preparations of nucleic acid sequence having less than about 30%, 20%, 10%, or 5% (by dry weight) of other nucleic acids. The term “substantially free of culture medium” includes preparations of nucleic acid sequence in which the culture medium represents less than about 50%, 20%, 10%, or 5% of the volume of the preparation. The term “substantially free of chemical precursors or other chemicals” includes preparations in which the nucleic acid sequence is separated from chemical precursors or other chemicals which are involved in the synthesis of the nucleic acid sequence. In specific embodiments, such preparations of the nucleic acid sequence have less than about 50%, 30%, 20%, 10%, 5% (by dry weight) of chemical precursors or compounds other than the nucleic acid sequence of interest.

### 5.3 Expression of Influenza Virus Neuraminidase Polypeptide

Provided herein are vectors, including expression vectors, containing a nucleic acid sequence comprising a nucleotide sequence encoding a mutated influenza virus neuraminidase (NA) polypeptide described herein. In a specific embodiment, the vector is an expression vector that is capable of directing the expression of a nucleic acid sequence encoding a mutated influenza virus neuraminidase (NA) polypeptide. Non-limiting examples of expression vectors include, but are not limited to, plasmids and viral vectors, such as replication defective retroviruses, adenoviruses, vesicular stomatitis virus (VSV), herpes virues, Newcastle disease virus (NDV), vaccinia virus (e.g., Modified Vaccinia Ankara virus), adeno-associated viruses, plant viruses, and baculoviruses. Techniques known to one of skill in the art may be used to engineer such viral vectors to express a mutated influenza virus neuraminidase (NA) polypeptide described herein. Expression vectors also may include, without limitation, transgenic animals and non-mammalian cells/organisms, e.g., mammalian cells/organisms that have been engineered to perform mammalian N-linked glycosylation.

In some embodiments, provided herein are expression vectors encoding components of a mutated influenza virus neuraminidase (NA) polypeptide (e.g., the stem domain and the head domain, or portions of either domain). Such vectors may be used to express the components in one or more host cells and the components may be isolated and conjugated together with a linker using techniques known to one of skill in the art.

An expression vector comprises a nucleic acid sequence comprising a nucleotide sequence encoding a mutated influenza virus neuraminidase (NA) polypeptide described herein and in a form suitable for expression of the nucleic acid sequence in a host cell. In a specific embodiment, an expression vector includes one or more regulatory sequences, selected on the basis of the host cells to be used for expression, which is operably linked to the nucleic acid to be expressed. Within an expression vector, “operably linked” is intended to mean that a nucleic acid sequence of interest is linked to the regulatory sequence(s) in a manner which allows for expression of the nucleic acid sequence (e.g., in an in vitro transcription/translation system or in a host cell when the vector is introduced into the host cell). Regulatory sequences include promoters, enhancers and other expression control elements (e.g., polyadenylation signals). Regulatory sequences include those which direct constitutive expression of a nucleic acid in many types of host cells, those which direct expression of the nucleic acid sequence only in certain host cells (e.g., tissue-specific regulatory sequences), and those which direct the expression of the nucleic acid sequence upon stimulation with a particular agent (e.g., inducible regulatory sequences). It will be appreciated by those skilled in the art that the design of the expression vector can depend on such factors as the choice of the host cell to be transformed, the level of expression of protein desired, etc. The term “host cell” is intended to include a particular subject cell transformed or transfected with a nucleic acid sequence and the progeny or potential progeny of such a cell. Progeny of such a cell may not be identical to the parent cell transformed or transfected with the nucleic acid sequence due to mutations or environmental influences that may occur in succeeding generations or integration of the nucleic acid sequence into the host cell genome. In specific embodiments, the host cell is a cell line.

Expression vectors can be designed for expression of an influenza virus neuraminidase polypeptide (e.g., a mutated influenza virus neuraminidase (NA) polypeptide) described herein using prokaryotic (e. g., E. coli) or eukaryotic cells (e.g., insect cells (using baculovirus expression vectors, see, e.g., Treanor et al., 2007, JAMA, 297(14):1577-1582 incorporated by reference herein in its entirety), yeast cells, plant cells, algae, avian, or mammalian cells). Examples of yeast host cells include, but are not limited to S. pombe and S. cerevisiae and examples, infra. An example of avian cells includes, but is not limited to EB66 cells. Examples of mammalian host cells include, but are not limited to, A549 cells, Crucell Per.C6 cells, Vero cells, CHO cells, VERO cells, BHK cells, HeLa cells, COS cells, MDCK cells, 293 cells, 3T3 cells or WI38 cells. In certain embodiments, the hosts cells are myeloma cells, e.g., NS0 cells, 45.6 TG1.7 cells, AF-2 clone 9B5 cells, AF-2 clone 9B5 cells, J558L cells, MOPC 315 cells, MPC-11 cells, NCI-H929 cells, NP cells, NS0/1 cells, P3 NS1 Ag4 cells, P3/NS1/1-Ag4-1 cells, P3U1 cells, P3X63Ag8 cells, P3X63Ag8.653 cells, P3X63Ag8U.1 cells, RPMI 8226 cells, Sp20-Ag14 cells, U266B1 cells, X63AG8.653 cells, Y3.Ag.1.2.3 cells, and YO cells. Non-limiting examples of insect cells include Sf9, Sf21, Trichoplusia ni, Spodoptera frugiperda and Bombyx mori. In a particular embodiment, a mammalian cell culture system (e.g. Chinese hamster ovary or baby hamster kidney cells) is used for expression of an influenza virus neuraminidase polypeptide (e.g., a mutated influenza virus neuraminidase (NA) polypeptide). In another embodiment, a plant cell culture system is used for expression of an influenza virus neuraminidase polypeptide (e.g., a mutated influenza virus neuraminidase (NA) polypeptide). See, e.g., U.S. Pat. Nos. 7,504,560; 6,770,799; 6,551,820; 6,136,320; 6,034,298; 5,914,935; 5,612,487; and 5,484,719, and U.S. patent application publication Nos. 2009/0208477, 2009/0082548, 2009/0053762, 2008/0038232, 2007/0275014 and 2006/0204487 for plant cells and methods for the production of proteins utilizing plant cell culture systems. In specific embodiments, plant cell culture systems are not used for expression of an influenza virus neuraminidase polypeptide (e.g., a mutated influenza virus neuraminidase (NA) polypeptide). The host cells comprising the nucleic acids that encode the influenza virus neuraminidase (NA) polypeptides described herein (e.g., the mutated influenza virus neuraminidase (NA) polypeptides described herein) can be isolated, i.e., the cells are outside of the body of a subject. In certain embodiments, the cells are engineered to express nucleic acids that encode a mutated influenza virus neuraminidase (NA) polypeptides described herein.

In some embodiments, the cells are engineered to express a mutated influenza virus neuraminidase (NA) polypeptides described herein. In specific embodiments, the host cells are cells from a cell line. In certain embodiments, provided herein are host cells (e.g., cell lines) containing a nucleic acid sequence encoding a mutated influenza virus NA polypeptide described herein. In some embodiments, provided herein are host cells (e.g., cell lines) engineered to express a mutated influenza virus NA described herein. In accordance with such embodiments, the host cells may be isolated.

An expression vector can be introduced into host cells via conventional transformation or transfection techniques. Such techniques include, but are not limited to, calcium phosphate or calcium chloride co-precipitation, DEAE-dextran-mediated transfection, lipofection, and electroporation. Suitable methods for transforming or transfecting host cells can be found in Sambrook et al., 1989, Molecular Cloning—A Laboratory Manual, 2nd Edition, Cold Spring Harbor Press, New York, and other laboratory manuals. In certain embodiments, a host cell is transiently transfected with an expression vector containing a nucleic acid sequence encoding a mutated influenza virus neuraminidase (NA) polypeptide. In other embodiments, a host cell is stably transfected with an expression vector containing a nucleic acid sequence encoding a mutated influenza virus neuraminidase (NA) polypeptide.

For stable transfection of mammalian cells, it is known that, depending upon the expression vector and transfection technique used, only a small fraction of cells may integrate the foreign DNA into their genome. In order to identify and select these integrants, a nucleic acid that encodes a selectable marker (e.g., for resistance to antibiotics) is generally introduced into the host cells along with the nucleic acid of interest. Examples of selectable markers include those which confer resistance to drugs, such as G418, hygromycin and methotrexate. Cells stably transfected with the introduced nucleic acid sequence can be identified by drug selection (e.g., cells that have incorporated the selectable marker gene will survive, while the other cells die).

As an alternative to recombinant expression of a mutated influenza virus neuraminidase (NA) polypeptide using a host cell, an expression vector containing a nucleic acid sequence encoding a mutated influenza virus neuraminidase (NA) polypeptide can be transcribed and translated in vitro using, e.g., T7 promoter regulatory sequences and T7 polymerase. In a specific embodiment, a coupled transcription/translation system, such as Promega TNT®, or a cell lysate or cell extract comprising the components necessary for transcription and translation may be used to produce a mutated influenza virus neuraminidase (NA) polypeptide.

Once a mutated influenza virus neuraminidase (NA) polypeptide has been produced, it may be isolated or purified by any method known in the art for isolation or purification of a protein, for example, by chromatography (e.g., ion exchange, affinity, particularly by affinity for the specific antigen, by Protein A, and sizing column chromatography), centrifugation, differential solubility, or by any other standard technique for the isolation or purification of proteins.

Accordingly, provided herein are methods for producing a mutated influenza virus neuraminidase (NA) polypeptide. In one embodiment, the method comprises culturing a host cell containing a nucleic acid sequence comprising a nucleotide sequence encoding the polypeptide in a suitable medium such that the polypeptide is produced. In some embodiments, the method further comprises isolating the polypeptide from the medium or the host cell.

In one embodiment, provided herein are methods for producing a virus (e.g., an influenza virus (see Section 5.4, infra) or a non-influenza virus vector (e.g., a baculovirus)) described herein, comprising propagating the virus in any substrate that allows the virus to grow to titers that permit their use in accordance with the methods described herein. Also provided herein are methods for producing a virus (e.g., an influenza virus (see Section 5.4, infra) or a non-influenza virus vector (e.g., a baculovirus)) comprising a mutated influenza virus neuraminidase (NA) polypeptide described herein, comprising propagating the virus in any substrate that allows the virus to grow to titers that permit their use in accordance with the methods described herein. In some embodiments, the methods further comprise isolating or purifying the virus. In one embodiment, the substrate allows the viruses to grow to titers comparable to those determined for the corresponding wild-type viruses. In a specific embodiment, the virus is propagated in embryonated eggs (e.g., chicken eggs). In a specific embodiment, the virus is propagated in 8 day old, 9-day old, 8-10 day old, 10 day old, 11-day old, 10-12 day old, or 12-day old embryonated eggs (e.g., chicken eggs). In some embodiments, the virus is propagated in embryonated eggs (e.g., chicken eggs) that are interferon (IFN)-deficient. In certain embodiments, the virus is propagated in MDCK cells, Vero cells, 293T cells, or other cell lines known in the art. See, e.g., Section 5.3, supra, for examples of cell lines. In certain embodiments, the virus is propagated in cells derived from embryonated eggs. In certain embodiments, the virus is propagated in an embryonated egg (e.g., chicken eggs) and then in MDCK cells, Vero cells, 293T cells, or other cell lines known in the art.

### 5.4 Influenza Viruses

In one aspect, provided herein are influenza viruses containing a mutated influenza virus neuraminidase (NA) polypeptide described herein. In specific embodiments, the influenza viruses described are recombinantly produced. In a specific embodiment, a mutated influenza virus neuraminidase (NA) polypeptide is incorporated into the virion of the influenza virus. The influenza viruses may be conjugated to moieties that target the viruses to particular cell types, such as immune cells. In some embodiments, the virions of the influenza virus have incorporated into them or express a heterologous polypeptide in addition to a mutated influenza virus neuraminidase (NA) polypeptide. The heterologous polypeptide may be a polypeptide that has immunopotentiating activity, or that targets the influenza virus to a particular cell type, such as an antibody that binds to an antigen on a specific cell type or a ligand that binds a specific receptor on a specific cell type.

Influenza viruses containing a mutated influenza virus neuraminidase (NA) polypeptide may be produced by supplying in trans the mutated influenza virus neuraminidase (NA) polypeptide during production of virions using techniques known to one skilled in the art, such as reverse genetics and helper-free plasmid rescue. Alternatively, the replication of a parental influenza virus comprising a genome engineered to express a mutated influenza virus neuraminidase (NA) polypeptide in cells susceptible to infection with the virus, wherein neuraminidase function is provided in trans will produce progeny influenza viruses containing the mutated influenza virus neuraminidase (NA) polypeptide.

In another aspect, provided herein are influenza viruses comprising a genome engineered to express a mutated influenza virus neuraminidase (NA) polypeptide. In a specific embodiment, the genome of a parental influenza virus is engineered to encode a mutated influenza virus neuraminidase (NA) polypeptide, which is expressed by progeny influenza virus. In another specific embodiment, the genome of a parental influenza virus is engineered to encode a mutated influenza virus neuraminidase (NA) polypeptide, which is expressed and incorporated into the virions of progeny influenza virus. Thus, the progeny influenza virus resulting from the replication of the parental influenza virus contain a mutated influenza virus neuraminidase (NA) polypeptide. In specific embodiments, the parental influenza virus is an influenza A virus. In other specific embodiments, the parental influenza virus is an influenza B virus.

In some embodiments, the virions of the parental influenza virus have incorporated into them a heterologous polypeptide. In certain embodiments, the genome of a parental influenza virus is engineered to encode a heterologous polypeptide and a mutated influenza virus neuraminidase (NA) polypeptide, which are expressed by progeny influenza virus. In specific embodiments, the mutated influenza virus neuraminidase (NA) polypeptide, the heterologous polypeptide or both are incorporated into virions of the progeny influenza virus.

Since the genome of influenza A and B viruses consist of eight (8) single-stranded, negative sense segments, the genome of a parental influenza virus may be engineered to express a mutated influenza virus neuraminidase (NA) polypeptide (and any other polypeptide, such as a heterologous polypeptide) using a recombinant segment and techniques known to one skilled in the art, such a reverse genetics and helper-free plasmid rescue. In one embodiment, the recombinant segment comprises a nucleic acid sequence encoding the mutated influenza virus neuraminidase (NA) polypeptide as well as the 3′ and 5′ incorporation signals which are required for proper replication, transcription and packaging of the vRNAs (Fujii et al., 2003, Proc. Natl. Acad. Sci. USA 100:2002-2007; Zheng, et al., 1996, Virology 217:242-251, International Publication No. WO 2011/014645, all of which are incorporated by reference herein in their entireties). In a specific embodiment, the recombinant segment uses the 3′ and 5′ noncoding and/or nontranslated sequences of segments of influenza viruses that are from a different or the same type, subtype/lineage or strain as the parental influenza virus. In some embodiments, the recombinant segment comprises the 3′ noncoding region of an influenza virus NA polypeptide, the untranslated regions of an influenza virus NA polypeptide, and the 5′ non-coding region of an influenza virus NA polypeptide. In specific embodiments, the recombinant segment comprises packaging signals, such as the 5′ and 3′ non-coding regions and signal peptide of the NA segment of an influenza virus, from the same type, lineage, or strain as the influenza virus backbone. For example, if the mutated influenza virus neuraminidase (NA) polypeptide is engineered to be expressed from an influenza A virus, then the nucleotide sequence encoding the mutated influenza virus neuraminidase (NA) polypeptide comprises the 5′ and 3′ non-coding regions of the NA segment of the influenza A virus. In another example, if the mutated influenza virus neuraminidase (NA) polypeptide is engineered to be expressed from an influenza A virus, then the nucleotide sequence encoding the mutated influenza virus neuraminidase (NA) polypeptide comprises the 5′ and 3′ non-coding regions and the nucleotide sequence encoding the signal peptide of the NA segment of the influenza A virus. In certain embodiments, the recombinant segment encoding the mutated influenza virus neuraminidase (NA) polypeptide may replace the NA segment of a parental influenza virus.

In some embodiments, an NA gene segment encodes a mutated influenza virus neuraminidase (NA) polypeptide. In specific embodiments, the influenza virus NA gene segment and at least one other influenza virus gene segment comprise packaging signals that enable the influenza virus NA gene segment and at least one other gene segment to segregate together during replication of a recombinant influenza virus (see, Gao & Palese 2009, PNAS 106:15891-15896; U.S. Pat. No. 8,828,406; and International Application Publication No. WO 2011/014645, each of which is incorporated herein by reference in its entirety).

In a specific aspect, an NA segment provided herein that encodes a mutated influenza NA comprises the packaging signals of another influenza virus gene segment. In a specific embodiment, an NA segment provided herein that encodes a mutated influenza NA comprises the packaging signals of an influenza virus hemagglutinin (HA) gene segment. In another specific embodiment, an NA segment provided herein that encodes a mutated influenza NA comprises the packaging signals found in the 3′ non-coding region, 3′ proximal coding region sequence, the 5′ proximal coding region sequence and the 5′ non-coding region of an influenza virus HA gene segment, wherein any start codon in the 3′ proximal coding region of the first type of influenza virus gene segment is mutated, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, an NA segment provided herein that encodes a mutated influenza virus NA comprises the packaging signals described in FIGS. 4A-4B of International Patent Application Publication No. WO 2011/014645 and U.S. Pat. No. 8,828,406, each of which is incorporated herein in its entirety. In another specific embodiment, an NA segment provided herein, which encodes a mutated influenza virus NA, comprises the sequence set forth in SEQ ID NO: 27. In another specific embodiment, an NA segment provided herein, which encodes a mutated influenza virus NA, comprises the sequence set forth in SEQ ID NO: 28.

In a specific aspect, provided herein are influenza viruses comprising a first chimeric influenza virus gene segment and a second chimeric influenza virus gene segment, wherein (a) the first chimeric influenza virus gene segment encodes a mutated influenza virus NA described herein and the first chimeric influenza virus gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the mutated influenza virus NA polypeptide, (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the 3′ proximal nucleotides, the 5′ proximal nucleotides, or both in the open reading frames are mutated (e.g., substituted). In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. A person skilled in the art would be able to determine the non-coding regions, proximal coding regions, open reading frames, the proximal nucleotides of the influenza virus NA and HA gene segments using techniques and information known to one of skill in the art, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, any start codon in the 3′ proximal coding region of the NA or HA influenza virus gene segment is mutated from ATG to TTG. In a specific embodiment, the NA open reading frame and HA open reading frame are from one strain or subtype of influenza virus and the packaging signals of the chimeric gene segments comprising those open reading frames are from a different strain or subtype of influenza virus. For example, the NA and HA open reading frames may be from A/Hong Kong/4801/2014 (HK14) and the packaging signals may be from A/Puerto Rico/8/1934 (PR8), such as described in Section 6, infra. See, e.g., International Patent Application Publication No. WO 2011/014645; Gao & Palese 2009, PNAS 106:15891-15896; U.S. Pat. No. 8,828,406 for methods of producing such influenza viruses, each of which is incorporated herein in its entirety. In one embodiment, provided herein is an influenza virus comprising a first chimeric influenza virus gene segment and a second chimeric influenza virus gene segment, wherein the first chimeric influenza virus gene segment comprises the packaging signals described in FIGS. 4A-4B of International Patent Application Publication No. WO 2011/014645 and the open reading frame of a mutated influenza virus NA polypeptide described herein, and wherein the second chimeric influenza virus gene segment comprises the packaging signals described in FIGS. 32A-32C of International Patent Application Publication No. WO 2011/014645 and the open reading frame encoding for an influenza virus HA. In a specific embodiment, provided herein is an influenza virus comprising a first chimeric influenza virus gene segment and a second chimeric influenza virus gene segment, wherein the first chimeric influenza virus gene segment comprises the nucleotide sequence set forth in SEQ ID NO:27, and wherein the second chimeric influenza virus gene segment comprises the nucleotide sequence set forth in SEQ ID NO:23. In another specific embodiment, provided herein is an influenza virus comprising a first chimeric influenza virus gene segment and a second chimeric influenza virus gene segment, wherein the first chimeric influenza virus gene segment comprises the nucleotide sequence set forth in SEQ ID NO:28, and wherein the second chimeric influenza virus gene segment comprises the nucleotide sequence set forth in SEQ ID NO:25. In specific embodiments, the influenza virus described is recombinantly produced. In another specific embodiment, a mutated influenza virus neuraminidase (NA) polypeptide is incorporated into the virion of the influenza virus.

In one embodiment, provided herein are influenza viruses comprising a first chimeric influenza virus gene segment, a second chimeric influenza virus gene segment, and influenza virus NS, PB1, PB2, PA, M, and NP gene segments, wherein (a) the first chimeric influenza virus gene segment encodes a mutated influenza virus NA described herein and the first chimeric influenza virus gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the mutated influenza virus NA polypeptide, (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the 3′ proximal nucleotides, the 5′ proximal nucleotides, or both in the open reading frames are mutated (e.g., substituted). In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. A person skilled in the art would be able to determine the non-coding regions, proximal coding regions, open reading frames, the proximal nucleotides of the influenza virus NA and HA gene segments using techniques and information known to one of skill in the art, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, any start codon in the 3′ proximal coding region of the NA or HA influenza virus gene segment is mutated from ATG to TTG. In another specific embodiment, the NA open reading frame and HA open reading frame are from one strain or subtype of influenza virus and the packaging signals of the chimeric gene segments comprising those open reading frames are from a different strain or subtype of influenza virus. For example, the NA and HA open reading frames may be from A/Hong Kong/4801/2014 (HK14) and the packaging signals may be from A/Puerto Rico/8/1934 (PR8), such as described in Section 6, infra. In a particular embodiment, the NA open reading frame and HA open reading frame are from one strain or subtype of influenza virus and the packaging signals of the chimeric gene segments comprising those open reading frames are from a different strain or subtype of influenza virus and those packaging signals from the same strain or subtype of influenza virus as influenza virus NS, PB1, PB2, PA, M, and NP gene segments. See, e.g., International Patent Application Publication No. WO 2011/014645; Gao & Palese 2009, PNAS 106:15891-15896; U.S. Pat. No. 8,828,406 for methods of producing such influenza viruses, each of which is incorporated herein in its entirety. In a specific embodiment, provided herein is an influenza virus comprising a first chimeric influenza virus gene segment, a second chimeric influenza virus gene segment and influenza virus NS, PB1, PB2, PA, M, and NP gene segments, wherein the first chimeric influenza virus gene segment comprises the nucleotide sequence set forth in SEQ ID NO:27, and wherein the second chimeric influenza virus gene segment comprises the nucleotide sequence set forth in SEQ ID NO:23. In another specific embodiment, provided herein is an influenza virus comprising a first chimeric influenza virus gene segment, a second chimeric influenza virus gene segment and influenza virus NS, PB1, PB2, PA, M, and NP gene segments, wherein the first chimeric influenza virus gene segment comprises the nucleotide sequence set forth in SEQ ID NO:28, and wherein the second chimeric influenza virus gene segment comprises the nucleotide sequence set forth in SEQ ID NO:25. In specific embodiments, the influenza virus described is recombinantly produced. In another specific embodiment, a mutated influenza virus neuraminidase (NA) polypeptide is incorporated into the virion of the influenza virus. In another specific embodiment, the influenza virus HA is incorporated into the virion of the influenza virus.

In a specific aspect, provided herein are influenza viruses comprising a first chimeric influenza virus gene segment and a second chimeric influenza virus gene segment, wherein (a) the first chimeric influenza virus gene segment encodes an influenza virus NA polypeptide and the first chimeric influenza virus gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the influenza virus NA polypeptide, (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the 3′ proximal nucleotides, the 5′ proximal nucleotides, or both in the open reading frames are mutated (e.g., substituted). In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. A person skilled in the art would be able to determine the non-coding regions, proximal coding regions, open reading frames, the proximal nucleotides of the influenza virus NA and HA gene segments using techniques and information known to one of skill in the art, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, any start codon in the 3′ proximal coding region of the NA or HA influenza virus gene segment is mutated from ATG to TTG. In another specific embodiment, the NA open reading frame and HA open reading frame are from one strain or subtype of influenza virus and the packaging signals of the chimeric gene segments comprising those open reading frames are from a different strain or subtype of influenza virus. For example, the NA and HA open reading frames may be from A/Hong Kong/4801/2014 (HK14) and the packaging signals may be from A/Puerto Rico/8/1934 (PR8), such as described in Section 6, infra. In a particular embodiment, the NA open reading frame and HA open reading frame are from one strain or subtype of influenza virus and the packaging signals of the chimeric gene segments comprising those open reading frames are from a different strain or subtype of influenza virus. See, e.g., International Patent Application Publication No. WO 2011/014645; Gao & Palese 2009, PNAS 106:15891-15896; U.S. Pat. No. 8,828,406 for methods of producing such influenza viruses, each of which is incorporated herein in its entirety. In one embodiment, provided herein is an influenza virus comprising a first chimeric influenza virus gene segment and a second chimeric influenza virus gene segment, wherein the first chimeric influenza virus gene segment comprises the packaging signals described in FIGS. 4A-4B of International Patent Application Publication No. WO 2011/014645 and the open reading frame of an influenza virus NA, and wherein the second chimeric influenza virus gene segment comprises the packaging signals described in FIGS. 32A-32C of International Patent Application Publication No. WO 2011/014645 and the open reading frame encoding for an influenza virus HA polypeptide. In a specific embodiment, provided herein is an influenza virus comprising a first chimeric influenza virus gene segment and a second chimeric influenza virus gene segment, wherein the first chimeric influenza virus gene segment comprises the nucleotide sequence set forth in SEQ ID NO:24, and wherein the second chimeric influenza virus gene segment comprises the nucleotide sequence set forth in SEQ ID NO:23. In another specific embodiment, provided herein is an influenza virus comprising a first chimeric influenza virus gene segment and a second chimeric influenza virus gene segment, wherein the first chimeric influenza virus gene segment comprises the nucleotide sequence set forth in SEQ ID NO:26, and wherein the second chimeric influenza virus gene segment comprises the nucleotide sequence set forth in SEQ ID NO:25. In specific embodiments, the influenza virus described is recombinantly produced. In another specific embodiment, the influenza virus neuraminidase (NA) polypeptide is incorporated into the virion of the influenza virus. In another specific embodiment, the influenza virus HA is incorporated into the virion of the influenza virus.

In one embodiment, provided herein are influenza viruses comprising a first chimeric influenza virus gene segment, a second chimeric influenza virus gene segment, and influenza virus NS, PB1, PB2, PA, M, and NP gene segments, wherein (a) the first chimeric influenza virus gene segment encodes an influenza virus NA polypeptide and the first chimeric influenza virus gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the influenza virus NA polypeptide, (iv) a 5′ proximal coding region of the HA influenza virus gene segment; and (v) the 5′ non-coding region of the HA influenza virus gene segment; and (b) the second chimeric influenza virus gene segment encodes an influenza virus HA and the second chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, the term “3′ proximal coding region” in context of an influenza virus gene segment refers to the first 5 to 450 nucleotides from the 3′ end of the coding region of an influenza virus gene segment, or any integer between 5 and 450. In certain embodiments, the term “5′ proximal coding region” in context of an influenza virus gene segment refers to the first 5 to 450 nucleotides from the 5′ end of the coding region of an influenza virus gene segment, or any integer between 5 and 450. In certain embodiments, the 3′ proximal nucleotides, the 5′ proximal nucleotides, or both in the open reading frames are mutated (e.g., substituted). In certain embodiments, the term “3′ proximal nucleotides” refers to 1, 2, 3, 4, 5, 6, 7, 8, 9, 10, 11, 12, 13, 14, 15, 16, 17, 18, 19, 20 or more nucleotides within the first 20 to 250 nucleotides of an open reading frame beginning from the start codon towards the 5′ end of the open reading frame. In certain embodiments, the term “5′ proximal nucleotides” refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides within the first 30 to 250 nucleotides of an open reading frame beginning from the stop codon towards the 3′ end of the open reading frame. In a specific embodiment, the mutations introduced into the 3′ and/or 5′ proximal nucleotides of the open reading frame of the influenza virus gene segment(s) are silent or synonymous mutations. In particular embodiments, the silent or synonymous mutations are in regions implicated in genome packaging in order to abrogate their residual packaging function. A person skilled in the art would be able to determine the non-coding regions, proximal coding regions, open reading frames, the proximal nucleotides of the influenza virus NA and HA gene segments using techniques and information known to one of skill in the art, such as described in, e.g., International Patent Application Publication No. WO 2011/014645; Gao & Palese 2009, PNAS 106:15891-15896; U.S. Pat. No. 8,828,406, each of which is incorporated herein in its entirety. In another specific embodiment, any start codon in the 3′ proximal coding region of the NA or HA influenza virus gene segment is mutated from ATG to TTG. In another specific embodiment, the NA open reading frame and HA open reading frame are from one strain or subtype of influenza virus and the packaging signals of the chimeric gene segments comprising those open reading frames are from a different strain or subtype of influenza virus. For example, the NA and HA open reading frames may be from A/Hong Kong/4801/2014 (HK14) and the packaging signals may be from A/Puerto Rico/8/1934 (PR8), such as described in Section 6, infra. In a particular embodiment, the NA open reading frame and HA open reading frame are from one strain or subtype of influenza virus and the packaging signals of the chimeric gene segments comprising those open reading frames are from a different strain or subtype of influenza virus and those packaging signals from the same strain or subtype of influenza virus as influenza virus NS, PB1, PB2, PA, M, and NP gene segments. See, e.g., International Patent Application Publication No. WO 2011/014645; Gao & Palese 2009, PNAS 106:15891-15896; U.S. Pat. No. 8,828,406 for methods of producing such influenza viruses, each of which is incorporated herein in its entirety. In one embodiment, provided herein is an influenza virus comprising a first chimeric influenza virus gene segment and a second chimeric influenza virus gene segment, wherein the first chimeric influenza virus gene segment comprises the packaging signals described in FIGS. 4A-4B of International Patent Application Publication No. WO 2011/014645 and the open reading frame of an influenza virus NA, and wherein the second chimeric influenza virus gene segment comprises the packaging signals described in FIGS. 32A-32C of International Patent Application Publication No. WO 2011/014645 and the open reading frame encoding for an influenza virus HA polypeptide. In a specific embodiment, provided herein is an influenza virus comprising a first chimeric influenza virus gene segment and a second chimeric influenza virus gene segment, wherein the first chimeric influenza virus gene segment comprises the nucleotide sequence set forth in SEQ ID NO:24, and wherein the second chimeric influenza virus gene segment comprises the nucleotide sequence set forth in SEQ ID NO:23. In another specific embodiment, provided herein is an influenza virus comprising a first chimeric influenza virus gene segment and a second chimeric influenza virus gene segment, wherein the first chimeric influenza virus gene segment comprises the nucleotide sequence set forth in SEQ ID NO:26, and wherein the second chimeric influenza virus gene segment comprises the nucleotide sequence set forth in SEQ ID NO:25. In specific embodiments, the influenza virus described is recombinantly produced. In another specific embodiment, the influenza virus neuraminidase (NA) polypeptide is incorporated into the virion of the influenza virus. In another specific embodiment, the influenza virus HA is incorporated into the virion of the influenza virus.

In a specific embodiment, provided herein is an influenza virus comprising the segments described in Section 6.2. In a specific embodiment, provided herein is an influenza virus comprising the segments described in Section 6.1 or 6.3. In a specific embodiment, provided herein is an influenza virus comprising the segments described in Section 6.4. In another embodiment, provided herein is an influenza virus described in Section 6, infra.

In some embodiments, the genome of a parental influenza virus may be engineered to express a mutated influenza virus neuraminidase (NA) polypeptide using a recombinant segment that is bicistronic. Bicistronic techniques allow the engineering of coding sequences of multiple proteins into a single mRNA through the use of internal ribosome entry site (IRES) sequences. IRES sequences direct the internal recruitment of ribosomes to the RNA molecule and allow downstream translation in a cap independent manner. Briefly, a coding region of one protein is inserted into the open reading frame (ORF) of a second protein. The insertion is flanked by an IRES and any untranslated signal sequences necessary for proper expression and/or function. The insertion must not disrupt the ORF, polyadenylation or transcriptional promoters of the second protein (see, e.g., Garcia-Sastre et al., 1994, J. Virol. 68:6254-6261 and Garcia-Sastre et al., 1994 Dev. Biol. Stand. 82:237-246, each of which is hereby incorporated by reference in its entirety). See also, e.g., U.S. Pat. Nos. 6,887,699, 6,001,634, 5,854,037 and 5,820,871, each of which is incorporated herein by reference in its entirety. Any IRES known in the art or described herein may be used in accordance with the invention (e.g., the IRES of BiP gene, nucleotides 372 to 592 of GenBank database entry HUMGRP78; or the IRES of encephalomyocarditis virus (EMCV), nucleotides 1430-2115 of GenBank database entry CQ867238.). Thus, in certain embodiments, a parental influenza virus is engineered to contain a bicistronic RNA segment that expresses the mutated influenza virus neuraminidase (NA) polypeptide and another polypeptide, such as a gene expressed by the parental influenza virus. In some embodiments, the parental influenza virus gene is the NA gene.

Techniques known to one skilled in the art may be used to produce an influenza virus containing an influenza virus neuraminidase polypeptide (e.g., mutated influenza virus neuraminidase (NA) polypeptide) and an influenza virus comprising a genome engineered to express an influenza virus neuraminidase polypeptide (e.g., mutated influenza virus neuraminidase (NA) polypeptide). For example, reverse genetics techniques may be used to generate such an influenza virus. Briefly, reverse genetics techniques generally involve the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in European Patent Publication EP 0702085A1, published Feb. 20, 1996; in U.S. patent application Ser. No. 09/152,845; in International Patent Publications PCT WO 97/12032 published Apr. 3, 1997; WO 96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

Alternatively, helper-free plasmid technology may be used to produce an influenza virus containing an influenza virus neuraminidase polypeptide (e.g., mutated influenza virus neuraminidase (NA) polypeptide) and an influenza virus comprising a genome engineered to express an influenza virus neuraminidase polypeptide (e.g., mutated influenza virus neuraminidase (NA) polypeptide). Briefly, full length cDNAs of viral segments are amplified using PCR with primers that include unique restriction sites, which allow the insertion of the PCR product into the plasmid vector (Flandorfer et al., 2003, J. Virol. 77:9116-9123; Nakaya et al., 2001, J. Virol. 75:11868-11873; both of which are incorporated herein by reference in their entireties). The plasmid vector is designed so that an exact negative (vRNA sense) transcript is expressed. For example, the plasmid vector may be designed to position the PCR product between a truncated human RNA polymerase I promoter and a hepatitis delta virus ribozyme sequence such that an exact negative (vRNA sense) transcript is produced from the polymerase I promoter. Separate plasmid vectors comprising each viral segment as well as expression vectors comprising necessary viral proteins may be transfected into cells leading to production of recombinant viral particles. In another example, plasmid vectors from which both the viral genomic RNA and mRNA encoding the necessary viral proteins are expressed may be used. For a detailed description of helper-free plasmid technology see, e.g., International Publication No. WO 01/04333; U.S. Pat. Nos. 6,951,754, 7,384,774, 6,649,372, and 7,312,064; Fodor et al., 1999, J. Virol. 73:9679-9682; Quinlivan et al., 2005, J. Virol. 79:8431-8439; Hoffmann et al., 2000, Proc. Natl. Acad. Sci. USA 97:6108-6113; and Neumann et al., 1999, Proc. Natl. Acad. Sci. USA 96:9345-9350, each of which is incorporated herein by reference in its entirety. In a specific embodiment, a method analogous to that described in Section 6 is used to construct a mutated influenza virus neuraminidase (NA) polypeptide. In a specific embodiment, a method analogous to that described in Section 6 is used to construct an influenza virus containing and expressing an influenza virus neuraminidase (NA) polypeptide. In a specific embodiment, a method analogous to that described in Section 6 is used to construct and propagate a mutated influenza virus neuraminidase (NA) polypeptide. In another specific embodiment, a method analogous to that described in Section 6.1, 6.2, 6.3 or 6.4 is used to construct and propagate an influenza virus.

In some embodiments, a recombinant influenza virus is produced by reverse genetics, using a DNA plasmid(s) that expresses a mutated influenza virus neuraminidase polypeptide, which is co-transfected with plasmids for the other 7 genes of influenza virus in a mammalian cell line, such as, e.g., HEK293T cells or other mammalian cell lines described herein. In a specific embodiment, the recombinant influenza virus replicates in embryonated chicken eggs without apparent disadvantages over the influenza viruses that do not have a mutated influenza virus neuraminidase polypeptide. In certain embodiments, a recombinant influenza virus is produced by reverse genetics, using a DNA plasmid(s) comprising a first chimeric influenza virus gene segment described herein and a DNA plasmid(s) comprising a second chimeric influenza virus gene segment described herein, which is co-transfected with plasmids for the other 6 genes of influenza virus in a mammalian cell line, such as, e.g., HEK293T cells or other mammalian cell lines described herein. In a specific embodiment, the recombinant influenza virus replicates in embryonated chicken eggs without apparent disadvantages over the influenza viruses that do not have packaging signals of the NA and HA gene segments swapped.

The influenza viruses described herein may be propagated in any substrate that allows the virus to grow to titers that permit their use in accordance with the methods described herein. Thus, in certain embodiments, provided herein is a method for producing a virus described herein comprising propagating the virus in a substrate. In one embodiment, the substrate allows the viruses to grow to titers comparable to those determined for the corresponding wild-type viruses. In certain embodiments, the substrate is one which is biologically relevant to the influenza virus or to the virus from which the NA function is derived. In a specific embodiment, an attenuated influenza virus by virtue of, e.g., a mutation in the NS1 gene, may be propagated in an IFN-deficient substrate. For example, a suitable IFN-deficient substrate may be one that is defective in its ability to produce or respond to interferon, or is one which an IFN-deficient substrate may be used for the growth of any number of viruses which may require interferon-deficient growth environment. See, for example, U.S. Pat. No. 6,573,079, issued Jun. 3, 2003, U.S. Pat. No. 6,852,522, issued Feb. 8, 2005, and U.S. Pat. No. 7,494,808, issued Feb. 24, 2009, the entire contents of each of which is incorporated herein by reference in its entirety. In a specific embodiment, the virus is propagated in embryonated eggs (e.g., chicken eggs). In a specific embodiment, the virus is propagated in 8 day old, 9-day old, 8-10 day old, 10 day old, 11-day old, 10-12 day old, or 12-day old embryonated eggs (e.g., chicken eggs). In some embodiments, the virus is propagated in embryonated eggs (e.g., chicken eggs) that are IFN-deficient. In certain embodiments, the virus is propagated in MDCK cells, Vero cells, 293T cells, or other cell lines known in the art. See, e.g., Section 5.3, supra, for examples of cell lines. In certain embodiments, the virus is propagated in cells derived from embryonated eggs.

The influenza viruses described herein may be isolated and purified by any method known to those of skill in the art. In one embodiment, the virus is removed from cell culture and separated from cellular components, typically by well known clarification procedures, e.g., such as gradient centrifugation and column chromatography, and may be further purified as desired using procedures well known to those skilled in the art, e.g., plaque assays.

In certain embodiments, the influenza viruses, or influenza virus polypeptides, genes or genome segments for use as described herein are obtained or derived from an influenza A virus. In certain embodiments, the influenza viruses, or influenza virus polypeptides, genes or genome segments for use as described herein are obtained or derived from a single influenza A virus subtype/lineage or strain. In other embodiments, the influenza viruses, or influenza virus polypeptides, genes or genome segments for use as described herein are obtained or derived from two or more influenza A virus subtypes or strains. In a specific embodiment, the influenza A virus is an influenza virus of the H1, H2, H3, H4, H5, H6, H7, H8, H9, H10, H11, H12, H13, H14, H15, H16, H17, or H18 subtype. In a specific embodiment, the influenza A virus is an influenza virus of the H2, H4, H5, H6, H7, H8, H9, H10, H11, H12, H13, H14, H15, H16, H17, or H18 subtype. In a specific embodiment, the influenza A virus is an influenza virus of the H1 or H3 subtype. In a specific embodiment, the influenza A virus is an influenza virus of the H5, H7, H9 or H10 subtype.

Non-limiting examples of influenza A viruses include subtype H10N4, subtype H10N5, subtype H10N8, subtype, H14N5, subtype H10N7, subtype H10N8, subtype H10N9, subtype H11N1, subtype H11N13, subtype H11N2, subtype H11N4, subtype H11N6, subtype H11N8, subtype H11N9, subtype H12N1, subtype H12N4, subtype H12N5, subtype H12N8, subtype H13N2, subtype H13N3, subtype H13N6, subtype H13N7, subtype H14N5, subtype H14N6, subtype H15N8, subtype H15N9, subtype H16N3, subtype H1N1, subtype H1N2, subtype H1N3, subtype H1N6, subtype H1N9, subtype H2N1, subtype H2N2, subtype H2N3, subtype H2N5, subtype H2N7, subtype H2N8, subtype H2N9, subtype H3N1, subtype H3N2, subtype H3N3, subtype H3N4, subtype H3N5, subtype H3N6, subtype H3N8, subtype H3N9, subtype H4N1, subtype H4N2, subtype H4N3, subtype H4N4, subtype H4N5, subtype H4N6, subtype H4N8, subtype H4N9, subtype H5N1, subtype H5N2, subtype H5N3, subtype H5N4, subtype H5N6, subtype H5N7, subtype H5N8, subtype H5N9, subtype H6N1, subtype H6N2, subtype H6N3, subtype H6N4, subtype H6N5, subtype H6N6, subtype H6N7, subtype H6N8, subtype H6N9, subtype H7N1, subtype H7N2, subtype H7N3, subtype H7N4, subtype H7N5, subtype H7N7, subtype H7N8, subtype H7N9, subtype H8N4, subtype H8N5, subtype H9N1, subtype H9N2, subtype H9N3, subtype H9N5, subtype H9N6, subtype H9N7, subtype H9N8, and subtype H9N9. In certain embodiments, an influenza A virus is of subtype H6N1, subtype H7N1, subtype H7N3, or subtype H9N2.

Specific examples of strains of influenza A virus include, but are not limited to: A/Victoria/361/2011 (H3N2); A/California/4/2009 (H1N1); A/California/7/2009 (H1N1); A/Perth/16/2009 (H3N2); A/Brisbane/59/2007 (H1N1); A/Brisbane/10/2007 (H3N2); A/sw/Iowa/15/30 (H1N1); A/WSN/33 (H1N1); A/eq/Prague/1/56 (H7N7); A/PR/8/34; A/mallard/Potsdam/178-4/83 (H2N2); A/herring gull/DE/712/88 (H16N3); A/sw/Hong Kong/168/1993 (H1N1); A/mallard/Alberta/211/98 (H1N1); A/shorebird/Delaware/168/06 (H16N3); A/sw/Netherlands/25/80 (H1N1); A/sw/Germany/2/81 (H1N1); A/sw/Hannover/1/81 (H1N1); A/sw/Potsdam/1/81 (H1N1); A/sw/Potsdam/15/81 (H1N1); A/sw/Potsdam/268/81 (H1N1); A/sw/Finistere/2899/82 (H1N1); A/sw/Potsdam/35/82 (H3N2); A/sw/Cote d'Armor/3633/84 (H3N2); A/sw/Gent/1/84 (H3N2); A/sw/Netherlands/12/85 (H1N1); A/sw/Karrenzien/2/87 (H3N2); A/sw/Schwerin/103/89 (H1N1); A/turkey/Germany/3/91 (H1N1); A/sw/Germany/8533/91 (H1N1); A/sw/Belgium/220/92 (H3N2); A/sw/Gent/V230/92 (H1N1); A/sw/Leipzig/145/92 (H3N2); A/sw/Re220/92 hp (H3N2); A/sw/Bakum/909/93 (H3N2); A/sw/Schleswig-Holstein/1/93 (H1N1); A/sw/Scotland/419440/94 (H1N2); A/sw/Bakum/5/95 (H1N1); A/sw/Best/5C/96 (H1N1); A/sw/England/17394/96 (H1N2); A/sw/Jena/5/96 (H3N2); A/sw/Oedenrode/7C/96 (H3N2); A/sw/Lohne/1/97 (H3N2); A/sw/Cote d'Armor/790/97 (H1N2); A/sw/Bakum/1362/98 (H3N2); A/sw/Italy/1521/98 (H1N2); A/sw/Italy/1553-2/98 (H3N2); A/sw/Italy/1566/98 (H1N1); A/sw/Italy/1589/98 (H1N1); A/sw/Bakum/8602/99 (H3N2); A/sw/Cotes d'Armor/604/99 (H1N2); A/sw/Cote d'Armor/1482/99 (H1N1); A/sw/Gent/7625/99 (H1N2); A/Hong Kong/1774/99 (H3N2); A/sw/Hong Kong/5190/99 (H3N2); A/sw/Hong Kong/5200/99 (H3N2); A/sw/Hong Kong/5212/99 (H3N2); A/sw/Ille et Villaine/1455/99 (H1N1); A/sw/Italy/1654-1/99 (H1N2); A/sw/Italy/2034/99 (H1N1); A/sw/Italy/2064/99 (H1N2); A/sw/Berlin/1578/00 (H3N2); A/sw/Bakum/1832/00 (H1N2); A/sw/Bakum/1833/00 (H1N2); A/sw/Cote d'Armor/800/00 (H1N2); A/sw/Hong Kong/7982/00 (H3N2); A/sw/Italy/1081/00 (H1N2); A/sw/Belzig/2/01 (H1N1); A/sw/Belzig/54/01 (H3N2); A/sw/Hong Kong/9296/01 (H3N2); A/sw/Hong Kong/9745/01 (H3N2); A/sw/Spain/33601/01 (H3N2); A/sw/Hong Kong/1144/02 (H3N2); A/sw/Hong Kong/1197/02 (H3N2); A/sw/Spain/39139/02 (H3N2); A/sw/Spain/42386/02 (H3N2); A/Switzerland/8808/2002 (H1N1); A/sw/Bakum/1769/03 (H3N2); A/sw/Bissendorf/IDT1864/03 (H3N2); A/sw/Ehren/IDT2570/03 (H1N2); A/sw/Gescher/IDT2702/03 (H1N2); A/sw/Haselünne/2617/03 hp (H1N1); A/sw/Löningen/IDT2530/03 (H1N2); A/sw/IVD/IDT2674/03 (H1N2); A/sw/Nordkirchen/IDT1993/03 (H3N2); A/sw/Nordwalde/IDT2197/03 (H1N2); A/sw/Norden/IDT2308/03 (H1N2); A/sw/Spain/50047/03 (H1N1); A/sw/Spain/51915/03 (H1N1); A/sw/Vechta/2623/03 (H1N1); A/sw/Visbek/IDT2869/03 (H1N2); A/sw/Waltersdorf/IDT2527/03 (H1N2); A/sw/Damme/IDT2890/04 (H3N2); A/sw/Geldern/IDT2888/04 (H1N1); A/sw/Granstedt/IDT3475/04 (H1N2); A/sw/Greven/IDT2889/04 (H1N1); A/sw/Gudensberg/IDT2930/04 (H1N2); A/sw/Gudensberg/IDT2931/04 (H1N2); A/sw/Lohne/IDT3357/04 (H3N2); A/swNortrup/IDT3685/04 (H1N2); A/sw/Seesen/IDT3055/04 (H3N2); A/sw/Spain/53207/04 (H1N1); A/sw/Spain/54008/04 (H3N2); A/sw/Stolzenau/IDT3296/04 (H1N2); A/sw/Wedel/IDT2965/04 (H1N1); A/sw/Bad Griesbach/IDT4191/05 (H3N2); A/sw/Cloppenburg/IDT4777/05 (H1N2); A/sw/Dötlingen/IDT3780/05 (H1N2); A/sw/Dötlingen/IDT4735/05 (H1N2); A/sw/Egglham/IDT5250/05 (H3N2); A/sw/Harkenblek/IDT4097/05 (H3N2); A/sw/Hertzen/IDT4317/05 (H3N2); A/sw/Krogel/IDT4192/05 (H1N1); A/sw/Laer/IDT3893/05 (H1N1); A/sw/Laer/IDT4126/05 (H3N2); A/sw/Merzen/IDT4114/05 (H3N2); A/sw/Muesleringen-S./DT4263/05 (H3N2); A/sw/Osterhofen/IDT4004/05 (H3N2); A/sw/Sprenge/IDT3805/05 (H1N2); A/sw/Stadtlohn/IDT3853/05 (H1N2); A/sw/Voglarn/IDT4096/05 (H1N1); A/sw/Wohlerst/IDT4093/05 (H1N1); A/sw/Bad Griesbach/IDT5604/06 (H1N1); A/sw/Herzlake/IDT5335/06 (H3N2); A/sw/Herzlake/IDT5336/06 (H3N2); A/sw/Herzlake/IDT5337/06 (H3N2); and A/wild boar/Germany/R169/2006 (H3N2).

Other specific examples of strains of influenza A virus include, but are not limited to: A/Toronto/3141/2009 (H1N1); A/Regensburg/D6/2009 (H1N1); A/Bayern/62/2009 (H1N1); A/Bayern/62/2009 (H1N1); A/Bradenburg/19/2009 (H1N1); A/Bradenburg/20/2009 (H1N1); A/Distrito Federal/2611/2009 (H1N1); A/Mato Grosso/2329/2009 (H1N1); A/Sao Paulo/1454/2009 (H1N1); A/Sao Paulo/2233/2009 (H1N1); A/Stockholm/37/2009 (H1N1); A/Stockholm/41/2009 (H1N1); A/Stockholm/45/2009 (H1N1); A/swine/Alberta/OTH-33-1/2009 (H1N1); A/swine/Alberta/OTH-33-14/2009 (H1N1); A/swine/Alberta/OTH-33-2/2009 (H1N1); A/swine/Alberta/OTH-33-21/2009 (H1N1); A/swine/Alberta/OTH-33-22/2009 (H1N1); A/swine/Alberta/OTH-33-23/2009 (H1N1); A/swine/Alberta/OTH-33-24/2009 (H1N1); A/swine/Alberta/OTH-33-25/2009 (H1N1); A/swine/Alberta/OTH-33-3/2009 (H1N1); A/swine/Alberta/OTH-33-7/2009 (H1N1); A/Beijing/502/2009 (H1N1); A/Firenze/10/2009 (H1N1); A/Hong Kong/2369/2009 (H1N1); A/Italy/85/2009 (H1N1); A/Santo Domingo/572N/2009 (H1N1); A/Catalonia/385/2009 (H1N1); A/Catalonia/386/2009 (H1N1); A/Catalonia/387/2009 (H1N1); A/Catalonia/390/2009 (H1N1); A/Catalonia/394/2009 (H1N1); A/Catalonia/397/2009 (H1N1); A/Catalonia/398/2009 (H1N1); A/Catalonia/399/2009 (H1N1); A/Sao Paulo/2303/2009 (H1N1); A/Akita/1/2009 (H1N1); A/Castro/JXP/2009 (H1N1); A/Fukushima/1/2009 (H1N1); A/Israel/276/2009 (H1N1); A/Israel/277/2009 (H1N1); A/Israel/70/2009 (H1N1); A/Iwate/1/2009 (H1N1); A/Iwate/2/2009 (H1N1); A/Kagoshima/1/2009 (H1N1); A/Osaka/180/2009 (H1N1); A/Puerto Montt/Bio87/2009 (H1 N1); A/Sao Paulo/2303/2009 (H1N1); A/Sapporo/1/2009 (H1N1); A/Stockholm/30/2009 (H1N1); A/Stockholm/31/2009 (H1N1); A/Stockholm/32/2009 (H1N1); A/Stockholm/33/2009 (H1N1); A/Stockholm/34/2009 (H1N1); A/Stockholm/35/2009 (H1N1); A/Stockholm/36/2009 (H1N1); A/Stockholm/38/2009 (H1N1); A/Stockholm/39/2009 (H1N1); A/Stockholm/40/2009 (H1N1) A/Stockholm/42/2009 (H1N1); A/Stockholm/43/2009 (H1N1); A/Stockholm/44/2009 (H1N1); A/Utsunomiya/2/2009 (H1N1); A/WRAIR/0573N/2009 (H1N1); and A/Zhejiang/DTID-ZJU01/2009 (H1N1).

In certain embodiments, the influenza viruses, or influenza virus polypeptides, genes or genome segments for use as described herein are obtained or derived from an influenza B virus. In certain embodiments, the influenza viruses, or influenza virus polypeptides, genes or genome segments for use as described herein are obtained or derived from a single influenza B virus subtype/lineage or strain. In other embodiments, the influenza viruses, or influenza virus polypeptides, genes or genome segments for use as described herein are obtained or derived from two or more influenza B virus subtypes or strains.

Non-limiting examples of influenza B viruses include strain Aichi/5/88, strain B/Brisbane/60/2008; Akita/27/2001, strain Akita/5/2001, strain Alaska/16/2000, strain Alaska/1777/2005, strain Argentina/69/2001, strain Arizona/146/2005, strain Arizona/148/2005, strain Bangkok/163/90, strain Bangkok/34/99, strain Bangkok/460/03, strain Bangkok/54/99, strain Barcelona/215/03, strain Beijing/15/84, strain Beijing/184/93, strain Beijing/243/97, strain Beijing/43/75, strain Beijing/5/76, strain Beijing/76/98, strain Belgium/WV106/2002, strain Belgium/WV107/2002, strain Belgium/WV109/2002, strain Belgium/WV114/2002, strain Belgium/WV122/2002, strain Bonn/43, strain Brazil/952/2001, strain Bucharest/795/03, strain Buenos Aires/161/00), strain Buenos Aires/9/95, strain Buenos Aires/SW16/97, strain Buenos Aires/VL518/99, strain Canada/464/2001, strain Canada/464/2002, strain Chaco/366/00, strain Chaco/R113/00, strain Cheju/303/03, strain Chiba/447/98, strain Chongqing/3/2000, strain clinical isolate SA1 Thailand/2002, strain clinical isolate SA10 Thailand/2002, strain clinical isolate SA100 Philippines/2002, strain clinical isolate SA101 Philippines/2002, strain clinical isolate SA110 Philippines/2002), strain clinical isolate SA112 Philippines/2002, strain clinical isolate SA113 Philippines/2002, strain clinical isolate SA114 Philippines/2002, strain B/Phuket/3073/2013, strain B/Malaysia/2506/2004, strain clinical isolate SA2 Thailand/2002, strain clinical isolate SA20 Thailand/2002, strain clinical isolate SA38 Philippines/2002, strain clinical isolate SA39 Thailand/2002, strain clinical isolate SA99 Philippines/2002, strain CNIC/27/2001, strain Colorado/2597/2004, strain Cordoba/VA418/99, strain Czechoslovakia/16/89, strain Czechoslovakia/69/90, strain Daeku/10/97, strain Daeku/45/97, strain Daeku/47/97, strain Daeku/9/97, strain B/Du/4/78, strain B/Durban/39/98, strain Durban/43/98, strain Durban/44/98, strain B/Durban/52/98, strain Durban/55/98, strain Durban/56/98, strain England/1716/2005, strain England/2054/2005), strain England/23/04, strain Finland/154/2002, strain Finland/159/2002, strain Finland/160/2002, strain Finland/161/2002, strain Finland/162/03, strain Finland/162/2002, strain Finland/162/91, strain Finland/164/2003, strain Finland/172/91, strain Finland/173/2003, strain Finland/176/2003, strain Finland/184/91, strain Finland/188/2003, strain Finland/190/2003, strain Finland/220/2003, strain Finland/WV5/2002, strain Fujian/36/82, strain Geneva/5079/03, strain Genoa/11/02, strain Genoa/2/02, strain Genoa/21/02, strain Genova/54/02, strain Genova/55/02, strain Guangdong/05/94, strain Guangdong/08/93, strain Guangdong/5/94, strain Guangdong/55/89, strain Guangdong/8/93, strain Guangzhou/7/97, strain Guangzhou/86/92, strain Guangzhou/87/92, strain Gyeonggi/592/2005, strain Hannover/2/90, strain Harbin/07/94, strain Hawaii/10/2001, strain Hawaii/1990/2004, strain Hawaii/38/2001, strain Hawaii/9/2001, strain Hebei/19/94, strain Hebei/3/94), strain Henan/22/97, strain Hiroshima/23/2001, strain Hong Kong/110/99, strain Hong Kong/1115/2002, strain Hong Kong/112/2001, strain Hong Kong/123/2001, strain Hong Kong/1351/2002, strain Hong Kong/1434/2002, strain Hong Kong/147/99, strain Hong Kong/156/99, strain Hong Kong/157/99, strain Hong Kong/22/2001, strain Hong Kong/22/89, strain Hong Kong/336/2001, strain Hong Kong/666/2001, strain Hong Kong/9/89, strain Houston/1/91, strain Houston/1/96, strain Houston/2/96, strain Hunan/4/72, strain Ibaraki/2/85, strain ncheon/297/2005, strain India/3/89, strain India/77276/2001, strain Israel/95/03, strain Israel/WV187/2002, strain Japan/1224/2005, strain Jiangsu/10/03, strain Johannesburg/1/99, strain Johannesburg/96/01, strain Kadoma/1076/99, strain Kadoma/122/99, strain Kagoshima/15/94, strain Kansas/22992/99, strain Khazkov/224/91, strain Kobe/1/2002, strain, strain Kouchi/193/99, strain Lazio/1/02, strain Lee/40, strain Leningrad/129/91, strain Lissabon/2/90), strain Los Angeles/1/02, strain Lusaka/270/99, strain Lyon/1271/96, strain Malaysia/83077/2001, strain Maputo/1/99, strain Mar del Plata/595/99, strain Maryland/1/01, strain Memphis/1/01, strain Memphis/12/97-MA, strain Michigan/22572/99, strain Mie/1/93, strain Milano/1/01, strain Minsk/318/90, strain Moscow/3/03, strain Nagoya/20/99, strain Nanchang/1/00, strain Nashville/107/93, strain Nashville/45/91, strain Nebraska/2/01, strain Netherland/801/90, strain Netherlands/429/98, strain New York/1/2002, strain NIB/48/90, strain Ningxia/45/83, strain Norway/1/84, strain Oman/16299/2001, strain Osaka/1059/97, strain Osaka/983/97-V2, strain Oslo/1329/2002, strain Oslo/1846/2002, strain Panama/45/90, strain Paris/329/90, strain Parma/23/02, strain Perth/211/2001, strain Peru/1364/2004, strain Philippines/5072/2001, strain Pusan/270/99, strain Quebec/173/98, strain Quebec/465/98, strain Quebec/7/01, strain Roma/1/03, strain Saga/S172/99, strain Seoul/13/95, strain Seoul/37/91, strain Shangdong/7/97, strain Shanghai/361/2002), strain Shiga/T30/98, strain Sichuan/379/99, strain Singapore/222/79, strain Spain/WV27/2002, strain Stockholm/10/90, strain Switzerland/5441/90, strain Taiwan/0409/00, strain Taiwan/0722/02, strain Taiwan/97271/2001, strain Tehran/80/02, strain Tokyo/6/98, strain Trieste/28/02, strain Ulan Ude/4/02, strain United Kingdom/34304/99, strain USSR/100/83, strain Victoria/103/89, strain Vienna/1/99, strain Wuhan/356/2000, strain WV194/2002, strain Xuanwu/23/82, strain Yamagata/1311/2003, strain Yamagata/K500/2001, strain Alaska/12/96, strain GA/86, strain NAGASAKI/1/87, strain Tokyo/942/96, strain B/Wisconsin/1/2010; and strain Rochester/02/2001. In a specific embodiment, an influenza B virus is influenza B virus B/Phuket/3073/2013 or B/Maylasia/2506/2004.

Other examples of influenza viruses may be found elsewhere in the application, such as in, e.g., Section 6 below. In a specific embodiment, a seasonal influenza virus strain may be used.

In certain embodiments, the influenza viruses provided herein have an attenuated phenotype. In specific embodiments, the attenuated influenza virus is based on influenza A virus. In specific embodiments, the attenuated influenza virus comprises, encodes, or both, a mutated influenza virus NA polypeptide and has a backbone of an influenza A virus. In some embodiments, the attenuated influenza virus is based on influenza B virus. In specific embodiments, the attenuated influenza virus comprises, encodes, or both, a mutated influenza virus NA polypeptide and has a backbone of an influenza B virus.

In specific embodiments, attenuation of influenza virus is desired such that the virus remains, at least partially, infectious and can replicate in vivo, but only generate low titers resulting in subclinical levels of infection that are non-pathogenic. Such attenuated viruses are especially suited for embodiments described herein wherein the virus or an immunogenic composition thereof is administered to a subject to induce an immune response. Attenuation of the influenza virus can be accomplished according to any method known in the art, such as, e.g., selecting viral mutants generated by chemical mutagenesis, mutation of the genome by genetic engineering, selecting reassortant viruses that contain segments with attenuated function (e.g., truncated NS1 protein (see, e.g., Hai et al., 2008, Journal of Virology 82(21):10580-10590, which is incorporated by reference herein in its entirety) or NS1 deletion (see, e.g., Wressnigg et al., 2009, Vaccine 27:2851-2857 and U.S. Pat. Nos. 9,387,240, 8,765,139, 8,057,803, 7,588,768, 6,669,943, 10,098,945, 9,549,975, 8,999,352, 6,573,079, and 6,468,544, each of which is incorporated by reference herein in its entirety)), or selecting for conditional virus mutants (e.g., cold-adapted viruses, see, e.g., Alexandrova et al., 1990, Vaccine, 8:61-64, which is incorporated by reference herein in its entirety). Alternatively, naturally occurring attenuated influenza viruses may be used as influenza virus backbones for the influenza virus vectors.

In a specific embodiment, the influenza A virus A/Puerto Rico/8/34 strain is used as the backbone to express an influenza virus NA polypeptide described herein (e.g., a mutated influenza virus NA polypeptide described herein). In another specific embodiment, the virion of the influenza A virus A/Puerto Rico/8/34 strain contains a mutated influenza virus NA polypeptide described herein. In another specific embodiment, the influenza A virus A/Puerto Rico/8/34 strain is used to express a mutated influenza virus NA polypeptide described herein and the virion of the A/Puerto Rico/8/34 strain contains the mutated influenza virus NA polypeptide.

In a specific embodiment, an influenza A virus lacking the NS1 protein (e.g., a delNS1 virus, such as described, e.g., in U.S. Pat. No. 6,468,544; Garcia-Sastre et al., 1998, Virology 252: 324; or Mossier et al., 2013, Vaccine 31: 6194) is used as the backbone to express a mutated influenza virus NA polypeptide described herein. In another specific embodiment, the virion of an influenza virus lacking the NS1 protein (e.g., a delNS1 virus, such as described, e.g., in U.S. Pat. No. 6,468,544; Garcia-Sastre et al., 1998, Virology 252: 324; or Mossier et al., 2013, Vaccine 31: 6194) contains a mutated influenza virus NA polypeptide described herein. In another specific embodiment, an influenza virus lacking the NS1 protein (e.g., a delNS1 virus, such as described, e.g., in U.S. Pat. No. 6,468,544; Garcia-Sastre et al., 1998, Virology 252: 324; or Mossier et al., 2013, Vaccine 31: 6194) is used to express a mutated influenza virus NA polypeptide described herein and the virion of such a virus contains the mutated influenza virus NA polypeptide.

In a specific embodiment, an influenza A virus containing a truncated NS1 protein (such as described, e.g., in U.S. Pat. Nos. 9,387,240, 8,765,139, 8,057,803, 7,588,768, 6,669,943, 10,098,945, 9,549,975, 8,999,352, and 6,573,079, each of which is incorporated herein by reference in its entirety) is used as the backbone to express a mutated influenza virus NA polypeptide described herein. In another specific embodiment, the virion of an influenza virus a truncated NS1 protein (e.g., U.S. Pat. Nos. 9,387,240, 8,765,139, 8,057,803, 7,588,768, 6,669,943, 10,098,945, 9,549,975, 8,999,352, 6,573,079, each of which is incorporated herein by reference in its entirety) contains a mutated influenza virus NA polypeptide described herein. In another specific embodiment, an influenza virus lacking the NS1 protein (such as described, e.g., in U.S. Pat. Nos. 9,387,240, 8,765,139, 8,057,803, 7,588,768, 6,669,943, 10,098,945, 9,549,975, 8,999,352, and 6,573,079, each of which is incorporated herein by reference in its entirety) is used to express a mutated influenza virus NA polypeptide described herein and the virion of such a virus contains the mutated influenza virus NA polypeptide.

In a specific embodiment, a cold-adapted influenza A virus strain is used as the backbone to express an influenza virus NA polypeptide described herein (e.g., a mutated influenza virus NA polypeptide described herein). In another specific embodiment, the virion of the cold-adapted strain contains a mutated influenza virus NA polypeptide described herein. In another specific embodiment, the cold-adapted influenza A virus is used to express a mutated influenza virus NA polypeptide described herein and the virion of the cold-adapted influenza virus contains the mutated influenza virus NA polypeptide. In one embodiment, the cold-adapted influenza A virus is A/Ann Arbor/6/60. In another embodiment, the cold-adapted influenza A virus is A/Leningrad/134/17/57. In another embodiment, a seasonal influenza virus strain is used as the backbone to express a mutated influenza virus NA polypeptide described herein.

In a specific embodiment, an influenza B virus strain is used as the backbone to express an influenza virus NA polypeptide described herein (e.g., a mutated influenza virus NA polypeptide described herein). In another specific embodiment, the virion of the influenza B virus strain contains a mutated influenza virus NA polypeptide described herein. In another specific embodiment, the influenza B virus is used to express a mutated influenza virus NA polypeptide described herein and the virion of the influenza B virus contains the mutated influenza virus NA polypeptide. In one embodiment, the cold-adapted influenza A virus is B/Malyasia/2506/2004. In a specific embodiment, the influenza B virus is attenuated.

In a specific embodiment, a seasonal influenza virus strain is used as the backbone to express an influenza virus NA polypeptide described herein (e.g., a mutated influenza virus NA polypeptide described herein). In another specific embodiment, the virion of the seasonal influenza virus strain contains a mutated influenza virus NA polypeptide described herein. In another specific embodiment, the seasonal influenza virus strain is used to express a mutated influenza virus NA polypeptide described herein and the virion of the influenza B virus contains the mutated influenza virus NA polypeptide. In a specific embodiment, the seasonal influenza virus strain is attenuated.

In certain embodiments, an influenza virus comprising an influenza virus NA polypeptide described herein (e.g., a mutated influenza virus NA polypeptide described herein) has one, two, or more of the functions of an influenza virus comprising a wild-type influenza virus NA. A non-limiting example of a function of a wild-type influenza virus NA include cleavage of sialic acid. In a specific embodiment, an influenza virus comprising a mutated influenza virus NA polypeptide described herein cleaves sialic acid. Assays known to one skilled in the art can be utilized to assess the ability of an influenza virus NA polypeptide described herein (e.g., a mutated influenza virus NA polypeptide described herein) a mutated influenza virus NA polypeptide to cleave sialic acid.

### 5.5 Compositions

In one aspect, provided herein are compositions comprising a mutated influenza virus neuraminidase polypeptide. An influenza virus comprising a mutated influenza virus neuraminidase polypeptide described herein may be incorporated into a composition. In a particular embodiment, an influenza virus described herein (e.g., in Section 5.4 or 6) is incorporated into a composition. In another embodiment, a nucleic acid sequence comprising a nucleotide sequence encoding a mutated influenza virus NA polypeptide described herein or a NA segment (such as, e.g., described herein) comprising an open reading frame encoding a mutated influenza virus NA described herein is incorporated into a composition. In another embodiment, a nucleic acid sequence comprising a nucleotide sequence encoding a chimeric influenza virus segment is incorporated into a composition, wherein the chimeric influenza virus gene segment comprises: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) the open reading frame encoding for the influenza virus NA polypeptide, (iv) a 5′ proximal coding region of the HA influenza virus gene segment; and (v) the 5′ non-coding region of the HA influenza virus gene segment. In certain embodiments, regions of the termini of the NA open reading frame implicated in genome packaging comprise serial synonymous mutations in order to abrogate their residual packaging function. In another embodiment, a nucleic acid sequence comprising a nucleotide sequence encoding a chimeric influenza virus segment is incorporated into a composition, wherein the chimeric influenza virus gene segment comprises: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) the open reading frame of the HA influenza virus gene segment, (iv) a 5′ proximal coding region of the NA influenza virus gene segment; and (v) the 5′ non-coding region of the NA influenza virus influenza gene segment. In certain embodiments, regions of the termini of the NA open reading frame implicated in genome packaging comprise serial synonymous mutations in order to abrogate their residual packaging function. In another specific embodiment, any start codon in the 3′ proximal coding region of the NA influenza virus gene segment is mutated from ATG to TTG. In another embodiment, a composition comprises a nucleic acid sequence comprising SEQ ID NO: 23, 24, 25, 26, 27 or 28. In a specific embodiment, a composition is a pharmaceutical composition, such as an immunogenic composition (e.g., a vaccine formulation). The pharmaceutical composition (e.g., immunogenic composition) may comprise a pharmaceutically acceptable carrier. The pharmaceutical composition (e.g., immunogenic composition) may comprise an adjuvant, another therapy or both. The pharmaceutical compositions provided herein can be in any form that allows for the composition to be administered to a subject. In a specific embodiment, the pharmaceutical compositions are suitable for veterinary and/or human administration. The compositions may be used in methods of preventing an influenza virus disease. The compositions may be used in methods to induce an immune response against influenza virus. The compositions may be used in methods to immunize against influenza virus. The compositions may be used in methods to enhance a humoral immune response against influenza virus NA (e.g., clinically relevant influenza virus NA). The compositions may be used in methods to induced an immune response against influenza virus NA (e.g., clinically relevant influenza virus NA). The compositions may be used in methods to increase the concentration of antibody that binds to influenza virus NA.

In another specific embodiment, a pharmaceutical composition (e.g., immunogenic composition) comprises an influenza virus described herein, and optionally an adjuvant. In a specific embodiment, a pharmaceutical composition (e.g., immunogenic composition) comprises an adjuvant (e.g., an adjuvant described herein) and an influenza virus described herein, in an admixture with a pharmaceutically acceptable carrier.

In a specific embodiment, a pharmaceutical composition (e.g., immunogenic composition) comprises an influenza virus comprising a mutated influenza virus neuraminidase polypeptide described herein, and optionally an adjuvant. In another specific embodiment, a pharmaceutical composition (e.g., immunogenic composition) comprises an influenza virus comprising a mutated influenza virus neuraminidase polypeptide described herein in an admixture with a pharmaceutically acceptable carrier. In a specific embodiment, a pharmaceutical composition (e.g., immunogenic composition) comprises an adjuvant (e.g., an adjuvant described herein) and an influenza virus comprising a mutated influenza virus neuraminidase described herein, in an admixture with a pharmaceutically acceptable carrier. In another embodiment, a pharmaceutical composition (e.g., immunogenic composition) comprises an adjuvant (e.g., an adjuvant described herein) and a nucleic acid sequence comprising a nucleotide sequence encoding a mutated influenza virus NA polypeptide described herein, or a NA segment (such as, e.g., described herein) comprising an open reading frame encoding a mutated influenza virus NA described herein.

In a specific embodiment, provided herein is a composition comprising an antibody that binds to influenza virus neuraminidase, which was generated using a mutated influenza virus NA polypeptide or an influenza virus described herein.

In some embodiments, a pharmaceutical composition (e.g., an immunogenic composition) may comprise one or more other therapies in addition to a therapy that utilizes an influenza virus described herein.

In some embodiments, a pharmaceutical composition (e.g., an immunogenic composition) may comprise one or more other therapies in addition to a therapy that utilizes an influenza virus comprising a mutated influenza virus neuraminidase polypeptide described herein. In certain embodiments, a pharmaceutical composition (e.g., an immunogenic composition) may comprise one or more other therapies in addition to a therapy that utilizes a mutated influenza virus neuraminidase polypeptide described herein, a nucleic acid sequence comprising a nucleotide sequence encoding a mutated influenza virus NA polypeptide described herein, or a NA segment (such as, e.g., described herein) comprising an open reading frame encoding a mutated influenza virus NA described herein.

As used herein, the term “pharmaceutically acceptable” means approved by a regulatory agency of the Federal or a state government or listed in the U.S. Pharmacopeia or other generally recognized pharmacopeiae for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, pharmaceutical compositions (e.g., immunogenic compositions) are formulated to be suitable for the intended route of administration to a subject. For example, the pharmaceutical composition may be formulated to be suitable for parenteral, oral, intradermal, intranasal, transdermal, colorectal, intraperitoneal, and rectal administration. In a specific embodiment, the pharmaceutical composition (e.g., an immunogenic composition) may be formulated for intravenous, oral, intraperitoneal, intranasal, intratracheal, subcutaneous, intramuscular, topical, intradermal, transdermal or pulmonary administration. In a specific embodiment, the pharmaceutical composition (e.g., an immunogenic composition) may be formulated for intramuscular administration. In a specific embodiment, the pharmaceutical composition (e.g., an immunogenic composition) may be formulated for subcutaneous administration.

In specific embodiments, immunogenic compositions described herein are monovalent formulations. In other embodiments, immunogenic compositions described herein are multivalent formulations. In one example, a multivalent formulation comprises more than one influenza virus comprising a mutated influenza virus neuraminidase described herein.

An immunogenic composition described herein may be used to immunize a subject against influenza virus. An immunogenic composition described herein may also be used to prevent an influenza virus disease in a subject. In a specific embodiment, an immunogenic composition described herein may be used in a method described herein.

In certain embodiments, the pharmaceutical compositions (e.g., immunogenic compositions) described herein additionally comprise one or more components used to inactivate a virus, e.g., formalin or formaldehyde or a detergent such as sodium deoxycholate, octoxynol 9 (Triton X-100), and octoxynol 10. In other embodiments, the pharmaceutical compositions described herein do not comprise any components used to inactivate a virus.

In certain embodiments, the pharmaceutical compositions (e.g., immunogenic compositions) described herein additionally comprise one or more buffers, e.g., phosphate buffer and sucrose phosphate glutamate buffer. In other embodiments, the pharmaceutical compositions described herein do not comprise buffers.

The pharmaceutical compositions (e.g., immunogenic compositions) described herein can be included in a container, pack, or dispenser together with instructions for administration.

The pharmaceutical compositions (e.g., immunogenic compositions) described herein can be stored before use, e.g., the pharmaceutical compositions can be stored frozen (e.g., at about −20° C. or at about −70° C.); stored in refrigerated conditions (e.g., at about 4° C.); or stored at room temperature (see International Application No. PCT/IB2007/001149 published as International Publication No. WO 07/110776, which is herein incorporated by reference in its entirety, for methods of storing compositions comprising influenza vaccines without refrigeration).

In a specific embodiment, an immunogenic composition is an inactivated vaccine comprising an adjuvant (e.g., an adjuvant described in Section 5.5.3 below). In a specific embodiment, an immunogenic composition is an inactivated vaccine comprising an adjuvant (e.g., an adjuvant described in Section 5.5.3 below) and a mutated influenza virus neuraminidase (NA) polypeptide. The inactivated vaccine may be a whole virus inactivated vaccine or split virion vaccine. Techniques for producing such vaccines are known to one of skill in the art. In a specific embodiment, an immunogenic composition comprises formalin-inactivated whole virus particles for vaccination through the intramuscular route.

### 5.5.1 Live Virus Vaccines

In one aspect, provided herein are immunogenic compositions comprising a live virus described herein. In particular embodiments, the live virus is an influenza virus, such as described in Section 5.4 or 6. In some embodiments, the live virus is attenuated.

In one embodiment, provided herein are immunogenic compositions (e.g., vaccines) comprising live virus containing a mutated influenza virus neuraminidase polypeptide. In another embodiment, provided herein are immunogenic compositions (e.g., vaccines) comprising live virus that is engineered to encode a mutated influenza virus neuraminidase polypeptide, which is expressed by progeny virus produced in the subjects administered the compositions. In specific embodiments, the mutated influenza virus neuraminidase polypeptide is membrane-bound. In other specific embodiments, the mutated influenza virus neuraminidase polypeptide is not membrane-bound, i.e., it is soluble. In particular embodiments, the live virus is an influenza virus, such as described in Section 5.4. In some embodiments, the live virus is attenuated.

In a specific embodiment, the live virus is propagated in embryonated chicken eggs before its use in an immunogenic composition described herein. In another specific embodiment, the live virus is not propagated in embryonated chicken eggs before its use in an immunogenic composition described herein. In another specific embodiment, the live virus is propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) before its use in an immunogenic composition described herein.

In a specific embodiment, the live virus that contains a mutated influenza virus neuraminidase polypeptide is propagated in embryonated chicken eggs before its use in an immunogenic composition described herein. In another specific embodiment, the live virus that contains a mutated influenza virus neuraminidase polypeptide is not propagated in embryonated chicken eggs before its use in an immunogenic composition described herein. In another specific embodiment, the live virus that contains a mutated influenza virus neuraminidase polypeptide is propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) before its use in an immunogenic composition described herein.

An immunogenic composition comprising a live virus for administration to a subject may be preferred because multiplication of the virus in the subject may lead to a prolonged stimulus of similar kind and magnitude to that occurring in natural infections, and therefore, confer substantial, long lasting immunity.

### 5.5.2 Inactivated Virus Vaccines

In one aspect, provided herein are immunogenic compositions comprising an inactivated virus described herein. In particular embodiments, the inactivated virus is an influenza virus, such as described in Section 5.4 or 6. In certain embodiments, the immunogenic composition further comprises one or more adjuvants.

In one embodiment, provided herein are immunogenic compositions (e.g., vaccines) comprising an inactivated virus containing a mutated influenza virus neuraminidase polypeptide. In specific embodiments, the mutated influenza virus neuraminidase polypeptide is membrane-bound. In particular embodiments, the inactivated virus is an influenza virus, such as described in Section 5.4 or 6. In certain embodiments, the inactivated virus immunogenic compositions comprise one or more adjuvants.

Techniques known to one of skill in the art may be used to inactivate viruses. Techniques known to one of skill in the art may be used to inactivate viruses containing a mutated influenza virus neuraminidase polypeptide. Common methods use formalin, heat, or detergent for inactivation. See, e.g., U.S. Pat. No. 6,635,246, which is herein incorporated by reference in its entirety. Other methods include those described in U.S. Pat. Nos. 5,891,705; 5,106,619 and 4,693,981, which are incorporated herein by reference in their entireties.

In a specific embodiment, an immunogenic composition described herein is a split vaccine. Techniques for producing split virus vaccines are known to those skilled in the art. By way of non-limiting example, an influenza virus split vaccine may be prepared using inactivated particles disrupted with detergents. One example of a split virus vaccine that can be adapted for use in accordance with the methods described herein is the Fluzone®, Influenza Virus Vaccine (Zonal Purified, Subvirion) for intramuscular use, which is formulated as a sterile suspension prepared from influenza viruses propagated in embryonated chicken eggs. The virus-containing fluids are harvested and inactivated with formaldehyde. Influenza virus is concentrated and purified in a linear sucrose density gradient solution using a continuous flow centrifuge. The virus is then chemically disrupted using a nonionic surfactant, octoxinol-9, (Triton® X-100—A registered trademark of Union Carbide, Co.) producing a “split virus.” The split virus is then further purified by chemical means and suspended in sodium phosphate-buffered isotonic sodium chloride solution.

In a specific embodiment, the inactivated virus that contains an influenza virus NA polypeptide described herein (e.g., a mutated influenza virus NA polypeptide described herein) was propagated in embryonated chicken eggs before its inactivation and subsequent use in an immunogenic composition described herein. In another specific embodiment, the inactivated virus that contains an influenza virus NA polypeptide described herein (e.g., a mutated influenza virus NA polypeptide described herein) was not propagated in embryonated chicken eggs before its inactivation and subsequent use in an immunogenic composition described herein. In another specific embodiment, the inactivated virus that contains an influenza virus NA polypeptide described herein (e.g., a mutated influenza virus NA polypeptide described herein) was propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) before its inactivation and subsequent use in an immunogenic composition described herein.

### 5.5.3 Adjuvants

In certain embodiments, the compositions described herein comprise, or are administered in combination with, an adjuvant. The adjuvant for administration in combination with a composition described herein may be administered before, concomitantly with, or after administration of said composition. In some embodiments, the adjuvant enhance or boosts an immune response to influenza virus and does not produce an allergy or other adverse reaction. Adjuvants can enhance an immune response by several mechanisms including, e.g., lymphocyte recruitment, stimulation of B and/or T cells, and stimulation of macrophages.

In certain embodiments, an adjuvant augments the intrinsic response to a mutated influenza virus neuraminidase polypeptide without causing conformational changes in the polypeptide that affect the qualitative form of the response. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as polyglutamic acid or polylysine, or other immunopotentiating agents.

### 5.6 Prophylactic and Therapeutic Uses

In one aspect, provided herein are methods for inducing an immune response in a subject utilizing an influenza virus containing, engineered to express a mutated influenza virus NA polypeptide described herein, or both, or a composition described herein In a specific embodiment, a method for inducing an immune response to an influenza virus neuraminidase polypeptide in a subject comprises administering to a subject in need thereof an effective amount of an immunogenic composition described herein. In a specific embodiment, a method for inducing an immune response to an influenza virus hemagglutinin polypeptide in a subject comprises administering to a subject in need thereof an effective amount of an immunogenic composition described herein. In another embodiment, a method for inducing an immune response to an influenza virus NA in a subject comprises administering to a subject in need thereof an effective amount of an influenza virus containing, engineered to express a mutated influenza virus NA polypeptide described herein, or both, or an immunogenic composition thereof.

In a specific embodiment, a method for inducing an immune response to an influenza virus in a subject comprises administering to a subject in need thereof a live virus vaccine described herein. In particular embodiments, the live virus vaccine comprises an attenuated virus. In another embodiment, a method for inducing an immune response to an influenza virus in a subject comprises administering to a subject in need thereof an inactivated virus vaccine described herein. In another embodiment, a method for inducing an immune response to an influenza virus in a subject comprises administering to a subject in need thereof a split virus vaccine described herein.

In a specific embodiment, a method for inducing an immune response to an influenza virus in a subject comprises administering to a subject in need thereof an influenza virus described herein or an immunogen composition described herein. In some embodiments, the influenza virus is a live attenuated influenza virus. In other embodiments, the influenza virus is inactivated.

In another aspect, provided herein are methods for inducing an immune response against influenza virus NA, the methods comprising administering to a subject (e.g., human subject) a recombinant influenza virus described herein or an immunogenic composition described herein.

In another aspect, provided herein are methods for enhancing a humoral immune response against influenza virus NA (e.g., clinically relevant influenza virus NA), comprising administering to a subject (e.g., human subject) a recombinant influenza virus described herein or an immunogenic composition described herein. In a specific embodiment, the humoral immune response against influenza virus NA is enhanced relative to the humoral response against influenza virus NA elicited following administration of a recombinant influenza virus in which the packaging signals of the influenza virus NA gene segment have not been exchanged with the packaging signals of influenza virus HA gene segment. In another embodiment, the humoral immune response against influenza virus NA is enhanced relative to the humoral response against influenza virus NA elicited following administration of a recombinant influenza virus in which the NA has not been mutated as described herein. In a specific embodiment, the enhanced humoral response against influenza virus NA is a stronger inhibition of neuraminidase enzymatic activity as assessed by a technique known in the art or described herein (e.g., Section 6.4, infra), higher antibody-dependent cellular cytotoxicity activity as assessed by a technique known in the art or described herein (see, e.g., Section 6.4, infra), or both. In certain embodiments, a stronger inhibition of neuraminidase enzymatic activity is 1.2, 1.3, 1.5, 1.75, 2, 2.5, 3, 3.5, 4, 4.5 fold or higher inhibition of neuraminidase enzymatic activity. In certain embodiments, higher ADCC is 1.2, 1.3, 1.5, 1.75, 2, 2.5, 3, 3.5, 4, 4.5 fold or higher ADCC activity. In some embodiments, the enhanced humoral response against influenza virus NA is a stronger inhibition of neuraminidase enzymatic activity, higher antibody-dependent cellular cytotoxicity activity, or both as described herein (see, e.g., Section 6.4, infra). In certain embodiments, the enhanced humoral response against influenza virus NA is an overall stronger anti-NA humoral response as described in Section 6.4, infra. In a specific embodiment, the subject is a human subject.

In another aspect, provided herein are methods for increasing the concentration of antibody that binds to influenza virus NA, the methods comprising administering to a subject (e.g., human subject) a recombinant influenza virus described herein or an immunogenic composition described herein. In a specific embodiment, the concentration of antibody that binds to influenza virus NA is increased relative to the concentration of antibody that binds to influenza virus NA elicited following administration of a recombinant influenza virus in which the packaging signals of the influenza virus NA gene segment have not been exchanged with the packaging signals of influenza virus HA gene segment. In another embodiment, the concentration of antibody that binds to influenza virus NA is increased relative to the concentration of antibody that binds to influenza virus NA elicited following administration of a recombinant influenza virus in which the NA has not been mutated as described herein. In certain embodiments, the concentration of antibody that binds to influenza virus NA is 1.5, 1.75, 2, 2.5, 3. 3.5, 4, 4.5 fold or higher than the concentration of antibody that binds to NA elicited following administration of a recombinant influenza virus in which the packaging signals of the influenza virus NA gene segment have not been exchanged with the packaging signals of influenza virus HA gene segment. In specific embodiments, the concentration of antibody that binds to influenza virus HA is decreased relative to the concentration of antibody that binds to HA elicited following administration of a recombinant influenza virus in which the packaging signals of the influenza virus NA gene segment have not been exchanged with the packaging signals of influenza virus HA gene segment, such as described in Section 6.4, infra. In certain embodiments, the concentration of antibody that binds to influenza virus HA is 1.25, 1.5, 1.75, 2, 2.5, 3. 3.5, 4, 4.5 fold or lower than the concentration of antibody that binds to HA elicited following administration of a recombinant influenza virus in which the packaging signals of the influenza virus NA gene segment have not been exchanged with the packaging signals of influenza virus HA gene segment.

In another aspect, provided herein are methods for immunizing against influenza virus comprising administering an immunogenic composition described herein to a subject. In one embodiment, provided herein is a method for immunizing against influenza virus in a subject, comprising administering to the subject an immunogenic composition described herein (e.g., in Section 5.5 above). In another embodiment, provided herein is a method for immunizing against influenza virus in a subject, comprising administering to the subject an immunogenic composition comprising an effective amount of an influenza virus described herein (e.g., in Section 5.4 or 6). In some embodiments, the immunogenic composition comprises an influenza virus containing, engineered to express a mutated influenza virus NA polypeptide described herein, or both, and optionally an adjuvant described herein. In another embodiment, provided herein is a method for immunizing against influenza virus in a subject, comprising administering to the subject an effective amount of an influenza virus containing, engineered to express a mutated influenza virus NA polypeptide described herein, or both, or a composition described herein, or an immunogenic composition thereof.

In another aspect, provided herein is a method for immunizing against influenza virus in a subject, comprising administering to the subject an immunogenic composition described herein (e.g., in Section 5.5 above) and administering to the subject an adjuvant described herein. In one embodiment, provided herein is a method for immunizing against influenza virus in a subject, comprising administering to the subject an immunogenic composition described herein (e.g., in Section 5.5 above) in combination with an adjuvant described herein. The immunogenic composition may be administered to the subject concurrently with, prior to (e.g., less than 5 minutes, less than 10 minutes, less than 15 minutes, less than 30 minutes, less than 45 minutes, less than 60 minutes, less than 1.5 hours, or less than 2 hours prior to), or subsequent to (e.g., less than 5 minutes, less than 10 minutes, less than 15 minutes, less than 30 minutes, less than 45 minutes, less than 60 minutes, less than 1.5 hours, or less than 2 hours after) the administration of an adjuvant described herein. In a specific embodiment, the immunogenic composition and the adjuvant described herein are administered via the same route of administration. In other embodiments, the immunogenic composition and the adjuvant are administered via different routes of administration. In a specific embodiment, the immunogenic composition comprises an inactivated influenza virus containing a mutated influenza virus NA polypeptide described herein. In another specific embodiment, the immunogenic composition comprises a split influenza virus, wherein the split influenza virus comprises a mutated influenza virus NA polypeptide described herein. In some embodiments, the immunogenic composition does not comprise an adjuvant.

In another embodiment, provided herein are immunization regimens involving a first immunization (e.g., priming) with an immunogenic composition (e.g., a vaccine) described herein followed by one, two, or more additional immunizations (e.g., boostings) with an immunogenic composition (e.g., a vaccine). In a specific embodiment, the immunogenic composition (e.g., a vaccine) used in the first immunization is the same type of an immunogenic composition (e.g., a vaccine) used in one, two or more additional immunizations. For example, if the immunogenic composition (e.g., vaccine) used in the first immunization is an inactivated influenza virus vaccine formulation, the immunogenic composition (e.g., vaccine) used for the one, two or more additional immunizations may be the same type of vaccine formulation, i.e., an inactivated influenza virus vaccine formulation. In other specific embodiments, the immunogenic composition (e.g., vaccine) used in the first immunization is different from the type of immunogenic composition (e.g., vaccine) used in one, two or more additional immunizations. For example, if the immunogenic composition (e.g., vaccine) used in the first immunization is a live influenza virus vaccine formulation, the immunogenic composition (e.g., vaccine) used in the one, two or more additional immunizations is another type of vaccine formulation, such as an inactivated influenza virus. In another example, if the immunogenic composition (e.g., vaccine) used in the first immunization is a live attenuated influenza virus vaccine formulation, the immunogenic composition (e.g., vaccine) used in the one, two or more additional immunizations is another type of vaccine formulation, such as an inactivated influenza virus. In certain embodiments, the vaccine formulation used in the additional immunizations changes. For example, if a live attenuated influenza virus vaccine formulation is used for one additional immunization, then one or more additional immunizations may use a different vaccine formulation, such as an inactivated vaccine formulation. In a particular embodiment, a live influenza virus vaccine formulation is administered to a subject followed by an inactivated vaccine formulation (e.g., split virus vaccine or subunit vaccine).

In a specific embodiment, a subject is immunized in accordance with a method described herein prior, during or both flu season. In a specific embodiment, flu season in the U.S. may be from September or October of one year through March or April of the next year.

In some embodiments, the immune response induced by an immunogenic composition described herein is effective to prevent an influenza virus disease caused by one, two, or more subtypes of influenza A virus. In some embodiments, the immune response induced by an immunogenic composition described herein is effective to prevent an influenza virus disease caused by one, two, three or more strains of influenza virus. In certain embodiments, the immune response induced by an immunogenic composition described herein is effective to prevent an influenza virus disease caused by a subtype of influenza virus that belongs to one NA group and not another NA group. In some embodiments, the immune response induced by an immunogenic composition described herein is effective to prevent an influenza virus disease caused by one or more variants within the same subtype of influenza A virus. In certain embodiments, the immune response induced by an immunogenic composition described herein is effective to prevent an influenza virus disease caused by one, two, three or more strains within the same subtype of influenza A virus.

In some embodiments, the immune response induced by an immunogenic composition described herein is effective to reduce the number of symptoms resulting from an influenza virus disease/infection. In certain embodiments, the immune response induced by an immunogenic composition described herein is effective to reduce the duration of one or more symptoms resulting from an influenza virus disease/infection. In some embodiments, the immune response induced by an immunogenic composition described herein is effective to reduce the number of symptoms of an influenza virus infection/disease and reduce the duration of one or more symptoms of an influenza virus infection/disease. Symptoms of influenza virus disease/infection include, but are not limited to, body aches (especially joints and throat), fever, nausea, headaches, irritated eyes, fatigue, sore throat, reddened eyes or skin, and abdominal pain.

In some embodiments, the immune response induced by an immunogenic composition described herein is effective to reduce the hospitalization of a subject suffering from an influenza virus disease/infection. In some embodiments, the immune response induced by an immunogenic composition described herein is effective to reduce the duration of hospitalization of a subject suffering from an influenza virus disease/infection.

In a specific embodiment, the immune response induced by an immunogenic composition described herein induces NA-specific antibodies (e.g., IgG). In another specific embodiment, the immune response induced by an immunogenic composition described herein induces antibodies with one, two or more of the characteristics of the antibodies described in Section 6, infra. In another specific embodiment, the immune response induced by an immunogenic composition described herein induces antibodies with ADCC activity as assessed by a technique known to one of skill in the art or described herein (see, e.g., Section 6, infra). In another specific embodiment, the immune response induced by an immunogenic composition described herein induces antibodies with neuraminidase inhibition activity as assessed by a technique known to one of skill in the art or described herein (see, e.g., Section 6, infra). In another specific embodiment, the immune response induced by an immunogenic composition described herein induces antibodies with (1) ADCC activity as assessed by a technique known to one of skill in the art or described herein (see, e.g., Section 6, infra); and (2) neuraminidase inhibition activity as assessed by a technique known to one of skill in the art or described herein (see, e.g., Section 6, infra).

In another aspect, provided herein are methods for preventing an influenza virus disease in a subject utilizing an immunogenic composition described herein. In a specific embodiment, provided herein is a method for preventing an influenza virus disease in a subject utilizing an effective amount of an immunogenic composition described herein. In a specific embodiment, a method for preventing an influenza virus disease in a subject comprises administering to a subject in need thereof a live virus vaccine, an inactivated virus vaccine, or a split virus vaccine described herein. In a specific embodiment, a method for preventing an influenza virus disease in a subject comprises administering to a subject in need thereof an effective amount of a live virus vaccine, an inactivated virus vaccine, or a split virus vaccine described herein. In a specific embodiment, a method for preventing an influenza virus disease in a subject comprises administering to a subject in need thereof a live virus vaccine described herein. In particular embodiments, the live virus vaccine comprises an attenuated virus. In another embodiment, a method for preventing an influenza virus disease in a subject comprises administering to a subject in need thereof an inactivated virus vaccine described herein. In another embodiment, a method for preventing or an influenza virus disease in a subject comprises administering to a subject in need thereof a split virus vaccine described herein.

In another aspect, provided herein are methods for preventing an influenza virus disease, or treating an influenza virus infection or an influenza virus disease in a subject comprising administering to a subject an anti-influenza virus NA antibody(ies), wherein the anti-influenza virus NA antibody(ies) was generated utilizing an immunogenic composition described herein. For example, an immunogenic composition described herein may be administered to a non-human subject (e.g., a non-human subject that expresses or is capable of expression human antibody) to generate anti-influenza virus NA antibody(ies). In a specific embodiment, provided herein is a method for preventing an influenza virus disease in a human subject comprising administering the subject a human or humanized anti-influenza virus NA antibody(ies), wherein the anti-influenza virus NA antibody(ies) was generated utilizing an immunogenic composition described herein.

In certain embodiments, the methods for preventing an influenza virus disease, or treating an influenza virus infection or an influenza virus disease in a subject (e.g., a human or non-human animal) provided herein result in a reduction in the replication of the influenza virus in the subject as measured by in vivo and in vitro assays known to those of skill in the art and described herein. In some embodiments, the replication of the influenza virus is reduced by approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. In specific embodiments, the methods for preventing an influenza virus disease, or treating an influenza virus infection or an influenza virus disease in a subject (e.g., a human or non-human animal) provided herein result in a reduction of the titer of an influenza virus detected in the subject. In specific embodiments, the methods for preventing an influenza virus disease, or treating an influenza virus infection or an influenza virus disease in a subject results in one, two, or more of the following: (1) reduces the number of symptoms of the infection/disease, (2) reduces the severity of the symptoms of the infection/disease, (3) reduces the length of the infection/disease, (4) reduces hospitalization or complications resulting from the infection/disease, (5) reduces the length of hospitalization of the subject, (6) reduces organ failure associated with the influenza virus infection/disease, and (7) increases survival of the subject. In a specific embodiment, the methods for preventing an influenza virus disease, or treating an influenza virus infection or an influenza virus disease in a subject inhibits the development or onset of an influenza virus disease or one or more symptoms thereof.

In certain embodiments, provided herein are methods for generating antibodies comprising administering an influenza virus or composition described herein (e.g., in Section 5.4, 5.5 or 6) to a subject (e.g., a non-human subject). In particular, provided herein are methods for generating anti-influenza virus NA antibodies comprising administering an influenza virus or composition described herein (e.g., in Section 5.4, 5.5 or 6) to a subject (e.g., a non-human subject). In certain embodiments, provided herein are methods for generating antibodies comprising administering an influenza virus containing, engineered to express a mutated influenza virus NA polypeptide described herein, or both, or composition described herein administered to a subject (e.g., a non-human subject). In some embodiments, an influenza virus containing, engineered to express a mutated influenza virus NA polypeptide described herein, or both, or composition described herein may be administered to a subject (e.g., a non-human subject) and the antibodies may be isolated. The isolated antibodies may be cloned. The antibodies may be humanized and/or optimized. In some embodiments, hybridomas are produced which produce a particular antibody of interest. Techniques for isolating, cloning, humanizing, optimizing and for generating hybridomas are known to one of skill in the art. In a specific embodiment, antibodies generated by a method described herein may be utilized in assays (e.g., assays described herein) as well as in passive immunization of a subject (e.g., a human subject). Thus, provided herein, in certain embodiments, are methods for treating influenza virus infection or influenza virus disease, or preventing influenza virus disease, comprising administering antibodies generated by a method described herein.

### 5.6.1 Combination Therapies

In certain embodiments, an influenza virus containing, engineered to express a mutated influenza virus NA polypeptide described herein, or both may be administered to a subject in combination with one or more other therapies (e.g., an antiviral, antibacterial, or immunomodulatory therapies). In various embodiments, an influenza virus containing, engineered to express a mutated influenza virus NA polypeptide described herein, or both may be administered to a subject in combination with one or more other therapies (e.g., an antiviral, antibacterial, or immunomodulatory therapies). In some embodiments, a pharmaceutical composition (e.g., an immunogenic composition) described herein may be administered to a subject in combination with one or more therapies (e.g., an antiviral, antibacterial, or immunomodulatory therapies). The one or more other therapies may be beneficial in the prevention of an influenza virus disease or may ameliorate a symptom or condition associated with an influenza virus disease. The one or more other therapies may be in administered in a form (e.g., a pharmaceutical composition) that is approved by a regulatory agency (e.g., FDA) or as in clinical trials. In certain embodiments, the one or more other therapies are administered to a subject (e.g., a human subject) in the same composition as an influenza virus described herein (e.g., in Section 5.4 or 6). In other embodiment, the one or more other therapies are not administered to a subject (e.g., a human subject) in a different composition than an influenza virus described herein (e.g., in Section 5.4 or 6). In some embodiments, the one or more other therapies are pain relievers, anti-fever medications, or therapies that alleviate or assist with breathing. In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In specific embodiments, two or more therapies are administered within the same patient visit.

### 5.6.2 Patient Populations

In certain embodiments, an influenza virus or composition described herein (e.g., in Section 5.4, 5.5, or 6) may be administered to a naïve subject, i.e., a subject that does not have a disease caused by influenza virus infection or has not been and is not currently infected with an influenza virus infection. In one embodiment, an influenza virus or composition described herein (e.g., in Section 5.4, 5.5, or 6) is administered to a naïve subject that is at risk of acquiring an influenza virus infection. In another embodiment, an influenza virus or composition described herein (e.g., in Section 5.4, 5.5, or 6) is administered to a subject that does not have a disease caused by the specific influenza virus, or has not been and is not infected with the specific influenza virus to which the influenza virus NA polypeptide induces an immune response. an influenza virus or composition described herein (e.g., in Section 5.4, 5.5, or 6) may also be administered to a subject that is, has been, or is and has been infected with the influenza virus or another type, subtype/lineage or strain of the influenza virus to which the mutated influenza virus NA polypeptide induces an immune response.

In certain embodiments, an influenza virus or composition described herein (e.g., in Section 5.4, 5.5, or 6) is administered to a patient who has been diagnosed with an influenza virus infection. In some embodiments, an influenza virus or composition described herein (e.g., in Section 5.4, 5.5, or 6) is administered to a patient infected with an influenza virus before symptoms manifest or symptoms become severe (e.g., before the patient requires hospitalization).

In some embodiments, a subject to be administered an influenza virus or composition described herein (e.g., in Section 5.4, 5.5, or 6) is an animal. In certain embodiments, the animal is a bird. In certain embodiments, the animal is a canine. In certain embodiments, the animal is a feline. In certain embodiments, the animal is a horse. In certain embodiments, the animal is a cow. In certain embodiments, the animal is a mammal, e.g., a horse, swine, mouse, or primate, preferably a human.

In specific embodiments, a subject to be administered an influenza virus or composition described herein (e.g., in Section 5.4, 5.5, or 6) is a human infant. As used herein, the term “human infant” refers to a newborn to 1 year old human. In specific embodiments, a subject to be administered an influenza virus or composition described herein (e.g., in Section 5.4, 5.5, or 6) is a human child. As used herein, the term “human child” refers to a human that is 1 year to 18 years old. In specific embodiments, a subject to be administered an influenza virus or composition described herein (e.g., in Section 5.4, 5.5, or 6) is a human adult. As used herein, the term “human adult” refers to a human that is 18 years or older. In specific embodiments, a subject to be administered an influenza virus or composition described herein (e.g., in Section 5.4, 5.5, or 6) is an elderly human. As used herein, the term “elderly human” refers to a human 65 years or older.

In some embodiments, the human subject to be administered an influenza virus or composition described herein (e.g., Section 5.4, 5.5, or 6) is any individual at increased risk of influenza virus infection or disease resulting from influenza virus infection (e.g., an immunocompromised or immunodeficient individual). In some embodiments, the human subject to be administered an influenza virus or composition described herein (e.g., Section 5.4, 5.5, or 6) is any individual in close contact with an individual with increased risk of influenza virus infection or disease resulting from influenza virus infection (e.g., immunocompromised or immunosuppressed individuals).

In some embodiments, the human subject to be administered an influenza virus or composition described herein (e.g., Section 5.4, 5.5, or 6) is an individual affected by any condition that increases susceptibility to influenza virus infection or complications or disease resulting from influenza virus infection. In other embodiments, an influenza virus or composition described herein (e.g., Section 5.4, 5.5, or 6) is administered to a subject in whom an influenza virus infection has the potential to increase complications of another condition that the individual is affected by, or for which they are at risk.

In certain embodiments, an influenza virus containing, engineered to express a mutated influenza virus NA polypeptide described herein, or both, or composition described herein may be administered to a naïve subject, i.e., a subject that does not have a disease caused by influenza virus infection or has not been and is not currently infected with an influenza virus infection. In one embodiment, an influenza virus containing, engineered to express a mutated influenza virus NA polypeptide described herein, or both, or composition described herein is administered to a naïve subject that is at risk of acquiring an influenza virus infection. In another embodiment, an influenza virus containing, engineered to express a mutated influenza virus NA polypeptide described herein, or both, or composition described herein is administered to a subject that does not have a disease caused by the specific influenza virus, or has not been and is not infected with the specific influenza virus to which the mutated influenza virus NA polypeptide induces an immune response. An influenza virus containing, engineered to express a mutated influenza virus NA polypeptide described herein, or both, or composition described herein may also be administered to a subject that is, has been, or is and has been infected with the influenza virus or another type, subtype/lineage or strain of the influenza virus to which the mutated influenza virus NA polypeptide induces an immune response.

In certain embodiments, an influenza virus containing, engineered to express a mutated influenza virus NA polypeptide described herein, or both, or composition described herein is administered to a patient who has been diagnosed with an influenza virus infection. In some embodiments, an influenza virus containing, engineered to express a mutated influenza virus NA polypeptide described herein, or both, or composition described herein is administered to a patient infected with an influenza virus before symptoms manifest or symptoms become severe (e.g., before the patient requires hospitalization).

In some embodiments, a subject to be an influenza virus containing, engineered to express a mutated influenza virus NA polypeptide described herein, or both, or composition described herein is an animal. In certain embodiments, the animal is a bird. In certain embodiments, the animal is a canine. In certain embodiments, the animal is a feline. In certain embodiments, the animal is a horse. In certain embodiments, the animal is a cow. In certain embodiments, the animal is a mammal, e.g., a horse, swine, mouse, or primate, preferably a human.

In specific embodiments, a subject administered an influenza virus containing, engineered to express a mutated influenza virus NA polypeptide described herein, or both, or composition described herein is a human infant. As used herein, the term “human infant” refers to a newborn to 1 year old human. In specific embodiments, a subject administered an influenza virus containing, engineered to express a mutated influenza virus NA polypeptide described herein, or both, or composition described herein is a human child. As used herein, the term “human child” refers to a human that is 1 year to 18 years old. In specific embodiments, a subject administered an influenza virus containing, engineered to express a mutated influenza virus NA polypeptide described herein, or both, or composition described herein is a human adult. As used herein, the term “human adult” refers to a human that is 18 years or older. In specific embodiments, a subject administered an influenza virus containing, engineered to express a mutated influenza virus NA polypeptide described herein, or both, or composition described herein is an elderly human. As used herein, the term “elderly human” refers to a human 65 years or older.

In some embodiments, the human subject to be administered an influenza virus containing, engineered to express a mutated influenza virus NA polypeptide described herein, or both, or composition described herein is any individual at increased risk of influenza virus infection or disease resulting from influenza virus infection (e.g., an immunocompromised or immunodeficient individual). In some embodiments, the human subject to be administered an influenza virus containing, engineered to express a mutated influenza virus NA polypeptide described herein, or both, or composition described herein is any individual in close contact with an individual with increased risk of influenza virus infection or disease resulting from influenza virus infection (e.g., immunocompromised or immunosuppressed individuals).

In some embodiments, the human subject to be administered an influenza virus containing, engineered to express a mutated influenza virus NA polypeptide described herein, or both, or composition described herein is an individual affected by any condition that increases susceptibility to influenza virus infection or complications or disease resulting from influenza virus infection. In other embodiments, an influenza virus containing, engineered to express a mutated influenza virus NA polypeptide described herein, or both, or composition described herein is administered to a subject in whom an influenza virus infection has the potential to increase complications of another condition that the individual is affected by, or for which they are at risk.

### 5.7 Modes of Administration

### 5.7.1 Routes of Delivery

An influenza virus or composition described herein (e.g. in Section 5.4, 5.5 or 6) may be delivered to a subject by a variety of routes. An influenza virus containing, engineered to express or both a mutated influenza virus NA polypeptide described herein, or composition described herein may be delivered to a subject by a variety of routes. These include, but are not limited to, intranasal, intratracheal, oral, intradermal, intramuscular, intraperitoneal, transdermal, intravenous, conjunctival and subcutaneous routes. In some embodiments, a composition is formulated for topical administration, for example, for application to the skin. In specific embodiments, the route of administration is nasal, e.g., as part of a nasal spray. In certain embodiments, a composition is formulated for intramuscular administration. In some embodiments, a composition is formulated for subcutaneous administration. In certain embodiments, a composition is not formulated for administration by injection. In specific embodiments for live virus vaccines, the vaccine is formulated for administration by a route other than injection.

In one embodiment, a live attenuated influenza virus vaccine is administered intranasally. In another embodiment, an inactivated influenza virus vaccine (e.g., an inactivated whole virus vaccine or a split influenza virus vaccine) is administered intramuscularly.

### 5.7.2 Dosage

The amount of an influenza virus or composition described herein (e.e., Section 5.4, 5.5. or 6) which will be effective in the prevention of an influenza virus disease will depend on the nature of the disease, and can be determined by standard clinical techniques. The amount of an influenza virus containing, engineered to express or both a mutated influenza virus NA polypeptide described herein, or composition described herein which will be effective in the prevention of an influenza virus disease will depend on the nature of the disease, and can be determined by standard clinical techniques.

The precise dose to be employed in the formulation will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, health), whether the patient is human or an animal, other medications administered, and whether treatment is prophylactic or therapeutic. Usually, the patient is a human but non-human mammals including transgenic mammals can also be treated. Treatment dosages are optimally titrated to optimize safety and efficacy.

As used herein, the term “effective amount” in the context of administering a therapy to a subject refers to the amount of a therapy which may have a prophylactic effect(s), therapeutic effect(s), or both a prophylactic and therapeutic effect(s). In certain embodiments, an “effective amount” in the context of administration of a therapy to a subject refers to the amount of a therapy which is sufficient to achieve one, two, three, four, or more of the following effects: (i) reduce or ameliorate the severity of an influenza virus infection, disease or symptom associated therewith; (ii) reduce the duration of an influenza virus infection, disease or symptom associated therewith; (iii) prevent the progression of an influenza virus infection, disease or symptom associated therewith; (iv) cause regression of an influenza virus infection, disease or symptom associated therewith; (v) prevent the development or onset of an influenza virus infection, disease or symptom associated therewith; (vi) prevent the recurrence of an influenza virus infection, disease or symptom associated therewith; (vii) reduce or prevent the spread of an influenza virus from one cell to another cell, one tissue to another tissue, or one organ to another organ; (viii) prevent or reduce the spread of an influenza virus from one subject to another subject; (ix) reduce organ failure associated with an influenza virus infection; (x) reduce hospitalization of a subject; (xi) reduce hospitalization length; (xii) increase the survival of a subject with an influenza virus infection or disease associated therewith; (xiii) eliminate an influenza virus infection or disease associated therewith; (xiv) inhibit or reduce influenza virus replication; (xv) inhibit or reduce the entry of an influenza virus into a host cell(s); (xvi) inhibit or reduce replication of the influenza virus genome; (xvii) inhibit or reduce synthesis of influenza virus proteins; (xviii) inhibit or reduce assembly of influenza virus particles; (xix) inhibit or reduce release of influenza virus particles from a host cell(s); (xx) reduce influenza virus titer; and/or (xxi) enhance or improve the prophylactic or therapeutic effect(s) of another therapy.

In certain embodiments, the effective amount does not result in complete protection from an influenza virus disease, but results in a lower titer or reduced number of influenza viruses compared to an untreated subject with an influenza virus infection. In certain embodiments, the effective amount results in a 0.5 fold, 1 fold, 1.5 fold, 2 fold, 3 fold, 4 fold, 5 fold, 6 fold, 7 fold, 8 fold, 9 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titer of influenza virus relative to an untreated subject with an influenza virus infection. In some embodiments, the effective amount results in a reduction in titer of influenza virus relative to an untreated subject with an influenza virus infection of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. Benefits of a reduction in the titer, number or total burden of influenza virus include, but are not limited to, less severe symptoms of the infection, fewer symptoms of the infection and a reduction in the length of the disease associated with the infection.

In certain embodiments, an effective amount of a therapy (e.g., a composition thereof, such as an influenza virus or a mutated influenza virus NA polypeptide described herein) results in an anti-influenza virus NA titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-influenza virus NA titer in a blood sample from the subject prior to immunization. In certain embodiments, an effective amount of a therapy (e.g., a composition thereof, such as an influenza virus or a mutated influenza virus NA polypeptide described herein) results in an anti-influenza virus NA stalk titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-influenza virus NA stalk titer in a blood sample from the subject prior to immunization.

In certain embodiments, the dose of an influenza virus described herein may be 10⁴ plaque forming units (PFU) to 10⁸ PFU. In some embodiments, an inactivated vaccine is formulated such that it contains 15 μg of hemagglutinin (HA) polypeptide described herein. In certain embodiments, an inactivated vaccine is formulated such that it contains 5 to 15 or 5 μg, 10 μg, 15 μg of hemagglutinin (HA) polypeptide described herein. In some embodiments, an inactivated vaccine is formulated such that it contains 5 to 15 μg, or 5 μg, 10 μg, 15 μg of NA polypeptide described herein. In certain embodiments, a composition described herein contains 5 to 15 μg, or 5 μg, 10 μg, 15 μg of NA polypeptide described herein. In some embodiments, composition described herein contains 5 to 100 μg of a nucleic acid sequence comprising a nucleotide sequence encoding a mutated influenza virus NA polypeptide described herein.

### 5.8 Biological Assays

In another aspect, provided herein are biological assays that may be used to characterize an influenza virus described herein or a composition described herein. Also provided herein are biological assays that may be used to characterize a mutated influenza virus NA polypeptide, and viruses containing, expressing, or both such mutated influenza virus NA polypeptide. See, also, Section 6. In a specific embodiment, an assay described in Section 6 is used to characterize a mutated influenza virus NA polypeptide or virus containing, expressing, or both such a mutated influenza virus NA polypeptide. In another specific embodiment, an assay described in Section 6 is used to characterize the ADCC activity of antibodies induced by an immunogenic composition described herein. In another specific embodiment, an assay described in Section 6 is used to characterize the neuraminidase inhibition activity of antibodies induced by an immunogenic composition described herein. In another specific embodiment, the immunogenicity or effectiveness of an immunogenic composition described herein is assessed using one, two, or more assays described in Section 6. In another specific embodiment, the ADCC activity of antibody induced following administration of an influenza virus or composition described herein (e.g., in Section 5.4, 5.5. or 6) may be characterized using techniques known to one of skill in the art or as described herein (e.g., in Section 6). In another specific embodiment, the inhibition of neuraminidase enzymatic activity of antibody induced following administration of an influenza virus or composition described herein (e.g., in Section 5.4, 5.5. or 6) may be characterized using techniques known to one of skill in the art or as described herein (e.g., in Section 6).

### 5.8.1 Assays for Testing Activity of Influenza Virus Neuraminidase Polypeptides

Assays for testing the expression of a mutated influenza virus neuraminidase polypeptide in an influenza virus disclosed herein may be conducted using any assay known in the art. For example, an assay for incorporation into a viral vector comprises growing the virus as described herein, purifying the viral particles by centrifugation through a sucrose cushion, and subsequent analysis for a mutated influenza virus neuraminidase polypeptide expression by an immunoassay, such as Western blotting, using methods well known in the art.

In another embodiment, a mutated influenza virus neuraminidase polypeptide disclosed herein is assayed for proper folding by determination of the structure or conformation of the influenza virus neuraminidase polypeptide using any method known in the art such as, e.g., NMR, X-ray crystallographic methods, or secondary structure prediction methods, e.g., circular dichroism.

In addition, assays for testing the expression and activity of influenza virus neuraminidase polypeptide may be conducted using any assay known in the art or described herein (e.g., in Section 6).

### 5.9 Kits

In one aspect, provided herein is a pharmaceutical pack or kit for immunizing against an influenza virus in a subject comprising one or more containers filled with one or more of the ingredients of a pharmaceutical composition described herein (e.g., an immunogenic composition described herein), such as an influenza virus (e.g., a live attenuated influenza virus or an inactivated virus) or a mutated influenza virus NA polypeptide. Optionally associated with such container(s) can be a notice in the form prescribed by a governmental agency regulating the manufacture, use or sale of pharmaceuticals or biological products, which notice reflects approval by the agency of manufacture, use or sale for human administration. In another embodiment, provided herein is a pharmaceutical pack or kit comprising one or more containers filled with one or more of the ingredients of a pharmaceutical composition described herein (e.g., an immunogenic composition described herein), such as a nucleic acid sequence comprising a nucleotide sequence encoding a mutated influenza virus NA polypeptide described herein. In another embodiment, provided herein is a kit comprising a container filled with an NA segment described herein. In another embodiment, provided herein is a kit comprising one or more containers filled with an NA segment and an HA segment described herein (e.g., chimeric NA and HA segments described herein). In a specific embodiment, provided herein is a kit comprising a container filled with a first chimeric influenza virus gene comprising a nucleotide sequence encoding an influenza virus NA polypeptide described herein and a container filled with a second chimeric influenza virus gene comprising a nucleotide sequence encoding an influenza virus HA polypeptide described herein. See, e.g., Section 5.2, 5.4, and 6. In some embodiments, a kit comprises a container comprising an NA segment, and a container comprising an HA segment, wherein the NA segment comprises the following and allows for the insertion of a NA open reading frame: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) a placeholder that allows for insertion of an influenza virus NA open reading frame; (iv) a 5′ proximal coding region of the HA influenza virus gene segment; and (v) the 5′ non-coding region of the HA influenza virus gene segment; and wherein the HA segment comprises the following and allows for the insertion of an HA open reading frame: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) a placeholder that allows for insertion of an influenza virus HA open reading frame, (iv) a 5′ proximal coding region of the NA influenza virus gene segment; and (v) the 5′ non-coding region of the NA influenza virus influenza gene segment. In certain embodiments, a kit comprises one, two, three, four, five or more containers filled with influenza virus NS, PB1, PB2, PA, M, and NP gene segments, a container comprising an NA segment, and a container comprising an HA segment, wherein the NA segment comprises the following and allows for the insertion of a NA open reading frame: (i) a 3′ non-coding region of an HA influenza virus gene segment; (ii) a 3′ proximal coding region of the HA influenza virus gene segment, wherein any start codon in the 3′ proximal coding region of the HA influenza virus gene segment is mutated; (iii) a placeholder that allows for insertion of an influenza virus NA open reading frame; (iv) a 5′ proximal coding region of the HA influenza virus gene segment; and (v) the 5′ non-coding region of the HA influenza virus gene segment; and wherein the HA segment comprises the following and allows for the insertion of an HA open reading frame: (i) the 3′ non-coding region of an NA influenza virus gene segment; (ii) a 3′ proximal coding region of the NA influenza virus gene segment, wherein any start codon in the 3′ proximal coding region of the NA influenza virus gene segment is mutated; (iii) a placeholder that allows for insertion of an influenza virus HA open reading frame, (iv) a 5′ proximal coding region of the NA influenza virus gene segment; and (v) the 5′ non-coding region of the NA influenza virus influenza gene segment. The placeholders that allow for insertion of the open reading frames may be restriction sites. In addition, the 3′ proximal nucleotides, 5′ proximal nucleotides, or both in the open reading frames may comprise synonymous mutations to abrogate the packaging signals present.

The kits encompassed herein can be used in accordance with the methods described herein. In one embodiment, a kit comprises an influenza virus described herein containing a mutated influenza virus NA polypeptide (such as described in Section 5.1 above or Section 6), in one or more containers. In another embodiment, a kit comprises one or more immunogenic compositions described herein in one or more containers. In certain embodiments, a kit comprises a vaccine described herein, e.g., an inactivated influenza virus vaccine or a live influenza virus vaccine, wherein said vaccine comprises a mutated influenza virus NA polypeptide described herein and optionally, an adjuvant described herein (e.g., in Section 5.5.3 or Section 6).

In certain embodiments, a kit described herein comprises: (a) a first container comprising an immunogenic composition described herein (e.g., described in Section 5.1 or Section 6); and (b) a second container comprising an adjuvant described herein (e.g., in Section 5.5.3). In specific embodiments, the immunogenic composition is an inactivated whole virus vaccine. In specific embodiments, the immunogenic composition is a split virus vaccine. In specific embodiment, the immunogenic composition is a live attenuated virus vaccine.

SEQUENCES

HK14 N2-Del 25 Nucleotide (1391 bp)
AGCAAAAGCAGGAGTAAAGATGAATCCAAATCAAAAGATAATAACGATTGGCTCTG
TTTCTCTCACCATTTCCACAATATGCTTCTTCATGCAAATTGCCATTTTGATAACTAC
TGTAACATTGCATTTCAAGCAAATAGTGTATTTAACTAACACCACCATAGAGAAGGA
AATATGCCCCAAACCAGCAGAATACAGAAATTGGTCAAAACCGCAATGTGGCATTA
CAGGATTTGCACCTTTCTCTAAGGACAATTCGATTAGGCTTTCCGCTGGTGGGGACA
TCTGGGTGACAAGAGAACCTTATGTGTCATGCGATCCTGACAAGTGTTATCAATTTG
CCCTTGGACAGGGAACAACACTAAACAACGTGCATTCAAATAACACAGTACGTGAT
AGGACCCCTTATCGGACTCTATTGATGAATGAGTTGGGTGTTCCTTTCCATCTGGGG
ACCAAGCAAGTGTGCATAGCATGGTCCAGCTCAAGTTGTCACGATGGAAAAGCATG
GCTGCATGTTTGTATAACGGGGGATGATAAAAATGCAACTGCTAGCTTCATTTACAA
TGGGAGGCTTGTAGATAGTGTTGTTTCATGGTCCAAAGATATTCTCAGGACCCAGGA
GTCAGAATGCATTTGTATCAATGGAACTTGTACAGTAGTAATGACTGATGGAAGTGC
TTCAGGAAAAGCTGATACTAAAATACTATTCATTGAGGAGGGGAAAATCGTTCATA
CTAGCACATTGTCAGGAAGTGCTCAGCATGTCGAAGAGTGCTCTTGCTATCCTCGAT
ATCCTGGTGTCAGATGTGTCTGCAGAGACAACTGGAAGGGCTCCAATCGGCCCATCG
TAGATATAAACATAAAGGATCATAGCATTGTTTCCAGTTATGTGTGTTCAGGACTTG
TTGGAGACACACCCAGAAAAAACGACAGCTCCAGCAGTAGCCATTGTTTGGATCCT
AACAATGAAGAAGGTGGTCATGGAGTGAAAGGCTGGGCCTTTGATGATGGAAATGA
CGTGTGGATGGGAAGAACAATCAACGAGACGTCACGCTTAGGGTATGAAACCTTCA
AAGTCATTGAAGGCTGGTCCAACCCTAAGTCCAAATTGCAGACAAATAGGCAAGTC
ATAGTTGACAGAGGTGATAGGTCCGGTTATTCTGGTATTTTCTCTGTTGAAGGCAAA
AGCTGCATCAATCGGTGCTTTTATGTGGAGTTGATTAGGGGAAGAAAAGAGGAAAC
TGAAGTCTTGTGGACCTCAAACAGTATTGTTGTGTTTTGTGGCACCTCAGGTACATAT
GGAACAGGCTCATGGCCTGATGGGGCGGACCTCAATCTCATGCCTATATAAGCTTTC
GCAATTTTAGAAAAAACTCCTTGTTTCTACT (SEQ ID NO: 1)
HK14 N2-Del 25 Amino Acid
MNPNQKIITIGSVSLTISTICFFMQIAILITTVTLHFKQIVYLTNTTIEKEICPKPAEYRNWSK
PQCGITGFAPFSKDNSIRLSAGGDIWVTREPYVSCDPDKCYQFALGQGTTLNNVHSNNT
VRDRTPYRTLLMNELGVPFHLGTKQVCIAWSSSSCHDGKAWLHVCITGDDKNATASFI
YNGRLVDSVVSWSKDILRTQESECICINGTCTVVMTDGSASGKADTKILFIEEGKIVHTST
LSGSAQHVEECSCYPRYPGVRCVCRDNWKGSNRPIVDINIKDHSIVSSYVCSGLVGDTPR
KNDSSSSSHCLDPNNEEGGHGVKGWAFDDGNDVWMGRTINETSRLGYETFKVIEGWS
NPKSKLQTNRQVIVDRGDRSGYSGIFSVEGKSCINRCFYVELIRGRKEETEVLWT
SNSIVVFCGTSGTYGTGSWPDGADLNLMPI (SEQ ID NO: 2)
HK14 N2-Ins15 Nucleotide (1511 bp)
AGCAAAAGCAGGAGTAAAGATGAATCCAAATCAAAAGATAATAACGATTGGCTCTG
TTTCTCTCACCATTTCCACAATATGCTTCTTCATGCAAATTGCCATTTTGATAACTAC
TGTAACATTGCATTTCAAGCAATATGAATTCAACTCCCCCCCAAACAACCAAGTGAT
GCTGTGTGAACCAACAATAATAGAAAGAAACATAACAGAAATAGTGTATTTAACTA
ATCAGACATATGTTAACATCAGCAACACCAACTTTGCTGCTGGAAACACCACCATAG
AGAAGGAAATATGCCCCAAACCAGCAGAATACAGAAATTGGTCAAAACCGCAATGT
GGCATTACAGGATTTGCACCTTTCTCTAAGGACAATTCGATTAGGCTTTCCGCTGGT
GGGGACATCTGGGTGACAAGAGAACCTTATGTGTCATGCGATCCTGACAAGTGTTAT
CAATTTGCCCTTGGACAGGGAACAACACTAAACAACGTGCATTCAAATAACACAGT
ACGTGATAGGACCCCTTATCGGACTCTATTGATGAATGAGTTGGGTGTTCCTTTCCAT
CTGGGGACCAAGCAAGTGTGCATAGCATGGTCCAGCTCAAGTTGTCACGATGGAAA
AGCATGGCTGCATGTTTGTATAACGGGGGATGATAAAAATGCAACTGCTAGCTTCAT
TTACAATGGGAGGCTTGTAGATAGTGTTGTTTCATGGTCCAAAGATATTCTCAGGAC
CCAGGAGTCAGAATGCATTTGTATCAATGGAACTTGTACAGTAGTAATGACTGATGG
AAGTGCTTCAGGAAAAGCTGATACTAAAATACTATTCATTGAGGAGGGGAAAATCG
TTCATACTAGCACATTGTCAGGAAGTGCTCAGCATGTCGAAGAGTGCTCTTGCTATC
CTCGATATCCTGGTGTCAGATGTGTCTGCAGAGACAACTGGAAGGGCTCCAATCGGC
CCATCGTAGATATAAACATAAAGGATCATAGCATTGTTTCCAGTTATGTGTGTTCAG
GACTTGTTGGAGACACACCCAGAAAAAACGACAGCTCCAGCAGTAGCCATTGTTTG
GATCCTAACAATGAAGAAGGTGGTCATGGAGTGAAAGGCTGGGCCTTTGATGATGG
AAATGACGTGTGGATGGGAAGAACAATCAACGAGACGTCACGCTTAGGGTATGAAA
CCTTCAAAGTCATTGAAGGCTGGTCCAACCCTAAGTCCAAATTGCAGACAAATAGGC
AAGTCATAGTTGACAGAGGTGATAGGTCCGGTTATTCTGGTATTTTCTCTGTTGAAG
GCAAAAGCTGCATCAATCGGTGCTTTTATGTGGAGTTGATTAGGGGAAGAAAAGAG
GAAACTGAAGTCTTGTGGACCTCAAACAGTATTGTTGTGTTTTGTGGCACCTCAGGT
ACATATGGAACAGGCTCATGGCCTGATGGGGCGGACCTCAATCTCATGCCTATATAA
GCTTTCGCAATTTTAGAAAAAACTCCTTGTTTCTACT (SEQ ID NO: 3)
HK14 N2-Ins15 Amino Acid
MNPNQKIITIGSVSLTISTICFFMQIAILITTVTLHFKQYEFNSPPNNQVMLCEPTIIERNITEI
VYLTNQTYVNISNTNFAAGNTTIEKEICPKPAEYRNWSKPQCGITGFAPFSKDNSIRLSAG
GDIWVTREPYVSCDPDKCYQFALGQGTTLNNVHSNNTVRDRTPYRTLLMNELGVPFHL
GTKQVCIAWSSSSCHDGKAWLHVCITGDDKNATASFIYNGRLVDSVVSWSKDILRTQES
ECICINGTCTVVMTDGSASGKADTKILFIEEGKIVHTSTLSGSAQHVEECSCYPRYPGVRC
VCRDNWKGSNRPIVDINIKDHSIVSSYVCSGLVGDTPRKNDSSSSSHCLDPNNEEGGHGV
KGWAFDDGNDVWMGRTINETSRLGYETFKVIEGWSNPKSKLQTNRQVIVDRGDRSGYS
GIFSVEGKSCINRCFYVELIRGRKEETEVLWTSNSIVVFCGTSGTYGTGSWPDGADLNLM
PI (SEQ ID NO: 4)
HK14 NA Nucleotide (1466 bp)
AGCAAAAGCAGGAGTAAAGATGAATCCAAATCAAAAGATAATAACGATTGGCTCTG
TTTCTCTCACCATTTCCACAATATGCTTCTTCATGCAAATTGCCATTTTGATAACTAC
TGTAACATTGCATTTCAAGCAATATGAATTCAACTCCCCCCCAAACAACCAAGTGAT
GCTGTGTGAACCAACAATAATAGAAAGAAACATAACAGAAATAGTGTATTTAACTA
ACACCACCATAGAGAAGGAAATATGCCCCAAACCAGCAGAATACAGAAATTGGTCA
AAACCGCAATGTGGCATTACAGGATTTGCACCTTTCTCTAAGGACAATTCGATTAGG
CTTTCCGCTGGTGGGGACATCTGGGTGACAAGAGAACCTTATGTGTCATGCGATCCT
GACAAGTGTTATCAATTTGCCCTTGGACAGGGAACAACACTAAACAACGTGCATTCA
AATAACACAGTACGTGATAGGACCCCTTATCGGACTCTATTGATGAATGAGTTGGGT
GTTCCTTTCCATCTGGGGACCAAGCAAGTGTGCATAGCATGGTCCAGCTCAAGTTGT
CACGATGGAAAAGCATGGCTGCATGTTTGTATAACGGGGGATGATAAAAATGCAAC
TGCTAGCTTCATTTACAATGGGAGGCTTGTAGATAGTGTTGTTTCATGGTCCAAAGA
TATTCTCAGGACCCAGGAGTCAGAATGCATTTGTATCAATGGAACTTGTACAGTAGT
AATGACTGATGGAAGTGCTTCAGGAAAAGCTGATACTAAAATACTATTCATTGAGG
AGGGGAAAATCGTTCATACTAGCACATTGTCAGGAAGTGCTCAGCATGTCGAAGAG
TGCTCTTGCTATCCTCGATATCCTGGTGTCAGATGTGTCTGCAGAGACAACTGGAAG
GGCTCCAATCGGCCCATCGTAGATATAAACATAAAGGATCATAGCATTGTTTCCAGT
TATGTGTGTTCAGGACTTGTTGGAGACACACCCAGAAAAAACGACAGCTCCAGCAG
TAGCCATTGTTTGGATCCTAACAATGAAGAAGGTGGTCATGGAGTGAAAGGCTGGG
CCTTTGATGATGGAAATGACGTGTGGATGGGAAGAACAATCAACGAGACGTCACGC
TTAGGGTATGAAACCTTCAAAGTCATTGAAGGCTGGTCCAACCCTAAGTCCAAATTG
CAGACAAATAGGCAAGTCATAGTTGACAGAGGTGATAGGTCCGGTTATTCTGGTATT
TTCTCTGTTGAAGGCAAAAGCTGCATCAATCGGTGCTTTTATGTGGAGTTGATTAGG
GGAAGAAAAGAGGAAACTGAAGTCTTGTGGACCTCAAACAGTATTGTTGTGTTTTGT
GGCACCTCAGGTACATATGGAACAGGCTCATGGCCTGATGGGGCGGACCTCAATCT
CATGCCTATATAAGCTTTCGCAATTTTAGAAAAAACTCCTTGTTTCTACT (SEQ ID
NO: 5)
HK14 NA Amino Acid
MNPNQKIITIGSVSLTISTICFFMQIAILITTVTLHFKQYEFNSPPNNQVMLCEPTIIERNITEI
VYLTNTTIEKEICPKPAEYRNWSKPQCGITGFAPFSKDNSIRLSAGGDIWVTREPYVSCDP
DKCYQFALGQGTTLNNVHSNNTVRDRTPYRTLLMNELGVPFHLGTKQVCIAWSSSSCH
DGKAWLHVCITGDDKNATASFIYNGRLVDSVVSWSKDILRTQESECICINGTCTVVMTD
GSASGKADTKILFIEEGKIVHTSTLSGSAQHVEECSCYPRYPGVRCVCRDNWKGSNRPIV
DINIKDHSIVSSYVCSGLVGDTPRKNDSSSSSHCLDPNNEEGGHGVKGWAFDDGNDVW
MGRTINETSRLGYETFKVIEGWSNPKSKLQTNRQVIVDRGDRSGYSGIFSVEGKSCINRC
FYVELIRGRKEETEVLWTSNSIVVFCGTSGTYGTGSWPDGADLNLMPI (SEQ ID NO: 6)
PR8 N1-Ins15 Nucleotide
agcgaaagcaggggtttaaaATGAATCCAAATCAGAAAATAACAACCATTGGATCAATCTGTCT
GGTAGTCGGACTAATTAGCCTAATATTGCAAATAGGGAATATAATCTCAATATGGAT
TAGCCATTCAATTCAAACTGGAAGTCAAAACCATACTGGAATATGCAACCAAAACA
TCATTACCTATAAAAATAGCACCTGGGTAAATCAGACATATGTTAACATCAGCAACA
CCAACTTTGCTGCTGGAAAGGACACAACTTCAGTGATATTAACCGGCAATTCATCTC
TTTGTCCCATCCGTGGGTGGGCTATATACAGCAAAGACAATAGCATAAGAATTGGTT
CCAAAGGAGACGTTTTTGTCATAAGAGAGCCCTTTATTTCATGTTCTCACTTGGAAT
GCAGGACCTTTTTTCTGACCCAAGGTGCCTTACTGAATGACAAGCATTCAAATGGGA
CTGTTAAGGACAGAAGCCCTTATAGGGCCTTAATGAGCTGCCCTGTCGGTGAAGCTC
CGTCCCCGTACAATTCAAGATTTGAATCGGTTGCTTGGTCAGCAAGTGCATGTCATG
ATGGCATGGGCTGGCTAACAATCGGAATTTCAGGTCCAGATAATGGAGCAGTGGCT
GTATTAAAATACAACGGCATAATAACTGAAACCATAAAAAGTTGGAGGAAGAAAAT
ATTGAGGACACAAGAGTCTGAATGTGCCTGTGTAAATGGTTCATGTTTTACTATAAT
GACTGATGGCCCGAGTGATGGGCTGGCCTCGTACAAAATTTTCAAGATCGAAAAGG
GGAAGGTTACTAAATCAATAGAGTTGAATGCACCTAATTCTCACTATGAGGAATGTT
CCTGTTACCCTGATACCGGCAAAGTGATGTGTGTGTGCAGAGACAACTGGCATGGTT
CGAACCGGCCATGGGTGTCTTTCGATCAAAACCTGGATTATCAAATAGGATACATCT
GCAGTGGGGTTTTCGGTGACAACCCGCGTCCCGAAGATGGAACAGGCAGCTGTGGT
CCAGTGTATGTTGATGGAGCAAACGGAGTAAAGGGATTTTCATATAGGTATGGTAAT
GGTGTTTGGATAGGAAGGACCAAAAGTCACAGTTCCAGACATGGGTTTGAGATGAT
TTGGGATCCTAATGGATGGACAGAGACTGATAGTAAGTTCTCTGTTAGGCAAGATGT
TGTGGCAATGACTGATTGGTCAGGGTATAGCGGAAGTTTCGTTCAACATCCTGAGCT
AACAGGGCTAGACTGTATGAGGCCGTGCTTCTGGGTTGAATTAATCAGGGGACGAC
CTAAAGAAAAAACAATCTGGACTAGTGCGAGCAGCATTTCTTTTTGTGGCGTGAATA
GTGATACTGTAGATTGGTCTTGGCCAGACGGTGCTGAGTTGCCATTCAGCATTGACA
AGTAGtctgttcaaaaaactccttgtttctact (SEQ ID NO: 7)
PR8 N1-Ins15 Amino Acid
KMNPNQKITTIGSICLVVGLISLILQIGNIISIWISHSIQTGSQNHTGICNQNIITYKNSTWVN
QTYVNISNTNFAAGKDTTSVILTGNSSLCPIRGWAIYSKDNSIRIGSKGDVFVIREPFISCS
HLECRTFFLTQGALLNDKHSNGTVKDRSPYRALMSCPVGEAPSPYNSRFESVAWSASAC
HDGMGWLTIGISGPDNGAVAVLKYNGIITETIKSWRKKILRTQESECACVNGSCFTIMTD
GPSDGLASYKIFKIEKGKVTKSIELNAPNSHYEECSCYPDTGKVMCVCRDNWHGSNRP
WVSFDQNLDYQIGYICSGVFGDNPRPEDGTGSCGPVYVDGANGVKGFSYRYGNGVWIG
RTKSHSSRHGFEMIWDPNGWTETDSKFSVRQDVVAMTDWSGYSGSFVQHPELTGLDC
MRPCFWVELIRGRPKEKTIWTSASSISFCGVNSDTVDWSWPDGAELPFSIDK (SEQ ID
NO: 8)
PR8 N1-Ins30 Nucleotide
agcgaaagcaggggtttaaaATGAATCCAAATCAGAAAATAACAACCATTGGATCAATCTGTCT
GGTAGTCGGACTAATTAGCCTAATATTGCAAATAGGGAATATAATCTCAATATGGAT
TAGCCATTCAATTCAAACTGGAAGTCAAAACCATACTGGAATATGCAACCAAAACA
TCATTACCTATAAAAATAGCACCTGGGTAAATCAGACATATGTTAACATCAGCAACA
CCAACTTTGCTGCTGGAAACACAACAGAGATAGTGTATCTGACCAACACCACCATA
GAGAAGAAGGACACAACTTCAGTGATATTAACCGGCAATTCATCTCTTTGTCCCATC
CGTGGGTGGGCTATATACAGCAAAGACAATAGCATAAGAATTGGTTCCAAAGGAGA
CGTTTTTGTCATAAGAGAGCCCTTTATTTCATGTTCTCACTTGGAATGCAGGACCTTT
TTTCTGACCCAAGGTGCCTTACTGAATGACAAGCATTCAAATGGGACTGTTAAGGAC
AGAAGCCCTTATAGGGCCTTAATGAGCTGCCCTGTCGGTGAAGCTCCGTCCCCGTAC
AATTCAAGATTTGAATCGGTTGCTTGGTCAGCAAGTGCATGTCATGATGGCATGGGC
TGGCTAACAATCGGAATTTCAGGTCCAGATAATGGAGCAGTGGCTGTATTAAAATAC
AACGGCATAATAACTGAAACCATAAAAAGTTGGAGGAAGAAAATATTGAGGACAC
AAGAGTCTGAATGTGCCTGTGTAAATGGTTCATGTTTTACTATAATGACTGATGGCC
CGAGTGATGGGCTGGCCTCGTACAAAATTTTCAAGATCGAAAAGGGGAAGGTTACT
AAATCAATAGAGTTGAATGCACCTAATTCTCACTATGAGGAATGTTCCTGTTACCCT
GATACCGGCAAAGTGATGTGTGTGTGCAGAGACAACTGGCATGGTTCGAACCGGCC
ATGGGTGTCTTTCGATCAAAACCTGGATTATCAAATAGGATACATCTGCAGTGGGGT
TTTCGGTGACAACCCGCGTCCCGAAGATGGAACAGGCAGCTGTGGTCCAGTGTATGT
TGATGGAGCAAACGGAGTAAAGGGATTTTCATATAGGTATGGTAATGGTGTTTGGAT
AGGAAGGACCAAAAGTCACAGTTCCAGACATGGGTTTGAGATGATTTGGGATCCTA
ATGGATGGACAGAGACTGATAGTAAGTTCTCTGTTAGGCAAGATGTTGTGGCAATG
ACTGATTGGTCAGGGTATAGCGGAAGTTTCGTTCAACATCCTGAGCTAACAGGGCTA
GACTGTATGAGGCCGTGCTTCTGGGTTGAATTAATCAGGGGACGACCTAAAGAAAA
AACAATCTGGACTAGTGCGAGCAGCATTTCTTTTTGTGGCGTGAATAGTGATACTGT
AGATTGGTCTTGGCCAGACGGTGCTGAGTTGCCATTCAGCATTGACAAGTAGtctgttcaa
aaaactccttgtttctact (SEQ ID NO: 9)
PR8 N1-Ins30 Amino Acid
MNPNQKITTIGSICLVVGLISLILQIGNIISIWISHSIQTGSQNHTGICNQNIITYKNSTWVNQ
TYVNISNTNFAAGNTTEIVYLTNTTIEKKDTTSVILTGNSSLCPIRGWAIYSKDNSIRIGSK
GDVFVIREPFISCSHLECRTFFLTQGALLNDKHSNGTVKDRSPYRALMSCPVGEAPSPYN
SRFESVAWSASACHDGMGWLTIGISGPDNGAVAVLKYNGIITETIKSWRKKILRTQESEC
ACVNGSCFTIMTDGPSDGLASYKIFKIEKGKVTKSIELNAPNSHYEECSCYPDTGKVMCV
CRDNWHGSNRPWVSFDQNLDYQIGYICSGVFGDNPRPEDGTGSCGPVYVDGANGVKG
FSYRYGNGVWIGRTKSHSSRHGFEMIWDPNGWTETDSKFSVRQDVVAMTDWSGYSGS
FVQHPELTGLDCMRPCFWVELIRGRPKEKTIWTSASSISFCGVNSDTVDWSWPDGAELP
FSIDK (SEQ ID NO: 10)
PR8 N1-wt Nucleotide
agcgaaagcaggggtttaaaATGAATCCAAATCAGAAAATAACAACCATTGGATCAATCTGTCT
GGTAGTCGGACTAATTAGCCTAATATTGCAAATAGGGAATATAATCTCAATATGGAT
TAGCCATTCAATTCAAACTGGAAGTCAAAACCATACTGGAATATGCAACCAAAACA
TCATTACCTATAAAAATAGCACCTGGGTAAAGGACACAACTTCAGTGATATTAACCG
GCAATTCATCTCTTTGTCCCATCCGTGGGTGGGCTATATACAGCAAAGACAATAGCA
TAAGAATTGGTTCCAAAGGAGACGTTTTTGTCATAAGAGAGCCCTTTATTTCATGTT
CTCACTTGGAATGCAGGACCTTTTTTCTGACCCAAGGTGCCTTACTGAATGACAAGC
ATTCAAATGGGACTGTTAAGGACAGAAGCCCTTATAGGGCCTTAATGAGCTGCCCTG
TCGGTGAAGCTCCGTCCCCGTACAATTCAAGATTTGAATCGGTTGCTTGGTCAGCAA
GTGCATGTCATGATGGCATGGGCTGGCTAACAATCGGAATTTCAGGTCCAGATAATG
GAGCAGTGGCTGTATTAAAATACAACGGCATAATAACTGAAACCATAAAAAGTTGG
AGGAAGAAAATATTGAGGACACAAGAGTCTGAATGTGCCTGTGTAAATGGTTCATG
TTTTACTATAATGACTGATGGCCCGAGTGATGGGCTGGCCTCGTACAAAATTTTCAA
GATCGAAAAGGGGAAGGTTACTAAATCAATAGAGTTGAATGCACCTAATTCTCACT
ATGAGGAATGTTCCTGTTACCCTGATACCGGCAAAGTGATGTGTGTGTGCAGAGACA
ACTGGCATGGTTCGAACCGGCCATGGGTGTCTTTCGATCAAAACCTGGATTATCAAA
TAGGATACATCTGCAGTGGGGTTTTCGGTGACAACCCGCGTCCCGAAGATGGAACA
GGCAGCTGTGGTCCAGTGTATGTTGATGGAGCAAACGGAGTAAAGGGATTTTCATAT
AGGTATGGTAATGGTGTTTGGATAGGAAGGACCAAAAGTCACAGTTCCAGACATGG
GTTTGAGATGATTTGGGATCCTAATGGATGGACAGAGACTGATAGTAAGTTCTCTGT
TAGGCAAGATGTTGTGGCAATGACTGATTGGTCAGGGTATAGCGGAAGTTTCGTTCA
ACATCCTGAGCTAACAGGGCTAGACTGTATGAGGCCGTGCTTCTGGGTTGAATTAAT
CAGGGGACGACCTAAAGAAAAAACAATCTGGACTAGTGCGAGCAGCATTTCTTTTT
GTGGCGTGAATAGTGATACTGTAGATTGGTCTTGGCCAGACGGTGCTGAGTTGCCAT
TCAGCATTGACAAGTAGtctgttcaaaaaactccttgtttctact (SEQ ID NO: 11)
PR 8 N1-wt Amino Acid
MNPNQKITTIGSICLVVGLISLILQIGNIISIWISHSIQTGSQNHTGICNQNIITYKNSTWVKD
TTSVILTGNSSLCPIRGWAIYSKDNSIRIGSKGDVFVIREPFISCSHLECRTFFLTQGALLND
KHSNGTVKDRSPYRALMSCPVGEAPSPYNSRFESVAWSASACHDGMGWLTIGISGPDN
GAVAVLKYNGIITETIKSWRKKILRTQESECACVNGSCFTIIVITDGPSDGLASYKIFKIEKG
KVTKSIELNAPNSHYEECSCYPDTGKVMCVCRDNWHGSNRPWVSFDQNLDYQIGYICS
GVFGDNPRPEDGTGSCGPVYVDGANGVKGFSYRYGNGVWIGRTKSHSSRHGFEMIWD
PNGWTETDSKFSVRQDVVAMTDWSGYSGSFVQHPELTGLDCMRPCFWVELIRG
RPKEKTIWTSASSISFCGVNSDTVDWSWPDGAELPFSIDK (SEQ ID NO: 12)
Sequencing Primer pDZ_forward (TACAGCTCCTGGGCAACGTGCTGG; SEQ ID NO: 13)
Sequencing Primer pDZ_reverse (AGGTGTCCGTGTCGCGCGTCGCC; SEQ ID NO: 14)
Primer PR8_NA_forward (CGAAAGCAGGGGTTTAAAATG; SEQ ID NO: 15)
Primer PR8_NA_reverse (TTTTTGAACAGACTACTTGTCAATG; SEQ ID NO: 16),
Primer PR8_HA_forward (CCGAAGTTGGGGGGGAGCAAAAGCAGGGGAAAATAA; SEQ
ID NO: 17)
Primer PR8_HA_reverse (GGCCGCCGGGTTATTAGTAGAAACAAGGGTGTTTTT; SEQ ID
NO: 18)
Primer HK14_NA_forward (GGGAGCAAAAGCAGGAGTAAAGATG; SEQ ID NO: 19)
Primer HK14_NA_reverse (TTATTAGTAGAAACAAGGAGTTTTTTCTAAAATTGCG; SEQ
ID NO: 20)
Primer HK14_HA_forward (GGGAGCAAAAGCAGGGGATAATTC; SEQ ID NO: 21)
Primer HK14_HA_reverse (GGGTTATTAGTAGAAACAAGGGTGTTTTTAATTAATG; SEQ
ID NO: 22)
PR8 swap virus
NA-HAmut-NA
AGCGAAAGCAGGGGTTTAAATTGAATCCAAATCAGAAAATAACAACCATTGGATCA
ATCTGTCTGGTAGTCGGACTAATTAGCCTAATATTGCAAATAGGGAATATAATCTCA
ATTTGGATTAGCCATTCAATTCAAACTGGAAGTCAAAACCATACTGGAATTTGCAAC
CAAGCTAGCATGAAAGCGAATTTGTTAGTTTTACTGTCCGCGTTGGCGGCCGCGGACGC
AGACACAATATGTATAGGCTACCATGCGAACAATTCAACCGACACTGTTGACACAG
TACTCGAGAAGAATGTGACAGTGACACACTCTGTTAACCTGCTCGAAGACAGCCAC
AACGGAAAACTATGTAGATTAAAAGGAATAGCCCCACTACAATTGGGGAAATGTAA
CATCGCCGGATGGCTCTTGGGAAACCCAGAATGCGACCCACTGCTTCCAGTGAGATC
ATGGTCCTACATTGTAGAAACACCAAACTCTGAGAATGGAATATGTTATCCAGGAG
ATTTCATCGACTATGAGGAGCTGAGGGAGCAATTGAGCTCAGTGTCATCATTCGAAA
GATTCGAAATATTTCCCAAAGAAAGCTCATGGCCCAACCACAACACAAACGGAGTA
ACGGCAGCATGCTCCCATGAGGGGAAAAGCAGTTTTTACAGAAATTTGCTATGGCTG
ACGGAGAAGGAGGGCTCATACCCAAAGCTGAAAAATTCTTATGTGAACAAAAAAGG
GAAAGAAGTCCTTGTACTGTGGGGTATTCATCACCCGCCTAACAGTAAGGAACAAC
AGAATATCTATCAGAATGAAAATGCTTATGTCTCTGTAGTGACTTCAAATTATAACA
GGAGATTTACCCCGGAAATAGCAGAAAGACCCAAAGTAAGAGATCAAGCTGGGAG
GATGAACTATTACTGGACCTTGCTAAAACCCGGAGACACAATAATATTTGAGGCAA
ATGGAAATCTAATAGCACCAATGTATGCTTTCGCACTGAGTAGAGGCTTTGGGTCCG
GCATCATCACCTCAAACGCATCAATGCATGAGTGTAACACGAAGTGTCAAACACCC
CTGGGAGCTATAAACAGCAGTCTCCCTTACCAGAATATACACCCAGTCACAATAGG
AGAGTGCCCAAAATACGTCAGGAGTGCCAAATTGAGGATGGTTACAGGACTAAGGA
ACACTCCGTCCATTCAATCCAGAGGTCTATTTGGAGCCATTGCCGGTTTTATTGAAG
GGGGATGGACTGGAATGATAGATGGATGGTATGGTTATCATCATCAGAATGAACAG
GGATCAGGCTATGCAGCGGATCAAAAAAGCACACAAAATGCCATTAACGGGATTAC
AAACAAGGTGAACACTGTTATCGAGAAAATGAACATTCAATTCACAGCTGTGGGTA
AAGAATTCAACAAATTAGAAAAAAGGATGGAAAATTTAAATAAAAAAGTTGATGAT
GGATTTCTGGACATTTGGACATATAATGCAGAATTGTTAGTTCTACTGGAAAATGAA
AGGACTCTGGATTTCCATGACTCAAATGTGAAGAATCTGTATGAGAAAGTAAAAAG
CCAATTAAAGAATAATGCCAAAGAAATCGGAAATGGATGTTTTGAGTTCTACCACA
AGTGTGACAATGAATGCATGGAAAGTGTAAGAAATGGGACTTATGATTATCCCAAA
TATTCAGAAGAGTCAAAGTTGAACAGGGAAAAGGTAGATGGAGTGAAATTGGAATC
AATGGGGATCTATCAGATTCTGGCGATCTACTCAACTGTCGCTTCCAGCTTAGTATTG
CTAGTTAGTTTAGGAGCGATTTCCTTTTGGATGTGCAGCAACGGGAGCCTACAATGTCGGA
TTTGTATTTGACTCGAGTGAGCTAACAGGGCTAGACTGTATGAGGCCGTGCTTCTGGG
TTGAATTAATCAGGGGACGACCTAAAGAAAAAACAATCTGGACTAGTGCGAGCAGC
ATTTCTTTTTGTGGCGTGAATAGTGATACTGTAGATTGGTCTTGGCCAGACGGTGCTG
AGTTGCCATTCAGCATTGACAAGTAGTCTGTTCAAAAAACTCCTTGTTTCTACT
(SEQ ID NO: 23)
Packaging signals underlined
Synonymous mutations italicized
PR8 swap virus
HA-NAmut-HA
AGCAAAAGCAGGGGAAAATAAAAACAACCAAATTGAAGGCAAACCTACTGGTCCT
GTTAAGTGCACTTGCAGCTGCAGTTGCAGACACAATTTGTATAGGCTAGCATGAACC
CGAACCAAAAGATCACGACTATCGGGAGCATTTGCTTAGTGGTTGGGTTGATCAGCCTAA
TATTGCAAATAGGGAATATAATCTCAATATGGATTAGCCATTCAATTCAAACTGGAA
GTCAAAACCATACTGGAATATGCAACCAAAACATCATTACCTATAAAAATAGCACC
TGGGTAAAGGACACAACTTCAGTGATATTAACCGGCAATTCATCTCTTTGTCCCATC
CGTGGGTGGGCTATATACAGCAAAGACAATAGCATAAGAATTGGTTCCAAAGGAGA
CGTTTTTGTCATAAGAGAGCCCTTTATTTCATGTTCTCACTTGGAATGCAGGACCTTT
TTTCTGACCCAAGGTGCCTTACTGAATGACAAGCATTCAAATGGGACTGTTAAGGAC
AGAAGCCCTTATAGGGCCTTAATGAGCTGCCCTGTCGGTGAAGCTCCGTCCCCGTAC
AATTCAAGATTTGAATCGGTTGCTTGGTCAGCAAGTGCATGTCATGATGGCATGGGC
TGGCTAACAATCGGAATTTCAGGTCCAGATAATGGAGCAGTGGCTGTATTAAAATAC
AACGGCATAATAACTGAAACCATAAAAAGTTGGAGGAAGAAAATATTGAGGACAC
AAGAGTCTGAATGTGCCTGTGTAAATGGTTCATGTTTTACTATAATGACTGATGGCC
CGAGTGATGGGCTGGCCTCGTACAAAATTTTCAAGATCGAAAAGGGGAAGGTTACT
AAATCAATAGAGTTGAATGCACCTAATTCTCACTATGAGGAATGTTCCTGTTACCCT
GATACCGGCAAAGTGATGTGTGTGTGCAGAGACAACTGGCATGGTTCGAACCGGCC
ATGGGTGTCTTTCGATCAAAACCTGGATTATCAAATAGGATACATCTGCAGTGGGGT
TTTCGGTGACAACCCGCGTCCCGAAGATGGAACAGGCAGCTGTGGTCCAGTGTATGT
TGATGGAGCAAACGGAGTAAAGGGATTTTCATATAGGTATGGTAATGGTGTTTGGAT
AGGAAGGACCAAAAGTCACAGTTCCAGACATGGGTTTGAGATGATTTGGGATCCTA
ATGGATGGACAGAGACTGATAGTAAGTTCTCTGTTAGGCAAGATGTTGTGGCAATG
ACTGATTGGTCAGGGTATAGCGGAAGTTTCGTTCAACATCCTGAGCTAACAGGGCTA
GACTGTATGAGGCCGTGCTTCTGGGTTGAATTAATCAGGGGACGACCTAAAGAAAA
AACAATCTGGACTAGTGCGAGCAGCATTTCTTTTTGTGGCGTGAATAGTGACACCGTA
GACTGGAGCTGGCCGGATGGCGCCGAACTACCGTTTTCTATCGATAAATAGCTCGAGATC
TACTCAACTGTCGCCAGTTCACTGGTGCTTTTGGTCTCCCTGGGGGCAATCAGTTTCT
GGATGTGTTCTAATGGATCTTTGCAGTGCAGAATATGCATCTGAGATTAGAATTTCA
GAAATATGAGGAAAAACACCCTTGTTTCTACT
(SEQ ID NO: 24)
Packaging signals underlined
Synonymous mutations italicized
HK14 swap virus
NA-HK14 HA ORF-NA
AGCGAAAGCAGGGGTTTAAATTGAATCCAAATCAGAAAATAACAACCATTGGATCA
ATCTGTCTGGTAGTCGGACTAATTAGCCTAATATTGCAAATAGGGAATATAATCTCA
ATTTGGATTAGCCATTCAATTCAAACTGGAAGTCAAAACCATACTGGAATTTGCAAC
CAAGCTAGCATGAAGACTATCATTGCTTTGAGCTACATTCTATGTCTGGTTTTCGCTC
AAAAAATTCCTGGAAATGACAATAGCACGGCAACGCTGTGCCTTGGGCACCATGCA
GTACCAAACGGAACGATAGTGAAAACAATCACGAATGACCGAATTGAAGTTACTAA
TGCTACTGAGCTGGTTCAGAATTCCTCAATAGGTGAAATATGCGACAGTCCTCATCA
GATCCTTGATGGAGAAAACTGCACACTAATAGATGCTCTATTGGGAGACCCTCAGTG
TGATGGCTTTCAAAATAAGAAATGGGACCTTTTTGTTGAACGAAGCAAAGCCTACAG
CAGCTGTTACCCTTATGATGTGCCGGATTATGCCTCCCTTAGGTCACTAGTTGCCTCA
TCCGGCACACTGGAGTTTAACAATGAAAGCTTCAATTGGACTGGAGTCACTCAAAAC
GGAACAAGTTCTGCTTGCATAAGGAGATCTAGTAGTAGTTTCTTTAGTAGATTAAAT
TGGTTGACCCACTTAAACTACAAATACCCAGCATTGAACGTGACTATGCCAAACAAT
GAACAATTTGACAAATTGTACATTTGGGGGGTTCACCACCCGGGTACGGACAAGGA
CCAAATCTTCCCGTATGCTCAATCATCAGGAAGAATCACAGTATCTACCAAAAGAAG
CCAACAAGCTGTAATCCCAAATATCGGATCTAGACCCAGAATAAGGAATATCCCTA
GCAGAATAAGCATCTATTGGACAATAGTAAAACCGGGAGACATACTTTTGATTAAC
AGCACAGGGAATCTAATTGCTCCTAGGGGTTACTTCAAAATACGAAGTGGGAAAAG
CTCAATAATGAGATCAGATGCACCCATTGGCAAATGCAAGTCTGAATGCATCACTCC
AAATGGAAGCATTCCCAATGACAAACCATTCCAAAATGTAAACAGGATCACATACG
GGGCCTGTCCCAGATATGTTAAGCATAGCACTCTGAAATTGGCAACAGGAATGCGA
AATGTACCAGAGAAACAAACTAGAGGCATATTTGGCGCAATAGCGGGTTTCATAGA
AAATGGTTGGGAGGGAATGGTGGATGGTTGGTACGGTTTCAGGCATCAAAATTCTG
AGGGAAGAGGACAAGCAGCAGATCTCAAAAGCACTCAAGCAGCAATCGATCAAAT
CAATGGGAAGCTGAATCGATTGATCGGGAAAACCAACGAGAAATTCCATCAGATTG
AAAAAGAATTCTCAGAAGTAGAAGGAAGAATTCAGGACCTTGAGAAATATGTTGAG
GACACTAAAATAGATCTCTGGTCATACAACGCGGAGCTTCTTGTTGCCCTGGAGAAC
CAACATACAATTGATCTAACTGACTCAGAAATGAACAAACTGTTTGAAAAAACAAA
GAAGCAACTGAGGGAAAATGCTGAGGATATGGGCAATGGTTGTTTCAAAATATACC
ACAAATGTGACAATGCCTGCATAGGATCAATAAGAAATGGAACTTATGACCACAAT
GTGTACAGGGATGAAGCATTAAACAACCGGTTCCAGATCAAGGGAGTTGAGCTGAA
GTCAGGGTACAAAGATTGGATCCTATGGATTTCCTTTGCCATATCATGTTTTTTGCTT
TGTGTTGCTTTGTTGGGGTTCATCATGTGGGCCTGCCAAAAGGGCAACATTAGGTGC
AACATTTGCATTTGACTCGAGTGAGCTAACAGGGCTAGACTGTATGAGGCCGTGCTT
CTGGGTTGAATTAATCAGGGGACGACCTAAAGAAAAAACAATCTGGACTAGTGCGA
GCAGCATTTCTTTTTGTGGCGTGAATAGTGATACTGTAGATTGGTCTTGGCCAGACG
GTGCTGAGTTGCCATTCAGCATTGACAAGTAGTCTGTTCAAAAAACTCCTTGTTTCTA
CT
(SEQ ID NO: 25)
Packaging signals underlined
HK14 swap virus
HA-HK14 NA ORF-HA
AGCAAAAGCAGGGGAAAATAAAAACAACCAAATTGAAGGCAAACCTACTGGTCCT
GTTAAGTGCACTTGCAGCTGCAGTTGCAGACACAATTTGTATAGGCTAGCATGAATC
CAAATCAAAAGATAATAACGATTGGCTCTGTTTCTCTCACCATTTCCACAATATGCTT
CTTCATGCAAATTGCCATTTTGATAACTACTGTAACATTGCATTTCAAGCAATATGA
ATTCAACTCCCCCCCAAACAACCAAGTGATGCTGTGTGAACCAACAATAATAGAAA
GAAACATAACAGAAATAGTGTATTTAACTAACACCACCATAGAGAAGGAAATATGC
CCCAAACCAGCAGAATACAGAAATTGGTCAAAACCGCAATGTGGCATTACAGGATT
TGCACCTTTCTCTAAGGACAATTCGATTAGGCTTTCCGCTGGTGGGGACATCTGGGT
GACAAGAGAACCTTATGTGTCATGCGATCCTGACAAGTGTTATCAATTTGCCCTTGG
ACAGGGAACAACACTAAACAACGTGCATTCAAATAACACAGTACGTGATAGGACCC
CTTATCGGACTCTATTGATGAATGAGTTGGGTGTTCCTTTCCATCTGGGGACCAAGC
AAGTGTGCATAGCATGGTCCAGCTCAAGTTGTCACGATGGAAAAGCATGGCTGCAT
GTTTGTATAACGGGGGATGATAAAAATGCAACTGCTAGCTTCATTTACAATGGGAGG
CTTGTAGATAGTGTTGTTTCATGGTCCAAAGATATTCTCAGGACCCAGGAGTCAGAA
TGCATTTGTATCAATGGAACTTGTACAGTAGTAATGACTGATGGAAGTGCTTCAGGA
AAAGCTGATACTAAAATACTATTCATTGAGGAGGGGAAAATCGTTCATACTAGCAC
ATTGTCAGGAAGTGCTCAGCATGTCGAAGAGTGCTCTTGCTATCCTCGATATCCTGG
TGTCAGATGTGTCTGCAGAGACAACTGGAAGGGCTCCAATCGGCCCATCGTAGATAT
AAACATAAAGGATCATAGCATTGTTTCCAGTTATGTGTGTTCAGGACTTGTTGGAGA
CACACCCAGAAAAAACGACAGCTCCAGCAGTAGCCATTGTTTGGATCCTAACAATG
AAGAAGGTGGTCATGGAGTGAAAGGCTGGGCCTTTGATGATGGAAATGACGTGTGG
ATGGGAAGAACAATCAACGAGACGTCACGCTTAGGGTATGAAACCTTCAAAGTCAT
TGAAGGCTGGTCCAACCCTAAGTCCAAATTGCAGACAAATAGGCAAGTCATAGTTG
ACAGAGGTGATAGGTCCGGTTATTCTGGTATTTTCTCTGTTGAAGGCAAAAGCTGCA
TCAATCGGTGCTTTTATGTGGAGTTGATTAGGGGAAGAAAAGAGGAAACTGAAGTC
TTGTGGACCTCAAACAGTATTGTTGTGTTTTGTGGCACCTCAGGTACATATGGAACA
GGCTCATGGCCTGATGGGGCGGACCTCAATCTCATGCCTATATAACTCGAGATCTAC
TCAACTGTCGCCAGTTCACTGGTGCTTTTGGTCTCCCTGGGGGCAATCAGTTTCTGGA
TGTGTTCTAATGGATCTTTGCAGTGCAGAATATGCATCTGAGATTAGAATTTCAGAA
ATATGAGGAAAAACACCCTTGTTTCTACT
(SEQ ID NO: 26)
Packaging signals underlined
PR8 NA long stalk ORF between PR8 HA packaging signals
Packaging signals are underlined, stalk insertion is italicized and double underlined
AGCAAAAGCAGGGGAAAATAAAAACAACCAAATTGAAGGCAAACCTACTGGTCCT
GTTAAGTGCACTTGCAGCTGCAGTTGCAGACACAATTTGTATAGGCTAGCATGAACC
CGAACCAAAAGATCACGACTATCGGGAGCATTTGCTTAGTGGTTGGGTTGATCAGCC
TAATATTGCAAATAGGGAATATAATCTCAATATGGATTAGCCATTCAATTCAAACTG
GAAGTCAAAACCATACTGGAATATGCAACCAAAACATCATTACCTATAAAAATAGC

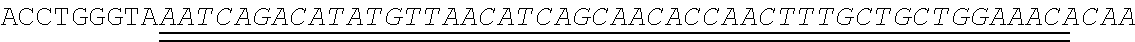

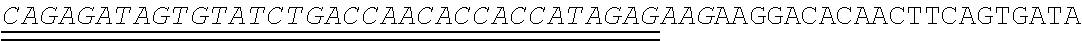

TTAACCGGCAATTCATCTCTTTGTCCCATCCGTGGGTGGGCTATATACAGCAAAGAC
AATAGCATAAGAATTGGTTCCAAAGGAGACGTTTTTGTCATAAGAGAGCCCTTTATT
TCATGTTCTCACTTGGAATGCAGGACCTTTTTTCTGACCCAAGGTGCCTTACTGAATG
ACAAGCATTCAAATGGGACTGTTAAGGACAGAAGCCCTTATAGGGCCTTAATGAGC
TGCCCTGTCGGTGAAGCTCCGTCCCCGTACAATTCAAGATTTGAATCGGTTGCTTGG
TCAGCAAGTGCATGTCATGATGGCATGGGCTGGCTAACAATCGGAATTTCAGGTCCA
GATAATGGAGCAGTGGCTGTATTAAAATACAACGGCATAATAACTGAAACCATAAA
AAGTTGGAGGAAGAAAATATTGAGGACACAAGAGTCTGAATGTGCCTGTGTAAATG
GTTCATGTTTTACTATAATGACTGATGGCCCGAGTGATGGGCTGGCCTCGTACAAAA
TTTTCAAGATCGAAAAGGGGAAGGTTACTAAATCAATAGAGTTGAATGCACCTAATT
CTCACTATGAGGAATGTTCCTGTTACCCTGATACCGGCAAAGTGATGTGTGTGTGCA
GAGACAACTGGCATGGTTCGAACCGGCCATGGGTGTCTTTCGATCAAAACCTGGATT
ATCAAATAGGATACATCTGCAGTGGGGTTTTCGGTGACAACCCGCGTCCCGAAGATG
GAACAGGCAGCTGTGGTCCAGTGTATGTTGATGGAGCAAACGGAGTAAAGGGATTT
TCATATAGGTATGGTAATGGTGTTTGGATAGGAAGGACCAAAAGTCACAGTTCCAG
ACATGGGTTTGAGATGATTTGGGATCCTAATGGATGGACAGAGACTGATAGTAAGTT
CTCTGTTAGGCAAGATGTTGTGGCAATGACTGATTGGTCAGGGTATAGCGGAAGTTT
CGTTCAACATCCTGAGCTAACAGGGCTAGACTGTATGAGGCCGTGCTTCTGGGTTGA
ATTAATCAGGGGACGACCTAAAGAAAAAACAATCTGGACTAGTGCGAGCAGCATTT
CTTTTTGTGGCGTGAATAGTGACACCGTAGACTGGAGCTGGCCGGATGGCGCCGAAC
TACCGTTTTCTATCGATAAATAGCTCGAGATCTACTCAACTGTCGCCAGTTCACTGGT
GCTTTTGGTCTCCCTGGGGGCAATCAGTTTCTGGATGTGTTCTAATGGATCTTTGCAG
TGCAGAATATGCATCTGAGATTAGAATTTCAGAAATATGAGGAAAAACACCCTTGTT
TCTACT
(SEQ ID NO: 27)
HK14 N2 long stalk ORF between PR8 HA packaging signals
Packaging signals are underlined, stalk insertion is italicized and double underlined
AGCAAAAGCAGGGGAAAATAAAAACAACCAAATTGAAGGCAAACCTACTGGTCCT
GTTAAGTGCACTTGCAGCTGCAGTTGCAGACACAATTTGTATAGGCTAGCATGAATC
CAAATCAAAAGATAATAACGATTGGCTCTGTTTCTCTCACCATTTCCACAATATGCTT
CTTCATGCAAATTGCCATTTTGATAACTACTGTAACATTGCATTTCAAGCAATATGA
ATTCAACTCCCCCCCAAACAACCAAGTGATGCTGTGTGAACCAACAATAATAGAAA

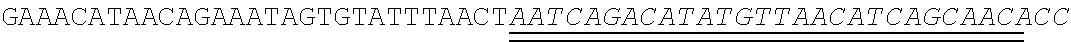

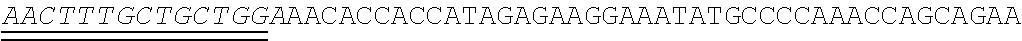

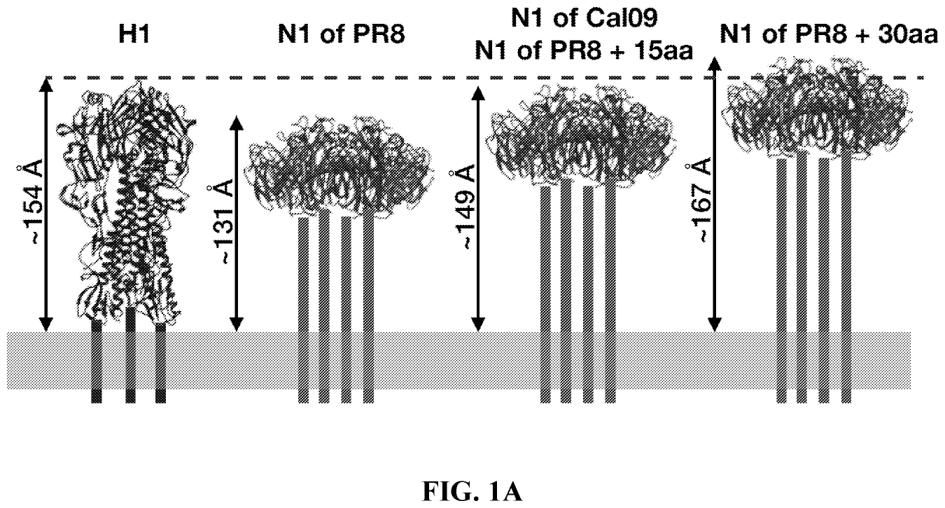

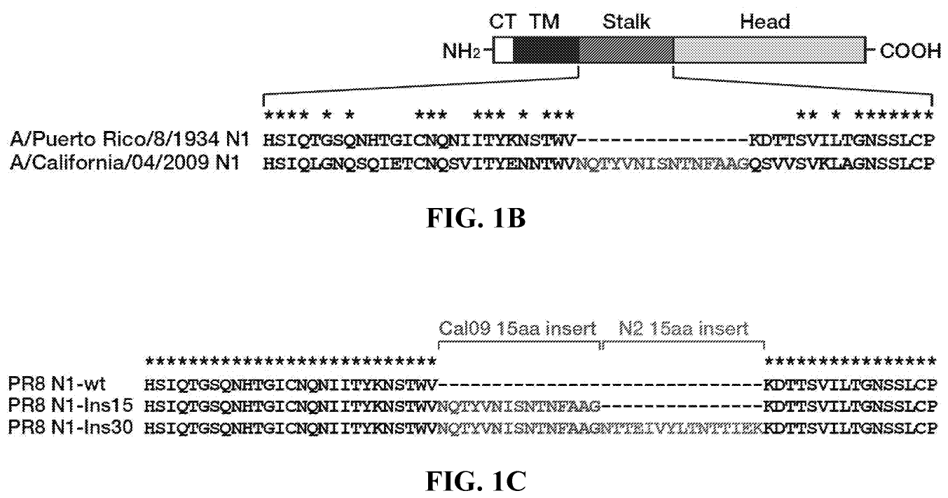

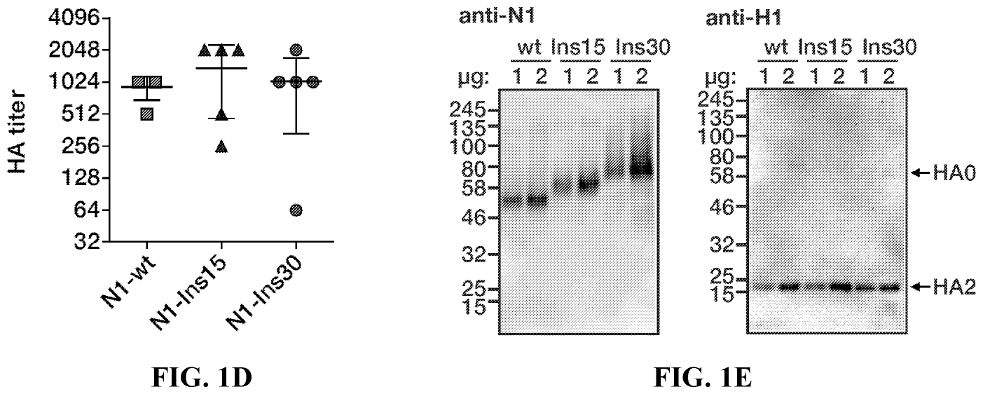

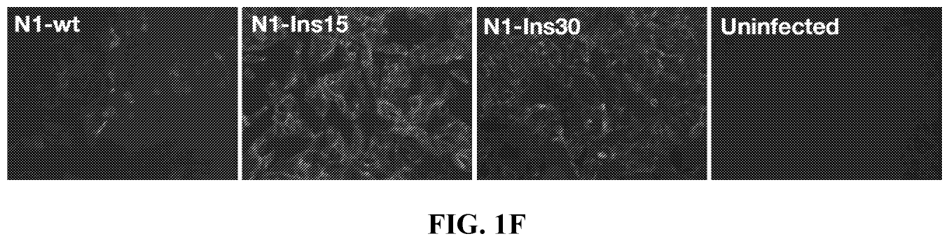

TACAGAAATTGGTCAAAACCGCAATGTGGCATTACAGGATTTGCACCTTTCTCTAAG
GACAATTCGATTAGGCTTTCCGCTGGTGGGGACATCTGGGTGACAAGAGAACCTTAT
GTGTCATGCGATCCTGACAAGTGTTATCAATTTGCCCTTGGACAGGGAACAACACTA
AACAACGTGCATTCAAATAACACAGTACGTGATAGGACCCCTTATCGGACTCTATTG
ATGAATGAGTTGGGTGTTCCTTTCCATCTGGGGACCAAGCAAGTGTGCATAGCATGG
TCCAGCTCAAGTTGTCACGATGGAAAAGCATGGCTGCATGTTTGTATAACGGGGGAT
GATAAAAATGCAACTGCTAGCTTCATTTACAATGGGAGGCTTGTAGATAGTGTTGTT
TCATGGTCCAAAGATATTCTCAGGACCCAGGAGTCAGAATGCATTTGTATCAATGGA
ACTTGTACAGTAGTAATGACTGATGGAAGTGCTTCAGGAAAAGCTGATACTAAAAT
ACTATTCATTGAGGAGGGGAAAATCGTTCATACTAGCACATTGTCAGGAAGTGCTCA
GCATGTCGAAGAGTGCTCTTGCTATCCTCGATATCCTGGTGTCAGATGTGTCTGCAG
AGACAACTGGAAGGGCTCCAATCGGCCCATCGTAGATATAAACATAAAGGATCATA
GCATTGTTTCCAGTTATGTGTGTTCAGGACTTGTTGGAGACACACCCAGAAAAAACG
ACAGCTCCAGCAGTAGCCATTGTTTGGATCCTAACAATGAAGAAGGTGGTCATGGA
GTGAAAGGCTGGGCCTTTGATGATGGAAATGACGTGTGGATGGGAAGAACAATCAA
CGAGACGTCACGCTTAGGGTATGAAACCTTCAAAGTCATTGAAGGCTGGTCCAACCC
TAAGTCCAAATTGCAGACAAATAGGCAAGTCATAGTTGACAGAGGTGATAGGTCCG
GTTATTCTGGTATTTTCTCTGTTGAAGGCAAAAGCTGCATCAATCGGTGCTTTTATGT
GGAGTTGATTAGGGGAAGAAAAGAGGAAACTGAAGTCTTGTGGACCTCAAACAGTA
TTGTTGTGTTTTGTGGCACCTCAGGTACATATGGAACAGGCTCATGGCCTGATGGGG
CGGACCTCAATCTCATGCCTATATAA CTCGAGATCTACTCAACTGTCGCCAGTTCAC
TGGTGCTTTTGGTCTCCCTGGGGGCAATCAGTTTCTGGATGTGTTCTAATGGATCTTT
GCAGTGCAGAATATGCATCTGAGATTAGAATTTCAGAAATATGAGGAAAAACACCC
TTGTTTCTACT
(SEQ ID NO: 28)
Nucleotide sequence encoding 15 amino acid sequence extension
AATCAGACATATGTTAACATCAGCAACACCAACTTTGCTGCTGGA
(SEQ ID NO: 29)
Bris08 HA
AGCAGAAGCAGAGCATTTTCTAATATCCACAAAATGAAGGCAATAATTGTACTACTC
ATGGTAGTAACATCCAATGCAGATCGAATCTGCACTGGGATAACATCGTCAAACTCA
CCACATGTCGTCAAAACTGCTACTCAAGGGGAGGTCAATGTGACTGGTGTAATACCA
CTGACAACAACACCCACCAAATCTCATTTTGCAAATCTCAAAGGAACAGAAACCAG
GGGGAAACTATGCCCAAAATGCCTCAACTGCACAGATCTGGACGTAGCCTTGGGCA
GACCAAAATGCACGGGGAAAATACCCTCGGCAAGAGTTTCAATACTCCATGAAGTC
AGACCTGTTACATCTGGGTGCTTTCCTATAATGCACGACAGAACAAAAATTAGACAG
CTGCCTAACCTTCTCCGAGGATACGAACATATCAGGTTATCAACCCATAACGTTATC
AATGCAGAAAATGCACCAGGAGGACCCTACAAAATTGGAACCTCAGGGTCTTGCCC
TAACATTACCAATGGAAACGGATTTTTCGCAACAATGGCTTGGGCCGTCCCAAAAAA
CGACAAAAACAAAACAGCAACAAATCCATTAACAATAGAAGTACCATACATTTGTA
CAGAAGGAGAAGACCAAATTACCGTTTGGGGGTTCCACTCTGACGACGAGACCCAA
ATGGCAAAGCTCTATGGGGACTCAAAGCCCCAGAAGTTCACCTCATCTGCCAACGG
AGTGACCACACATTACGTTTCACAGATTGGTGGCTTCCCAAATCAAACAGAAGACG
GAGGACTACCACAAAGTGGTAGAATTGTTGTTGATTACATGGTGCAAAAATCTGGG
AAAACAGGAACAATTACCTATCAAAGGGGTATTTTATTGCCTCAAAAGGTGTGGTGC
GCAAGTGGCAGGAGCAAGGTAATAAAAGGATCCTTGCCTTTAATTGGAGAAGCAGA
TTGCCTCCACGAAAAATACGGTGGATTAAACAAAAGCAAGCCTTACTACACAGGGG
AACATGCAAAGGCCATAGGAAATTGCCCAATATGGGTGAAAACACCCTTGAAGCTG
GCCAATGGAACCAAATATAGACCTCCTGCAAAACTATTAAAGGAAAGGGGTTTCTT
CGGAGCTATTGCTGGTTTCTTAGAAGGAGGATGGGAAGGAATGATTGCAGGTTGGC
ACGGATACACATCCCATGGGGCACATGGAGTAGCGGTGGCAGCAGACCTTAAGAGC
ACTCAAGAGGCCATAAACAAGATAACAAAAAATCTCAACTCTTTGAGTGAGCTGGA
AGTAAAGAATCTTCAAAGACTAAGCGGTGCCATGGATGAACTCCACAACGAAATAC
TAGAACTAGATGAGAAAGTGGATGATCTCAGAGCTGATACAATAAGCTCACAAATA
GAACTCGCAGTCCTGCTTTCCAATGAAGGAATAATAAACAGTGAAGATGAACATCT
CTTGGCGCTTGAAAGAAAGCTGAAGAAAATGCTGGGCCCCTCTGCTGTAGAGATAG
GGAATGGATGCTTTGAAACCAAACACAAGTGCAACCAGACCTGTCTCGACAGAATA
GCTGCTGGTACCTTTGATGCAGGAGAATTTTCTCTCCCCACCTTTGATTCACTGAATA
TTACTGCTGCATCTTTAAATGACGATGGATTGGATAATCATACTATACTGCTTTACTA
CTCAACTGCTGCCTCCAGTTTGGCTGTAACACTGATGATAGCTATCTTTGTTGTTTAT
ATGGTCTCCAGAGACAATGTTTCTTGCTCCATCTGTCTATAAGGGAAGTTAAGCCCT
GTATTTTCCTTTATTGTAGTGCTTGTTTACTTGTTGTCATTACAAAGAAACGTTATTG
AAAAATGCTCTTGTTACTACT
(SEQ ID NO: 30)
Bris08 wt NA
agcagaagcagagcatcttctcaaaaccgaagcaaataggccaaaaatgaacaatgaacaatgctaccttcaactatacaaacgttaaccct
atttctcacatcagggggagtattattatcactatatgtgtcagatcattatcatacttactatattcggatatattgctaaaattctcaccaacaga
aataactgcaccaacaatgccattggattgtgcaaacgcatcaaatgttcaggctgtgaaccgttctgcaacaaaaggggtgacacttcttctc
ccagaaccggagtggacatacccgcgtttatcttgcccgggctcaacctttcagaaagcactcctaattagccctcatagattcggagaaac
caaaggaaactcagctcccttgataataagggaaccttttattgcttgtggaccaaatgaatgcaaacactttgctctaacccattatgcagccc
aaccagggggatactacaatggaacaagaggagacagaaacaagctgaggcatctaatttcagtcaaattgggcaaaatcccaacagtag
aaaactccattttccacatggcagcatggagcgggtccgcgtgccatgatggtaaggaatggacatatatcggagttgatggccctgacaat
aatgcattgctcaaagtaaaatatggagaagcatatactgacacataccattcctatgcaaacaaaatcctaagaacacaagaaagtgcctgc
aattgcatcgggggaaattgttatcttatgataactgatggctcagcttcaggtgttagtgaatgcagatttcttaagattcgagagggccgaat
aataaaagaaatatttccaacaggaagagtaaaacacactgaggaatgcacatgcggatttgccagcaataaaaccatagaatgtgcctgta
gagataacagttacacagcaaaaagaccttttgtcaaattaaacgtggagactgatacagcagaaataagattgatgtgcacagatacttattt
ggacacccccagaccaaacgatggaagcataacaggcccttgtgaatctaatggggacaaagggagtggaggcatcaagggaggatttg
ttcatcaaagaatggaatccaagattggaaggtggtactctcgaacgatgtctaaaactgaaaggatggggatgggactgtatgtcaagtatg
atggagacccatgggctgacagtgatgccctagcttttagtggagtaatggtttcaatgaaagaacctggttggtactcctttggcttcgaaata
aaagataagaaatgcgatgtcccctgtattgggatagagatggtacatgatggtggaaaagagacttggcactcagcagcaacagccattta
ctgtttaatgggctcaggacagctgctgtgggacactgtcacaggtgttgacatggctctgtaatggaggaatggttgagtctgttctaaaccc
tttgttcctattttgtttgaacaattgtccttactgaacttaattgtttctgaaaaatgctcttgttactact
(SEQ ID NO: 31)
Bris08 long stalk NA (46aa insertion bold)
agcagaagcagagcatcttctcaaaaccgaagcaaataggccaaaaatgaacaatgaacaatgctaccttcaactatacaaacgttaaccct
atttctcacatcagggggagtattattatcactatatgtgtcagatcattatcatacttactatattcggatatattgctaaaattctcaccaacaC
AATATGAATTCAACTCCCCCCCAAACAACCAAGTGATGCTGTGTGAACCAACAA
TAATAGAAAGAAACATAACAGAAATAGTGTATTTAACTAATCAGACATATGTTA
ACATCAGCAACACCAACTTTGCTGCTgaaataactgcaccaacaatgccattggattgtgcaaacgcatcaaat
gttcaggctgtgaaccgttctgcaacaaaaggggtgacacttcttctcccagaaccggagtggacatacccgcgtttatcttgcccgggctca
acctttcagaaagcactcctaattagccctcatagattcggagaaaccaaaggaaactcagctcccttgataataagggaaccttttattgcttg
tggaccaaatgaatgcaaacactttgctctaacccattatgcagcccaaccagggggatactacaatggaacaagaggagacagaaacaa
gctgaggcatctaatttcagtcaaattgggcaaaatcccaacagtagaaaactccattttccacatggcagcatggagcgggtccgcgtgcc
atgatggtaaggaatggacatatatcggagttgatggccctgacaataatgcattgctcaaagtaaaatatggagaagcatatactgacacat
accattcctatgcaaacaaaatcctaagaacacaagaaagtgcctgcaattgcatcgggggaaattgttatcttatgataactgatggctcagc
ttcaggtgttagtgaatgcagatttcttaagattcgagagggccgaataataaaagaaatatttccaacaggaagagtaaaacacactgagga
atgcacatgcggatttgccagcaataaaaccatagaatgtgcctgtagagataacagttacacagcaaaaagaccttttgtcaaattaaacgt
ggagactgatacagcagaaataagattgatgtgcacagatacttatttggacacccccagaccaaacgatggaagcataacaggcccttgt
gaatctaatggggacaaagggagtggaggcatcaagggaggatttgttcatcaaagaatggaatccaagattggaaggtggtactctcgaa
cgatgtctaaaactgaaaggatggggatgggactgtatgtcaagtatgatggagacccatgggctgacagtgatgccctagatttagtgga
gtaatggtttcaatgaaagaacctggttggtactcctttggcttcgaaataaaagataagaaatgcgatgtcccctgtattgggatagagatggt
acatgatggtggaaaagagacttggcactcagcagcaacagccatttactgtttaatgggctcaggacagctgctgtgggacactgtcacag
gtgttgacatggctctgtaatggaggaatggttgagtctgttctaaaccctttgttcctattttgtttgaacaattgtccttactgaacttaattgtttct
gaaaaatgctcttgttactact
(SEQ ID NO: 32)
Nucleotide sequence encoding 46 amino acid sequence extension
CAATATGAATTCAACTCCCCCCCAAACAACCAAGTGATGCTGTGTGAACCAACAAT
AATAGAAAGAAACATAACAGAAATAGTGTATTTAACTAATCAGACATATGTTAACA
TCAGCAACACCAACTTTGCTGCT
(SEQ ID NO: 33)
Nucleotide sequence encoding 30 amino acid sequence extension
AATCAGACATATGTTAACATCAGCAACACCAACTTTGCTGCTGGAAACACAACAGA
GATAGTGTATCTGACCAACACCACCATAGAGAAG
(SEQ ID NO: 34)
SEQ ID NOS: 35-42 may be found in FIGS. 1B, 1C and 4A

### 6. EXAMPLES

### 6.1 Example 1: Extending the Stalk Enhances the Immunogenicity of Influenza Virus Neuraminidase

This example demonstrates the successful rescue of two recombinant influenza viruses based on the H1N1 strain A/Puerto Rico/8/1934 (PR8) with NA stalk domains extended by 15 or 30 amino acids. Vaccination studies in mice revealed that the virus with 30 amino acid-extended stalk induced significantly higher anti-NA IgG responses than the wild type PR8 virus, while anti-HA IgGs were induced to similar levels. No differences were observed in the NI activity of the antibody responses, but antisera raised with the 30 amino acid extended stalk exerted increased in vitro ADCC activity.

This example also demonstrates the successful generation of variants of the H3N2 A/Hong Kong/4801/2014 (HK14) virus that have a 15 amino acid extension or a 25 amino acid deletion in the N2 stalk. As with the N1 of PR8, this example shows that increasing the stalk length of N2 improves its immunogenicity. The results show that extending the stalk domain of the NA is an approach to enhance its immunogenicity and overcome the immunodominance of the HA, which could improve influenza virus vaccines.

### 6.1.1 Materials and Methods

Recombinant neuraminidase genes and cloning. The recombinant NA segments were based on the NA gene of the PR8 virus or the NA gene of the HK14 virus (50). The nucleotide sequences used for the 15 amino acid insertions were retrieved from the Influenza Research Database (https://www.fludb.org). They were derived from the NA sequences of the Cal09 (H1N1pdm09) virus (accession number FJ66084) and the A/New York/61/2012 (H3N2) virus (accession number KF90392). Sequences were aligned with Clustal X 2.0 (57). DNA fragments encoding the NA gene segments that contained 15 base pair cloning sites specific for the pDZ vector at the 5′ and 3′ ends were obtained as synthetic double-stranded DNAs from Integrated DNA Technologies, using the gBlocks® Gene Fragments service. The NA DNAs were cloned using the In-Fusion HD Cloning Kit (Clontech) into the ambisense pDZ vector that was digested with the SapI restriction enzyme (New England Biolabs). Sequences were confirmed by Sanger sequencing (Macrogen). Sequencing primers pDZ_forward (TACAGCTCCTGGGCAACGTGCTGG; SEQ ID NO: 13) and pDZ reverse (AGGTGTCCGTGTCGCGCGTCGCC; SEQ ID NO: 14) were obtained from Life Technologies.

Cell culture. HEK 293T cells were cultured in Dulbecco's Modified Eagle Medium (DMEM; Gibco) with 10% (v/v) fetal bovine serum (FBS) (Hyclone), 100 units/mL penicillin and 100 μg/mL streptomycin (Pen-Strep; Gibco). MDCK cells were maintained in Minimum Essential Medium (MEM; Gibco) with 10% (v/v) FBS, Pen-Strep, 2 mM L-glutamine (Gibco), 0.15% (w/v) sodium bicarbonate (Corning) and 20 mM 4-(2-hydroxyethyl)-1-piperazineethanesulfonic acid (HEPES, Gibco). Both cell lines were maintained at 37° C. with 5% CO₂.

Rescue of recombinant influenza viruses. Reassortant viruses were rescued by transfecting HEK 293T cells with 0.7 μg of NA-encoding pDZ plasmid, 0.7 μg of HA-encoding pDZ plasmid and 2.1 μg of a pRS-6 segment plasmid that drives ambisense expression of the six segments of PR8 virus except NA and HA and is described elsewhere (58), using the TransIT-LT1 transfection reagent (Minis Bio). After 48 hours, cells were treated for 30 min with 1 μg per mL tosyl phenylalanyl chloromethyl ketone (TPCK)-treated trypsin at 37° C. Supernatants were collected, clarified by low speed centrifugation, and injected into 8 to 10-day old specific pathogen-free embryonated chicken eggs (Charles River Laboratories) that were incubated at 37° C. After 48 hours, eggs were incubated at 4° C. overnight, allantoic fluids were harvested and clarified by low speed centrifugation. The presence of influenza virus in the allantoic fluids was determined by hemagglutination assays as described below. Positive virus cultures were plaque purified on confluent MDCK cell layers in the presence of TPCK-treated trypsin and expanded in embryonated chicken eggs. Sequences of the NA and HA genes were confirmed by isolating viral RNA from allantoic fluids with the High Pure Viral RNA Kit (Roche) followed by reverse-transcription PCR using the SuperScript® III One-Step RT-PCR System with Platinum® Taq High Fidelity DNA Polymerase (Thermo Fisher) and primers PR8_NA_forward (CGAAAGCAGGGGTTTAAAATG; SEQ ID NO: 15), PR8_NA_reverse (TTTTTGAACAGACTACTTGTCAATG; SEQ ID NO: 16), PR8_HA_forward (CCGAAGTTGGGGGGGAGCAAAAGCAGGGGAAAATAA; SEQ ID NO:17) and PR8_HA_reverse (GGCCGCCGGGTTATTAGTAGAAACAAGGGTGTTTTT; SEQ ID NO: 18), or HK14 NAjonvard (GGGAGCAAAAGCAGGAGTAAAGATG; SEQ ID NO: 19), HK14_NA_reverse (TTATTAGTAGAAACAAGGAGTTTTTTCTAAAATTGCG; SEQ ID NO: 20), HK14_HA_forward (GGGAGCAAAAGCAGGGGATAATTC; SEQ ID NO: 21) and HK14_HA_reverse (GGGTTATTAGTAGAAACAAGGGTGTTTTTAATTAATG; SEQ ID NO: 22) obtained from Integrated DNA Technologies. The PCR products were purified from a 1% agarose gel with the NucleoSpin® Gel and PCR Clean-up kit (Macherey-Nagel) and submitted for Sanger sequencing (Genewiz) with the primers described above. No egg-adaptive mutations were observed for any of the sequenced viral genes.

Preparation of formalin-inactivated viruses for vaccination. Plaque-purified and sequenced influenza viruses were expanded in 8 to 10-day old embryonated chicken eggs. Pooled allantoic fluids of 10-20 eggs were added on top of 3 mL of a 20% (w/v) sucrose solution in 0.1 M NaCl, 1 mM ethylenediaminetetraacetic acid (EDTA) and 10 mM Tris-HCl, pH 7.4, in 38.5 mL ultracentrifuge tubes (Denville). Following centrifugation at 25,000 rpm for 2 hours at 4° C. using an L7-65 ultracentrifuge (Beckman) equipped with an SW28 rotor, supernatants were carefully aspirated and pellets were recovered in 1 mL of PBS. After addition of 0.03% (v/v) formaldehyde, the virus suspensions were incubated for 48 hours at 4° C. while shaking. To remove the formaldehyde, virus suspensions were diluted with PBS and subjected to ultracentrifugation as described above. Pellets were resuspended in sterile PBS and the total protein concentration was determined with the Pierce BCA Protein Assay Kit (Thermo Fisher).

Western blots. Western blots. Purified virus particles were lysed in NP-40 lysis buffer (1% (v/v) NP-40, 150 mM NaCl, 50 mM Tris-HCl, pH 8.0, protease inhibitors (Halt™ Protein and Phosphatase Inhibitor Cocktail; Thermo Fisher) and 1 mM dithiothreitol (DTT)). After incubation on ice for 30 min, samples were centrifuged for 10 min at 12,000 rpm in a table-top centrifuge. The supernatants were transferred to new microcentrifuge tubes and the protein concentrations after lysis were determined with the Pierce BCA Protein Assay Kit (Thermo Fisher). Proteins (1 or 2 μg) were separated on 12.5% polyacrylamide gels under denaturing conditions in the presence of sodium dodecyl sulfate (SDS) and then transferred onto polyvinylidene difluoride (PVDF) membranes. As protein size marker, the Color Prestained Protein Standard, Broad Range (11-254 kDa) (New England Biolabs) was used. The membranes were blocked for 1 hour using PBS with 5% (w/v) skim milk powder and washed three times with PBS containing 0.05% (v/v) Tween-20. Primary antibodies were mouse anti-N1 monoclonal antibody 4A5 (10) that was used at 1 μg per mL, rabbit anti-H1 (Thermo Fisher; cat.no. PAS-34929) (1:5,000 dilution), anti-N2 polyclonal serum raised in guinea pigs generated in-house (1:2,000 dilution), anti-H3 monoclonal antibody 12D1 (59), and anti-NP (Invitrogen; cat.no. PAS-32242) (1:3,000 dilution). Primary antibodies were diluted in PBS with 1% (w/v) bovine serum albumin (BSA) and incubated on the membranes for 1 hour. The membranes were washed three times with PBS containing 0.05% (v/v) Tween-20 and were incubated for 1 hour with secondary HRP-labeled antibodies (anti-mouse, cat.no. NXA931V, or anti-rabbit, cat.no. NA9340V, both from GE Healthcare) diluted 1:3,000 in PBS with 1% (w/v) BSA according to the manufacturer's recommendations. After washing three times with PBS containing 0.05% (v/v) Tween-20, developing solution (Pierce™ ECL Western Blotting Substrate, Thermo Scientific) was added to the membranes that were subsequently developed in a ChemiDoc™ MP Imaging System (Bio-Rad). NP band intensities were determined by the software provided on the ChemiDoc™ MP Imaging System. Lanes were automatically detected with manual adjustment. Normalization factors were calculated by dividing the NP band intensity of one sample with the NP band intensity of N2-wt virus.

Immunization studies. Animal experiments were performed with 6-8 weeks old female BALB/c mice (Charles River) in accordance with protocols approved by the Institutional Animal Care and Use Committee (IACUC) of the Icahn School of Medicine at Mount Sinai. Formalin-inactivated viruses were administered intramuscularly at a dose of 10 μg total protein per mouse diluted in a total volume of 100 μL sterile PBS. Four weeks after the final immunization, mice were euthanized and blood was collected by cardiac puncture. Sera were prepared by removing red blood cells by centrifugation and were stored at −20° C. until use.

Enzyme-linked immunosorbent assays (ELISA). The trimeric recombinant PR8 HA protein and the tetrameric recombinant PR8 and HK14 NA proteins were produced as described (60, 61). Proteins were coated onto Immulon® 4 HBX 96-well microtiter plates (Thermo Scientific) at a concentration of 2 μg per mL in PBS (50 μL per well) for 16 hours at 4° C. After washing once using PBS with 0.1% (v/v) Tween-20 (PBS-T), wells were blocked for 1 hour with 5% (w/v) skim milk powder in PBS and washed once with PBS-T. Mouse sera diluted in PBS (50 μL per well) were added and incubated on the plates for 1 hour. After washing with PBS-T three times, wells were incubated with HRP-conjugated anti-mouse IgG antibody (GE Healthcare) diluted 1:5,000 in 5% (w/v) skim milk powder in PBS for 1 hour, washed three times with PBS-T and developed with 100 μL per well of SigmaFast OPD substrate (Sigma-Aldrich) for 20 min. Reactions were stopped by adding 100 μL per well of 3 M hydrochloric acid (HCl) and absorbance at 490 nm was determined on a Synergy 4 plate reader (BioTek). For each ELISA plate, the average plus three standard deviations of absorbance values of blank wells were used as a cutoff to calculate area under the curve (AUC) values in GraphPad Prism 5.03 (GraphPad Software).

Hemagglutination assays. Using PBS, serial two-fold dilutions of allantoic fluids were prepared in 96 V-bottom well microtiter plates to a final volume of 50 μL per well. To each well, 50 μL of a 0.5% suspension of turkey red blood cells (Lampire) in PBS were added. Plates were incubated at 4° C. until red blood cells in PBS control samples settled to the bottom of the wells. The hemagglutination titer was defined as the reciprocal of the highest dilution of allantoic fluid that caused hemagglutination of red blood cells.

Hemagglutination inhibition (HI) assays. One volume of mouse serum was treated with three volumes of receptor-destroying enzyme (RDE; Denka Seiken, Tokyo, Japan) at 37° C. for 16 hours.(50) Then, three volumes of a 2.5% sodium citrate solution were added. After incubation at 56° C. for 30 min, three volumes of PBS were added for a final dilution of 1:10. Two-fold dilutions (25 μL) of the RDE-treated sera in PBS were prepared in 96-well V-bottom microtiter plates and were combined with 25 μL per well of either PR8 wildtype virus or HK2014 wildtype virus (allantoic fluids) that were diluted in PBS to a final HA titer of 8 HA units per 50 μL. The samples were incubated for 30 min at room temperature to allow for HA-specific antibodies to bind to the virus particles. Then, 50 μL of a 0.5% suspension of turkey red blood cells (Lampire) that was washed once with PBS were added to each well. The plates were incubated at 4° C. until the red blood cells in PBS control samples settled to the bottom of the wells. The HI titers were defined as the reciprocal of the highest serum dilution causing inhibition of hemagglutination of red blood cells.

Enzyme-linked lectin assay (ELLA) to determine neuraminidase inhibition (NI). This assay was performed as previously described (62, 63). Microtiter 96-well plates (Immulon® 4 HBX; Thermo Fisher Scientific) were coated with 50 μg per mL (150 μL per well) of fetuin (Sigma) diluted in coating solution (SeraCare Life Sciences Inc.) and incubated overnight at 4° C. The next day, heat-inactivated (56° C., 30 min) serum samples were serially diluted 1:2 in PBS in separate 96-well plates (leaving the first column as virus only control and last column as the background), with a starting dilution of 1:20. The final volume of diluted serum samples was 75 μL per well. A recombinant influenza virus expressing a chimeric HA protein, cH4/3 (containing the H4 globular head domain from A/duck/Czech/1956 (H4N6) virus in combination with the H3 stalk domain from A/Perth/16/2009 (H3N2) virus (64)) and the remaining proteins of PR8 virus was diluted to the 90% effective concentration (EC₉₀) in PBS containing 1% BSA, and 75 μL per well were added to the serially diluted serum samples and virus only controls. Seventy-five microliters of PBS with 1% BSA were added to the background wells. The serum/virus plates were incubated for 2 hours at room temperature to allow for binding of antibodies to the virus particles. The fetuin-coated plates from the previous day were washed three times with PBS-T. One hundred microliters per well of the serum/virus mixtures were transferred to the washed fetuin-coated plates that were then incubated for 2 hours at 37° C. The plates were washed three times with PBS-T and 100 μL per well of peanut agglutinin-horseradish peroxidase conjugate (PNA-HRP; Sigma-Aldrich) diluted to 5 μg per mL in PBS were added. The plates were incubated in the dark for 1 hour at room temperature. After washing three times with PBS-T, 100 μL per well of SigmaFast OPD substrate (Sigma-Aldrich) were added and the plates were incubated for 10 min. Reactions were stopped by adding 100 μL per well of 3 M HCl and absorbance at 490 nm was determined on a Synergy 4 plate reader (BioTek). Serum sample reactivity was determined by subtracting background absorbance values (no virus, no serum) from the raw absorbance values of serum samples. The obtained values were divided by the average value from virus-only control wells and then multiplied by a factor of 100 to calculate the NA activity. Percent NI was determined by subtracting NA activity from 100%. Using GraphPad Prism, the percent NI was fitted to a nonlinear regression to determine the 50% inhibitory concentration (IC₅₀) of the serum samples.

Antibody-dependent cellular cytotoxicity (ADCC) reporter assays. ADCC reporter assays were performed as described previously (49). 96-well white flat-bottom plates (Costar Corning) were seeded with 2×10⁴ MDCK cells per well. After 18 hours of incubation at 37° C., the MDCK cells were washed once with PBS and then infected with either wildtype PR8 virus or a 7:1 reassortant virus expressing the HA protein of A/Hong Kong/4801/2014 (H3N2) virus and the remaining proteins of PR8 virus (50) at a multiplicity of infection (MOI) of 5 for single cycle replication. Alternatively, HEK 293T cells were plated in 96-well white flat-bottom plates treated with poly-D-lysine (Sigma-Aldrich) at a density of 2×10⁴ cells per well and, after incubation for 4 hours, were transfected with 100 ng per well of a pCAGGS plasmid expressing the NA of PR8 virus using the TransIT-LT1 transfection reagent (Minis Bio). Infected MDCK cells or transfected HEK 293T cells were incubated for 16 hours at 37° C. Then, the culture medium was aspirated and 25 μL of assay buffer (RPMI 1640 supplemented with 4% low-IgG FBS) was added to each well. Pooled sera were added in a volume of 25 μL at a starting dilution of 1:60 and serial 2-fold dilutions prepared in assay buffer in triplicates. The sera were incubated with the cells for 30 min at 37° C. Genetically modified Jurkat cells expressing the murine FcγRIV with a luciferase reporter gene under control of the nuclear factor-activated T cells (NFAT) promoter (Promega) were added at 7.5×10⁴ cells in 25 μL per well. After incubation for 6 hours at 37° C., 75 μL per well of Bio-Glo Luciferase assay reagent (Promega) was added and luminescence was quantified using a Synergy 4 plate reader (BioTek). Fold induction was measured in relative light units and calculated by subtracting the background signal from wells without effector cells, then dividing signals of wells with antibody by those with no antibody added.

Immunofluorescence microscopy. 96-well tissue culture plates were seeded with 2×10⁴ MDCK cells per well. After 24 hours of incubation at 37° C., the MDCK cells were washed once with PBS and then infected with either wild type PR8 virus, PR8 virus with N1-Ins15 NA, or PR8 virus with N1-Ins30 NA at an MOI of 5 for single cycle replication. Infected MDCK cells were incubated for 16 hours at 37° C. The culture medium was aspirated, the cells were washed twice with PBS and then fixed with a methanol-free 4% (v/v) paraformaldehyde in PBS solution for 15 min. After washing twice with PBS, the wells were blocked with 5% (w/v) skim milk powder in PBS for 30 min. The cells were washed once with PBS and then incubated with the broadly N1-reactive mAb 4A5 (11) at 10 μg per mL diluted in 5% (w/v) skim milk powder in PBS for 2 hours. After washing three times with PBS, the cells were incubated with fluorescence-labeled anti-mouse IgG Alexa Fluor 488 antibody (Life Technologies) diluted 1:2,000 in 5% (w/v) skim milk powder in PBS for 1 hour and then washed three times with PBS before pictures were taken on an EVOS fl inverted fluorescence microscope (AMG).

Statistics. Statistical data was generated with GraphPad Prism. Statistical significance between groups was determined by performing one-way analysis of variance (ANOVA) tests with Bonferroni correction for multiple comparisons. Levels of significance are indicated as follows: *P≤0.05, **P≤0.01, ***P≤0.001.

### 6.1.2 Results

Design, rescue and characterization of influenza viruses expressing NA proteins with extended stalk domains. PR8 was selected as a model influenza virus to study whether the length of the stalk domain of NA influences its immunogenicity. It was hypothesized that an extended stalk domain would increase the visibility of the NA protein on the surface of virus particles to the humoral immune system, thereby enhancing its immunogenicity.

Compared to circulating H1N1 strains (pre- and post-pandemic), the NA of the PR8 virus has a 15 amino acid deletion in the stalk domain (44). It has been estimated by molecular dynamics calculations that the NA protein of the H1N1pdm09 A/California/04/2009 (Cal09) virus extends from the membrane by 149 Å, which is slightly shorter than the estimated height of the HA protein (154 Å) (45) (FIG. 1A). It was also calculated that each amino acid in the stalk domain contributes to ˜1.2 Å of the total height of the NA protein (45). Consequently, the NA of PR8 virus has an estimated height of 131 Å. Adding 15 amino acids to the PR8 NA would increase its height to that of the Cal09 NA (149 Å) and inserting 30 amino acids would raise the height to 167 Å, which would be 13 Å taller than that of the HA protein (FIG. 1A). Since unrelated sequences could perturb the structure of the PR8 NA protein, stalk sequences of other NA proteins that, despite the variability of amino acid sequences, likely share structural features with those in the stalk of PR8 NA (41,42) were selected to be introduced.

Alignment of the NA protein sequences of the PR8 and Cal09 viruses revealed the position of the 15 amino acid deletion in the stalk of PR8 NA (FIG. 1B). At that position, the corresponding 15 amino acids of the Cal09 NA were inserted into the PR8 NA (FIG. 1C). This mutant was designated as N1-Ins15 (SEQ ID NO: 8). An additional sequence of 15 amino acids was derived from the NA stalk domain of the H3N2 A/New York/61/2012 (NY12) virus. A mutant of the PR8 NA that contained both the 15 amino acids of Cal09 NA and the 15 amino acids of the NY12 NA was designated as N1-Ins30 (FIG. 1C; SEQ ID NO: 10).

The nucleotide sequences of the NA gene segments from the Cal09 and NY12 viruses were used to create the recombinant RNAs encoding the N1-Ins15 and N1-Ins30 proteins. The modified segments were used to rescue viruses expressing these NAs in the PR8 backbone by reverse genetics. As a control, the wild type PR8 virus was rescued in parallel, whose NA was designated as N1-wt. After growing for 48 hours in embryonated chicken eggs, the plaque-purified and sequence-confirmed viruses grew to comparable hemagglutination titers (FIG. 1D). Thus, confirming previous reports (41-43), there was no evidence that the stalk insertions significantly affected viral growth. Western blots with proteins isolated from virus particles revealed distinct size shifts of the extended NA proteins compared to the wild type NA (FIG. 1E). NA and HA expression levels were comparable in the different viruses (FIG. 1E). The three viruses were able to infect Madin-Darby canine kidney (MDCK) cells which resulted in expression of NA on the surface (FIG. 1F).

In summary, two viruses in the PR8 backbone with NA stalk domains extended by 15 or 30 amino acids that replicated in eggs and MDCK cells were successfully rescued. On virus particles, the mutated NA proteins were expressed at comparable levels, and the levels of HA appeared to be unaffected by the mutations in the NA.

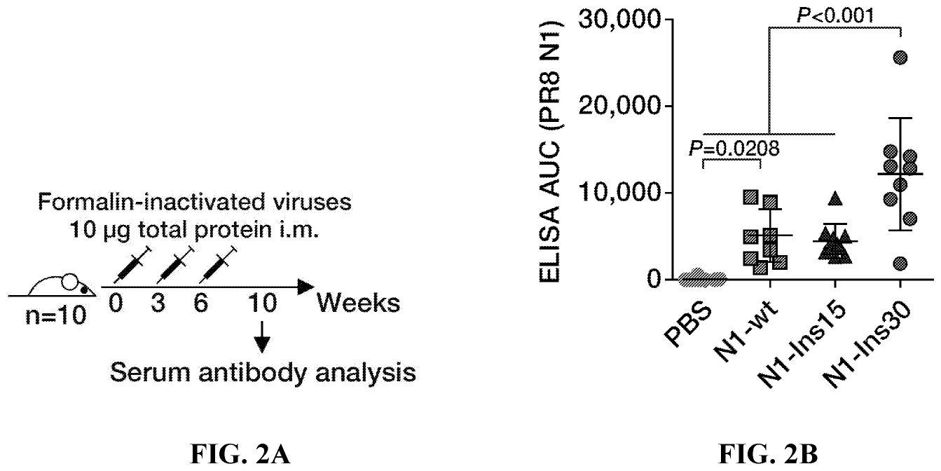

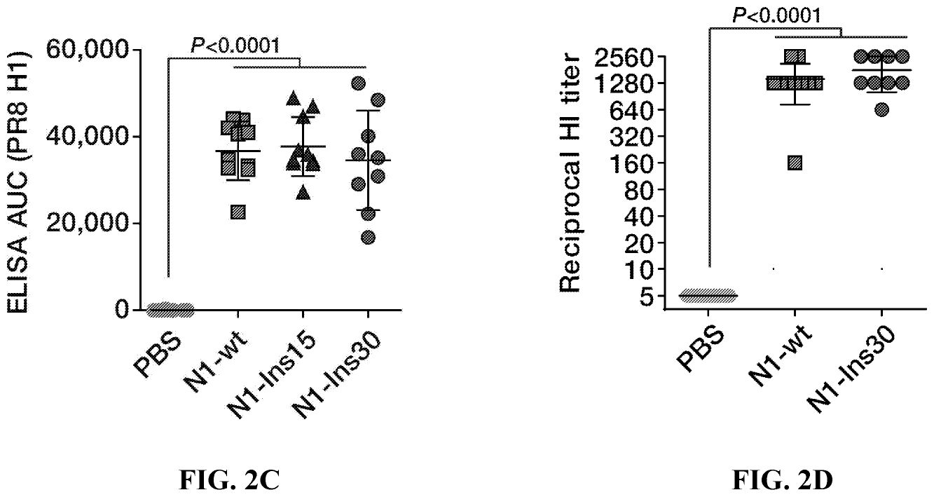

Extending the stalk domain by 30 amino acids enhances immunogenicity of the NA in mice. Next, it was assessed whether the length of the stalk domain influences the immunogenicity of the NA protein in the mouse model. Three groups of 10 BALB/c mice were immunized intramuscularly with formalin-inactivated viruses expressing the N1-wt, N1-Ins15 or N1-Ins30 proteins three times in three-week intervals with doses of 10 μg total protein (FIG. 2A). A fourth group of mice receiving phosphate-buffered saline (PBS) served as control. Four weeks after the third immunization, the mice were sacrificed and serum IgG responses were determined by enzyme-linked immunosorbent assays (ELISAs). Compared to the PBS controls, immunization with all three viruses induced significant IgG responses against recombinant NA protein from PR8 (FIG. 2B). While the viruses with N1-wt and N1-Ins15 NAs induced comparable levels of anti-NA IgG, the virus carrying the N1-Ins30 NA elicited significantly stronger (˜2.5-fold) anti-NA IgG responses. By contrast, the three viruses induced comparable IgG responses against recombinant PR8 HA protein (FIG. 2C). In addition, the NA stalk length did not affect hemagglutination inhibition (HI) titers (FIG. 2D). Thus, extending the stalk domain by 30 amino acid significantly enhanced the IgG responses against NA, without compromising anti-HA antibody levels.

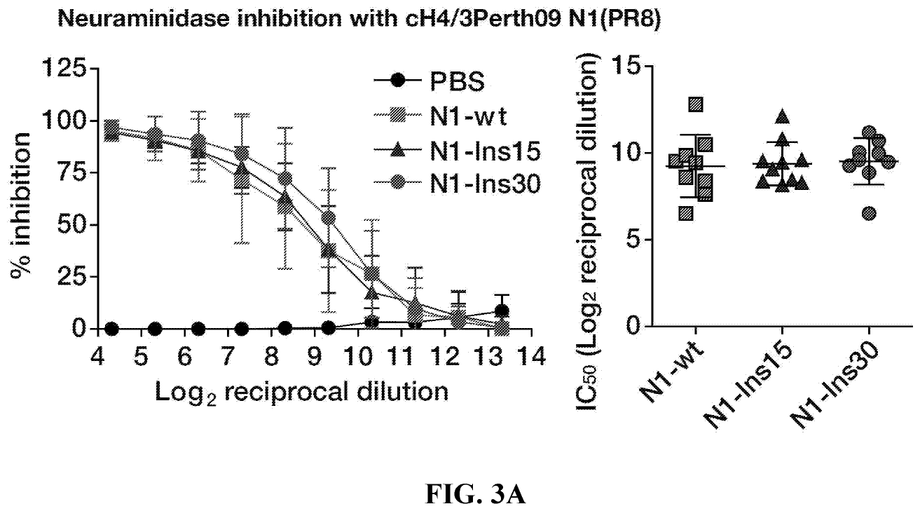

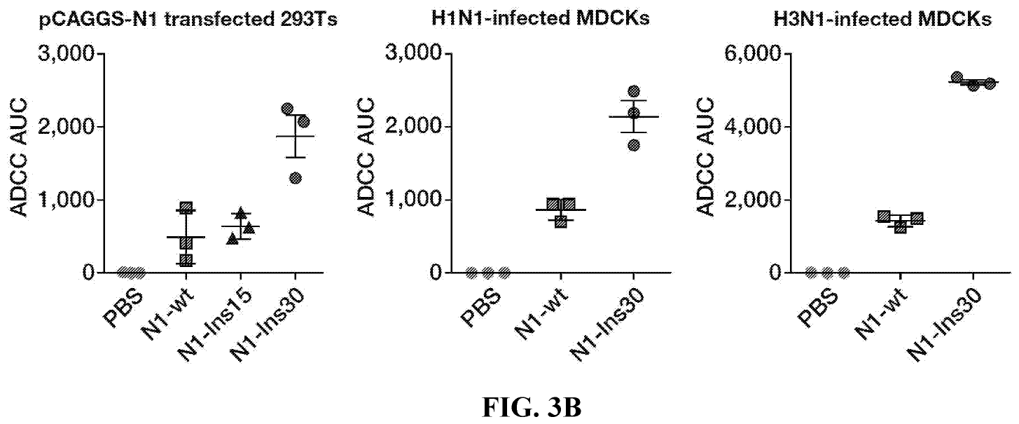

Stalk extension enhances the induction of antibodies with in vitro effector functions. Next, the functional properties of the antibodies elicited by the different viruses was assessed. In general, the majority of anti-NA antibodies are thought to prevent binding of the enzymatically active site to its substrate sialic acid (2). As these types of antibodies typically exert in vitro NI activity, NI assays were performed with the sera obtained from the immunized mice. Although the N1-Ins30 expressing virus elicited higher total anti-NA IgG titers than the other two viruses, as measured by ELISA (FIG. 2B), NI activities were similar between the groups of mice immunized with the three different viruses (FIG. 3A).

Another previously described function of anti-NA antibodies is the induction of Fc receptor-mediated effector functions, such as ADCC (29). To assess whether the induction of antibodies with effector functions was influenced by the stalk length of NA, the murine immune sera was subjected to an in vitro ADCC reporter assay (49). Using human embryonic kidney (HEK) 293T cells expressing the PR8 NA protein, sera raised with the N1-Ins30 expressing virus showed substantially higher ADCC activity than sera induced with viruses carrying N1-wt or N1-Ins15 (FIG. 3B). Similar results were obtained using MDCK cells that were infected either with wild type PR8 virus or an H3N1 virus expressing the PR8 NA and the HA of the HK14 H3N2 virus (50) (FIG. 3B).

In summary, extending the stalk domain of the NA enhanced antibody responses with in vitro ADCC activity, but not the induction of NI active antibodies.

Stalk extension improves immunogenicity of the NA of a recent clinically relevant H3N2 strain. Next, it was assessed if extending the stalk could improve NA immunogenicity in other influenza virus strains. To test this, viruses containing HK14 H3N2 HA and NA with PR8 internal segments were generated. Cryogenic electron micrographs of H3N2 virus show that the NA and HA both extend from the membrane by ˜150 Å with the NA being slightly taller than the HA (51). Thus, viruses containing NAs with no stalk changes (N2-wt), a 25 amino acid stalk deletion (N2-Del25; SEQ ID NO: 2), or a 15 amino acid stalk insertion derived from part of the N1 stalk of Cal09 (N2-Ins15; SEQ ID NO: 4) were generated (FIG. 4A).

These viruses were rescued by reverse genetics as described above. After growing for 72 hours in embryonated chicken eggs, the different plaque-purified and sequence-confirmed viruses achieved comparable hemagglutination titers (FIG. 4B). Western blot analyses revealed that the expression levels of the N2 and H3 glycoproteins varied between the N2-Del25, N2-wt and N2-Ins15 expressing viruses (FIG. 4C). Therefore, vaccination doses were normalized to the expression levels of the NP protein. Three groups of five BALB/c mice were immunized intramuscularly once with formalin-inactivated viruses expressing the N2-wt, N2-Del25, or N2-Ins15 NA proteins. Mice received an amount of formalin-inactivated virus equivalent to 10 μg of wild-type virus as determined by normalization to NP content. A fourth group of three mice receiving PBS served as control. Serum obtained four weeks post-vaccination was subjected to antibody analysis by ELISA against recombinant tetrameric HK14 N2 protein (FIGS. 4C, 4D). Immunization with N2-Ins15 virus elicited ˜3-fold and ˜4.5-fold stronger anti-NA IgG responses compared to immunization with N2-wt and N2-Del25 viruses, respectively (FIG. 4E). Thus, extending the stalk domain improved the immunogenicity of N2 on virus particles. Similar to the observations with the H1N1 virus above, the stalk length of N2 did not significantly affect anti-H3 antibody titers (FIG. 4F) or the levels of HI-reactive antibodies (FIG. 4G).

### 6.1.3 Discussion

Although other reasons for the poor immunogenicity of the NA of current seasonal vaccines are recognized, such as varying and unreliable amounts of NA proteins (34) and their inconsistent stability (52), it is established that anti-NA antibody responses are suppressed in current vaccines due to the immunodominance of the HA (31-34). The results in this example demonstrate that a simple extension of the NA stalk can significantly enhance the anti-NA immune response without compromising the immunogenicity of the HA. Consistent with previous studies (41-43), mutant viruses in the PR8 backbone carrying NA proteins with 15 or 30 amino acid extended NA stalk domains replicate in eggs and MDCK cells without any apparent growth disadvantages compared to wild type PR8 virus. In mice, immunization studies with formalin-inactivated virus particles reveal that a 30 amino acid extension to PR8 NA, but not a 15 amino acid extension, significantly enhance total anti-NA IgG responses without affecting IgG responses to the HA or the levels of HI-reactive antibodies. Based on published molecular dynamics simulations (45), a 30 amino acid extension in PR8 (but not a 15 amino acid extension) is predicted to increase the height of the NA such that it surpasses the height of the HA, suggesting that the improvement in immunogenicity is dependent on the visibility of the NA relative to the HA. Similarly, extending the stalk of the N2 of HK14 virus—the H3N2 strain used in the 2016-2017 and 2017-2018 seasonal vaccines (50)—significantly enhanced NA immunogenicity without affecting anti-HA IgG levels or HI titers, demonstrating that this approach is a viable strategy for improving immunogenicity.

This example demonstrates that stalk extension of PR8 NA did not affect the induction of NI reactive antibodies, but substantially increased the production of antibodies with in vitro ADCC activity. This suggests that the longer stalk makes novel epitopes accessible that are targeted by ADCC active antibodies but not by NI active antibodies, while the immunogenicity of regions recognized by NI reactive antibodies is preserved. Of note, it has been shown that ADCC active IgGs recognizing the stalk domain of the HA (53, 54) or the NA protein (55) can protect against lethal influenza virus infection in mice, in an Fc gamma receptor-dependent manner. A recent study also showed that ADCC-active and NI inactive anti-NA mAbs targeting the lateral surface of the head domain could confer protection in mice (56). Without being bound by any theory or hypothesis, extending the stalk may enhance the exposure of epitopes below the head domain and/or on the lateral surface of the head domain, thereby increasing the induced antibody repertoire. Moreover, broadly reactive anti-NA antibodies that target conserved epitopes are often ADCC active (29). Therefore, extending the NA stalk domain may not only increase the immunogenicity of the NA on virus particles, but also enhance the breadth of protection afforded by the induced anti-NA antibodies.

Unlike other pursued approaches to enhance anti-NA immunity that are based on isolated or recombinant NA proteins (11-13, 37), DNA plasmids (10), virus-like and replicon particles (23, 25) or virus-vectored vaccines (38), the NA stalk extension described here may be implemented in existing manufacturing processes for seasonal influenza virus vaccines, as the mutated NAs can be expressed on virus particles that efficiently replicate in eggs. Moreover, the data herein provides evidence that the subdominance of the NA results in part from the height of to protein relative to the HA. Without being bound by any theory or hypothesis, immunodominance is associated with viral epitopes being most distal from the surface of the virus or the infected cell and that immunodominance may simply be a question of being more easily recognized by B-cell receptors of the infected host.

### 6.1.4 References Cited in, e.g., Example 1

1. Krammer F, et al. 2018. Influenza. Nat Rev Dis Primers 4: 3.
2. Krammer F, et al. 2018. NAction! How can neuraminidase-based immunity contribute to better influenza virus vaccines? mBio 9: e02332-17.
3. Marcelin G, et al. 2012. Contribution of antibody production against neuraminidase to the protection afforded by influenza vaccines. Rev Med Virol 22, 267-279.
4. Kilbourne E D. 1976. Comparative efficacy of neuraminidase-specific and conventional influenza virus vaccines in induction of antibody to neuraminidase in humans. J Infect Dis 134: 384-394.
5. Powers D C, et al. 1996. Neuraminidase-specific antibody responses to inactivated influenza virus vaccine in young and elderly adults. Clin Diagn Lab Immunol 3: 511-516.
6. Sandbulte M R, et al. 2011. Discordant antigenic drift of neuraminidase and hemagglutinin in H1N1 and H3N2 influenza viruses. Proc Natl Acad Sci USA 108: 20748-20753.
7. Couch R B, et al. 2012. Randomized comparative study of the serum antihemagglutinin and antineuraminidase antibody responses to six licensed trivalent influenza vaccines. Vaccine 31: 190-195.
8. Laguio-Vila M R, et al. 2015. Comparison of serum hemagglutinin and neuraminidase inhibition antibodies after 2010-2011 trivalent inactivated influenza vaccination in healthcare personnel. Open Forum Infect Dis 2: ofu115.
9. Krammer F. 2019. The human antibody response to influenza A virus infection and vaccination. Nat Rev Immunol 2019 Mar. 5 (Epub ahead of print).
10. Sandbulte M R, et al. 2007. Cross-reactive neuraminidase antibodies afford partial protection against H5N1 in mice and are present in unexposed humans. PLoS Med 4: e59.
11. Wohlbold T J, et al. 2015. Vaccination with adjuvanted recombinant neuraminidase induces broad heterologous, but not heterosubtypic, cross-protection against influenza virus infection in mice. mBio 6: e02556.
12. Liu W C, et al. 2015. Cross-reactive neuraminidase-inhibiting antibodies elicited by immunization with recombinant neuraminidase proteins of H5N1 and pandemic H1N1 influenza A viruses. J Virol 89: 7224-7234.
13. Job E R, et al. 2018. Broadened immunity against influenza by vaccination with computationally designed influenza virus N1 neuraminidase constructs. NPJ Vaccines 3: 55.
14. Chen Y Q, et al. 2018. Influenza infection in humans induces broadly cross-reactive and protective neuraminidase-reactive antibodies. Cell 173: 417-429.e10.
15. Murphy B R, et al. 1972. Association of serum anti-neuraminidase antibody with resistance to influenza in man. N Engl J Med 286: 1329-1332.
16. Couch R B, et al. 1974. Induction of partial immunity to influenza by a neuraminidase-specific vaccine. J Infect Dis 129: 411-420.
17. Clements M L, et al. 1986. Serum and nasal wash antibodies associated with resistance to experimental challenge with influenza A wild-type virus. J Clin Microbiol 24: 157-160.
18. Memoli M J, et al. 2016. Evaluation of antihemagglutinin and antineuraminidase antibodies as correlates of protection in an influenza A/H1N1 virus healthy human challenge model. mBio 7: e00417-16.
19. Couch R B, et al. 2013. Antibody correlates and predictors of immunity to naturally occurring influenza in humans and the importance of antibody to neuraminidase. J Infect Dis 207: 974-981.
20. Monto A S, et al. 2015. Antibody to influenza virus neuraminidase: an independent correlate of protection. J Infect Dis 212: 1191-1199.
21. Kilbourne E D, et al. 1990. Independent and disparate evolution on nature of influenza A virus hemagglutinin and neuraminidase glycoproteins. Proc Natl Acad Sci USA 87: 786-790.
22. Gao J, et al. 2019. Antigenic drift of the influenza A(H1N1)pdm09 virus neuraminidase results in reduced effectiveness of A/California/7/2009 (H1N1pdm09)-specific antibodies. mBio 10: e00307-19.
23. Easterbrook J D, et al. 2012. Protection against a lethal H5N1 influenza challenge by intranasal immunization with virus-like particles containing 2009 pandemic H1N1 neuraminidase in mice. Virology 432: 39-44.
24. Wan H, et al. 2013. Molecular basis for broad neuraminidase immunity: conserved epitopes in seasonal and pandemic H1N1 as well as H5N1 influenza viruses. J Virol 87: 9290-9300.
25. Halbherr S J, et al. 2015. Biological and protective properties of immune sera directed to the influenza virus neuraminidase. J Virol 89: 1550-1563.
26. Mendez-Legaza J M, et al. 2019. Heterosubtypic neuraminidase antibodies against different A(H1N1) strains are elicited after seasonal influenza vaccination. Vaccines (Basel) 7: pii: E30.
27. Schulman J L. 1969. The role of antineuraminidase antibody to immunity to influenza virus infection. Bull World Health Organ 41: 647-650.
28. Kilbourne E D, et al. 1995. Purified influenza A virus N2 neuraminidase vaccine is immunogenic and non-toxic in humans. Vaccine 13: 1799-1803.
29. Wohlbold T J, et al. 2017. Broadly protective murine monoclonal antibodies against influenza B virus target highly conserved neuraminidase epitopes. Nat Microbiol 2: 1415-1424.
30. Piepenbrink M S, et al. 2019. Broad and protective influenza B virus neuraminidase antibodies in humans after vaccination and their clonal persistence as plasma cells. mBio 10: e00066-19.
31. Johansson B E, et al. 1987a. Antigen-presenting B cells and helper T cells cooperatively mediate intravirionic antigenic competition between influenza A virus surface glycoproteins. Proc Natl Acad Sci USA 84: 6869-6873.
32. Johansson B E, et al. 1987. Immunologic response to influenza virus neuraminidase is influenced by prior experience with the associated viral hemagglutinin. II.
Sequential infection of mice simulates human experience. J Immunol 139: 2010-2014.
33. Johansson B E, Kilbourne E D. 1993. Dissociation of influenza virus hemagglutinin and neuraminidase eliminates their intravirionic antigenic competition. J Virol 67: 5721-5723.
34. Wohlbold T J, Krammer F. 2014. In the shadow of hemagglutinin: a growing interest in influenza viral neuraminidase and its role as a vaccine antigen. Viruses 6: 2465-2494.
35. Johansson B E, et al. 1989. Purified influenza virus hemagglutinin and neuraminidase are equivalent in stimulation of antibody response but induce contrasting types of immunity to infection. J Virol 63: 1239-1246.
36. Johansson B E, et al. 1998. Supplementation of conventional influenza A vaccine with purified viral neuraminidase results in a balanced and broadened immune response. Vaccine 16: 1009-1015.
37. Wohlbold T J, Hirsh A, Krammer F. 2015. An H10N8 influenza virus vaccine strain and mouse challenge model based on the human isolate A/Jiangxi-Donghu/346/13. Vaccine 33: 1102-1106.
38. Meseda C A, et al. 2018. Immunogenicity and protection against influenza H7N3 in mice by modified vaccinia virus Ankara vectors expressing influenza virus hemagglutinin or neuraminidase. Sci Rep 8: 5364.
39. Krammer F, Palese P. 2015. Advances in the development of influenza virus vaccines. Nat Rev Drug Discov 14: 167-182.
40. Li J, et al. 2011. Emergence and genetic variation of neuraminidase stalk deletions in avian influenza viruses. PLoS One 6: e14722.
41. Castrucci M R, Kawaoka Y. 1993. Biologic importance of neuraminidase stalk length in influenza A virus. J Virol 67: 759-764.
42. Luo G, et al. 1993. Alterations of the stalk of the influenza virus neuraminidase: deletions and insertions. Virus Res 29: 141-153.
43. Kosik I, Angeletti D, Gibbs J S, Angel M, Takeda K, Kosikova M, Nair V, Hickman H D, Xie H, Brooke C B, Yewdell J W. 2019. Neuraminidase inhibition contributes to influenza A virus neutralization by anti-hemagglutinin stem antibodies. J Exp Med 216: 304-316.
44. Steuler H, et al. 1984. Sequence of the neuraminidase gene of an avian influenza A virus (A/parrot/ulster/73, H7N1). Virology 135: 118-124.
45. Durrant J D, et al. 2016. Microsecond molecular dynamics simulations of influenza neuraminidase suggest a mechanism for the increased virulence of stalk-deletion mutants. J Phys Chem B 120: 8590-8599.
46. Gamblin S J, et al. 2004. The structure and receptor binding properties of the 1918 influenza hemagglutinin. Science 303: 1838-1842.
47. Vavricka C J, et al. 2011. Structural and functional analysis of laninamivir and its octanoate prodrug reveals group specific mechanisms for influenza NA inhibition. PLoS Pathog 7: e1003349.
48. Pettersen E F, et al. 2004. UCSF Chimera—a visualization system for exploratory research and analysis. J Comput Chem 25: 1605-1612.
49. Bailey M J, et al. 2018. A Method to Assess Fc-mediated Effector Functions Induced by Influenza Hemagglutinin Specific Antibodies. J Vis Exp 132: e56256.
50. Broecker F, et al. 2018. Immunodominance of antigenic site B in the hemagglutinin of the current H3N2 influenza virus in humans and mice. J Virol 92: e01100-18.
51. Harris A, et al. 2006. Influenza virus pleiomorphy characterized by cryoelectron tomography. Proc Natl Acad Sci USA 103: 19123-19127.
52. Sultana I, et al. 2014. Stability of neuraminidase in inactivated influenza vaccines. Vaccine 32: 2225-2230.
53. DiLillo D J, et al. 2014. Broadly neutralizing hemagglutinin stalk-specific antibodies require FcγR interactions for protection against influenza virus in vivo. Nat Med 20: 143-151.
54. DiLillo D J, et al. 2016. Broadly neutralizing anti-influenza antibodies require Fc receptor engagement for in vivo protection. 126: 605-610.
55. Henry-Dunand C J, et al. 2016. Both neutralizing and non-neutralizing human H7N9 influenza vaccine-induced monoclonal antibodies confer protection. Cell Host Microbe 19: 800-813.
56. Yasuhara A, Yamayoshi S, Kiso M, Sakai-Tagawa Y, Koga M, Adachi E, Kikuchi T, Wang I H, Yamada S, Kawaoka Y. 2019. Antigenic drift originating from changes to the lateral surface of the neuraminidase head of influenza A virus. Nat Microbiol 2019 Mar. 18 (Epub ahead of print).
57. Larkin M A, et al. 2007. Clustal W and Clustal X version 2.0. Bioinformatics 23: 2947-2948.
58. Fulton B O, et al. 2018. The influenza B virus hemagglutinin head domain is less tolerant to transposon mutagenesis than that of the influenza A virus. J Virol 92: e00754-18.
59. Wang, T T, et al. 2010. Broadly protective monoclonal antibodies against H3 influenza viruses following sequential immunization with different hemagglutinins. PLoS Pathog 6: e1000796.
60. Krammer F, et al. 2012. A carboxy-terminal trimerization domain stabilizes conformational epitopes on the stalk domain of soluble recombinant hemagglutinin substrates. PLoS One 7: e43603.
61. Margine I, et al. 2013a. Expression of functional recombinant hemagglutinin and neuraminidase proteins from the novel H7N9 influenza virus using the baculovirus expression system. J Vis Exp 81: e51112.
62. Gao J, et al. 2016. Measuring influenza virus neuraminidase inhibition antibody titers by enzyme-linked lectin assay. J Vis Exp (115).
63. Rajendran M, Nachbagauer R, Ermler M E, Bunduc P, Amanat F, Izikson R, Cox M, Palese P, Eichelberger M, Krammer F. 2017. Analysis of Anti-Influenza Virus Neuraminidase Antibodies in Children, Adults, and the Elderly by ELISA and Enzyme Inhibition: Evidence for Original Antigenic Sin. MBio 8(2). pii: e02281-16.
64. Margine I, et al. 2013b. Hemagglutinin stalk-based universal vaccine constructs protect against group 2 influenza A viruses. J Virol 87: 10435-10446.

### 6.2 Example 2: Swapping Packaging Signals to Enhance Immunogenicity of Immunosubdominant Glycoproteins

This example demonstrates that immunization with influenza viruses in which the packaging signals of the influenza virus neuraminidase (NA) gene segment were swapped with the packaging signals of the influenza virus hemagglutinin (HA) gene segment elicits higher anti-NA antibody levels compared to wild-type virus in which the packaging signals have not been swapped. This example also demonstrates that immunization with influenza viruses in which the packaging signals of the influenza virus neuraminidase gene segment were swapped with the packaging signals of the influenza virus hemagglutinin gene segment decreases the anti-HA antibody levels compared to wild-type virus. This example further demonstrates that immunization with influenza viruses in which the packaging signals of the influenza virus neuraminidase gene segment were swapped with the packaging signals of the influenza virus hemagglutinin gene segment and the length of the stalk of the neuraminidase encoded by the influenza virus gene segment was increased resulted in a significant decrease the anti-HA antibody levels compared to wild-type virus.

Influenza viruses are negative-sense RNA viruses that have segmented genomes. Each genomic segment is flanked at the 5′ and 3′ ends by unique stretches of RNA that serve as packaging signals, which allow for the segments to associate during viral replication and budding. It has been previously demonstrated that rewiring these packaging signals such that a genomic segment codes for one protein but is flanked by the packaging signals of another and vice versa is possible and potentially useful for controlling reassortment (Gao Q, Palese P. Rewiring the RNAs of influenza virus to prevent reassortment. Proc Natl Acad Sci USA. 2009; 106(37): 15891-15896).

It was hypothesized that by swapping the packaging signals, the expression levels of the proteins encoded on the rewired segments could be altered. In turn, it was also hypothesized that by altering the expression levels of viral surface glycoproteins, the immunogenicity of these proteins in the context of whole virus vaccination could change.

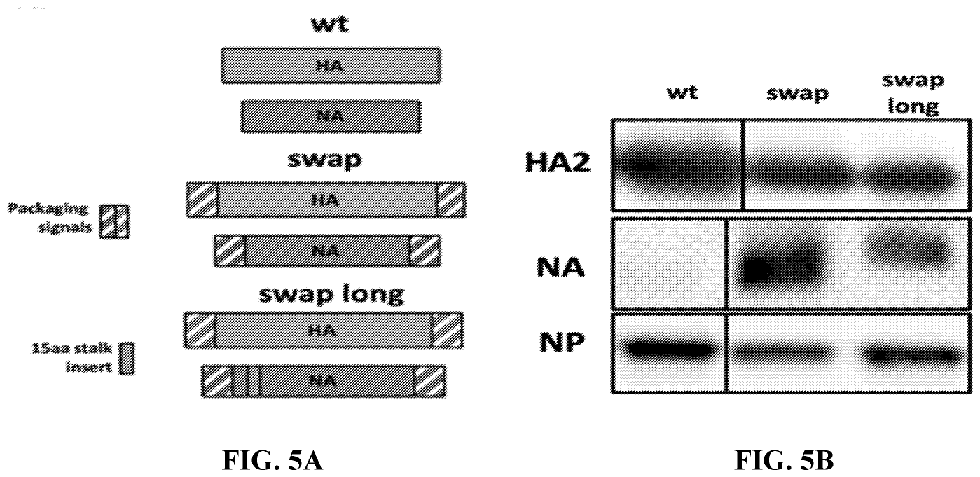

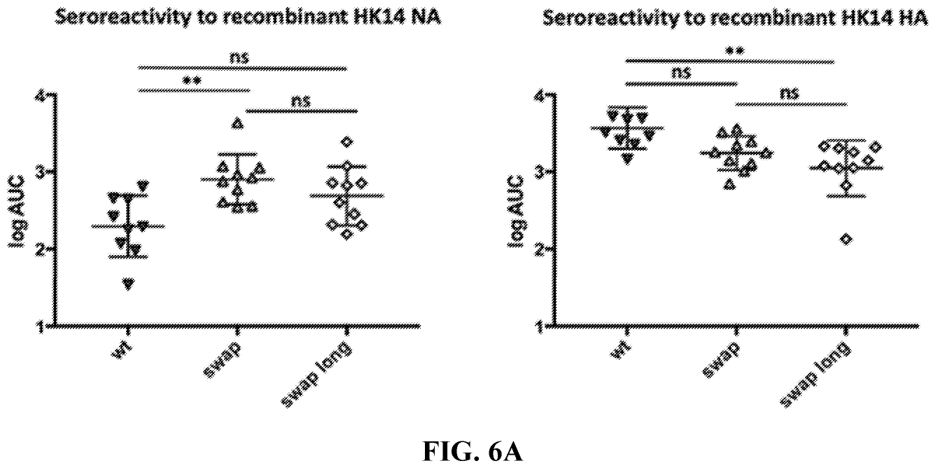

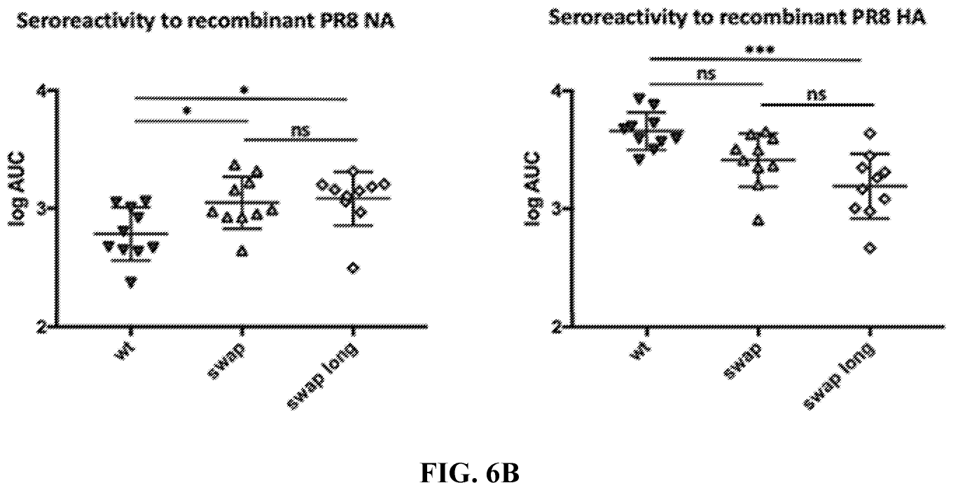

First, a rewired segment was designed where the open reading frame (ORF) of A/Hong Kong/4801/2014 (HK14) HA was flanked by the NA packaging signals of A/Puerto Rico/8/1934 (PR8) and a rewired segment where the ORF of HK14 NA was flanked by PR8 HA packaging signals. See the sequences set forth in SEQ ID Nos: 25 and 26 for the sequences of the rewired segments. A virus with these rewired segments in a PR8 backbone (swap) was rescued. In addition, a wild-type (wt) counterpart with unmodified segments encoding HK14 HA and NA was rescued. Additionally, a rewired virus with a 15 amino acid stalk extension in the HK14 NA (swap long) in a PR8 backbone was rescued (FIG. 5A). See SEQ ID NO: 29 for the nucleotide sequence encoding the 15 amino acid stalk extension sequence and SEQ ID NO: 28 for the nucleotide sequence comprising the rewired NA segment with the insertion encoding the 15 amino acid stalk extension. These viruses were plaque purified, grown in embryonated chicken eggs, inactivated with formaldehyde, and purified by ultracentrifugation through a sucrose gradient. Protein content was determined by BCA assay. Western blot for HK14 HA, HK14 NA, and PR8 NP show more NA and less HA in the swap viruses compared to wild-type (FIG. 5B). Three groups of BalB/c mice were immunized intramuscularly with inactivated HK14 wt, swap, or swap long virus twice at 4 week intervals with 10 μg total protein. Sera was isolated 4 weeks after the second immunization to assess seroreactivity against recombinant HK14 HA and HK14 NA by ELISA. Swap virus immunization elicited a significantly higher anti-NA immune response compared to wild-type. There was no added benefit to increasing the stalk length of the NA in enhancing immunogenicity (FIG. 6A). Swap virus immunization decreased anti-HA immune response, but only to a significant degree with the swap longstalk virus.

The effect of rewiring packaging signals for PR8 HA and PR8 NA expressing viruses as well was assessed. Viruses with chimeric gene segments containing PR8 HA ORF flanked by PR8 NA packaging signals and PR8 NA ORF flanked by PR8 HA packaging signals were rescued in a PR8 backbone. See the sequences set forth in SEQ ID Nos: 23 and 24 for the sequences of the rewired segments. Importantly, serial synonymous mutations were made in the 5′ and 3′ proximal regions of the ORF to abrogate residual packaging function of these regions. In addition, a wild-type counterpart with unmodified segments encoding PR8 NA and HA was rescued. Further, a rewired virus with 30 amino acid extension in the PR8 NA (swap long) in a PR8 backbone was rescued. See SEQ ID NO: 34 for the nucleotide sequence encoding the 30 amino acid stalk extension sequence and SEQ ID NO: 27 for the nucleotide sequence comprising the rewired segment with the insertion encoding the 30 amino acid stalk extension. Mice were immunized intramuscularly with 10 μg of inactivated, purified PR8 wt, swap, or swap long viruses and bled 4 weeks post immunization for sera isolation. Similar results to immunization with HK14 viruses were seen. Swap virus immunization elicited significantly higher anti-NA immune response compared to wild-type, and there was no added benefit to increasing stalk length. Again, swap virus immunization decreased anti-HA immune response, but only to a significant degree with the swap longstalk virus (FIG. 6B).

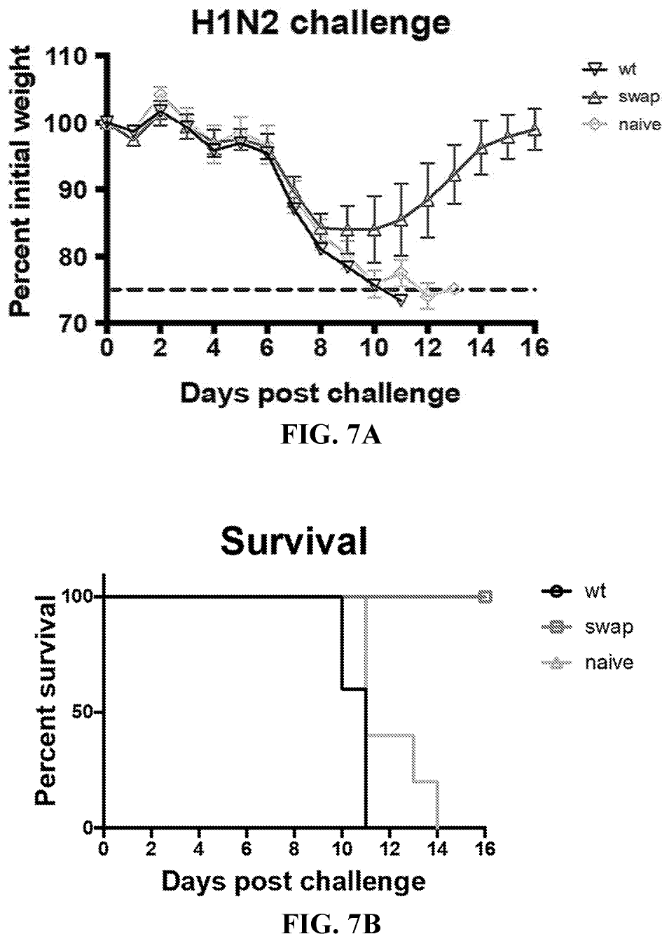

To compare the protective efficacy of the anti-NA antibody response elicited by immunization with HK14 wt and swap viruses, a passive transfer experiment was performed. An H1N2 virus with PR8 HA and HK14 N2 was rescued for challenge. Mice immunized with PBS or inactivated HK14 wt or swap virus were terminally bled 4 weeks after the second immunization. Sera from each group was pooled and 50 μl were injected into each of 5 mice per group. Mice were then challenged with 5× LD50 of H1N2 virus. Passive transfer of swap sera significantly reduced mortality compared to passive transfer of wt sera as measured by weight loss (FIG. 7A) and survival (FIG. 7B).

### 6.3 Example 3: Recombinant Influenza B Virus Expressing NA with an Extended Stalk

This example demonstrates that immunization with an influenza B virus in which the stalk of the neuraminidase encoded by the influenza virus NA gene segment has been extended significantly increases the anti-NA immune response compared to immunization with an influenza B virus with a wild-type stalk.

To assess the effect of NA stalk extension for influenza B viruses, recombinant influenza B viruses expressing B/Brisbane/60/2008 (Bris08) HA and either wild-type (wt) or long stalk Bris08 NA proteins in a B/Malaysia/2506/2004 backbone were generated. For the long stalk virus, 46 amino acids derived from both the stalk of H3N2 virus A/Hong Kong/4801/2014 N2 and the stalk of H1N1 virus A/California/04/2009 N1 were inserted into the stalk of Bris08 NA. See SEQ ID NO: 33 for the nucleotide sequence encoding the 46 amino acid extension sequence and SEQ ID NO: 32 for the nucleotide sequence encoding the rewired segment encoding the 46 amino acid stalk extension. See SEQ ID NO: 30 for the nucleotide sequence of B/Brisbane/60/2008 (Bris08) HA and SEQ ID NO: 31 for the nucleotide sequence of wild-type B/Brisbane/60/2008 (Bris08) NA. Wild-type and long stalk viruses were plaque purified, grown in embryonated chicken eggs, inactivated with formaldehyde, and purified by ultracentrifugation through a sucrose gradient. Protein content was determined by BCA assay.

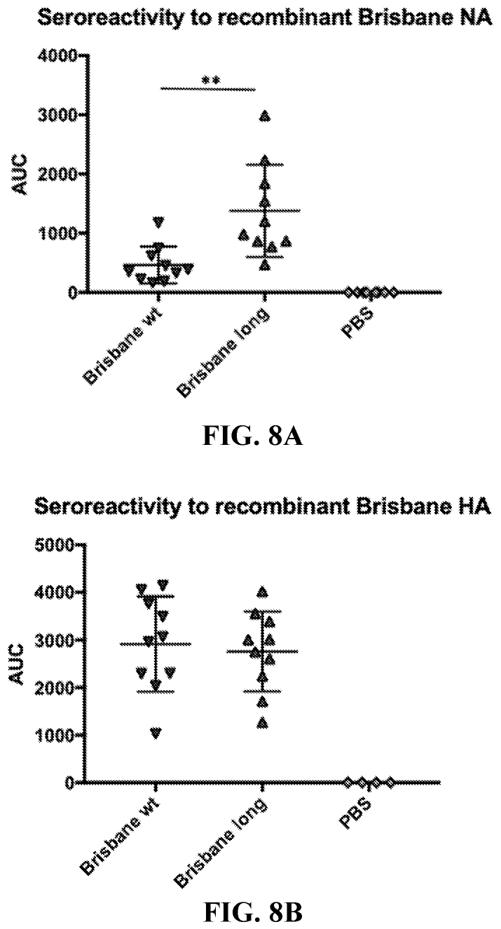

Two groups of BalB/c mice were immunized intramuscularly with inactivated Bris08 wt or long stalk virus twice at 4 week intervals with 10 μg total protein per dose. Sera was isolated 4 weeks after the second immunization to assess seroreactivity against recombinant Bris08 HA and NA by ELISA. Long stalk virus immunization elicited a significantly higher anti-NA immune response compared to wild-type (FIG. 8A). Long stalk virus immunization did not significantly decrease the anti-HA immune response (FIG. 8B).

### 6.4 Example 4: Enhancing Neuraminidase Immunogenicity of Influenza a Viruses by Rewiring RNA Packaging Signals

Humoral immune protection against influenza virus infection is mediated largely by antibodies against hemagglutinin (HA) and neuraminidase (NA), the two major glycoproteins on the virus surface. While influenza virus vaccination efforts have focused mainly on the HA, NA-based immunity has been shown to reduce disease severity and provide heterologous protection. Current seasonal vaccines do not elicit strong anti-NA responses—in part due to the immunodominance of the HA protein. The data presented in this example demonstrates that by swapping the 5′ and 3′ terminal packaging signals of the HA and NA genomic segments, which contain the RNA promoters, influenza viruses that express more NA and less HA were able to be rescued. Vaccination with formalin-inactivated, “rewired” viruses significantly enhances the anti-NA antibody response compared to vaccination with unmodified viruses. Passive transfer of sera from mice immunized with rewired virus vaccines shows better protection against influenza virus challenge. The results presented in this example provide evidence that the immunodominance of HA stems in part from its abundance on the viral surface, and that rewiring viral packaging signals—thereby increasing the NA content on viral particles—is a viable strategy for improving the immunogenicity of NA in an influenza virus vaccine. In particular, this example demonstrates the efficacy of rewiring influenza virus packaging signals for creating vaccines with more neuraminidase content which provide better NA-based protection.

### 6.4.1 Introduction

Influenza virus entry and egress is mediated predominantly by the two major surface glycoproteins, hemagglutinin (HA) and neuraminidase (NA). These two proteins function antagonistically—HA is responsible for sialic acid binding while NA cleaves sialic acid (1). Current seasonal influenza virus vaccination strategies focus heavily on eliciting an immune response against the viral HA, as anti-HA antibodies are often neutralizing, and hemagglutination-inhibition is an established correlate of protection (2, 3). Antigenic drift of the HA head domain necessitates constant reformulation of seasonal vaccines, and annual vaccine effectiveness is highly variable (4).

Anti-NA antibody titers have been shown to correlate with reductions in both viral shedding and infection severity (3, 5, 6), and small molecules which inhibit NA currently serve as first-line therapeutics for active influenza virus infection (7). The amino acid drift rates for NA are lower than those for HA (8, 9), and substantial evidence exists for the ability of humoral NA antibody responses to confer heterologous protection (10-15).

Despite strong evidence that NA-based immunity is protective, current seasonal vaccines are only required to contain 15 μg of HA, without standardization of NA content (16). Recent work has demonstrated that, in contrast to natural infection, seasonal vaccination fails to induce robust anti-NA immune responses (17). While a number of platforms like recombinant NA protein (13-15) or NA-only VLPs (18, 19) have been put forth as vaccine candidates, few strategies exist for boosting the host immune response against NA in the context of influenza virus vaccines that also induce HA immunity. HA is the predominant glycoprotein on the virus surface, outnumbering the NA at estimates ranging from 4-14:1 (20, 21), and its immunodominance over the NA has been well characterized during both vaccination and infection (22, 23). In this study, by rewiring the terminal 5′ and 3′ packaging signals of the HA and NA genomic segments, it is demonstrated that viruses can be rescued that express more NA and less HA. Vaccination with these viruses induces stronger anti-NA humoral responses that protect mice in passive transfer studies against influenza virus challenge.

### 6.4.2 Materials and Methods

Cell Culture. Human embryonic kidney 293 Ts (HEK 293 Ts) were maintained with Dulbecco's Modified Eagle's medium (DMEM; Gibco) containing 10% (vol/vol) fetal bovine serum (FBS; Hyclone) and 100 units/ml of penicillin/100 μg/ml streptomycin (PS; Gibco). Madin-Darby canine kidney cells (MDCKs) were maintained with Minimum Essential Medium (MEM; Gibco) containing 10% (vol/vol) FBS, 0.15% (w/vol) sodium bicarbonate (Corning), 20 mM 2-[4-(2-hydroxyethyl)piperazin-1-yl] ethanesulfonic acid (HEPES; Gibco), 2 mM L-glutamine (Gibco), and 100 units/ml/100 μg/ml PS. All cells were maintained at 37° C. and 5% CO₂.

Rewired segment design, plasmids, and cloning. The rewired segments were designed based on the nucleotide sequences of the HA and NA genes of PR8 H1N1 and HK14 H3N2 viruses (40). Rewired segments were designed with NheI and XhoI restriction enzyme sites flanking the 3′ and 5′ ends respectively of the HA and NA ORFs for ease of future cloning. Segments were ordered as synthetic double-stranded DNA fragments (gBlocks; Integrated DNA Technologies) and cloned into an ambisense pDZ vector (41) using the In-Fusion HD cloning kit (Clontech). Products were transformed in DH5α competent cells (Invitrogen) and plasmids were obtained using the QIAprep Spin Miniprep kit (Qiagen). Plasmids were sequence-confirmed by Sanger-sequencing (Macrogen). The pRS PR8 6-segment plasmid used here for viral rescue drives ambisense expression of the PR8 PB1, PB2, PA, NP, M, and NS segments. The construction of the pRS PR8 6-segment plasmid employed a similar approach as the construction of the pRS PR8 7-segment plasmid that has been described previously (42).

Rescue of viruses. Viruses were rescued by transfection of HEK 293T cells in six-well plates. HEK 293T cells were transfected with 2.1 μg of PRS PR8 6-segment, 0.7 μg of pDZ HA segment, and 0.7 μg of pDZ NA segment plasmids using TransIT LT1 transfection reagent (Mirus Bio). Transfected cells were cultured at 37° C. for 48 hours post-transfection and supernatant was harvested. Eight-day old embryonated chicken eggs (Charles River) were injected with 200 μl transfection supernatant and incubated at 33° C. for 72 hours. Eggs were cooled after incubation at 4° C. overnight and the allantoic fluids were collected for screening by HA analysis. HA-positive samples were used to plaque-purify virus. Virus was grown on MDCK cells, plaques picked, and resuspended in PBS for injection into 10-day old embryonated eggs. Allantoic fluid was subjected to RNA isolation through QIAamp Viral RNA Mini Kit (Qiagen). One step RT-PCR using the Superscript™ III One-Step-RT-PCR system with Platinum™ Taq DNA Polymerase (Invitrogen) was performed on isolated RNA with primers specific to the 5′ and 3′ termini of segments 4 and 6 of PR8 and HK14 (PR8 HA forward: CCGAAGTTGGGGGGGAGCAAAAGCAGGGGAAAATAA (SEQ ID NO: 17); PR8 HA reverse: GGCCGCCGGGTTATTAGTAGAAACAAGGGTGTTTTT (SEQ ID NO: 18); PR8 NA forward: CGAAAGCAGGGGTTTAAAATG (SEQ ID NO: 15); PR8 NA reverse: TTTTTGAACAGACTACTTGTCAATG (SEQ ID NO: 16); HK14 HA forward: GGGAGCAAAAGCAGGGGATAATTC (SEQ ID NO: 21); HK14HA reverse: GGGTTATTAGTAGAAACAAGGGTGTTTTTAATTAATG (SEQ ID NO: 22); HK14 NA forward: GGGAGCAAAAGCAGGAGTAAAGATG (SEQ ID NO: 19); HK14 NA reverse: TTATTAGTAGAAACAAGGAGTTTTTTCTAAAATTGCG (SEQ ID NO: 20); Thermo Fisher) to amplify DNA of the genomic segments of interest for sequence confirmation. DNA was isolated by gel-purification and sequenced by Sanger sequencing (Genewiz).

Hemagglutination Assay (HA). HA was performed using 96-well V-bottom plates. Allantoic fluid was serially diluted twofold in PBS to a volume of 50 μl/well. A 0.5% suspension of turkey red blood cells (Lampire) in PBS was prepared and 50 μl added to each well. Plates were incubated at 4° C. and read once red blood cells in the negative control settled to the bottom of the well. HA titer was defined as the highest reciprocal dilution of allantoic fluid that caused agglutination of red blood cells.

Formalin-inactivation and purification of viruses. Plaque-purified, HA and NA sequence-confirmed influenza viruses were grown in 10-day-old embryonated chicken eggs at 33° C. for 72 hours. Eggs were refrigerated at 4° C. overnight and allantoic fluids were pooled from 10 to 20 eggs per virus. Pooled allantoic fluid was treated with 0.03% (vol/vol) formalin and incubated at 4° C. for 72 hours with shaking to inactivate the virus. Inactivation was confirmed by negative plaque assay. Inactivated allantoic fluid was added onto 5 ml of 30% (w/vol) sucrose solution in 0.1 M NaCl/10 mM Tris-HCl/1 mM EDTA (pH 7.4) in ultracentrifuge tubes (Denville). Samples were spun at 4° C. and 25,000 rpm for 2 hours with an sw28 rotor in an L7-65 ultracentrifuge (Beckman). Supernatant was aspirated out and the pellet containing virus was resuspended in 1 ml PBS. Virus was aliquoted and stored at −80° C. Protein concentration of virus preparation was assessed by Pierce bicinchoninic acid (BCA) protein assay kit (Thermo Fisher).

Immunoblot. Immunoblot was performed on formalin-inactivated, purified virus. One μg protein per sample was prepared in Tris-glycine-SDS sample buffer (Invitrogen) and NuPAGE sample reducing agent (Invitrogen) and boiled at 100° C. for five minutes before being loaded onto 10% Mini-PROTEAN TGX precast gels (Bio-Rad) and run under denaturing conditions in the presence of sodium dodecyl sulfate (SDS). After running, blots were transferred onto polyvinylidene difluoride (PVDF) membranes. Color Prestained Protein Standard, Broad Range (New England Biolabs) was used as a protein size marker. PBS with 5% (w/vol) fat-free milk powder was used to block membranes for one hour. Membranes were washed three times with PBS containing 0.05% (vol/vol) Tween-20 (PBS-T). For detection of PR8 proteins, the following antibodies were used: mouse anti-N1 monoclonal antibody 4A5 (43) (1 μg/ml), rabbit anti-H1 (Thermo Fisher; PAS-34929; 1:3000), and rabbit anti-NP (Invitrogen; PAS-32242; 1:3000). For detection of HK14 proteins, the following antibodies were used: mouse anti-H3 monoclonal antibody 12D1 (44) and anti-N2 polyclonal guinea pig sera raised against recombinant N2 protein (generated in house; 1:2000). Primary antibodies were diluted in PBS with 1% (w/vol) bovine serum albumin (BSA) and membranes were incubated overnight at 4° C. Membranes were washed three times with PBS-T and incubated with secondary HRP-conjugated antibodies (anti-mouse, GE Healthcare, NXA931V; anti-rabbit, GE Healthcare, NA9340V; anti-guinea pig, Invitrogen, 61-4620) for one hour at room temperature. All secondary antibodies were diluted 1:3000 in PBS with 1% (w/vol) BSA. After washing three times with PBS-T, blots were developed using Pierce™ ECL Western Blotting Substrate (Thermo Scientific) and imaged in a ChemiDoc® MP Imaging System (Bio-Rad).

Cryo-electron Tomography. C-Flat 2/2-3C grids were glow discharged for 30 seconds at 25 m A in a Pelco easiglow. Solution containing purified virus was diluted with 10 nm colloidal gold and 2 μl was applied to each grid. Grids were back-side blotted and frozen in liquid ethane using a Leica EM GP2 Plunge Freezer, Grids were stored in liquid nitrogen until imaging. Imaging was performed on a FEI Titan Krios operated at 300 kV, equipped with a Gatan BioQuantum K3 direct detector using a 20 eV slit width. Tomograms were acquired using SerialEM-3.7.0, collecting between −60° and +60° in a dose-symmetric scheme with a 3° angular increment. The nominal magnification was 53 kx, giving a pixel size of 1.7 Å at the specimen level.

Quantification of amounts of HA and NA in tomograms. 12 tomograms of HK14-wt virus and 12 tomograms of HK14-swap virus data were pooled into one dataset, with the identity of each tomogram blinded to the person doing the analysis. For each virus particle, the distribution of HA and NA on the surface was visually assessed based on the characteristic morphology and symmetry of the proteins. Each particle was assigned to a class: >75% HA, >75% NA or a mix of HA and NA. A total of 141 virus particles, were analyzed: 79 HK14-wt particles and 62 HK14-swap particles. After analysis the identity of the tomogram was revealed, and the data presented as histograms.

Mouse immunization and passive transfer studies. Six- to eight-week old female BALB/c mice (Charles River) were immunized intramuscularly with formalin-inactivated purified virus at a dose of 10 μg per mouse after diluting to 100 μl in PBS. Four weeks after the final immunization dose, mice were euthanized and blood was collected by cardiac puncture. Sera were isolated after centrifugation of blood. For passive transfer, equal amounts of sera were taken from each mouse and pooled. Fifty μl pooled sera were transferred intraperitoneally to six- to eight-week old female BALB/c mice. Two hours later, mice were anesthetized with a cocktail of ketamine/xylazine and then infected intranasally with five times the LD₅₀ of an H1N2 virus expressing PR8 HA and HK14 NA in a PR8 backbone. Weight loss and survival were monitored for 16 days post-infection. All animal experiments were performed in accordance with procedures approved by the Institutional Animal Care and Use Committee of the Icahn School of Medicine at Mount Sinai.

Enzyme-linked Immunosorbent Assay (ELISA). ELISAs were used to assess seroreactivity to viral proteins. Area Under the Curve (AUC) was used as readout. Purified recombinant trimeric PR8 and HK14 HA and tetrameric PR8 and HK14 NA were produced as described previously (45, 46). Immulon 4 HBX 96-well plates (Thermo Scientific) were coated overnight at 4° C. with 2 μg/ml of purified recombinant protein in coating buffer (SeraCare Life Sciences Inc.) at 50 μl per well. The following day, plates were washed three times with 225 μl PBS-T and incubated with 220 μl blocking buffer (3% goat serum, 0.5% non-fat dried milk powder in PBS-T) in each well for 1 hour at room temperature (RT). Next, plates were incubated for 2 hours at RT with sera that was serially diluted three-fold in blocking buffer with a starting dilution of 1:50 for NA and 1:100 for HA. The first and last columns were used as plate blanks. Plates were washed with PBS-T three times and incubated with 50 μl/well of anti-mouse IgG-horseradish peroxidase (HRP) conjugated antibody (GE Healthcare), anti-mouse IgG1-HRP conjugated antibody (Abcam), or anti-mouse IgG2a-HRP conjugated antibody (Abcam) at 1:3000 dilution in blocking buffer for 1 hour. Plates were washed four times with PBS-T before adding 100 μl/well o-phenylenediamine dihydrochloride (SigmaFast OPD; Sigma) substrate. The reaction was quenched with 50 μl 3M HCl after 10 minutes and optical density (OD) was measured at 492 nm with a Synergy 4 plate reader (BioTek). The average OD value of the plate blanks plus three standard deviations for each plate was less than 0.07 for all plates. Baseline signal for each plate was set at a value of 0.07 for AUC calculations. AUC was log transformed and graphed using Prism 7.0 (GraphPad). Log₁₀ AUC values are reported as mean with standard deviation.

Enzyme-linked lectin assay (ELLA). This assay was performed in accordance with previous reports (47, 48). Immulon 4 HBX 96-well plates (Thermo Scientific) were coated overnight at 4° C. with 50 μg per mL of fetuin (Sigma) in coating buffer (SeraCare Life Sciences Inc.). The next day, in a separate 96-well plate, serum samples that had been heat-inactivated at 56° C. for 30 minutes were serially diluted twofold in PBS starting with a 1:20 dilution and a final volume of 75 μl/well. The first column was left as a virus-only control and the last column was left for background. H1N2 virus expressing HK14 N2 was diluted in PBS containing 1% BSA to a 90% effective concentration (EC₉₀), and 75 μl/well were added to serially diluted samples and the virus-only control column. The background column received 75 μl/well of PBS with 1% BSA. The plates with serum/virus mixture were incubated at RT for two hours. One hundred μl of the serum/virus mixture per well were transferred to the fetuin-coated plates after they had been washed three times with PBS-T. After two-hour incubation at 37° C., plates were washed three times with PBS-T and 100 μl/well of peanut agglutinin-horseradish peroxidase conjugate (Sigma) at 5 μg/ml in PBS were added. Plates were incubated in the dark for one hour before SigmaFast OPD substrate (Sigma) was added. Substrate reaction was quenched with 50 μl 3M HCl after 10 minutes and OD was measured at 492 nm with a Synergy 4 plate reader (BioTek). The values of the average of the background wells were subtracted from the values of the rest of the plate. The new values were divided by the average of the virus-only wells and multiplied by 100 to get a percentage of NA activity. Percent NI was calculated by subtracting the NA activity from 100%. The 50% inhibitory concentration (IC₅₀) of each serum sample was calculated in Prism 7.0 (GraphPad) by fitting a nonlinear regression. Reciprocal IC₅₀ values were log-transformed and statistical significance was assessed by unpaired t-test since only two groups were compared.

Antibody-dependent cell-mediated cytotoxicity (ADCC) reporter assay. The capacity of serum antibodies to elicit ADCC was measured using the ADCC Reporter Bioassay Kit (Promega Life Sciences). MDCK cells were seeded in a 96-well dish to a total of 2.5×10⁴ cells per well in 100 μl complete DMEM with 100 units/ml/100 μg/ml of PS (Gibco) and incubated overnight at 37° C. and 5% CO₂. Media was removed and cells were rinsed with PBS. Cells were then infected with H1N2 virus expressing HK14 NA at a multiplicity of infection of five. Twenty-four hours post-infection, virus was removed from cells and 25 μl diluted pooled serum was added to each well. Murine ADCC effector cells expressing FcγRIV (Promega) were diluted to add 7.5×10⁴ cells per well in Roswell Park Memorial Institute 1640 media (Gibco) containing 4% Ultra Low IgG FBS (Gibco) in 25 μl. The mixture was allowed to incubate at 37° C. and 5% CO₂ for 6 hours. After allowing the plate to equilibrate to room temperature, 75 μl of Bio-Glo Luciferase (Promega) was added and luminescence was immediately read on a Synergy 4 plate reader (BioTek). Fold change was calculated as relative luminescence values divided by the average of background wells plus three times the standard deviation. AUC of background subtracted values was determined using Prism 7.0 (GraphPad) and log₁₀ values are reported as mean of technical duplicates.

Statistics. All statistical analysis was performed using Prism 7.0 (GraphPad). Statistical differences from all ELISA assays were determined using one-way analysis of variance tests with Bonferroni correction for multiple comparisons on log-transformed AUC values. Statistical difference from the ELLA assay was determined by unpaired t-test on log-transformed reciprocal IC₅₀ values.

### 6.4.3 Results

Design of Rewired PR8 Virus

Prior studies have demonstrated that segments coding for foreign proteins like green fluorescent protein (GFP) can be efficiently packaged with the influenza virus genome by flanking the open reading frames (ORFs) of these proteins with variable stretches of nucleotides taken from the 5′ and 3′ termini of influenza virus gene segments (24-30). These terminal stretches, comprised of both the untranslated regions (UTRs) and a portion of the ORFs, provide each segment with a unique packaging identity, and suggest an explanation for how influenza viruses can efficiently and consistently incorporate their whole genomes into budding virions (31). Previous work from has demonstrated that the packaging signals of two genomic segments can be swapped to rescue rewired viruses that grow to high titers (32).

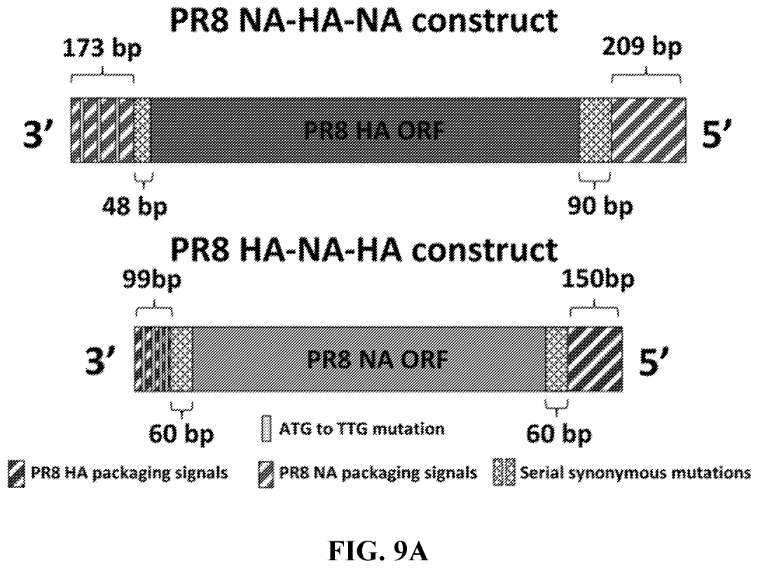

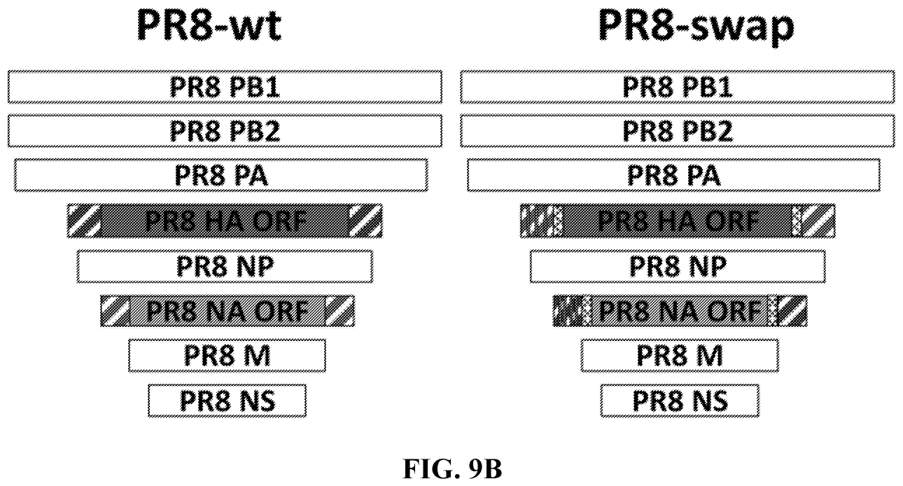

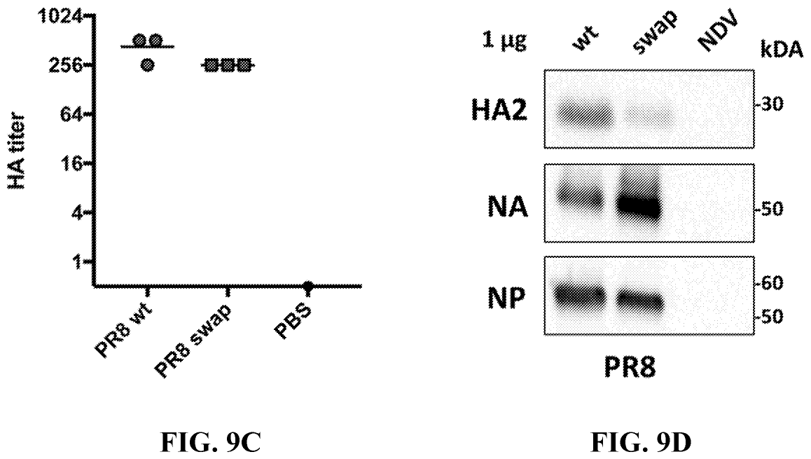

In this study, the packaging signals of segments 4 and 6, the HA and NA genes respectively, of A/Puerto Rico/8/1934 (PR8) H1N1 virus were swapped such that segment 4 was comprised of the PR8 H1 ORF flanked by segment 6 packaging signals, and segment 6 was comprised of the PR8 N1 ORF flanked by segment 4 packaging signals (FIG. 9A). The nucleotides utilized as segment 6 packaging signals were the 3′ terminal 173 base pairs (bp) and the 5′ terminal 209 bp of the PR8 NA gene segment. The nucleotides utilized as segment 4 packaging signals were the 3′ terminal 99 bp and 5′ terminal 150 bp of the PR8 HA gene segment. The specific nucleotides used as packaging signals were determined based on previous literature (32). Serial synonymous mutations were made to the regions of the termini of the HA and NA ORFs implicated in genome packaging in order to abrogate their residual packaging function. The ATGs located in the coding portions of the introduced packaging signals were mutated to TTGs in order to prevent premature translation of the viral protein. These chimeric segments, termed PR8 NA-HA-NA and PR8 HA-NA-HA respectively, were used to rescue rewired PR8 virus (PR8-swap) by reverse genetics. Wild-type PR8 virus (PR8-wt) was rescued in parallel (FIG. 9B). PR8-swap virus grew to slightly lower HA titers than PR8-wt virus in embryonated chicken eggs after plaque purification (FIG. 9C).

Since the promoter regions that drive expression of the viral proteins are located in the UTRs of the genomic segments (1), it was assessed if rewiring the packaging signals of segments 4 and 6 would alter the abundance of HA and NA on viral particles. Immunoblotting of purified, formalin-inactivated PR8-wt and PR8-swap viruses for HA and NA demonstrates clear differences in virion glycoprotein abundance (FIG. 9D). Less HA and more NA is detected in purified PR8-swap virus compared to purified PR8-wt virus, suggesting that rewiring the HA and NA packaging signals alters the expression levels of these proteins and their abundance on the viral surface. Purified Newcastle Disease Virus (NDV) was used as a negative control.

Immunization with Rewired PR8 Virus Induces Stronger Anti-NA Humoral Response

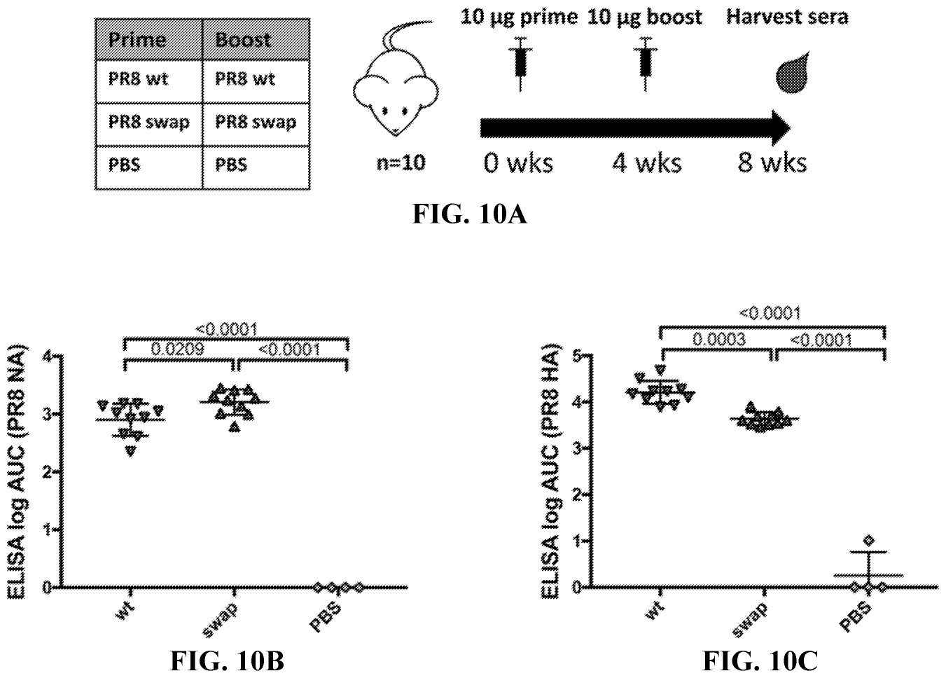

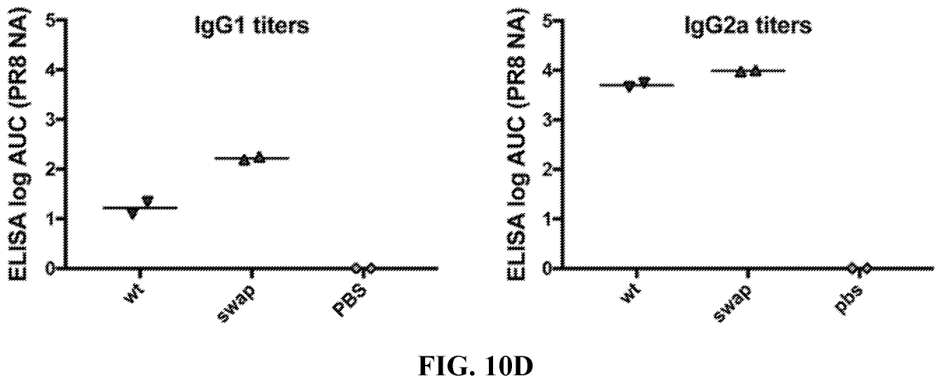

Given the altered relative abundance of HA and NA seen in virus with rewired packaging signals, it was hypothesized that immunization with PR8-swap virus would elicit stronger anti-NA and weaker anti-HA antibody responses than immunization with PR8-wt virus. Two groups of 10 six- to eight-week-old BALB/c mice received two 10 μg doses of either formalin-inactivated, purified PR8-wt or PR8-swap virus intramuscularly 4 weeks apart. One additional group of mice received two doses of 100 μl phosphate-buffered saline (PBS) as a control. Mice were euthanized four weeks after the second dose and sera were harvested for downstream analysis (FIG. 10A).

Enzyme-linked immunosorbent assays (ELISAs) were performed to determine serum IgG responses against recombinant PR8 H1 and PR8 N1 protein. PR8-swap virus immunization induced a significantly stronger (˜1.9-fold) anti-NA IgG response and a significantly weaker (˜4.1-fold) anti-HA IgG response compared to PR8-wt virus immunization. Both vaccination strategies elicited significantly higher antibody titers against HA and NA compared to the PBS control group (FIGS. 10B, 10C). Pooled sera were used for IgG subtype analysis by ELISA. Swap virus immunization elicited higher IgG1 (˜9.7-fold) and IgG2a responses (˜1.9-fold) against recombinant PR8 NA protein compared to wild-type virus immunization (FIG. 10D). High IgG2a titers suggest that these antibodies have undergone more extensive affinity maturation and can better elicit Fc-effector functions. These results indicate that, in the context of inactivated influenza virus vaccination, relative abundance is a major determinant of immunogenicity.

Design of Clinically Relevant H3N2-Expressing Viruses with Rewired Packaging Signals

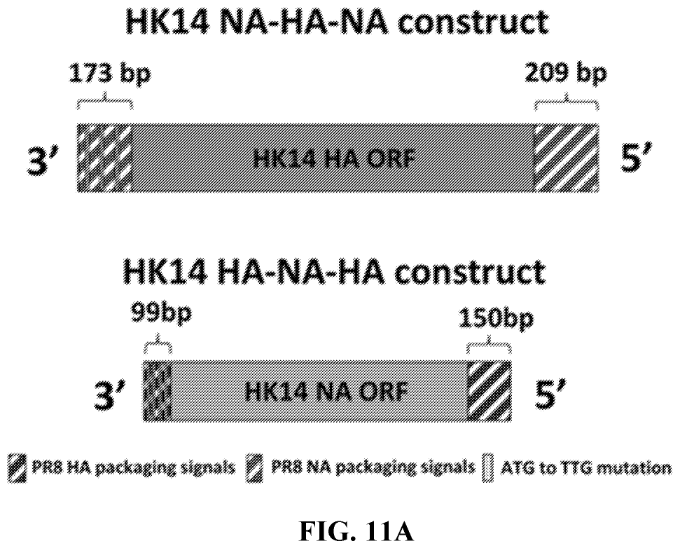

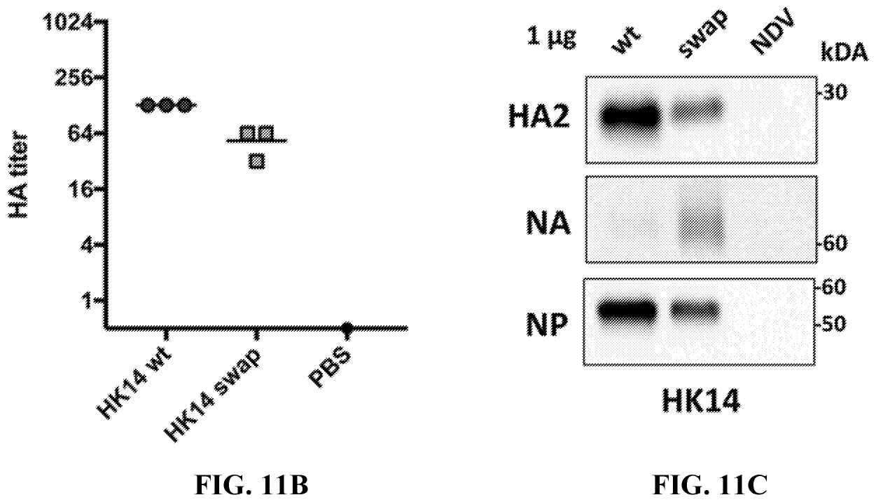

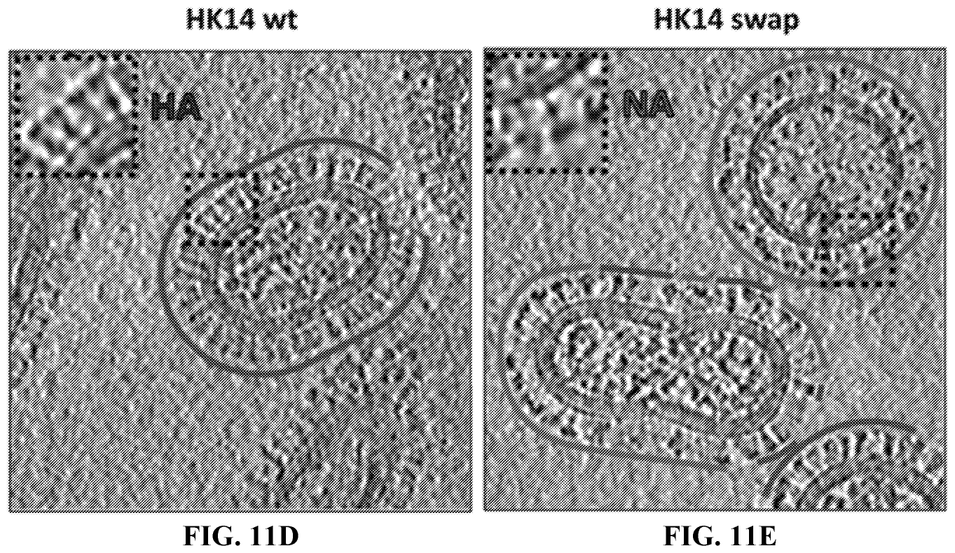

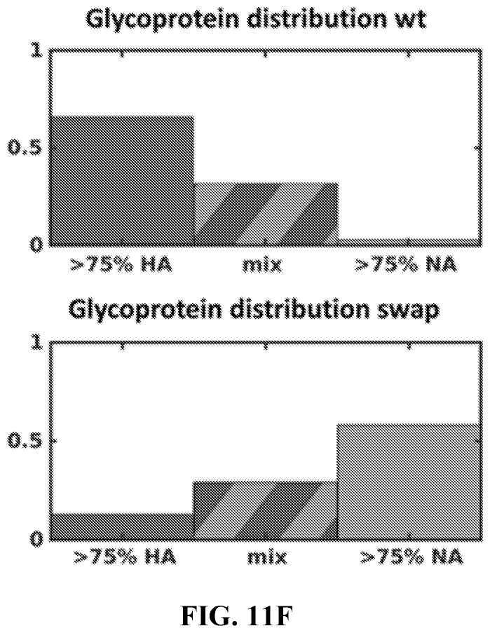

The manufacture of inactivated seasonal vaccines typically involves generation of reassortant influenza viruses expressing HAs and NAs of circulating seasonal strains in a PR8 backbone (33). The H3N2 strain used for the 2016-2017 and 2017-2018 seasonal vaccines was A/Hong Kong/4801/2014 (HK14). To determine if the strategy could be applied to a clinically relevant H3N2-expressing virus, genomic segments encoding HK14 H3 and N2 with the swapped packaging signals described above were first designed (FIG. 11A). PR8 packaging signals were used for optimal incorporation of these segments into the PR8 backbone. Modified HK14 segment 4 (HK14 NA-HA-NA) was comprised of the ORF of HK14 H3 flanked by the packaging signals of the PR8 NA gene. Modified HK14 segment 6 (HK14 HA-NA-HA) was comprised of the ORF of HK14 N2 flanked by the packaging signals of the PR8 HA gene. ATGs located in the coding portions of the introduced packaging signals were mutated to TTGs in order to prevent premature translation of viral protein as before. These chimeric segments were used to rescue rewired HK14 H3N2-expressing virus (HK14-swap) in a PR8 backbone using reverse-genetics. Wild-type HK14 segments 4 and 6 were used to rescue recombinant virus expressing HK14 HA and NA (HK14-wt) in a PR8 backbone, as is consistent with current vaccine design. Similar to wild-type and rewired PR8 viruses, the HK14-swap virus grew to slightly lower HA titers than HK14-wt virus in embryonated chicken eggs and expressed more NA and less HA than HK14-wt virus by immunoblot of formalin-inactivated, purified viral particles (FIGS. 11B, 11C).

To confirm the differences observed in HA and NA expression by immunoblot, purified, inactivated HK14-wt and HK14-swap viruses were subjected to cryoelectron tomography. Representative tomogram sections show that growth of HK14-swap virus led to the release of particles displaying more NA glycoproteins and fewer HA glycoproteins on their surfaces compared to HK14-wt virus (FIGS. 11D, 11E). HA molecules are distinguished by their characteristic “peanut” shape, and NA molecules are distinguished by their denser, shorter head region (20). To assess the relative abundance of HA and NA on viral particles, quantification of surface glycoproteins was performed on 79 HK14-wt particles and 62 HK14-swap particles. For the majority of analyzed HK14-wt particles, HA comprises more than 75% of observed surface glycoproteins. In contrast, for the majority of analyzed HK14-swap particles, NA comprises more than 75% of observed surface glycoproteins (FIG. 11F).

Immunization with Rewired HK14 Virus Enhances Both NA-Inhibiting and Anti-NA Fc Effector Function-Active Antibody Responses

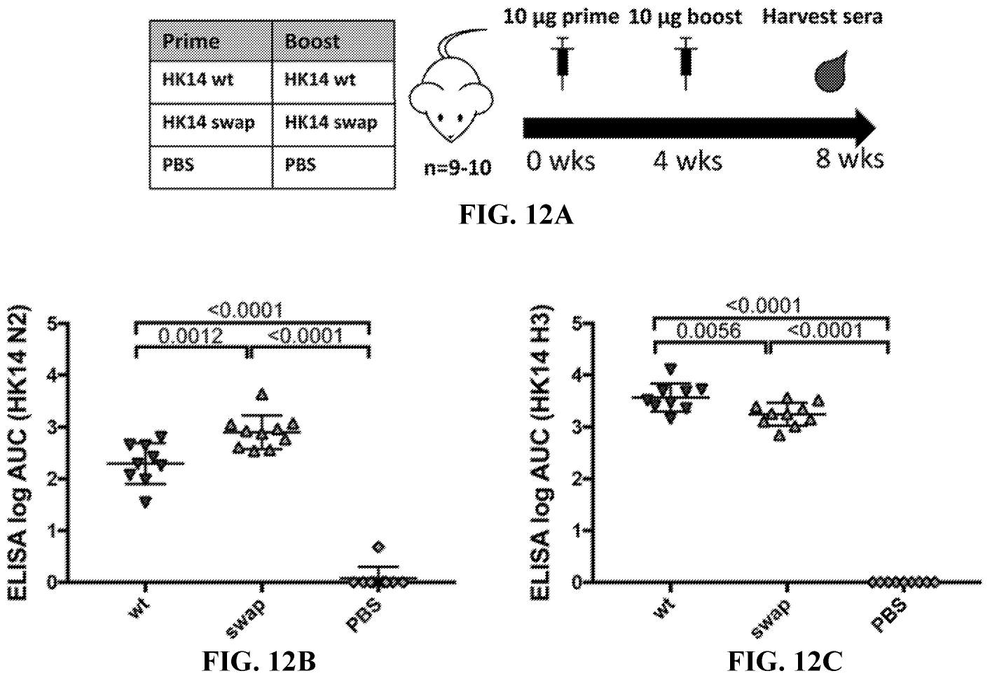

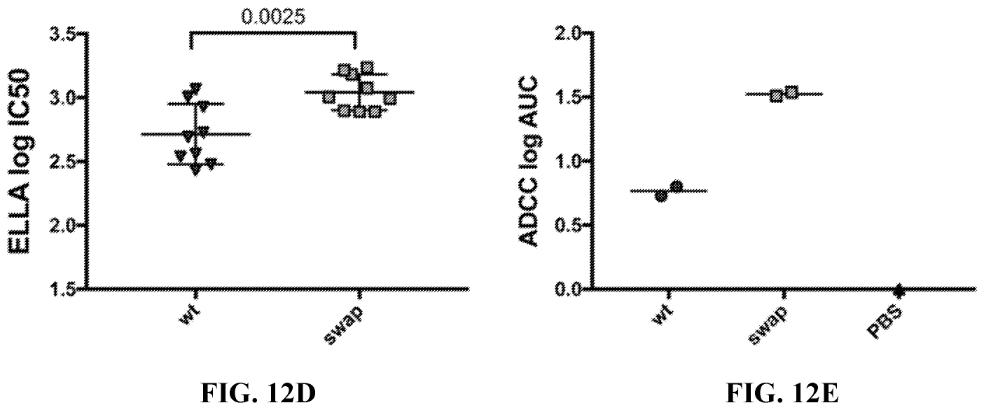

Next, a comparison of the humoral responses elicited by these viruses upon immunization was assessed. As before, two groups of 10 six- to eight-week-old BALB/c mice received two 10 μg doses of either formalin-inactivated, purified HK14-wt or HK14-swap virus intramuscularly 4 weeks apart. A control group of mice received two 100 μl injections of PBS (naïve). Mice were euthanized four weeks after the second dose and their sera were isolated for downstream analysis (FIG. 12A).

ELISAs were performed with either recombinant HK14 N2 or HK14 H3 protein to assess IgG responses. Consistent with the data on PR8 viruses, HK14-swap virus immunization induced a significantly stronger (˜4-fold) anti-NA IgG response and a significantly weaker (˜2.3-fold) anti-HA IgG response than HK14-wt virus immunization. Both immunization regimens elicited significantly higher anti-H3 and anti-N2 responses than PBS (FIGS. 12B, 12C).

Next, it was sought to characterize the functionality of these antibodies in terms of their abilities to inhibit neuraminidase activity and induce Fc-receptor-mediated effector functions. A PR8 H1 HK14 N2-expressing virus (H1N2) was used in order to characterize the NA-specific humoral response. An enzyme-linked lectin assay (ELLA) was performed using H1N2 virus to assess the capacity of immunized mouse sera to inhibit the enzymatic activity of HK14 N2. Sera raised against HK14-swap virus showed significantly stronger inhibition of N2 activity than sera raised against HK14-wt virus (FIG. 12D).

While inhibition of the viral neuraminidase is the classic mechanism by which anti-NA antibodies are known to function, some antibodies are also able to induce Fc effector functions like antibody-dependent cellular cytotoxicity (ADCC) (14). An in vitro ADCC reporter assay on immunized mouse sera using Madin-Darby canine kidney cells (MDCKs) infected with H1N2 virus was performed. Pooled sera raised against HK14-swap virus showed higher ADCC activity (˜5.7 fold) than pooled sera raised against HK14-wt virus (FIG. 12E). These data suggest that the rewired viruses are able to elicit a stronger overall anti-NA humoral response that is better able to both inhibit neuraminidase activity and induce ADCC activity.

Humoral Response Elicited by Rewired Virus Immunization Protects Against Influenza Virus Challenge

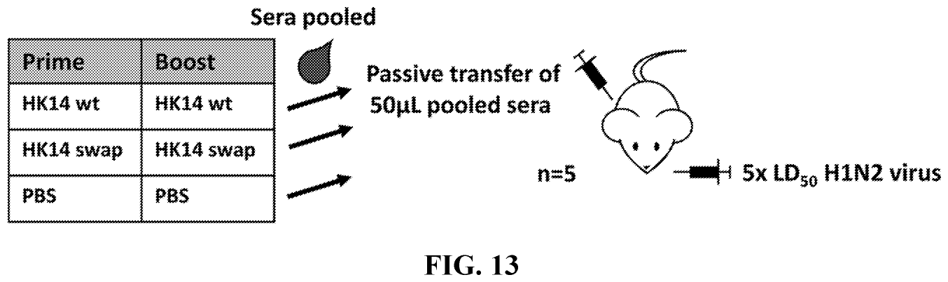

Next passive transfer studies were performed with these sera in order to investigate the protective efficacy of the anti-NA antibody response elicited by the rewired and wild-type HK14 viruses. The same quantity of sera collected from individual immunized mice was pooled for each group. Three groups of five mice received either HK14-wt, HK14-swap, or naïve sera by passive transfer. Each mouse was injected intraperitoneally (IP) with pooled sera. In order to see the differences in protective efficacy between HK14-wt and HK14-swap sera, only 50 μl of pooled sera were transferred per mouse. Two hours post-transfer, mice were challenged intranasally with five times the median lethal dose (LD₅₀) of an NA-matched H1N2 virus expressing HK14 NA and PR8 HA in order to specifically assess the protection conferred by NA-based immunity (FIG. 13). Weight loss and survival were measured for 16 days post-challenge. Mice were euthanized upon reaching 75% of their initial body weight. All of the mice that received HK14-swap sera survived, whereas all of the mice that received HK14-wt or naïve sera succumbed to the infection (FIGS. 7A, 7B). Thus, immunization with the rewired virus significantly enhances NA-based humoral protection for a clinically relevant NA.

### 6.4.4 Discussion

Here, novel chimeric influenza virus genomic segments were designed for which the segment encoding for HA has NA packaging signals and the segment encoding for NA has HA packaging signals. These constructs can be used to rescue viruses by reverse genetics that express less HA and more NA on the viral surface than viruses with unmodified segments. The effect of this rewiring on the expression of other viral proteins has not been examined.

Consistent with the change in relative abundance of HA and NA, rewired viruses grow to slightly lower but comparable titers. Evidence is provided that this change in surface glycoprotein abundance challenges the immunodominance of the HA. By ELISA, there is stronger seroreactivity to purified NA protein and weaker seroreactivity to purified HA protein after immunization with swap virus compared to wild-type virus. It is likely that this is due to increased antigenic visibility of the NA in conjunction with decreased antigenic visibility of the HA. This effect is seen for both H1N1- and H3N2-expressing viruses, suggesting a broad applicability for this platform. The stronger NA-specific humoral response is reflected in an increase in both NI- and ADCC-active antibodies that provides better protection against virus challenge.

Previous work has demonstrated that extending the NA stalk domain such that the NA protrudes farther than the HA can also increase immunogenicity, suggesting that HA immunodominance is not solely a function of abundance (34). Whether or not package signal rewiring can be combined with stalk extension to further enhance antigenic visibility and immunogenicity of the NA remains to be seen.

While a number of studies have provided evidence that a functional balance between HA and NA abundance is essential for viral fitness (35-37), the data provided herein suggest that influenza viruses are viable over a large range of HA to NA expression ratios. This is supported by a recent study demonstrating that the relative abundance of viral proteins can vary widely between individual virions (21). It is likely that the functional HA/NA balance is relevant for transmission dynamics, which is unaccounted for when passaging viruses in eggs or tissue culture.

As efforts to improve the breadth and protective efficacy of influenza virus vaccines have redirected focus away from the variable HA head, there has been renewed interest in exploring NA as a target antigen (38, 39). Strategies that are currently being explored include the use of recombinant NA proteins (13-15), virus-like particles (18, 19), and RNA or DNA (12) vaccination approaches. Similar to work describing extension of the NA stalk as a feasible method for improving NA immunogenicity (34), a strategy for strengthening NA-based immunity in the context of influenza virus vaccination is described herein and its protective efficacy in mice is demonstrated. The weaker HA-based humoral response that is elicited can be addressed by supplementation with recombinant HA protein in future vaccine candidates. Importantly, it is unclear if changing the immunogenicity of NA in seasonal vaccines will increase immunological pressure on the NA and thus affect NA drift rates. This work provides evidence that rewiring HA and NA packaging signals is a viable platform for developing influenza virus vaccines that elicit stronger, more protective NA-based humoral responses.

### 6.4.5 References Cited in Example 4

1. Shaw M L, Palese P. 2013. Orthomyxoviridae, p. 1151-1185. In Knipe, D M, Howley, P M (eds.), Fields Virology Sixth Edition. Lippincott Williams & Wilkins.
2. Wohlbold T J, Krammer F. 2014. In the shadow of hemagglutinin: A growing interest in influenza viral neuraminidase and its role as a vaccine antigen. Viruses 6:2465-2494.
3. Memoli M J, Shaw P A, Han A, Czajkowski L, Reed S, Athota R, Bristol T, Fargis S, Risos K, Powers J H, Davey R T, Taubenberger J K. 2016. Evaluation of antihemagglutinin and antineuraminidase antibodies as correlates of protection in an influenza A/H1N1 virus healthy human challenge model. MBio 7:1-12.
4. Wei C-J, Crank M C, Shiver J, Graham B S, Mascola J R, Nabel G J. 2020. Next-generation influenza vaccines: opportunities and challenges. Nat Rev Drug Discov. 19:239-252.
5. Couch R B, Atmar R L, Franco L M, Quarles J M, Wells J, Arden N, Nino D, Belmont J W. 2013. Antibody correlates and predictors of immunity to naturally occurring influenza in humans and the importance of antibody to the neuraminidase. J Infect Dis 207:974-981.
6. Monto A S, Petrie J G, Cross R T, Johnson E, Liu M, Zhong W, Levine M, Katz J M, Ohmit S E. 2015. Antibody to influenza virus neuraminidase: An independent correlate of protection. J Infect Dis 212:1191-1199.
7. Johansson B E, Cox M M J. 2011. Influenza viral neuraminidase: The forgotten antigen. Expert Rev Vaccines 10:1683-1695.
8. Abed Y, Hardy I, Li Y, Boivin G. 2002. Divergent evolution of hemagglutinin and neuraminidase genes in recent influenza A:H3N2 viruses isolated in Canada. J Med Virol 67:589-595.
9. Sandbulte M R, Westgeest K B, Gao J, Xu X, Klimov A I, Russell C A, Burke D F, Smith D J, Fouchier R A M, Eichelberger M C. 2011. Discordant antigenic drift of neuraminidase and hemagglutinin in H1N1 and H3N2 influenza viruses. Proc Natl Acad Sci USA 108:20748-20753.
10. Chen Z, Kadowaki S E, Hagiwara Y, Yoshikawa T, Matsuo K, Kurata T, Tamura S I. 2000. Cross-protection against a lethal influenza virus infection by DNA vaccine to neuraminidase. Vaccine 18:3214-3222.
11. Sylte M J, Hubby B, Suarez D L. 2007. Influenza neuraminidase antibodies provide partial protection for chickens against high pathogenic avian influenza infection. Vaccine 25:3763-3772.
12. Xie H, Li X, Gao J, Lin Z, Jing X, Plant E, Zoueva O, Eichelberger M C, Ye Z. 2011. Revisiting the 1976 “swine flu” vaccine clinical trials: Cross-reactive hemagglutinin and neuraminidase antibodies and their role in protection against the 2009 H1N1 pandemic virus in mice. Clin Infect Dis 53:1179-1187.
13. Wohlbold T J, Nachbagauer R, Xu H, Tan G S, Hirsh A, Brokstad K A, Cox R J, Palese P. 2015. Vaccination with Adjuvanted Recombinant Neuraminidase Induces Broad Heterologous, but Not Heterosubtypic, Cross-Protection against Influenza Virus Infection in Mice. MBio 6:1-13.
14. Wohlbold T J, Podolsky K A, Chromikova V, Kirkpatrick E, Falconieri V, Meade P, Amanat F, Tan J, Tenoever B R, Tan G S, Subramaniam S, Palese P, Krammer F. 2017. Broadly protective murine monoclonal antibodies against influenza B virus target highly conserved neuraminidase epitopes. Nat Microbiol 2:1415-1424.
15. McMahon M, Kirkpatrick E, Stadlbauer D, Strohmeier S, Bouvier N M, Krammer F. 2019. Mucosal immunity against neuraminidase prevents influenza B virus transmission in Guinea pigs. MBio 10:1-12.
16. Air G M. 2012. Influenza neuraminidase. Influenza Other Respi Viruses 6:245-256.
17. Chen Y Q, Wohlbold T J, Zheng N Y, Huang M, Huang Y, Neu K E, Lee J, Wan H, Rojas K T, Kirkpatrick E, Henry C, Palm A K E, Stamper C T, Lan L Y L, Topham D J, Treanor J, Wrammert J, Ahmed R, Eichelberger M C, Georgiou G, Krammer F, Wilson P C. 2018. Influenza Infection in Humans Induces Broadly Cross-Reactive and Protective Neuraminidase-Reactive Antibodies. Cell 173:417-429.e10.
18. Easterbrook J D, Schwartzman L M, Gao J, Kash J C, Morens D M, Couzens L, Wan H, Eichelberger M C, Taubenberger J K. 2012. Protection against a lethal H5N1 influenza challenge by intranasal immunization with virus-like particles containing 2009 pandemic H1N1 neuraminidase in mice. Virology 432:39-44.
19. Smith G E, Sun X, Bai Y, Liu Y V., Massare M J, Pearce M B, Belser J A, Maines T R, Creager H M, Glenn G M, Flyer D, Pushko P, Levine M Z, Tumpey T M. 2017. Neuraminidase-based recombinant virus-like particles protect against lethal avian influenza A(H5N1) virus infection in ferrets. Virology 509:90-97.
20. Harris A, Cardone G, Winkler D C, Heymann J B, Brecher M, White J M, Steven A C. 2006. Influenza virus pleiomorphy characterized by cryoelectron tomography. Proc Natl Acad Sci 103:19123-19127.
21. Vahey M D, Fletcher D A. 2019. Low-Fidelity Assembly of Influenza A Virus Promotes Escape from Host Cells. Cell 176:281-294.e19.
22. Johansson B E, Moran T M, Bona C A, Popple S W, Kilbourne E D. 1987. Immunologic response to influenza virus neuraminidase is influenced by prior experience with the associated viral hemagglutinin. II. Sequential infection of mice simulates human experience. J Immunol. 139:2010-2014.
23. Kilbourne E D. 1976. Comparative efficacy of neuraminidase specific and conventional influenza virus vaccines in induction of antibody to neuraminidase in humans. J Infect Dis 134:384-394.
24. Fujii Y, Goto H, Watanabe T, Yoshida T, Kawaoka Y. 2003. Selective incorporation of influenza virus RNA segments into virions. Proc Natl Acad Sci USA 100:2002-7.
25. Muramoto Y, Takada A, Fujii K, Noda T, Iwatsuki-Horimoto K, Watanabe S, Horimoto T, Kida H, Kawaoka Y. 2006. Hierarchy among viral RNA (vRNA) segments in their role in vRNA incorporation into influenza A virions. J Virol 80:2318-25.
26. Marsh G A, Hatami R, Palese P. 2007. Specific Residues of the Influenza A Virus Hemagglutinin Viral RNA Are Important for Efficient Packaging into Budding Virions. J Virol 81:9727-9736.
27. Goto H, Muramoto Y, Noda T, Kawaoka Y. 2013. The Genome-Packaging Signal of the Influenza A Virus Genome Comprises a Genome Incorporation Signal and a Genome-Bundling Signal. J Virol 87:11316-11322.
28. Ozawa M, Maeda J, Iwatsuki-Horimoto K, Watanabe S, Goto H, Horimoto T, Kawaoka Y. 2009. Nucleotide Sequence Requirements at the 5′ End of the Influenza A Virus M RNA Segment for Efficient Virus Replication. J Virol 83:3384-3388.
29. Ozawa M, Fujii K, Muramoto Y, Yamada S, Yamayoshi S, Takada A, Goto H, Horimoto T, Kawaoka Y. 2007. Contributions of Two Nuclear Localization Signals of Influenza A Virus Nucleoprotein to Viral Replication. J Virol 81:30-41.
30. Liang Y, Hong Y, Parslow T G. 2005. cis-Acting Packaging Signals in the Influenza Virus PB1, PB2, and PA Genomic RNA Segments cis-Acting Packaging Signals in the Influenza Virus PB1, PB2, and PA Genomic RNA Segments. J Virol 79:10348-10355.
31. Hutchinson E C, von Kirchbach J C, Gog J R, Digard P. 2010. Genome packaging in influenza A virus. J Gen Virol 91:313-328.
32. Gao Q, Palese P. 2009. Rewiring the RNAs of influenza virus to prevent reassortment. Proc Natl Acad Sci USA 106:15891-15896.
33. Ping J, Lopes T J S, Nidom C A, Ghedin E, MacKen C A, Fitch A, Imai M, Maher E A, Neumann G, Kawaoka Y. 2015. Development of high-yield influenza A virus vaccine viruses. Nat Commun 6:1-15.
34. Broecker F, Zheng A, Suntrongwong N, Sun W, Bailey M J, Krammer F, Palese P. 2019. Extending the stalk enhances immunogenicity of the influenza virus neuraminidase. J Virol JVI.00840-19.
35. Wagner R, Matrosovich M, Klenk H D. 2002. Functional balance between haemagglutinin and neuraminidase in influenza virus infections. Rev Med Virol 12:159-166.
36. Yen H-L, Liang C-H, Wu C-Y, Forrest H L, Ferguson A, Choy K-T, Jones J, Wong D D-Y, Cheung P P-H, Hsu C-H, Li O T, Yuen K M, Chan R W Y, Poon L L M, Chan M C W, Nicholls J M, Krauss S, Wong C-H, Guan Y, Webster R G, Webby R J, Peiris M. 2011. Hemagglutinin-neuraminidase balance confers respiratory-droplet transmissibility of the pandemic H1N1 influenza virus in ferrets. Proc Natl Acad Sci 108:14264-14269.
37. Gaymard A, Le Briand N, Frobert E, Lina B, Escuret V. 2016. Functional balance between neuraminidase and haemagglutinin in influenza viruses. Clin Microbiol Infect 22:975-983.
38. Krammer F, Fouchier RAM, Eichelberger M C, Webby R J, Shaw-Saliba K, Wan H, Wilson P C, Compans R W, Skountzou I, Monto A S. 2018. NAction! how can neuraminidase-based immunity contribute to better influenza virus vaccines? MBio 9:1-12.
39. Krammer F, Li L, Wilson P C. 2019. Emerging from the Shadow of Hemagglutinin: Neuraminidase Is an Important Target for Influenza Vaccination. Cell Host Microbe 26:712-713.
40. Broecker F, Liu S T H, Sun W, Krammer F, Simon V, Palese P. 2018. Immunodominance of Antigenic Site B in the Hemagglutinin of the Current H3N2 Influenza Virus in Humans and Mice. J Virol 92:1-13.
41. Martinez-Sobrido L, Garcia-sastre A. 2010. Generation of Recombinant Influenza Virus from Plasmid DNA. J Vis Exp e2057.
42. Fulton B O, Sun W, Heaton N S, Palese P. 2018. The Influenza B Virus Hemagglutinin Head Domain Is Less Tolerant to Transposon Mutagenesis than That of the Influenza A Virus. J Virol 92:e00754-18.
43. Sandbulte M R, Jimenez G S, Boon A C M, Smith L R, Treanor J J, Webby R J. 2007. Cross-reactive neuraminidase antibodies afford partial protection against H5N1 in mice and are present in unexposed humans. PLoS Med 4:0265-0272.
44. Wang T T, Tan G S, Hai R, Pica N, Petersen E, Moran T M, Palese P. 2010. Broadly protective monoclonal antibodies against H3 influenza viruses following sequential immunization with different hemagglutinins. PLoS Pathog 6:e10007996.
45. Margine I, Palese P, Krammer F. 2013. Expression of Functional Recombinant Hemagglutinin and Neuraminidase Proteins from the Novel H7N9 Influenza Virus Using the Baculovirus Expression System. JoVE e51112.
46. Krammer F, Margine I, Tan G S, Pica N, Krause J C, Palese P. 2012. A carboxy-terminal trimerization domain stabilizes conformational epitopes on the stalk domain of soluble recombinant Hemagglutinin substrates. PLoS One 7:e43603.
47. Gao J, Couzens L, Eichelberger M C. 2016. Measuring Influenza Neuraminidase Inhibition Antibody Titers by Enzyme-linked Lectin Assay. J Vis Exp e54573.
48. Rajendran M, Nachbagauer R, Ermler M E, Bunduc P, Amanat F, Izikson R, Cox M, Palese P, Eichelberger M, Krammer F. 2017. Analysis of anti-influenza virus neuraminidase antibodies in children, adults, and the elderly by ELISA and enzyme inhibition: Evidence for original antigenic sin. MBio 8:1-12.

### 7. EQUIVALENTS

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. An immunogenic composition comprising an influenza virus and an adjuvant, wherein the influenza virus comprises a mutated influenza virus neuraminidase polypeptide, and wherein the mutated influenza virus neuraminidase polypeptide comprises a first cytoplasmic domain, a first transmembrane domain, a first stalk domain, and a first globular head domain of a first neuraminidase of a first influenza A virus with an insertion of 15 to 45 amino acid residues in the first stalk domain of the first neuraminidase.

2. The immunogenic composition of claim 1, wherein the insertion is of 15 to 30 amino acid residues.

3. The immunogenic composition of claim 2, wherein the insertion is of 15 amino acid residues.

4. The immunogenic composition of claim 2, wherein the insertion is of 30 amino acid residues.

5. The immunogenic composition of any one of claims 1 to 4, wherein the amino acid residues inserted correspond to amino acid residues found in a second stalk domain of a second neuraminidase of a second influenza A virus, wherein the first influenza A virus is from a different subtype than second influenza A virus.

6. The immunogenic composition of any one of claims 1 to 4, wherein the amino acid residues inserted correspond to amino acid residues found in a second stalk domain of a second neuraminidase of a second influenza A virus, wherein the first influenza A virus is from a different strain than second influenza A virus.

7. The immunogenic composition of 5 or 6, wherein the second influenza A virus is influenza A virus H1N1pdm09 A/California/04/2009 (Cal09) virus.

8. The immunogenic composition of any one of claims 1 to 7, wherein the first influenza A virus neuraminidase is a neuraminidase of influenza A virus of subtype N1, N2, N3, N4, N5, N6, N7, N8, N9, N10, or N11.

9. The immunogenic composition of claims 1 to 8, wherein the first influenza A virus is influenza A virus H1N1 A/Puerto Rico/8/1934 (PR8) or influenza A virus H3N2 A/New York/61/2012 (NY12).

10. An immunogenic composition comprising an influenza virus, wherein the influenza virus comprises a mutated influenza virus neuraminidase polypeptide, and wherein the mutated influenza virus neuraminidase polypeptide comprises the amino acid sequence of SEQ ID NO: 4.

11. The immunogenic composition of claim 10, wherein the influenza virus is influenza A virus H3N2 A/New York/61/2012 (NY12).

12. An immunogenic composition comprising an influenza virus, wherein the influenza virus comprises a mutated influenza virus neuraminidase polypeptide, and wherein the mutated influenza virus neuraminidase polypeptide comprises the amino acid sequence of SEQ ID NO: 8 or 10.

13. The immunogenic composition of claim 10 or 12, wherein the influenza virus is influenza A virus H1N1 A/Puerto Rico/8/1934 (PR8).

14. The immunogenic composition of any one of claims 10 to 13, wherein the immunogenic composition further comprises an adjuvant.

15. The immunogenic composition of claim 14, wherein the adjuvant is an aluminum salt (alum), 3 De-O-acylated monophosphoryl lipid A (MPL), MF59 AS01, AS03, or AS04.

16. The immunogenic composition of any one of claims 1 to 9, wherein the adjuvant is an aluminum salt (alum), 3 De-O-acylated monophosphoryl lipid A (MPL), MF59 AS01, AS03, or AS04.

17. The immunogenic composition of any one of claims 1 to 16, wherein the influenza virus is inactivated.

18. The immunogenic composition of any one of claims 1 to 16, wherein the influenza virus is a live attenuated influenza virus.

19. A method for immunizing against influenza virus in a human subject, comprising administering to the subject a composition comprising an influenza virus, wherein the influenza virus comprises a mutated influenza virus neuraminidase polypeptide, and wherein the mutated influenza virus neuraminidase polypeptide comprises a first cytoplasmic domain, a first transmembrane domain, a first stalk domain, and a first globular head domain of a first neuraminidase of a first influenza A virus with an insertion of 15 to 45 amino acid residues in the first stalk domain of the first neuraminidase.

20. The method of claim 19, wherein the insertion is of 15 to 30 amino acid residues.

21. The method of claim 19, wherein the insertion is of 15 amino acid residues.

22. The method of claim 19, wherein the insertion is of 30 amino acid residues.

23. The method of any one of claims 19 to 22, wherein the amino acid residues inserted correspond to amino acid residues found in a second stalk domain of a second neuraminidase of a second influenza A virus, wherein the first influenza A virus is from a different subtype than second influenza A virus.

24. The method of any one of claims 19 to 22, wherein the amino acid residues inserted correspond to amino acid residues found in a second stalk domain of a second neuraminidase of a second influenza A virus, wherein the first influenza A virus is from a different strain than second influenza A virus.

25. The method of claim 23 or 24, wherein the second influenza A virus is influenza A virus H1N1pdm09 A/California/04/2009 (Cal09) virus.

26. The method of any one of claims 19 to 25, wherein the first influenza A virus neuraminidase is a neuraminidase of influenza A virus of subtype N1, N2, N3, N4, N5, N6, N7, N8, N9, N10, or N11.

27. The method of claims 19 to 26, wherein the first influenza virus is influenza A virus H1N1 A/Puerto Rico/8/1934 (PR8) or influenza A virus H3N2 A/New York/61/2012 (NY12).

28. The method of any one of claims 19 to 27, wherein the composition further comprises an adjuvant.

29. The method of claim 28, wherein the adjuvant is an aluminum salt (alum), 3 De-O-acylated monophosphoryl lipid A (MPL), MF59, AS01, AS03, or AS04.

30. A method for immunizing against influenza virus in a subject, comprising administering to the subject the immunogenic composition of any one of claims 10 to 18.

31. A method for preventing influenza virus disease in a subject, comprising administering to the subject the immunogenic composition of any one of claims 10 to 18.

32. The method of claim 30 or 31, wherein the subject is a human subject.

33. An influenza virus comprising a mutated influenza virus neuraminidase polypeptide, and wherein the mutated influenza virus neuraminidase polypeptide comprises the amino acid sequence of SEQ ID NO: 4, 8 or 10.

34. An influenza virus comprising a genome comprising a nucleotide sequence encoding a mutated influenza virus neuraminidase polypeptide, and wherein the mutated influenza virus neuraminidase polypeptide comprises the amino acid sequence of SEQ ID NO: 4, 8 or 10.

35. The influenza virus of claim 33 or 34, wherein the influenza virus is influenza A virus H3N2 A/New York/61/2012 (NY12) or H1N1 A/Puerto Rico/8/1934 (PR8).

36. A recombinant influenza virus comprising a first chimeric influenza virus gene segment, a second chimeric influenza virus gene segment, and influenza virus NS, PB1, PB2, PA, M, and NP gene segments, wherein:
(a) the first chimeric influenza virus gene segment encodes a mutated influenza virus neuraminidase (NA) polypeptide and the first chimeric influenza virus gene segment comprises:
(i) a 3′ non-coding region of a hemagglutinin (HA) influenza virus gene segment;
(ii) a 3′ proximal coding region of the HA influenza virus gene segment, wherein any start codon in the 3′ proximal coding region of the HA influenza virus gene segment is mutated;
(iii) the open reading frame encoding for the mutated influenza virus NA polypeptide, wherein the mutated influenza virus neuraminidase polypeptide comprises a first cytoplasmic domain, a first transmembrane domain, a first stalk domain, and a first globular head domain of a first neuraminidase of a first influenza A virus with an insertion of 15 to 50 amino acid residues in the first stalk domain of the first neuraminidase;
(iv) a 5′ proximal coding region of the HA influenza virus gene segment; and
(v) the 5′ non-coding region of the HA influenza virus gene segment; and
(b) the second chimeric influenza virus gene segment encodes an influenza virus hemagglutinin (HA) polypeptide and the second chimeric influenza virus gene segment comprises:
(i) the 3′ non-coding region of an NA influenza virus gene segment;
(ii) a 3′ proximal coding region of the NA influenza virus gene segment, wherein any start codon in the 3′ proximal coding region of the NA influenza virus gene segment is mutated;
(iii) the open reading frame of the HA influenza virus gene segment,
(iv) a 5′ proximal coding region of the NA influenza virus gene segment; and
(v) the 5′ non-coding region of the NA influenza virus influenza gene segment.

37. The recombinant influenza virus of claim 36, wherein synomyous mutations are introduced into the 3′ proximal nucleotides, the 5′ proximal nucleotides, or both in the open reading frames of the mutated influenza virus neuraminidase and HA.

38. The recombinant influenza virus of claim 36 or 37, wherein the amino acid residues inserted correspond to amino acid residues found in a second stalk domain of a second neuraminidase of a second influenza A virus, wherein the first influenza A virus is from a different subtype than second influenza A virus.

39. The recombinant influenza virus of claim 36 or 37, wherein the amino acid residues inserted correspond to amino acid residues found in a second stalk domain of a second neuraminidase of a second influenza A virus, wherein the first influenza A virus is from a different strain than second influenza A virus.

40. The recombinant influenza virus of any one of claims 36 to 39, wherein the first influenza A virus neuraminidase is a neuraminidase of influenza A virus of subtype N1, N2, N3, N4, N5, N6, N7, N8, N9, N10, or N11.

41. The recombinant influenza virus of any one claims 36 to 40, wherein the first influenza A virus is influenza A virus H1N1 A/Puerto Rico/8/1934 (PR8) or influenza A virus A/Hong Kong/4801/2014.

42. The recombinant influenza virus of claim 36, wherein the first chimeric influenza virus gene segment comprises the nucleotide sequence set forth in SEQ ID NO: 27 and the second chimeric influenza virus gene segment comprises the nucleotide sequence set forth in SEQ ID NO: 23.

43. The recombinant influenza virus of claim 36, wherein the first chimeric influenza virus gene segment comprises the nucleotide sequence set forth in SEQ ID NO: 28 and the second chimeric influenza virus gene segment comprises the nucleotide sequence set forth in SEQ ID NO: 25.

44. An immunogenic composition comprising an influenza virus of any one of claims 36 to 43.

45. The immunogenic composition of claim 44 which further comprises an adjuvant.

46. The immunogenic composition of claim 45, wherein the adjuvant is an aluminum salt (alum), 3 De-O-acylated monophosphoryl lipid A (MPL), MF59, AS01, AS03, or AS04.

47. A method for immunizing against influenza virus in a subject, comprising administering to the subject the immunogenic composition of any one of claims 44 to 46.

48. A method for preventing influenza virus disease in a subject, comprising administering to the subject the immunogenic composition of any one of claims 44 to 46.

49. The method of claim 47 or 48, wherein the subject is a human subject.

50. A recombinant influenza virus comprising a mutated influenza virus neuraminidase polypeptide, and wherein the mutated influenza virus neuraminidase polypeptide comprises a first cytoplasmic domain, a first transmembrane domain, a first stalk domain, and a first globular head domain of a first neuraminidase of a first influenza B virus with an insertion of 15 to 50 amino acid residues in the first stalk domain of the first neuraminidase.

51. A recombinant influenza virus comprising a genome that comprises an NA segment, wherein the NA segment comprises a nucleotide sequence encoding a mutated influenza virus neuraminidase polypeptide, and wherein the mutated influenza virus neuraminidase polypeptide comprises a first cytoplasmic domain, a first transmembrane domain, a first stalk domain, and a first globular head domain of a first neuraminidase of a first influenza B virus with an insertion of 15 to 50 amino acid residues in the first stalk domain of the first neuraminidase.

52. The recombinant influenza virus of claim 50 or 51, wherein the insertion is of 15 to 46 amino acid residues.

53. The recombinant influenza virus of claim 50 or 51, wherein the insertion is of 46 amino acid residues.

54. The recombinant influenza virus of any one of claims 50 to 53, wherein the amino acid residues inserted correspond to amino acid residues found in a second stalk domain of a second neuraminidase of a second influenza A virus.

55. The recombinant influenza virus of any one of claims 50 to 53, wherein the amino acid residues inserted correspond to amino acid residues found in a second stalk domain of a second neuraminidase of a second influenza A virus and amino acid residues found in a third stalk domain of a third neuraminidase of a third influenza A virus.

56. The recombinant influenza virus of claim 55, wherein the second influenza A virus is influenza virus A/Hong Kong/4801/2014 and the third influenza virus is influenza virus A/California/04/2009.

57. The recombinant influenza virus of any one of claims 50 to 53, wherein the inserted amino acid residues comprise the amino acid sequence encoded by the nucleotide sequence set forth in SEQ ID NO: 34.

58. The recombinant influenza virus of any one of claims 50 to 53, wherein the mutated influenza virus neuraminidase segment is encoded by a nucleotide sequence comprising the sequence set forth in SEQ ID NO: 32.

59. The recombinant influenza virus of any one of claims 50 to 58, wherein the first influenza B virus neuraminidase is a neuraminidase of influenza virus B/Brisbane/60/2008.

60. The recombinant influenza virus of any one of claims 50 to 59, wherein the recombinant influenza virus is an influenza virus B/Malaysia/2506/2004.

61. An immunogenic composition comprising the recombinant influenza virus of any one of claims 50 to 60.

62. The immunogenic composition of claim 61 further comprising an adjuvant.

63. The immunogenic composition of claim 62, wherein the adjuvant is an aluminum salt (alum), 3 De-O-acylated monophosphoryl lipid A (MPL), MF59, AS01, AS03, or AS04.

64. The immunogenic composition of any one of claims 60 to 63, wherein the influenza virus is inactivated.

65. The immunogenic composition of any one of claims 60 to 63, wherein the influenza virus is a live attenuated influenza virus.

66. A method for immunizing against influenza virus in a subject, comprising administering to the subject the immunogenic composition of any one of claims 61 to 65.

67. A method for preventing influenza virus disease in a subject, comprising administering to the subject the immunogenic composition of any one of claims 61 to 65.

68. The method of claim 66 or 67, wherein the subject is a human subject.

69. A recombinant influenza virus comprising a first chimeric influenza virus gene segment and a second chimeric influenza virus gene segment, wherein:
(a) the first chimeric influenza virus gene segment encodes an influenza virus NA polypeptide and the first chimeric influenza virus gene segment comprises:
(i) a 3′ non-coding region of an HA influenza virus gene segment;
(ii) a 3′ proximal coding region of the HA influenza virus gene segment, wherein any start codon in the 3′ proximal coding region of the HA influenza virus gene segment is mutated;
(iii) the open reading frame encoding for the influenza virus NA polypeptide,
(iv) a 5′ proximal coding region of the HA influenza virus gene segment; and
(v) the 5′ non-coding region of the HA influenza virus gene segment; and
(b) the second chimeric influenza virus gene segment encodes an influenza virus HA and the second chimeric influenza virus gene segment comprises:
(i) the 3′ non-coding region of an NA influenza virus gene segment;
(ii) a 3′ proximal coding region of the NA influenza virus gene segment, wherein any start codon in the 3′ proximal coding region of the NA influenza virus gene segment is mutated;
(iii) the open reading frame of the HA influenza virus gene segment,
(iv) a 5′ proximal coding region of the NA influenza virus gene segment; and
(v) the 5′ non-coding region of the NA influenza virus influenza gene segment.

70. The recombinant influenza virus of claim 69, wherein 3′ proximal nucleotides, 5′ proximal nucleotides, or both in the open reading frames comprise synonymous mutations.

71. The recombinant influenza virus of claim 69 or 70, wherein the NA open reading frame and HA open reading frame are from one strain or subtype of influenza virus and the packaging signals of the chimeric gene segments comprising those open reading frames are from a different strain or subtype of influenza virus and those packaging signals from the same strain or subtype of influenza virus as influenza virus NS, PB1, PB2, PA, M, and NP gene segments.

72. The recombinant influenza virus of claim 69, wherein the first chimeric influenza virus gene segment comprises the nucleotide sequence set forth in SEQ ID NO:24, and the second chimeric influenza virus gene segment comprises the nucleotide sequence set forth in SEQ ID NO:23.

73. The recombinant influenza virus of claim 69, wherein the first chimeric influenza virus gene segment comprises the nucleotide sequence set forth in SEQ ID NO:26, and the second chimeric influenza virus gene segment comprises the nucleotide sequence set forth in SEQ ID NO:25.

74. An immunogenic composition comprising the recombinant influenza virus of any one of claims 69 to 73.

75. The immunogenic composition of claim 74 further comprising an adjuvant.

76. The immunogenic composition of claim 75, wherein the adjuvant is an aluminum salt (alum), 3 De-O-acylated monophosphoryl lipid A (MPL), MF59 AS01, AS03, or AS04.

77. The immunogenic composition of any one of claims 74 to 76, wherein the influenza virus is inactivated.

78. The immunogenic composition of any one of claims 74 to 76, wherein the influenza virus is a live attenuated influenza virus.

79. A method for immunizing against influenza virus in a subject, comprising administering to the subject the immunogenic composition of any one of claims 74 to 78.

80. A method for preventing influenza virus disease in a subject, comprising administering to the subject the immunogenic composition of any one of claims 74 to 78.

81. A method for inducing an immune response against influenza virus NA, comprising administering to the subject the immunogenic composition of any one of claims 74 to 78.

82. A method for enhancing a humoral immune response against influenza virus NA, comprising administering to a subject in need thereof the immunogenic composition of any one of claims 74 to 78.

83. The method of any one of claims 79 to 82, wherein the subject is human.


SEQUENCE LISTING (42 sequences)

SEQ ID NO: 1 - DNA, 1391 bp, Artificial Sequence
  HK14 N2-Del 25 Nucleotide (1391 bp)
agcaaaagcaggagtaaagatgaatccaaatcaaaagataataacgattggctctgtttctctcaccatttccacaatatgcttcttcatgcaaattgccattttgataactactgtaacattgcatttcaagcaaatagtgtatttaactaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaaagatattctcaggacccaggagtcagaatgcatttgtatcaatggaacttgtacagtagtaatgactgatggaagtgcttcaggaaaagctgatactaaaatactattcattgaggaggggaaaatcgttcatactagcacattgtcaggaagtgctcagcatgtcgaagagtgctcttgctatcctcgatatcctggtgtcagatgtgtctgcagagacaactggaagggctccaatcggcccatcgtagatataaacataaaggatcatagcattgtttccagttatgtgtgttcaggacttgttggagacacacccagaaaaaacgacagctccagcagtagccattgtttggatcctaacaatgaagaaggtggtcatggagtgaaaggctgggcctttgatgatggaaatgacgtgtggatgggaagaacaatcaacgagacgtcacgcttagggtatgaaaccttcaaagtcattgaaggctggtccaaccctaagtccaaattgcagacaaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcctatataagctttcgcaattttagaaaaaactccttgtttctact

SEQ ID NO: 2 - protein, 444 aa, Artificial Sequence
  HK14 N2-Del 25 Amino Acid
MNPNQKIITIGSVSLTISTICFFMQIAILITTVTLHFKQIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 3 - DNA, 1511 bp, Artificial Sequence
  HK14 N2-Ins15 Nucleotide (1511 bp)
agcaaaagcaggagtaaagatgaatccaaatcaaaagataataacgattggctctgtttctctcaccatttccacaatatgcttcttcatgcaaattgccattttgataactactgtaacattgcatttcaagcaatatgaattcaactcccccccaaacaaccaagtgatgctgtgtgaaccaacaataatagaaagaaacataacagaaatagtgtatttaactaatcagacatatgttaacatcagcaacaccaactttgctgctggaaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaaagatattctcaggacccaggagtcagaatgcatttgtatcaatggaacttgtacagtagtaatgactgatggaagtgcttcaggaaaagctgatactaaaatactattcattgaggaggggaaaatcgttcatactagcacattgtcaggaagtgctcagcatgtcgaagagtgctcttgctatcctcgatatcctggtgtcagatgtgtctgcagagacaactggaagggctccaatcggcccatcgtagatataaacataaaggatcatagcattgtttccagttatgtgtgttcaggacttgttggagacacacccagaaaaaacgacagctccagcagtagccattgtttggatcctaacaatgaagaaggtggtcatggagtgaaaggctgggcctttgatgatggaaatgacgtgtggatgggaagaacaatcaacgagacgtcacgcttagggtatgaaaccttcaaagtcattgaaggctggtccaaccctaagtccaaattgcagacaaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcctatataagctttcgcaattttagaaaaaactccttgtttctact

SEQ ID NO: 4 - protein, 484 aa, Artificial Sequence
  HK14 N2-Ins15 Amino Acid
MNPNQKIITIGSVSLTISTICFFMQIAILITTVTLHFKQYEFNSPPNNQVMLCEPTIIERNITEIVYLTNQTYVNISNTNFAAGNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 5 - DNA, 1466 bp, Artificial Sequence
  HK14 NA Nucleotide (1466 bp)
agcaaaagcaggagtaaagatgaatccaaatcaaaagataataacgattggctctgtttctctcaccatttccacaatatgcttcttcatgcaaattgccattttgataactactgtaacattgcatttcaagcaatatgaattcaactcccccccaaacaaccaagtgatgctgtgtgaaccaacaataatagaaagaaacataacagaaatagtgtatttaactaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaaagatattctcaggacccaggagtcagaatgcatttgtatcaatggaacttgtacagtagtaatgactgatggaagtgcttcaggaaaagctgatactaaaatactattcattgaggaggggaaaatcgttcatactagcacattgtcaggaagtgctcagcatgtcgaagagtgctcttgctatcctcgatatcctggtgtcagatgtgtctgcagagacaactggaagggctccaatcggcccatcgtagatataaacataaaggatcatagcattgtttccagttatgtgtgttcaggacttgttggagacacacccagaaaaaacgacagctccagcagtagccattgtttggatcctaacaatgaagaaggtggtcatggagtgaaaggctgggcctttgatgatggaaatgacgtgtggatgggaagaacaatcaacgagacgtcacgcttagggtatgaaaccttcaaagtcattgaaggctggtccaaccctaagtccaaattgcagacaaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcctatataagctttcgcaattttagaaaaaactccttgtttctact

SEQ ID NO: 6 - protein, 469 aa, Artificial Sequence
  HK14 NA Amino Acid
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 7 - DNA, 1458 bp, Artificial Sequence
  PR8 N1-Ins15 Nucleotide
agcgaaagcaggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaatcagacatatgttaacatcagcaacaccaactttgctgctggaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaactggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgttaggcaagatgttgtggcaatgactgattggtcagggtatagcggaagtttcgttcaacatcctgagctaacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 8 - protein, 470 aa, Artificial Sequence
  PR8 N1-Ins15 Amino Acid
KMNPNQKITTIGSICLVVGLISLILQIGNIISIWISHSIQTGSQNHTGICNQNIITYKNSTWVNQTYVNISNTNFAAGKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 9 - DNA, 1503 bp, Artificial Sequence
  PR8 N1-Ins30 Nucleotide
agcgaaagcaggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaatcagacatatgttaacatcagcaacaccaactttgctgctggaaacacaacagagatagtgtatctgaccaacaccaccatagagaagaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaactggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgttaggcaagatgttgtggcaatgactgattggtcagggtatagcggaagtttcgttcaacatcctgagctaacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 10 - protein, 484 aa, Artificial Sequence
  PR8 N1-Ins30 Amino Acid
MNPNQKITTIGSICLVVGLISLILQIGNIISIWISHSIQTGSQNHTGICNQNIITYKNSTWVNQTYVNISNTNFAAGNTTEIVYLTNTTIEKKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 11 - DNA, 1413 bp, Artificial Sequence
  PR8 N1-wt Nucleotide
agcgaaagcaggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaactggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgttaggcaagatgttgtggcaatgactgattggtcagggtatagcggaagtttcgttcaacatcctgagctaacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 12 - protein, 454 aa, Artificial Sequence
  PR 8 N1-wt Amino Acid
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 13 - DNA, 24 bp, Artificial Sequence
  Sequencing Primer pDZ_forward
tacagctcctgggcaacgtgctgg

SEQ ID NO: 14 - DNA, 23 bp, Artificial Sequence
  Sequencing Primer pDZ_reverse
aggtgtccgtgtcgcgcgtcgcc

SEQ ID NO: 15 - DNA, 21 bp, Artificial Sequence
  Primer PR8_NA_forward
cgaaagcaggggtttaaaatg

SEQ ID NO: 16 - DNA, 25 bp, Artificial Sequence
  Primer PR8_NA_reverse
tttttgaacagactacttgtcaatg

SEQ ID NO: 17 - DNA, 36 bp, Artificial Sequence
  Primer PR8_HA_forward
ccgaagttgggggggagcaaaagcaggggaaaataa

SEQ ID NO: 18 - DNA, 36 bp, Artificial Sequence
  Primer PR8_HA_reverse
ggccgccgggttattagtagaaacaagggtgttttt

SEQ ID NO: 19 - DNA, 25 bp, Artificial Sequence
  Primer HK14_NA_forward
gggagcaaaagcaggagtaaagatg

SEQ ID NO: 20 - DNA, 37 bp, Artificial Sequence
  Primer HK14_NA_reverse
ttattagtagaaacaaggagttttttctaaaattgcg

SEQ ID NO: 21 - DNA, 24 bp, Artificial Sequence
  Primer HK14_HA_forward
gggagcaaaagcaggggataattc

SEQ ID NO: 22 - DNA, 37 bp, Artificial Sequence
  Primer HK14_HA_reverse
gggttattagtagaaacaagggtgtttttaattaatg

SEQ ID NO: 23 - DNA, 2092 bp, Artificial Sequence
  PR8 swap virus - NA-HAmut-NA
agcgaaagcaggggtttaaattgaatccaaatcagaaaataacaaccattggatcaatctgtctggtagtcggactaattagcctaatattgcaaatagggaatataatctcaatttggattagccattcaattcaaactggaagtcaaaaccatactggaatttgcaaccaagctagcatgaaagcgaatttgttagttttactgtccgcgttggcggccgcggacgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgcttccagcttagtattgctagttagtttaggagcgatttccttttggatgtgcagcaacgggagcctacaatgtcggatttgtatttgactcgagtgagctaacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 24 - DNA, 1626 bp, Artificial Sequence
  PR8 swap virus - HA-NAmut-HA
agcaaaagcaggggaaaataaaaacaaccaaattgaaggcaaacctactggtcctgttaagtgcacttgcagctgcagttgcagacacaatttgtataggctagcatgaacccgaaccaaaagatcacgactatcgggagcatttgcttagtggttgggttgatcagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaactggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgttaggcaagatgttgtggcaatgactgattggtcagggtatagcggaagtttcgttcaacatcctgagctaacagggctagactgtatgaggccgtgcttctgggttgaattaatcaggggacgacctaaagaaaaaacaatctggactagtgcgagcagcatttctttttgtggcgtgaatagtgacaccgtagactggagctggccggatggcgccgaactaccgttttctatcgataaatagctcgagatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 25 - DNA, 2095 bp, Artificial Sequence
  HK14 swap virus - NA - HK14 HA ORF - NA
agcgaaagcaggggtttaaattgaatccaaatcagaaaataacaaccattggatcaatctgtctggtagtcggactaattagcctaatattgcaaatagggaatataatctcaatttggattagccattcaattcaaactggaagtcaaaaccatactggaatttgcaaccaagctagcatgaagactatcattgctttgagctacattctatgtctggttttcgctcaaaaaattcctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcagctgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacaaatacccagcattgaacgtgactatgccaaacaatgaacaatttgacaaattgtacatttggggggttcaccacccgggtacggacaaggaccaaatcttcccgtatgctcaatcatcaggaagaatcacagtatctaccaaaagaagccaacaagctgtaatcccaaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgactcgagtgagctaacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 26 - DNA, 1671 bp, Artificial Sequence
  HK14 swap virus - HA - HK14 NA ORF - HA
agcaaaagcaggggaaaataaaaacaaccaaattgaaggcaaacctactggtcctgttaagtgcacttgcagctgcagttgcagacacaatttgtataggctagcatgaatccaaatcaaaagataataacgattggctctgtttctctcaccatttccacaatatgcttcttcatgcaaattgccattttgataactactgtaacattgcatttcaagcaatatgaattcaactcccccccaaacaaccaagtgatgctgtgtgaaccaacaataatagaaagaaacataacagaaatagtgtatttaactaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaaagatattctcaggacccaggagtcagaatgcatttgtatcaatggaacttgtacagtagtaatgactgatggaagtgcttcaggaaaagctgatactaaaatactattcattgaggaggggaaaatcgttcatactagcacattgtcaggaagtgctcagcatgtcgaagagtgctcttgctatcctcgatatcctggtgtcagatgtgtctgcagagacaactggaagggctccaatcggcccatcgtagatataaacataaaggatcatagcattgtttccagttatgtgtgttcaggacttgttggagacacacccagaaaaaacgacagctccagcagtagccattgtttggatcctaacaatgaagaaggtggtcatggagtgaaaggctgggcctttgatgatggaaatgacgtgtggatgggaagaacaatcaacgagacgtcacgcttagggtatgaaaccttcaaagtcattgaaggctggtccaaccctaagtccaaattgcagacaaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcctatataactcgagatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 27 - DNA, 1716 bp, Artificial Sequence
  PR8 NA long stalk ORF between PR8 HA packaging signals
agcaaaagcaggggaaaataaaaacaaccaaattgaaggcaaacctactggtcctgttaagtgcacttgcagctgcagttgcagacacaatttgtataggctagcatgaacccgaaccaaaagatcacgactatcgggagcatttgcttagtggttgggttgatcagcctaatattgcaaatagggaatataatctcaatatggattagccattcaattcaaactggaagtcaaaaccatactggaatatgcaaccaaaacatcattacctataaaaatagcacctgggtaaatcagacatatgttaacatcagcaacaccaactttgctgctggaaacacaacagagatagtgtatctgaccaacaccaccatagagaagaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaactggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgttaggcaagatgttgtggcaatgactgattggtcagggtatagcggaagtttcgttcaacatcctgagctaacagggctagactgtatgaggccgtgcttctgggttgaattaatcaggggacgacctaaagaaaaaacaatctggactagtgcgagcagcatttctttttgtggcgtgaatagtgacaccgtagactggagctggccggatggcgccgaactaccgttttctatcgataaatagctcgagatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 28 - DNA, 1716 bp, Artificial Sequence
  HK14 N2 long stalk ORF between PR8 HA packaging signals
agcaaaagcaggggaaaataaaaacaaccaaattgaaggcaaacctactggtcctgttaagtgcacttgcagctgcagttgcagacacaatttgtataggctagcatgaatccaaatcaaaagataataacgattggctctgtttctctcaccatttccacaatatgcttcttcatgcaaattgccattttgataactactgtaacattgcatttcaagcaatatgaattcaactcccccccaaacaaccaagtgatgctgtgtgaaccaacaataatagaaagaaacataacagaaatagtgtatttaactaatcagacatatgttaacatcagcaacaccaactttgctgctggaaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaaagatattctcaggacccaggagtcagaatgcatttgtatcaatggaacttgtacagtagtaatgactgatggaagtgcttcaggaaaagctgatactaaaatactattcattgaggaggggaaaatcgttcatactagcacattgtcaggaagtgctcagcatgtcgaagagtgctcttgctatcctcgatatcctggtgtcagatgtgtctgcagagacaactggaagggctccaatcggcccatcgtagatataaacataaaggatcatagcattgtttccagttatgtgtgttcaggacttgttggagacacacccagaaaaaacgacagctccagcagtagccattgtttggatcctaacaatgaagaaggtggtcatggagtgaaaggctgggcctttgatgatggaaatgacgtgtggatgggaagaacaatcaacgagacgtcacgcttagggtatgaaaccttcaaagtcattgaaggctggtccaaccctaagtccaaattgcagacaaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcctatataactcgagatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 29 - DNA, 45 bp, Artificial Sequence
  Nucleotide sequence encoding 15 amino acid sequence extension
aatcagacatatgttaacatcagcaacaccaactttgctgctgga

SEQ ID NO: 30 - DNA, 1885 bp, Artificial Sequence
  Bris08 HA
agcagaagcagagcattttctaatatccacaaaatgaaggcaataattgtactactcatggtagtaacatccaatgcagatcgaatctgcactgggataacatcgtcaaactcaccacatgtcgtcaaaactgctactcaaggggaggtcaatgtgactggtgtaataccactgacaacaacacccaccaaatctcattttgcaaatctcaaaggaacagaaaccagggggaaactatgcccaaaatgcctcaactgcacagatctggacgtagccttgggcagaccaaaatgcacggggaaaataccctcggcaagagtttcaatactccatgaagtcagacctgttacatctgggtgctttcctataatgcacgacagaacaaaaattagacagctgcctaaccttctccgaggatacgaacatatcaggttatcaacccataacgttatcaatgcagaaaatgcaccaggaggaccctacaaaattggaacctcagggtcttgccctaacattaccaatggaaacggatttttcgcaacaatggcttgggccgtcccaaaaaacgacaaaaacaaaacagcaacaaatccattaacaatagaagtaccatacatttgtacagaaggagaagaccaaattaccgtttgggggttccactctgacgacgagacccaaatggcaaagctctatggggactcaaagccccagaagttcacctcatctgccaacggagtgaccacacattacgtttcacagattggtggcttcccaaatcaaacagaagacggaggactaccacaaagtggtagaattgttgttgattacatggtgcaaaaatctgggaaaacaggaacaattacctatcaaaggggtattttattgcctcaaaaggtgtggtgcgcaagtggcaggagcaaggtaataaaaggatccttgcctttaattggagaagcagattgcctccacgaaaaatacggtggattaaacaaaagcaagccttactacacaggggaacatgcaaaggccataggaaattgcccaatatgggtgaaaacacccttgaagctggccaatggaaccaaatatagacctcctgcaaaactattaaaggaaaggggtttcttcggagctattgctggtttcttagaaggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataatcatactatactgctttactactcaactgctgcctccagtttggctgtaacactgatgatagctatctttgttgtttatatggtctccagagacaatgtttcttgctccatctgtctataagggaagttaagccctgtattttcctttattgtagtgcttgtttacttgttgtcattacaaagaaacgttattgaaaaatgctcttgttactact

SEQ ID NO: 31 - DNA, 1564 bp, Artificial Sequence
  Bris08 wt NA
agcagaagcagagcatcttctcaaaaccgaagcaaataggccaaaaatgaacaatgaacaatgctaccttcaactatacaaacgttaaccctatttctcacatcagggggagtattattatcactatatgtgtcagcttcattatcatacttactatattcggatatattgctaaaattctcaccaacagaaataactgcaccaacaatgccattggattgtgcaaacgcatcaaatgttcaggctgtgaaccgttctgcaacaaaaggggtgacacttcttctcccagaaccggagtggacatacccgcgtttatcttgcccgggctcaacctttcagaaagcactcctaattagccctcatagattcggagaaaccaaaggaaactcagctcccttgataataagggaaccttttattgcttgtggaccaaatgaatgcaaacactttgctctaacccattatgcagcccaaccagggggatactacaatggaacaagaggagacagaaacaagctgaggcatctaatttcagtcaaattgggcaaaatcccaacagtagaaaactccattttccacatggcagcatggagcgggtccgcgtgccatgatggtaaggaatggacatatatcggagttgatggccctgacaataatgcattgctcaaagtaaaatatggagaagcatatactgacacataccattcctatgcaaacaaaatcctaagaacacaagaaagtgcctgcaattgcatcgggggaaattgttatcttatgataactgatggctcagcttcaggtgttagtgaatgcagatttcttaagattcgagagggccgaataataaaagaaatatttccaacaggaagagtaaaacacactgaggaatgcacatgcggatttgccagcaataaaaccatagaatgtgcctgtagagataacagttacacagcaaaaagaccttttgtcaaattaaacgtggagactgatacagcagaaataagattgatgtgcacagatacttatttggacacccccagaccaaacgatggaagcataacaggcccttgtgaatctaatggggacaaagggagtggaggcatcaagggaggatttgttcatcaaagaatggaatccaagattggaaggtggtactctcgaacgatgtctaaaactgaaaggatggggatgggactgtatgtcaagtatgatggagacccatgggctgacagtgatgccctagcttttagtggagtaatggtttcaatgaaagaacctggttggtactcctttggcttcgaaataaaagataagaaatgcgatgtcccctgtattgggatagagatggtacatgatggtggaaaagagacttggcactcagcagcaacagccatttactgtttaatgggctcaggacagctgctgtgggacactgtcacaggtgttgacatggctctgtaatggaggaatggttgagtctgttctaaaccctttgttcctattttgtttgaacaattgtccttactgaacttaattgtttctgaaaaatgctcttgttactact

SEQ ID NO: 32 - DNA, 1699 bp, Artificial Sequence
  Bris08 long stalk NA (46aa insertion bold)
agcagaagcagagcatcttctcaaaaccgaagcaaataggccaaaaatgaacaatgaacaatgctaccttcaactatacaaacgttaaccctatttctcacatcagggggagtattattatcactatatgtgtcagcttcattatcatacttactatattcggatatattgctaaaattctcaccaacacaatatgaattcaactcccccccaaacaaccaagtgatgctgtgtgaaccaacaataatagaaagaaacataacagaaatagtgtatttaactaatcagacatatgttaacatcagcaacaccaactttgctgctgaaataactgcaccaacaatgccattggattgtgcaaacgcatcaaatgttcaggctgtgaaccgttctgcaacaaaaggggtgacacttcttctcccagaaccggagtggacatacccgcgtttatcttgcccgggctcaacctttcagaaagcactcctaattagccctcatagattcggagaaaccaaaggaaactcagctcccttgataataagggaaccttttattgcttgtggaccaaatgaatgcaaacactttgctctaacccattatgcagcccaaccagggggatactacaatggaacaagaggagacagaaacaagctgaggcatctaatttcagtcaaattgggcaaaatcccaacagtagaaaactccattttccacatggcagcatggagcgggtccgcgtgccatgatggtaaggaatggacatatatcggagttgatggccctgacaataatgcattgctcaaagtaaaatatggagaagcatatactgacacataccattcctatgcaaacaaaatcctaagaacacaagaaagtgcctgcaattgcatcgggggaaattgttatcttatgataactgatggctcagcttcaggtgttagtgaatgcagatttcttaagattcgagagggccgaataataaaagaaatatttccaacaggaagagtaaaacacactgaggaatgcacatgcggatttgccagcaataaaaccatagaatgtgcctgtagagataacagttacacagcaaaaagaccttttgtcaaattaaacgtggagactgatacagcagaaataagattgatgtgcacagatacttatttggacacccccagaccaaacgatggaagcataacaggcccttgtgaatctaatggggacaaagggagtggaggcatcaagggaggatttgttcatcaaagaatggaatccaagattggaaggtggtactctcgaacgatgtctaaaactgaaaggatggggatgggactgtatgtcaagtatgatggagacccatgggctgacagtgatgccctagcttttagtggagtaatggtttcaatgaaagaacctggttggtactcctttggcttcgaaataaaagataagaaatgcgatgtcccctgtattgggatagagatggtacatgatggtggaaaagagacttggcactcagcagcaacagccatttactgtttaatgggctcaggacagctgctgtgggacactgtcacaggtgttgacatggctctgtaatggaggaatggttgagtctgttctaaaccctttgttcctattttgtttgaacaattgtccttactgaacttaattgtttctgaaaaatgctcttgttactact

SEQ ID NO: 33 - DNA, 135 bp, Artificial Sequence
  Nucleotide sequence encoding 46 amino acid sequence extension
caatatgaattcaactcccccccaaacaaccaagtgatgctgtgtgaaccaacaataatagaaagaaacataacagaaatagtgtatttaactaatcagacatatgttaacatcagcaacaccaactttgctgct

SEQ ID NO: 34 - DNA, 90 bp, Artificial Sequence
  Nucleotide sequence encoding 30 amino acid sequence extension
aatcagacatatgttaacatcagcaacaccaactttgctgctggaaacacaacagagatagtgtatctgaccaacaccaccatagagaag

SEQ ID NO: 35 - protein, 43 aa, Artificial Sequence
  A/Puerto Rico/8/1934 N1
HSIQTGSQNHTGICNQNIITYKNSTWVKDTTSVILTGNSSLCP

SEQ ID NO: 36 - protein, 58 aa, Artificial Sequence
  A/California/04/2009 N1
HSIQLGNQSQIETCNQSVITYENNTWVNQTYVNISNTNFAAGQSVVSVKLAGNSSLCP

SEQ ID NO: 37 - protein, 43 aa, Artificial Sequence; identical to SEQ ID NO: 35
  PR8 N1-wt

SEQ ID NO: 38 - protein, 58 aa, Artificial Sequence
  PR8 N1-Ins15
HSIQTGSQNHTGICNQNIITYKNSTWVNQTYVNISNTNFAAGKDTTSVILTGNSSLCP

SEQ ID NO: 39 - protein, 73 aa, Artificial Sequence
  PR8 N1-Ins30
HSIQTGSQNHTGICNQNIITYKNSTWVNQTYVNISNTNFAAGNTTEIVYLTNTTIEKKDTTSVILTGNSSLCP

SEQ ID NO: 40 - protein, 17 aa, Artificial Sequence
  HK14 N2-Del25
TLHFKQIVYLTNTTIEK

SEQ ID NO: 41 - protein, 42 aa, Artificial Sequence
  HK14 N2-wt
TLHFKQYEFNSPPNNQVMLCEPTIIERNITEIVYLTNTTIEK

SEQ ID NO: 42 - protein, 57 aa, Artificial Sequence
  HK14 N2-Ins15
TLHFKQYEFNSPPNNQVMLCEPTIIERNITEIVYLTNQTYVNISNTNFAAGNTTIEK